(19) United States
(12) Patent Application Publication
(10) Pub. No.: US 2015/0174132 A1
(43) Pub. Date: Jun. 25, 2015

(54) METHOD FOR TREATING B CELL REGULATED AUTOIMMUNE DISORDERS

(71) Applicant: Synta Pharmaceuticals Corporation, Lexington, MA (US)

(72) Inventors: Kevin Foley, Waltham, MA (US); John Bertin, Winchester, MA (US); Ethan P. Grant, Morristown, NJ (US)

(21) Appl. No.: 14/539,714

(22) Filed: Nov. 12, 2014

Related U.S. Application Data
(63) Continuation of application No. 13/189,470, filed on Jul. 22, 2011, now abandoned.
Application No. 13/189,470 is a continuation of application No. 11/442,744, filed on May 26, 2006, now abandoned.
(60) Provisional application No. 60/685,077, filed on May 26, 2005.

(51) Int. Cl. A61K 31/5377; C12Q 1/68; G01N 33/53

(52) U.S. Cl. CPC A61K 31/5377; G01N 33/5308; C12Q 1/6883; G01N 2500/04; C12Q 2600/106

(57) ABSTRACT

The invention relates to a method for treating B-cell regulated autoimmune disorders using compounds that modulate the activity of c-Rel.

### RELATED APPLICATIONS

This application claims the benefit of U.S. Provisional Application No. 60/685,077, filed May 26, 2005, the entire teachings of which are incorporated herein by reference.

### BACKGROUND

Rel/NF-κB is a family of transcription factors that play a key role in inflammation, immunity, cell proliferation and apoptosis. Rel/NF-κB family members, including c-Rel, RelA (also referred to as “p65”), RelB, p50 and p52, exist mainly in the cytoplasm in an inactive form due to association with one or more members of a family of inhibitors known as IκB proteins (IκBα, IκBβ, IκBε, Bcl-3, p100, p105). The best characterized of the IkB proteins, IκBα, has a strong nuclear export sequence that keeps complexes of it and NF-κB proteins largely in the cytoplasm. Pro-inflammatory cytokines and other stimuli trigger phosphorylation of IκBα by IκB kinase (IKK) which marks it for subsequent ubiquitination and proteasomal degradation. Once liberated from association with IκB proteins, NF-κB proteins can accumulate in the nucleus and form homo- and heterodimers which activate the transcription of target genes, including those controlling cell proliferation and cell survival (anti-apoptotic genes). However, activation of NF-κB proteins is usually a transient process because one of the primary target genes of NF-κB is the gene encoding IκBα which can bind to NF-κB proteins and return them to their latent form in the cytoplasma.

c-Rel has been shown to play a role in the proliferation and survival of B-cells. The c-Rel protein is expressed at all stages of B-cell development, but is expressed at the highest levels in mature B-cells. c-Rel knockout mice develop normally and have no gross defects in hemopoiesis. However, they show immuno-deficiencies which primarily stem from defects in B-cells proliferation and survival in response to mitogenic activation, such as LPS, anti-IgM, antigens, and CD40. In addition, they show reduced antibody production in response to an antigen.

Since the primary role of c-Rel appears to be proliferation and survival of activated mature B-cell, agents that target c-Rel activity are useful for treating B-cell regulated autoimmune disorders. Therefore, a need exists for such agents.

### SUMMARY

The present invention addresses this need for agents that can be used to treat B-cell regulated autoimmune disorders. Without wishing to be bound by any theory, it is believed that the compounds of the invention selectively inhibit the activity of c-Rel without materially inhibiting the activity of other members of the Rel/NF-κB family.

In one aspect, the invention provides a method of treating or preventing a B-cell regulated autoimmune disorder in a subject in need thereof, comprising administering to the subject an effective amount of a compound of formula (I):

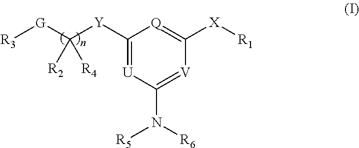

or a pharmaceutically acceptable salt, solvate, clathrate, hydrate, polymorph or prodrug thereof, wherein

R₁ is optionally substituted aryl, optionally substituted heteroaryl, or a group represented by the following formula:

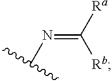

R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;

R₃ is R^{g};

R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;

X is O, S, S(O), S(O)₂, or NR^{k};

Y is (CH(R^{g}))ₘ, C(O), C(NR), O, S, S(O), S(O)₂, N(R^{k}), or absent;

G is a bond, —C(O)NR^{k}NR^{k}—, —NR^{k}NR^{k}C(O)—, —NR^{k}N═CR^{k}—, —CR^{k}═NNR^{k}—, —NR^{k}NR^{k}—, —N(OH)—, —NR^{k}O—, —ONR^{k}—, —C(O)—, —C(NR)—, —NR^{k}C(O)—, —C(O)NR^{k}—, —OC(O)—, —C(O)O—, —OC(O)O—, —NR^{k}C(O)O—, OC(O)NR^{k}—, —NR^{k}C(S)O—, —OC(S)NR^{k}—, —NR^{k}—C(NR)—NR^{k}—, —NR^{k}—C(O)—NR^{k}—, —NR^{k}—C(S)—NR^{k}—, —NR^{k}—S(O)₂—NR^{k}—, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k})—, —C(NR)—NR^{k}—, —NR^{k}—CR^{g}R^{g}—C(O)—, C(O)—ONR^{k}—, C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}—, —OC(NR)—NR^{k}NR^{k}, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c})NR^{k}—, —NR^{k}P(O)(R^{c})NR^{k}—, —P(O)(R^{c})NR^{k}—, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}—CHR^{g}—C(O)—, —NR^{k}—C(O)—CHR^{g}—, or —C(O)—NR^{k}—CHR^{g}—C(O)—; and

each of Q, U, and V are independently N or CR^{g}, wherein at least one of Q, U, or V is N; and each CR^{g} may be the same or different;

R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, —NR^{h}R^{j}, hydroxylalkyl, nitro, cyano, haloalkyl, aminoalkyl, or —S(O)₂R^{c};

each of R^{a} and R^{b}, independently, is H, optionally substituted alkyl, an optionally substituted cycloalkyl, an optionally substituted cyclyl, an optionally substituted heterocycloalkyl, an optionally substituted heterocyclyl, optionally substituted aryl, or optionally substituted heteroaryl;

R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;

R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;

R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;

R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;

n is 0, 1, 2, 3, 4, 5, 6 or 7; and

m is 0, 1, 2, 3, or 4.

In another aspect, the invention provides a method of treating or preventing a B-cell regulated autoimmune disorder in a subject in need thereof, comprising administering to the subject an effective amount of a compound of formula (II):

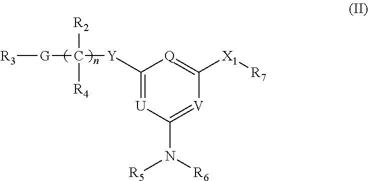

or a pharmaceutically acceptable salt, solvate, clathrate, hydrate or polymorph thereof, wherein G, Q, U, V, Y, R₂, R₃, R₄, R₅, R₆, and n are defined as for formula (I);

X₁ is represented by a formula selected from the group consisting of:

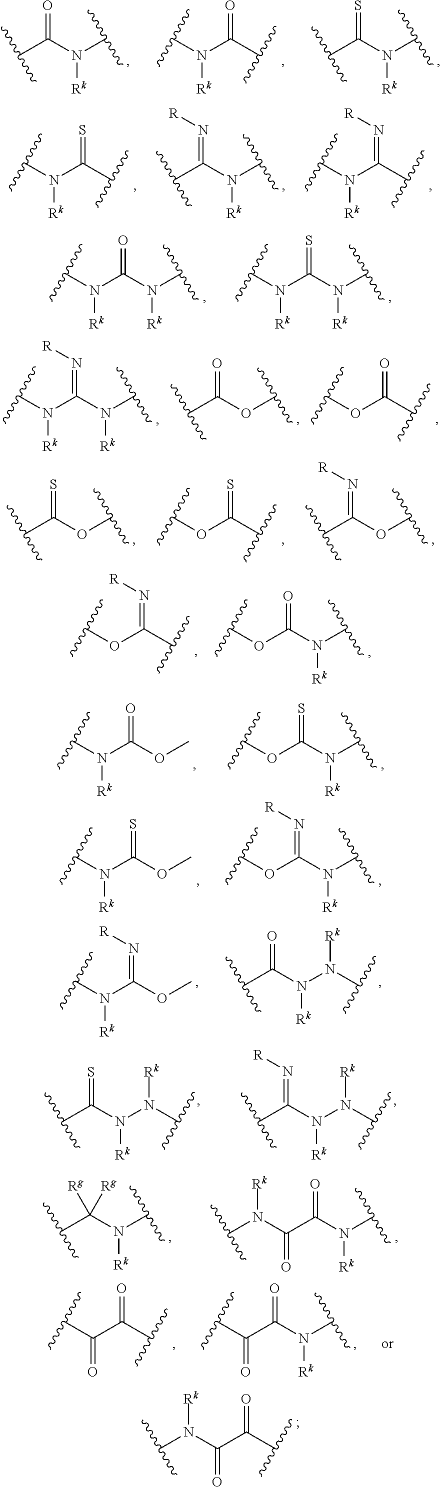

R and R^{k} are defined as for formula (I);

R₇ is an optionally substituted aryl or an optionally substituted heteroaryl.

In another aspect, the invention provides a method of treating or preventing B-cell regulated autoimmune disorder in a subject in need thereof, comprising administering to the subject an effective amount of a compound of formula (III):

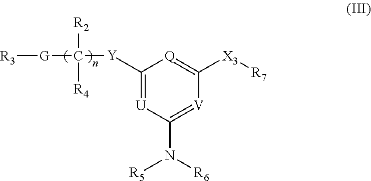

or a pharmaceutically acceptable salt, solvate, clathrate, hydrate, prodrug or polymorph thereof, wherein G, Q, U, V, Y, R₂, R₃, R₄, R₅, R₆, and n are defined as for formula (I);

R₇ is defined as for formula (II);

X₃ is —C(R^{g})═N-A-;

A is O, S, S(O), S(O)₂, C(CR^{g})₂, or NR^{k};

R^{g} and R^{k} are defined as for formula (I).

In another aspect, the invention provides a method of treating or preventing a B-cell regulated autoimmune disorder in a subject in need thereof, comprising administering to the subject an effective amount of a compound of formula (IV):

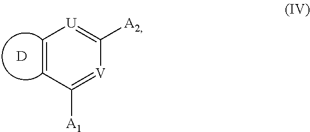

or a pharmaceutically acceptable salt, solvate, clathrate, hydrate, polymorph, or prodrugs thereof, wherein:

U and V are each, independently, N or CR^{g};

Ring D is a 5 to 9-membered aryl, 3 to 9-membered cycloalkyl, 3 to 9-membered cyclyl, 5 to 9-membered heteroaryl, 3 to 9-membered heterocycloalkyl, or a 3 to 9-membered heterocyclyl, each of which may be further substituted with one or more substituents;

one of A₁ and A₂ is —X₄—R′—L′—R″ and the other is a group represented by the following formula:

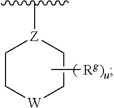

Z is N or CH;

W is O, S, S(O), S(O)₂, NR^{m}, or NC(O)R^{m}, wherein R^{m}, for each occurrence, is independently —H, alkyl, aryl, heteroaryl, cycloalkyl, heterocycloalkyl, or alkylcarbonyl;

u is 0, 1, 2, 3, or 4;

X₄ is O, S, S(O), S(O)₂, N(R^{k}), C(O), C(S), C(S)NR^{k}, C(NR), C(NR)NR^{k}, C(O)NR^{k}, C(O)NR^{k}NR^{k}, C(O)ONR^{k}, C(O)NR^{k}O, C(O)O, OC(O), OC(O)O, (C(R^{g})(R^{g}))_{q}, (C(R^{g})(R^{g}))_{q}NR^{k}, (C(R^{g})(R^{g}))_{q}O, (C(R^{g})(R^{g}))_{q}S(O)ₚ, (C(R^{g})(R^{g}))_{q}N═C(R^{g}), C(R^{g})═N, C(R^{g})═N—O, C(R^{g})═N—S(O)ₚ, C(R^{g})═N—NR^{k}, C(R^{g})═N—C(CR^{g})₂, (C(R^{g})(R^{g}))_{q}C(R^{g})═N, (C(R^{g})(R^{g}))_{q}N═N, (C(R^{g})(R^{g}))_{q}C(R^{g})═C(R^{g}), C(R^{g})═C(R^{g}), N═C(R^{g}), N(R^{k})N═C(R^{g}), N(R^{k})C(R^{g})═N, N(R^{k})C(R^{g})═C(R^{g}), N═N, N(R^{k})N═N, NR^{k}C(O)NR^{k}, NR^{k}C(S)NR^{k}, NR^{k}C(O), NR^{k}C(O)O, NR^{k}C(NR)NR^{k}, NR^{k}C(S)O, NR^{k}S(O)ₚNR^{k}, OC(O)NR^{k}, OC(S)NR^{k}, OC(NR)NR^{k}, OS(O)ₚNR^{k}, C(NR)O, S(O)ₚNR^{k}, or S(O)ₚNR^{k}NR^{k};

R′ is an optionally substituted cycloalkyl, an optionally substituted cyclyl, an optionally substituted heterocycloalkyl, an optionally substituted aryl, an optionally substituted heteroaryl, an optionally substituted aralkyl, an optionally substituted heteroaralkyl, or absent;

L′ is O, S, S(O), S(O)₂, N(R^{k}), C(O), C(S), C(S)NR^{k}, C(NR), C(NR)NR^{k}, C(O)NR^{k}, C(O)NR^{k}NR^{k}, C(O)ONR^{k}, C(O)NR^{k}O, C(O)O, OC(O), OC(O)O, (C(R^{g})(R^{g}))_{q}, (C(R^{g})(R^{g}))_{q}NR^{k}, (C(R^{g})(R^{g}))_{q}O, (C(R^{g})(R^{g}))_{q}S(O)ₚ, (C(R^{g})(R^{g}))_{q}N═C(R^{g}), C(R^{g})═N, C(R^{g})═N—O, C(R^{g})═N—S(O)ₚ, C(R^{g})═N—NR^{k}, C(R^{g})═N—C(CR^{g})₂, (C(R^{g})(R^{g}))_{q}C(R^{g})═N, (C(R^{g})(R^{g}))_{q}N═N, (C(R^{g})(R^{g}))_{q}C(R^{g})═C(R^{g}), C(R^{g})═C(R^{g}), N═C(R^{g}), N(R^{k})N═C(R^{g}), N(R^{k})C(R^{g})═N, N(R^{k})C(R^{g})═C(R^{g}), N═N, N(R^{k})N═N, NR^{k}C(O)NR^{k}, NR^{k}C(S)NR^{k}, NR^{k}C(O), NR^{k}C(O)O, NR^{k}C(NR)NR^{k}, NR^{k}C(S)O, NR^{k}S(O)ₚNR^{k}, OC(O)NR^{k}, OC(S)NR^{k}, OC(NR)NR^{k}, OS(O)ₚNR^{k}, C(NR)O, S(O)ₚNR^{k}, S(O)ₚNR^{k}NR^{k} or absent; and

R″ is H, an optionally substituted alkyl, an optionally substituted cycloalkyl, an optionally substituted cyclyl, an optionally substituted heterocycloalkyl, an optionally substituted heterocyclyl, an optionally substituted aryl, an optionally substituted heteroaryl, an optionally substituted aralkyl, an optionally substituted heteroaralkyl, N(R^{k})(CH₂)_{q}R^{g}, —OR^{k}, —SR^{k}, —NR^{h}R^{j}, hydroxylalkyl, —C(O)R^{c}, —C(S)R^{c}, —C(NR)R^{c}, halo, haloalkyl, aminoalkyl, mercaptoalkyl, cyano, nitro, —S(O)R^{c}, —S(O)₂R^{c}, —P(O)R^{c}R^{c}, —P(S)R^{c}R^{c}, or an optionally substituted alkylcarbonylalkyl;

q, for each occurrence, is independently 1, 2, 3, 4, 5, 6, 7, or 8;

p, for each occurrence, is independently 0, 1, or 2; and

R, R^{c}, R^{g}, R^{h}, R^{j}, and R^{k} are defined as for formula (I).

In another aspect, the invention provides a method of treating or preventing a B-cell regulated autoimmune disorder in a subject in need thereof, comprising administering to the subject an effective amount of a compound of formula (X):

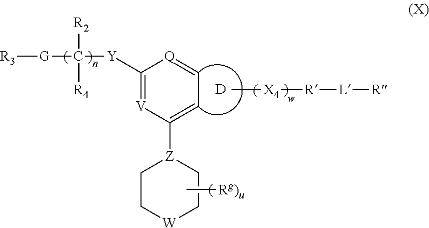

or a pharmaceutically acceptable salt, solvate, clathrate, hydrate, polymorph, prodrug thereof,
wherein:

G, Y, R₂, R₃, R₄, and n are defined as for formula (I);

L′, U, V, W, X4, Z, R′, R″, u, and Ring D are defined as for formula (IV); and

w is 0 or 1.

In another aspect, the invention provides a method of treating or preventing a B-cell regulated autoimmune disorder in a subject in need thereof, comprising administering to the subject an effective amount of a compound of formula (XIV):

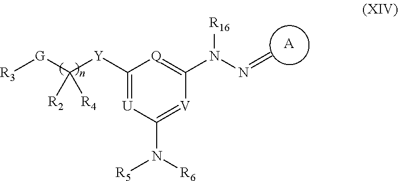

or a pharmaceutically acceptable salt, solvate, clathrate, hydrate, polymorph, or prodrug thereof, wherein:

G, Q, U, V, Y, R₂, R₃, R₄, R₅, R₆ and n are defined as for formula (I):

ring A is an optionally substituted cycloalkyl, an optionally substituted cyclyl, an optionally substituted heterocycloalkyl, or an optionally substituted heterocyclyl, wherein the cycloalkyl, cyclyl, heterocycloalkyl, and heterocyclyl are optionally fused to an optionally substituted cycloalkyl, an optionally substituted cyclyl, an optionally substituted heterocycloalkyl, an optionally substituted heterocyclyl, an optionally substituted aryl, or an optionally substituted heteroaryl; and

R₁₆, for each occurrence, is independently, H or a lower alkyl.

In one embodiment, the B-cell regulated autoimmune disorders include lystemic lupus erythematosis (SLE), Sjogren's syndrome, graft-versus-host disease, systemic sclerosis, myasthenia gravis, juvenile rheumatoid arthritis, osteoarthritis, psoriatic arthritis, dermatitis, atopic dermatitis, chronic autoimmune urticaria, polymyositis/dermatomyositis, toxic epidermal necrolysis, systemic scleroderma and sclerosis, respiratory distress syndrome, adult respiratory distress syndrome (ARDS), meningitis, allergic rhinitis, encephalitis, uveitis, colitis, glomerulonephritis, allergic conditions, eczema, asthma, atherosclerosis, autoimmune myocarditis, leukocyte adhesion deficiency, lupus (nephritis, non-renal, discoid, alopecia), allergic encephalomyelitis, tuberculosis, sarcoidosis, granulomatosis, Wegener's granulomatosis, agranulocytosis, vasculitis, aplastic anemia, Coombs positive anemia, Diamond Blackfan anemia, immune hemolytic anemia, hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, multiple organ injury syndrome, myasthenia gravis, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection, graft versus host disease (GVHD), pemphigoid bullous, pemphigus, vulgaris, foliaceus, autoimmune polyendocrinopathies, Reiter's disease, stiff-man syndrome, giant cell arteritis, immune complex nephritis, IgA nephropathy, IgM polyneuropathies, IgM mediated neuropathy, idiopathic thrombocytopenic purpura (ITP), thrombotic thrombocytopenic purpura (TTP), autoimmune thrombocytopenia, autoimmune orchitis, autoimmune oophoritis, primary hypothyroidism; autoimmune endocrine diseases, autoimmune thyroiditis, chronic thyroiditis (Hashimoto's Thyroiditis), subacute thyroiditis, idiopathic hypothyroidism, Addison's disease, Grave's disease, polyglandular endocrinopathy syndromes, Sheehan's syndrome, autoimmune hepatitis, Lymphoid interstitial pneumonitis (HIV), non-transplant bronchiolitis obliterans, Guillain-Barre' Syndrome, Large Vessel Vasculitis, Polymyalgia Rheumatica, Giant Cell (Takayasu's) Arteritis, Medium Vessel Vasculitis, Kawasaki's Disease, Polyarteritis Nodosa, ankylosing spondylitis, Berger's Disease, Rapidly Progressive Glomerulonephritis, Primary biliary cirrhosis, Celiac sprue, Cryoglobulinemia, ALS, and coronary artery disease.

In another embodiment, the B-Cell regulated autoimmune disorder is selected from the group consisting of systemic sclerosis, toxic epidermal necrolysis, encephalitis, glomerulonephritis, leukocyte adhesion deficiency, tuberculosis, agranulocytosis, Factor VIII deficiency, hemophilia A, pancytopenia, leukopenia, diseases involving leukocyte diapedesis, multiple organ injury syndrome, anti-glomerular basement membrane disease, allergic neuritis, Castleman's syndrome, Goodpasture's Syndrome, Lambert-Eaton Myasthenic Syndrome, Reynaud's syndrome, pemphigoid bullous, foliaceus, Reiter's disease, stiff-man syndrome, primary hypothyroidism, Sheehan's syndrome, non-transplant bronchiolitis obliterans, Polymyalgia Rheumatica, Kawasaki's Disease, Polyarteritis Nodosa, Berger's Disease, Rapidly Progressive Glomerulonephritis, Celiac sprue, Cryoglobulinemia, ALS, and coronary artery disease.

Other features, objects, and advantages of the invention will be apparent from the description and from the claims.

### BRIEF DESCRIPTION OF THE DRAWINGS

FIG. 1 is a western blot analysis of THP-1 nuclear extracts in stimulated and non-stimulated cells with regard to the presence of NFκB family members c-Rel, p65 or p50; α-tubulin is an internal control.

FIG. 2 is an immunofluorescent study indicating that compound 50 can block the accumulation of c-Rel in the nucleus of cells induced by LPS.

FIG. 3 is an immunofluorescent study indicating that compound 50 does not block the accumulation of p65 in the nucleus of cells induced by LPS.

FIG. 4 is an immunoblot that shows the effect of a test molecule on NF-kB p50 nuclear translocation.

FIG. 5 graphically presents the results of a densitometry showing the effect of a test molecule on p50 nuclear translocation.

FIG. 6 depicts an immunoblot demonstrating the effect of a test molecule on NF-kB p65 nuclear translocation.

FIG. 7 graphically presents the results of a densitometry showing the effect of a test molecule on p65 nuclear translocation.

FIG. 8 depicts an immunoblot demonstrating the effect of a test molecule on nuclear translocation of NF-kB members, including c rel.

FIG. 9 is an immunoblot showing the amount of phosphorylation of IKKβ in cells treated with Compound 50 and untreated cells 0 min., 5 min., 15 min., and 60 min. after stimulation with IFNγ/LPS.

FIG. 10 is an immunoblot showing the amount of phosphorylation of p65 in cells treated with Compound 50 and untreated cells 0 min., 30 min., 1 hour, and 6 hours after stimulation with IFNγ/LPS.

FIG. 11 is an immunoblot showing the amount of phosphorylation of p50 in cells treated with Compound 50 and untreated cells 0 min., 30 min., 1 hour, and 6 hours after stimulation with IFNγ/LPS.

FIG. 12 is an immunoblot showing that Compound 50 reduces the accumulation of c-Rel in the nucleus of Jurkat T cells after stimulation with PMA+ionomycin but does not significantly reduce the nuclear accumulation of p65 or p50.

FIG. 13 is a graph showing the DNA binding activity of c-Rel after stimulation with LPS/IFNγ in treated and untreated cells.

FIG. 14 is an immunoblot showing the levels of c-Rel in nuclear extracts and cytosolic extracts in treated and untreated cells after stimulation with LPS/INFγ.

FIG. 15 is a graph showing the densitometry measurement of the immunoblot in FIG. 14.

FIG. 16 is a graph showing the effects of compound 50 on the survival of B cells in the presence of BAFF.

FIG. 17 is a graph showing the effects of compound 50 on the survival of B cells in the presence of anti-CD40.

FIG. 18 is a graph showing the effects of compound 50 on the survival of B cells in the presence of LPS.

### DETAILED DESCRIPTION

In one aspect, the invention provides a method of treating or preventing a B-cell regulated autoimmune disorder in a subject in need thereof, comprising administering to the subject an effective amount of a compound of formula (I):

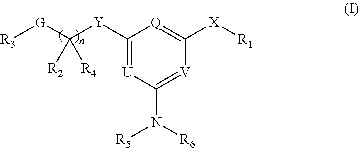

or a pharmaceutically acceptable salt, solvate, clathrate, hydrate, polymorph or prodrug thereof, wherein:

R₁ is optionally substituted aryl, optionally substituted heteroaryl, or a group represented by the following formula:

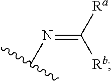

R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;

R₃ is R^{g};

R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;

X is O, S, S(O), S(O)₂, or NR^{k};

Y is (CH(R^{g}))ₘ, C(O), C(NR), O, S, S(O), S(O)₂, N(R^{k}), or absent;

G is a bond, —C(O)NR^{k}NR^{k}—, —NR^{k}NR^{k}C(O)—, —NR^{k}N═CR^{k}—, —CR^{k}═NNR^{k}—, —NR^{k}NR^{k}—, —N(OH)—, —NR^{k}O—, —ONR^{k}—, —C(O)—, —C(NR)—, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}—C(NR)—NR^{k}—, —NR^{k}—C(O)—NR^{k}—, —NR^{k}—C(S)—NR^{k}—, —NR^{k}—S(O)₂—NR^{k}—, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k})—, —C(NR)—NR^{k}—, —NR^{k}—CR^{g}R^{g}—C(O)—, —C(O)—ONR^{k}—, —C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}—, —OC(NR)—NR^{k}NR^{k}—, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c})NR^{k}—NR^{k}P(O)(R^{c})NR^{k}—, —P(O)(R^{c})NR^{k}—, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}—CHR^{g}—C(O)—, —NR^{k}—C(O)—CHR^{g}—, or —C(O)—NR^{k}—CHR^{g}—C(O)—; and

each of Q, U, and V are independently N or CR^{g}, wherein at least one of Q, U, or V is N; and each CR^{g} may be the same or different;

R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, —NR^{h}R^{j}, hydroxylalkyl, nitro, cyano, haloalkyl, aminoalkyl, or —S(O)₂R^{c};

each of R^{a} and R^{b}, independently, is H, optionally substituted alkyl, an optionally substituted cycloalkyl, an optionally substituted cyclyl, an optionally substituted heterocycloalkyl, an optionally substituted heterocyclyl, optionally substituted aryl, or optionally substituted heteroaryl;

R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;

R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;

R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;

R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;

n is 0, 1, 2, 3, 4, 5, 6 or 7; and

m is 0, 1, 2, 3, or 4.

In another aspect, the invention provides a method of treating or preventing a B-cell regulated autoimmune disorder in a subject in need thereof, comprising administering to the subject an effective amount of a compound of formula (II):

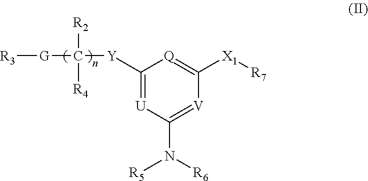

or a pharmaceutically acceptable salt, solvate, clathrate, hydrate, polymorph, or prodrug, thereof, wherein G, Q, U, V, Y, R₂, R₃, R₄, R₅, R₆, and n are defined as for formula (I);

X₁ is represented by a formula selected from the group consisting of:

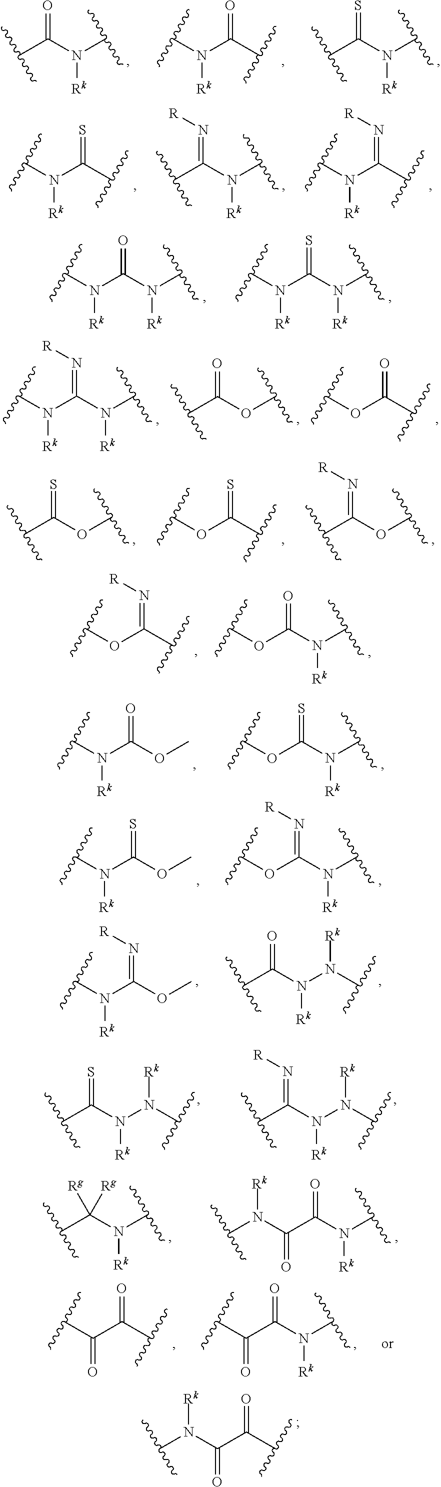

R and R^{k} are defined as for formula (I);

R₇ is an optionally substituted aryl or an optionally substituted heteroaryl.

In another aspect, the invention provides a method of treating or preventing a B-cell regulated autoimmune disorder in a subject in need thereof, comprising administering to the subject an effective amount of a compound of formula (III):

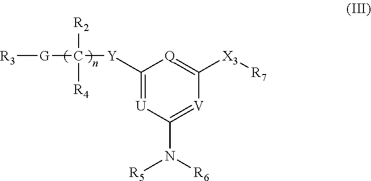

or a pharmaceutically acceptable salt, solvate, clathrate, hydrate, prodrug or polymorph thereof, wherein G, Q, U, V, Y, R₂, R₃, R₄, R₅, R₆, and n are defined as for formula (I);

R₇ is defined as for formula (II);

X₃ is —C(R^{g})═N-A-;

A is O, S, S(O), S(O)₂, C(CR^{g})₂, or NR^{k};

R^{g} and R^{k} are defined as for formula (I).

In another aspect, the invention provides a method of treating or preventing a B-cell regulated autoimmune disorder in a subject in need thereof, comprising administering to the subject an effective amount of a compound of formula (IV):

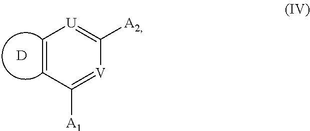

or a pharmaceutically acceptable salt, solvate, clathrate, hydrate, polymorph, or prodrugs thereof, wherein:

U and V are each, independently, N or CR^{g};

Ring D is a 5 to 9-membered aryl, 3 to 9-membered cycloalkyl, 3 to 9-membered cyclyl, 5 to 9-membered heteroaryl, 3 to 9-membered heterocycloalkyl, or a 3 to 9-membered heterocyclyl, each of which may be further substituted with one or more substituents;

one of A₁ and A₂ is —X₄—R′—L′—R″ and the other is a group represented by the following formula:

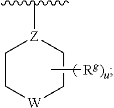

Z is N or CH;

W is O, S, S(O), S(O)₂, NR^{m}, or NC(O)R^{m}, wherein R^{m}, for each occurrence, is independently —H, alkyl, aryl, heteroaryl, cycloalkyl, heterocycloalkyl, or alkylcarbonyl;

u is 0, 1, 2, 3, or 4;

X₄ is O, S, S(O), S(O)₂, N(R^{k}), C(O), C(S), C(S)NR^{k}, C(NR), C(NR)NR^{k}, C(O)NR^{k}, C(O)NR^{k}NR^{k}, C(O)ONR^{k}, C(O)NR^{k}O, C(O)O, OC(O), OC(O)O, (C(R^{g})(R^{g}))_{q}, (C(R^{g})(R_{g}))_{q}NR^{k}, (C(R^{g})(R^{g}))_{q}O, (C(R^{g})(R^{g}))_{q}S(O)ₚ, (C(R^{g})(R^{g}))_{q}N═C(R^{g}), C(R^{g})═N, C(R^{g})═N—O, C(R^{g})═N—S(O)ₚ, C(R^{g})═N—NR^{k}, C(R^{g})═N—C(CR^{g})₂, (C(R^{g})(R^{g}))_{q}C(R^{g})═N, (C(R^{g})(R^{g}))_{q}N═N, (C(R^{g})(R^{g}))_{q}C(R^{g})═C(R^{g}), C(R^{g})═C(R^{g}), N═C(R^{g}), N(R^{k})N═C(R^{g}), N(R^{k})C(R^{g})═N, N(R^{k})C(R^{g})═C(R^{g}), N═N, N(R^{k})N═N, NR^{k}C(O)NR^{k}, NR^{k}C(S)NR^{k}, NR^{k}C(O), NR^{k}C(O)O, NR^{k}C(NR)NR^{k}, NR^{k}C(S)O, NR^{k}S(O)ₚNR^{k}, OC(O)NR^{k}, OC(S)NR^{k}, OC(NR)NR^{k}, OS(O)ₚNR^{k}, C(NR)O, S(O)ₚNR^{k}, or S(O)ₚNR^{k}NR^{k};

R′ is an optionally substituted cycloalkyl, an optionally substituted cyclyl, an optionally substituted heterocycloalkyl, an optionally substituted aryl, an optionally substituted heteroaryl, an optionally substituted aralkyl, an optionally substituted heteroaralkyl, or absent;

L′ is O, S, S(O), S(O)₂, N(R^{k}), C(O), C(S), C(S)NR^{k}, C(NR), C(NR)NR^{k}, C(O)NR^{k} C(O)NR^{k}NR^{k}, C(O)ONR^{k}, C(O)NR^{k}O, C(O)O, OC(O), OC(O)O, (C(R^{g})(R^{g}))_{q}, (C(R^{g})(R_{g}))_{q}NR^{k}, (C(R^{g})(R^{g}))_{q}O, (C(R^{g})(R^{g}))_{q}S(O)ₚ, (C(R^{g})(R^{g}))_{q}N═C(R^{g}), C(R^{g})═N, C(R^{g})═N—O, C(R^{g})═N—S(O)ₚ, C(R^{g})═N—NR^{k}, C(R^{g})═N—C(CR^{g})₂, (C(R^{g})(R^{g}))_{q}C(R^{g})═N, (C(R^{g})(R^{g}))_{q}N═N, (C(R^{g})(R^{g}))_{q}C(R^{g})═C(R^{g}), C(R^{g})═C(R^{g}), N═C(R^{g}), N(R^{k})N═C(R^{g}), N(R^{k})C(R^{g})═N, N(R^{k})C(R^{g}), N═N, N(R^{k})N═N, NR^{k}C(O)NR^{k}, NR^{k}C(S)NR^{k}, NR^{k}C(O), NR^{k}C(O), NR^{k}C(NR)NR^{k}, NR^{k}C(S)O, NR^{k}S(O)ₚNR^{k}, OC(O)NR^{k}, OC(S)NR^{k}, OC(NR)NR^{k}, OS(O)ₚNR^{k}, C(NR)O, S(O)ₚNR^{k}, S(O)ₚNR^{k}NR^{k} or absent; and

R″ is H, an optionally substituted alkyl, an optionally substituted cycloalkyl, an optionally substituted cyclyl, an optionally substituted heterocycloalkyl, an optionally substituted heterocyclyl, an optionally substituted aryl, an optionally substituted heteroaryl, an optionally substituted aralkyl, an optionally substituted heteroaralkyl, N(R^{k})(CH₂)_{q}R^{g}, —OR^{k}, —SR^{k}, —NR^{h}R^{j}, hydroxylalkyl, —C(O)R^{c}, —C(S)R^{c}, —C(NR)R^{c}, halo, haloalkyl, aminoalkyl, mercaptoalkyl, cyano, nitro, —S(O)R^{c}, —S(O)₂R^{c}, —P(O)R^{c}R^{c}, —P(S)R^{c}R^{c}, or an optionally substituted alkylcarbonylalkyl;

q, for each occurrence, is independently 1, 2, 3, 4, 5, 6, 7, or 8;

p, for each occurrence, is independently 0, 1, or 2; and

R, R^{c}, R^{g}, R^{h}, R^{j}, and R^{k} are defined as for formula (I).

In another aspect, the invention provides a method of treating or preventing a B-cell regulated autoimmune disorder in a subject in need thereof, comprising administering to the subject an effective amount of a compound of formula (X):

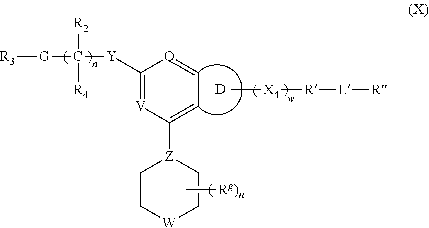

or a pharmaceutically acceptable salt, solvate, clathrate, hydrate, polymorph, prodrug thereof, wherein:

G, Y, R₂, R₃, R₄, and n are defined as for formula (I);

L′, U, V, W, X4, Z, R′, R″, u, and Ring D are defined as for formula (IV); and

w is 0 or 1.

In another aspect, the invention provides a method of treating or preventing a B-cell regulated autoimmune disorder in a subject in need thereof, comprising administering to the subject an effective amount of a compound of formula (XIV):

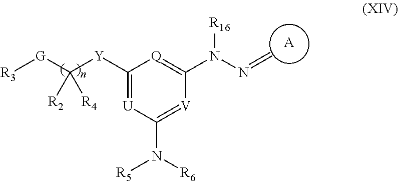

or a pharmaceutically acceptable salt, solvate, clathrate, hydrate, polymorph, or prodrug thereof, wherein:

G, Q, U, V, Y, R₂, R₃, R₄, R₅, R₆ and n are defined as for formula (I):

ring A is an optionally substituted cycloalkyl, an optionally substituted cyclyl, an optionally substituted heterocycloalkyl, or an optionally substituted heterocyclyl, wherein the cycloalkyl, cyclyl, heterocycloalkyl, and heterocyclyl are optionally fused to an optionally substituted cycloalkyl, an optionally substituted cyclyl, an optionally substituted heterocycloalkyl, an optionally substituted heterocyclyl, an optionally substituted aryl, or an optionally substituted heteroaryl; and

R₁₆, for each occurrence, is independently, H or a lower alkyl.

In some embodiments, in the compounds represented by formula (I), (II), (III), or (XIV), Q, U, and V are N.

In some embodiments, in the compounds represented by formula (I), (II), (III), or (XIV), one of Q, U, or V is CR^{g}, and the other two are N.

In some embodiments, in the compounds represented by formula (I), (II), (III), or (XIV), V is CR^{g}, Q and U are N.

In some embodiments, in the compounds represented by formula (I), (II), (III), or (XIV), Q is CR^{g}, V and U are N.

In some embodiments, in the compounds represented by formula (I), (II), (III), or (XIV), U is CR^{g}, V and Q are N.

In some embodiments, in the compounds represented by formula (I), (II), (III), or (XIV), one of Q, U, or V is N, and the other two are CR^{g}.

In some embodiments, in the compounds represented by formula (I), (II), (III), or (XIV), V is N, and Q and U are CR^{g}.

In some embodiments, in the compounds represented by formula (I), (II), (III), or (XIV), Q is N, and V and U are CR^{g}.

In some embodiments, in the compounds represented by formula (I), (II), (III), or (XIV), U is N and Q, and V are CR^{g}.

In some embodiments, in the compounds represented by formula (I), (II), (III), or (XIV), —NR₅R₆ is an optionally substituted morpholino, an optionally substituted thiomorpholino, an optionally substituted 1-oxo-thiomorpholino, an optionally substituted 1,1-dioxo-thiomorpholino, an optionally substituted piperidinyl, or an optionally substituted piperazinyl.

In some embodiments, in the compounds represented by formula (I), X is —NR^{k}—. In a preferred embodiment, the R^{k} of group X is —H or a lower alkyl.

In some embodiments, R₁ in the compounds represented by formula (I) or R₇ in the compounds represented by formula (II) or (III), is an optionally substituted aryl or an optionally substituted heteroaryl.

In some embodiments, R₁ in the compounds represented by formula (I) or R₇ in the compounds represented by formula (II) or (III), is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted carbazolyl, an optionally substituted 1,2,3,4-tetrahydro-carbazolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments, R₁ in the compounds represented by formula (I) or R₇ in the compounds represented by formula (II) or (III), is an optionally substituted phenyl, an optionally substituted indolyl, an optionally substituted indanyl, an optionally substituted carbazolyl, or an optionally substituted 1,2,3,4-tetrahydro-carbazolyl.

In some embodiments, R₁ in the compounds represented by formula (I) or R₇ in the compounds represented by formula (II) or (III) is a group represented by the following formula:

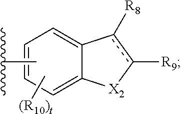

wherein:

the dashed line indicates a double or a single bond;

X₂ is —O—, —S(O)ₚ—, —N(R^{k})—, or —C(R^{g})(R^{g})—;

R₈ and R₉ are each, independently, R^{g}, —C(O)R^{c}, —C(S)R^{c}, —C(NR)R^{c}, —NR^{k}C(O)R^{c}, —OC(O)R^{c}, —SC(O)R^{c}, —NR^{k}C(S)R^{c}, —OC(S)R^{c}, —SC(S)R^{c}, —NR^{k}C(NR)R^{c}, —OC(NR)R^{c}, or —SC(NR)R^{c}; or R₈ and R₉, taken together with the carbons to which they are attached, form a 5- to 7-membered optionally substituted cycloalkyl, a 5- to 7-membered optionally substituted cyclyl, a 5- to 7-membered optionally substituted aryl, a 5- to 7-membered optionally substituted heterocycloalkyl, a 5- to 7-membered optionally substituted heterocyclyl, a 5- to 7-membered optionally substituted heteroaryl;

R₁₀, for each occurrence, is, independently, R^{g}, —C(O)R^{c}, —C(S)R^{c}, —C(NR)R^{c}, —NR^{k}C(O)R^{c}, —OC(O)R^{c}, —SC(O)R^{c}, —NR^{k}C(S)R^{c}, —OC(S)R^{c}, —SC(S)R^{c}, —NR^{k}C(NR)R^{c}, —OC(NR)R^{c}, or —SC(NR)R^{c};

p is 0, 1, or 2; and

t is 0, 1, 2, or, 3.

In some embodiments, R₁ in the compounds represented by formula (I) or R₇ in the compounds represented by formula (II) or (III) is (2,3-dimethyl-1H-indol-5-yl), (1H-indol-5-yl), or (6,7,8,9-tetrahydro-5H-carbazol-3-yl).

In some embodiments, in the compounds represented by formula (II) or (III), R₇ is a group represented by the following formula:

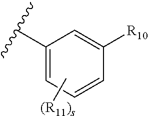

wherein:

R₁₁ and R₁₂, for each occurrence, are, independently, R^{g}, —C(O)R^{c}, —C(S)R^{c}, —C(NR)R^{c}, —NR^{k}C(O)R^{c}, —OC(O)R^{c}, —SC(O)R^{c}, —NR^{k}C(S)R^{c}, —OC(S)R^{c}, —SC(S)R^{c}, —NR^{k}C(NR)R^{c}, —OC(NR)R^{c}, or —SC(NR)R^{c}; and

s is 0, 1, 2, 3, or 4.

In some embodiments, in the compounds represented by formula (I), R₁ is a group represented by the following formula:

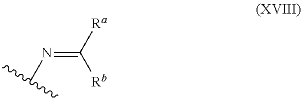

In some embodiments, when R₁ of formula (I) is group (XVIII), one of R^{a} or R^{b} is —H or a lower alkyl, and the other is an optionally substituted aryl or an optionally substituted heteroaryl.

In some embodiments, when R₁ of formula (I) is group (XVIII), one of R^{a} or R^{b} is —H or a lower alkyl, and the other is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted carbazolyl, an optionally substituted 1,2,3,4-tetrahydro-carbazolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments, when R₁ of formula (I) is group (XVIII), one of R^{a} or R^{b} is —H or a lower alkyl, and the other is an optionally substituted phenyl, an optionally substituted indolyl, an optionally substituted indanyl, an optionally substituted carbazolyl, or an optionally substituted 1,2,3,4-tetrahydro-carbazolyl.

In some embodiments, in the compounds represented by formula (I), (II), (III), (X) or (XIV), Y is O.

In some embodiments, in the compounds represented by formula (I), (II), (III), (X) or (XIV), Y is a covalent bond.

In some embodiments, in the compounds represented by formula (I), (II), (III), (X) or (XIV), R₃ is H.

In some embodiments, in the compounds represented by formula (I), (II), (III), (X) or (XIV), R₃ is an optionally substituted aryl or an optionally substituted heteroaryl.

In some embodiments, in the compounds represented by formula (I), (II), (III), (X) or (XIV), R₃ is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments, in the compounds represented by formula (I), (II), (III), (X) or (XIV), R₃ is an optionally substituted piperidinyl, an optionally substituted piperazinyl, an optionally substituted 2-oxopiperazinyl, an optionally substituted 2-oxopiperidinyl, an optionally substituted 2-oxopyrrolidinyl, an optionally substituted 4-piperidonyl, an optionally substituted tetrahydropyranyl, an optionally substituted oxazolidinyl, an optionally substituted 2-oxo-oxazolidinyl, an optionally substituted tetrahydrothiopyranyl, an optionally substituted tetrahydrothiopyranyl sulfone, an optionally substituted morpholinyl, an optionally substituted thiomorpholinyl, an optionally substituted thiomorpholinyl sulfoxide, an optionally substituted thiomorpholinyl sulfone, an optionally substituted 1,3-dioxolanyl, an optionally substituted [1,4]dioxanyl, an optionally substituted 2-oxo-imidazolidinyl, tetrahydrofuranyl, or an optionally substituted tetrahydrothienyl.

In some embodiments, in the compounds represented by formula (I), (II), (III), (X) or (XIV), R₃ is a hydroxy, an optionally substituted heterocycloalkyl, an optionally substituted heterocyclyl, or an optionally substituted heteroaryl.

In some embodiments, in the compounds represented by formula (I), (II), (III), (X) or (XIV), R₃ is a hydroxy, an optionally substituted pyridinyl, an optionally substituted morpholino, or an optionally substituted oxazolidin-2-one.

In some embodiments, in the compounds represented by formula (I), (II), (III), (X) or (XIV), R₃ is —OR^{k} or —NR^{h}R^{j}, and R^{f}, R^{h} and R^{j} are each, independently, H, an optionally substituted alkyl, an optionally substituted aryl, an optionally substituted heteroaryl, an optionally substituted cycloalkyl, an optionally substituted heterocycloalkyl, or —C(O)R^{c}.

In some embodiments, in the compounds represented by formula (I), (II), (III), (X) or (XIV), R₃ is —C(O)OR^{k}, —OC(O)R^{k}, —C(O)NR^{h}R^{j}, —NR^{k}C(O)R^{k}, —C(S)OR^{k}, —OC(S)R^{k}, —NR^{k}C(O)NR^{h}R^{j}, —NR^{k}C(S)NR^{h}R^{j}, —C(O)NRhRj, —S(O)₂R^{k}, —S(O)₂NR^{h}R^{j}, —OC(O)NR^{h}R^{j}, or —NR^{k}C(O)OR^{k}.

In some embodiments, in the compounds represented by formula (I), (II), (III), (X) or (XIV), each of R₂ and R₄ is, independently, H, an optionally substituted alkyl, an optionally substituted alkylcarbonyl, an optionally substituted aryl, an optionally substituted heteroaryl, an optionally substituted cycloalkyl, an optionally substituted cyclyl, an optionally substituted heterocycloalkyl, or an optionally substituted heterocyclyl.

In some embodiments, in the compounds represented by formula (I), (II), (III), (X) or (XIV), n is 1, 2, or 3, and R₂ and R₄, for each occurrence are, independently, H or a lower alkyl.

In some embodiments, in the compounds represented by formula (I), (II), (III), (X) or (XIV), G is absent.

In some embodiments, in the compounds represented by formula (I), (II), (III), (X) or (XIV), G is an optionally substituted heteroaryl or an optionally substituted heterocyclyl.

In some embodiments, in the compounds represented by formula (I), (II), (III), (X) or (XIV), G is —C(O)NHNH—, —NHNHC(O)—, —CH═N—NH—, —NH—N═CH—, —NHNH—, —NHO—, —O—NH—, —NR^{k}—O—, —CH═N—O—, —O—N═CH—, —O—C(S)—NH—, or —NH—C(S)—O—.

In some embodiments, in the compounds represented by formula (I), (II), (III), (X) or (XIV), G is —O—C(O)—NH—, —NH—C(NH)—NH—, —NR^{k}—C(NH)—NH—, —NR^{k}—C(NR^{k})—NH—, —NH—C(N(CN))—NH—, —NH—C(NSO₂R^{c})—NH—, —NR^{k}—C(NSO₂R)—NH—, —NH—C(NNO₂)—NH—, NH—C(NC(O)R^{c})—NH—, —NH—C(O)—NH—, or —NH—C(S)—NH—.

In some embodiments, in the compounds represented by formula (I), (II), (III), (X) or (XIV), G is —NH—S(O)₂—NH—, —NR^{k}—S(O)₂—O—, —P(O)(R^{c})—, —P(O)(R^{c})—O—, or —P(O)(R^{c})—NR^{k}—.

In some embodiments, in the compounds represented by formula (I), (II), (III), (X) or (XIV), G is an optionally substituted cyclyl, an optionally substituted cycloalkyl, an optionally substituted heterocycloalkyl or an optionally substituted heterocyclyl.

In some embodiments, in the compounds represented by formula (I), (II), (III), (X) or (XIV), G is an optionally substituted cyclopropyl, an optionally substituted cyclobutyl, an optionally substituted cyclopentyl, an optionally substituted cyclohexyl, an optionally substituted cycloheptyl, an optionally substituted aziridinyl, an optionally substituted oxiranyl, an optionally substituted azetidinyl, an optionally substituted oxetanyl, an optionally substituted morpholinyl, an optionally substituted piperazinyl or an optionally substituted piperidinyl.

In some embodiments, in the compounds represented by formula (I), (II), (III), (X) or (XIV), G is an optionally substituted aryl, an optionally substituted heteroaryl, an optionally substituted heteroaralkyl, —C(N—CN)—NH—, —Si(OH)₂—, —C(NH)—NR^{k}—, or —NR^{k}—CH₂—C(O)—.

In some embodiments, in the compounds represented by formula (I), (II), (III), (X) or (XIV), G is an optionally substituted imidazolyl, an optionally substituted imidazolidinone, an optionally substituted imidazolidineamine, an optionally substituted pyrrolidinyl, an optionally substituted pyrrolyl, an optionally substituted furanyl, an optionally substituted thienyl, an optionally substituted thiazolyl, an optionally substituted triazolyl, an optionally substituted oxadiazolyl, an optionally substituted thiadiazolyl, an optionally substituted pyrazolyl, an optionally substituted tetrazolyl, an optionally substituted oxazolyl, an optionally substituted isoxazolyl, an optionally substituted phenyl, an optionally substituted pyridyl, an optionally substituted pyrimidyl, an optionally substituted indolyl, or an optionally substituted benzothiazolyl.

In some embodiments, in the compounds represented by formula (I), (II), (III), (X) or (XIV), Y is O or CH₂; G is absent; and n is 0, 1, 2, 3 or 4.

In some embodiments, in the compounds represented by formula (I), (II), (III), (X) or (XIV), Y is absent, O, S, NR^{k}, or CH₂; and n is 0, 1, 2, 3, or 4.

In some embodiments, in the compounds represented by formula (II), X₁ is one of the following formulas:

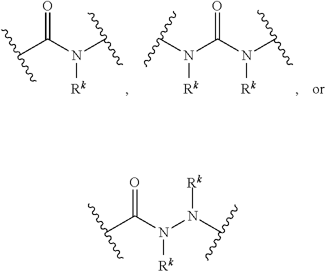

In some embodiments, in the compounds represented by formula (II), X₁ is represented by the following formula:

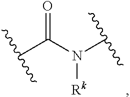

wherein R^{k} is —H or a lower alkyl.

In some embodiments, in the compounds represented by formula (II), X₁ is represented by the following formula:

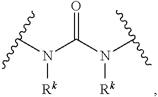

wherein R^{k} is —H or a lower alkyl.

In some embodiments, in the compounds represented by formula (II), X₁ is represented by the following formula:

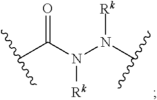

wherein R^{k} is —H or a lower alkyl.

In some embodiments, in the compounds represented by formula (III), X₃ is —C(R^{g})═N—NR^{k}—, wherein R^{g} and R^{k} of X₃ are each, independently, —H or a lower alkyl.

In some embodiments, in the compounds represented by formula (IV), the compound is represented by formula (V):

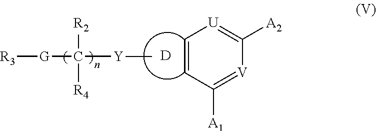

or a pharmaceutically acceptable salt, solvate, clathrate, hydrate, polymorph, or prodrug thereof, wherein:

G, Y, R₂, R₃, R₄, and n are defined as for formula I; and

Ring D, A₁, A₂, U, and V are defined as for formula (IV).

In some embodiments, in the compounds represented by formula (IV) or (V), the compound is represented by one of the following structural formulas:

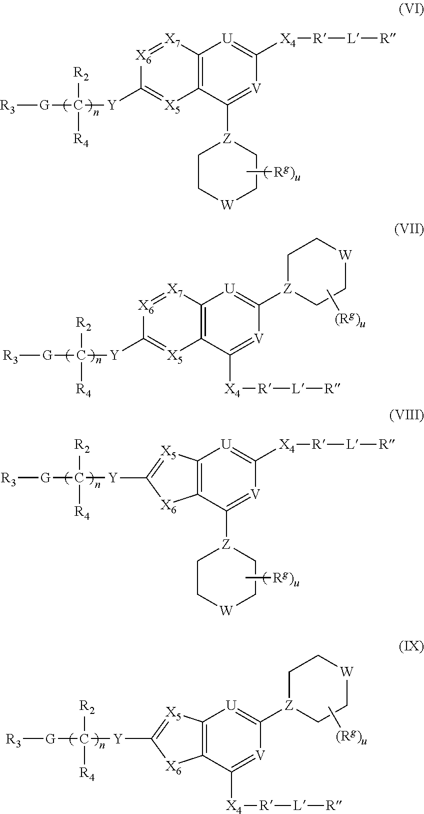

or a pharmaceutically acceptable salt, solvate, clathrate, hydrate, polymorph, or prodrug thereof, wherein:

G, Y, R₂, R₃, R₄, R^{g}, and n are defined as for formula I;

U, V, L, X₄, W, Z, R′, R″, and u are defined as for formula (IV);

X₅, X₆ and X₇ are each, independently, N or CR^{g};

X₈ is CR^{g}R^{g}, O, S(O)ₚ, or NR^{k}, wherein R^{k} is defined as for formula (I).

In some embodiments, in the compounds represented by formula (VI) or formula (VII), U and V are N; and X₅, X₆ and X₇ are CR^{g}.

In some embodiments, in the compounds represented by formula (IV), (V), (VI), (VII), (VIII), or (IX), R′ and L′ are absent.

In some embodiments, in the compounds represented by formula (IV), (V), (VI), (VII), (VIII), or (IX), R″ is an optionally substituted cycloalkyl, an optionally substituted cyclyl, an optionally substituted aryl, an optionally substituted heterocycloalkyl, an optionally substituted heterocyclyl, or an optionally substituted heteroaryl.

In some embodiments, in the compounds represented by formula (IV), (V), (VI), (VII), (VIII), or (IX), R″ is an optionally substituted aryl or an optionally substituted heteroaryl.

In some embodiments, in the compounds represented by formula (IV), (V), (VI), (VII), (VIII), or (IX), R″ is substituted with one or more substituent selected from the group consisting of a lower alkyl, cyano, halo, nitro, —NH₂, a lower alkylamino, a lower dialkylamino, a lower alkoxy, a lower haloalkyl, —S(O)ₚR^{c}, and —C(O)R^{c}.

In some embodiments, in the compounds represented by formula (IV), (V), (VI), (VII), (VIII), or (IX), Z is N and W is O.

In some embodiments, in the compounds represented by formula (V), (VI), (VII), (VIII), or (IX), Y is a covalent bond, O, S, N(R^{k}), or CH₂, and n is 0, 1, 2, 3, or 4.

In some embodiments, in the compounds represented by formula (V), (VI), (VII), (VIII), or (IX), G is absent.

In some embodiments, in the compounds represented by formula (V), (VI), (VII), (VIII), or (IX), G is >C═N—R, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}C(NR)NR^{k}—, —NR^{k}C(O)NR^{k}—, —NR^{k}C(S)NR^{k}—, —NR^{k}S(O)₂NR^{k}—, —C(NR)NR^{k}—, or —NR^{k}CR^{g}R^{g}C(O)—.

In some embodiments, in the compounds represented by formula (V), (VI), (VII), (VIII), or (IX), R₃ is an optionally substituted alkyl, an optionally substituted aryl, an optionally substituted heteroaryl, an optionally substituted cycloalkyl, an optionally substituted cyclyl, an optionally substituted heterocycloalkyl, an optionally substituted heterocyclyl, nitro, cyano, halo, OR^{k}, SR^{k}, or NR^{h}R^{j}.

In some embodiments, in the compounds represented by formula (V), (VI), (VII), (VIII), or (IX), R₃ is optionally substituted aryl or optionally substituted heteroaryl.

In some embodiments, in the compounds represented by formula (V), (VI), (VII), (VIII), or (IX), R₃ is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments, in the compounds represented by formula (V), (VI), (VII), (VIII), or (IX), R₃ is an optionally substituted heterocycloalkyl.

In some embodiments, in the compounds represented by formula (V), (VI), (VII), (VIII), or (IX), R₃ is an optionally substituted piperidinyl, an optionally substituted piperazinyl, an optionally substituted 2-oxopiperazinyl, an optionally substituted 2-oxopiperidinyl, an optionally substituted 2-oxopyrrolidinyl, an optionally substituted 4-piperidonyl, an optionally substituted tetrahydropyranyl, an optionally substituted oxazolidinyl, an optionally substituted 2-oxo-oxazolidinyl, an optionally substituted tetrahydrothiopyranyl, an optionally substituted tetrahydrothiopyranyl sulfone, an optionally substituted morpholinyl, an optionally substituted thiomorpholinyl, an optionally substituted thiomorpholinyl sulfoxide, an optionally substituted thiomorpholinyl sulfone, an optionally substituted 1,3-dioxolanyl, an optionally substituted [1,4]dioxanyl, an optionally substituted 2-oxo-imidazolidinyl, tetrahydrofuranyl, or an optionally substituted tetrahydrothienyl.

In some embodiments, in the compounds represented by formula (V), (VI), (VII), (VIII), or (IX), R₃ is —OR^{k} or —NR^{h}R^{j}, and R^{f}, R^{h} and R^{j} are each, independently, H, an optionally substituted alkyl, an optionally substituted aryl, an optionally substituted heteroaryl, an optionally substituted cycloalkyl, an optionally substituted heterocycloalkyl, or —C(O)R^{c}.

In some embodiments, in the compounds represented by formula (V), (VI), (VII), (VIII), or (IX), R₃ is —C(O)OR^{k}, —OC(O)R^{k}, —C(O)NR^{h}R^{j}, —NR^{k}C(O)R^{k}, —C(S)OR^{k}, —OC(S)R^{k}, —NR^{k}C(O)NR^{h}R^{j}, —NR^{k}C(S)NR^{h}R^{j}, —C(O)NRhRj, —S(O)₂R^{k}, —S(O)₂NR^{h}R^{j}, —OC(O)NR^{h}R^{j}, or —NR^{k}C(O)OR^{k}.

In some embodiments, in the compounds represented by formula (IV), the compound is represented by one of the following structural formulas:

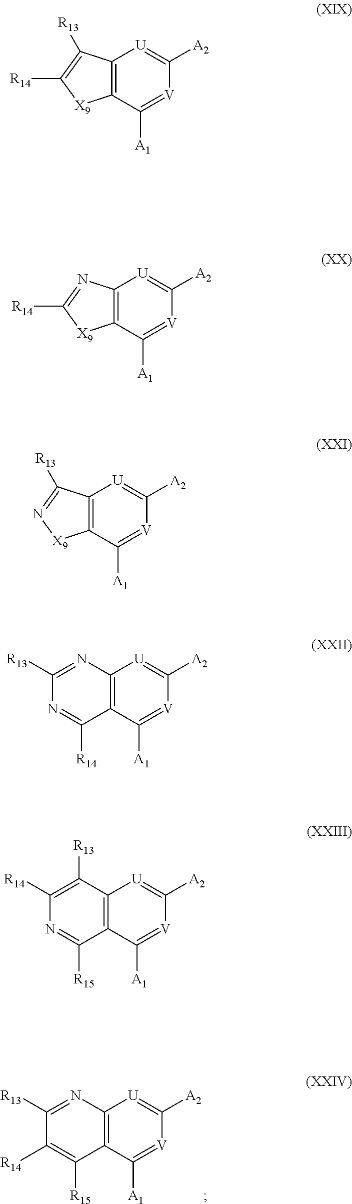

or a pharmaceutically acceptable salt, solvate, clathrate, hydrate, polymorph, or prodrug thereof, wherein;

U, V, A₁, and A₂ are defined as for formula (IV);

X₉ is CR^{g}R^{g}, O, S(O)ₚ, or NR^{k};

one of R₁₃, R₁₄ and R₁₅ is a group represented by the following structural formula:

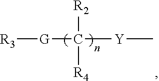

and the remainder of R₁₃, R₁₄ and R₁₅ are independently selected from H, R^{g}, or isothionitro; and

R₂, R₃, R₃, R₄, G, Y, R^{g}, R^{k} and n are defined as for formula (I).

In some embodiments, in the compounds represented by formula (XIX), (XX), (XXI), (XXII), (XXIII), or (XXIV), U and V are N.

In some embodiments, in the compounds represented by formula (XIX), (XX), (XXI), (XXII), (XXIII), or (XXIV), R′ and L′ are absent.

In some embodiments, in the compounds represented by formula (XIX), (XX), (XXI), (XXII), (XXIII), or (XXIV), R″ is an optionally substituted cycloalkyl, an optionally substituted cyclyl, an optionally substituted aryl, an optionally substituted heterocycloalkyl, an optionally substituted heterocyclyl, or an optionally substituted heteroaryl.

In some embodiments, in the compounds represented by formula (XIX), (XX), (XXI), (XXII), (XXIII), or (XXIV), R″ is an optionally substituted aryl or an optionally substituted heteroaryl.

In some embodiments, in the compounds represented by formula (XIX), (XX), (XXI), (XXII), (XXIII), or (XXIV), R″ is substituted with one or more substituent selected from the group consisting of a lower alkyl, cyano, halo, nitro, —NH₂, a lower alkylamino, a lower dialkylamino, a lower alkoxy, a lower haloalkyl, —S(O)ₚR^{c}, and —C(O)R^{c}.

In some embodiments, in the compounds represented by formula (XIX), (XX), (XXI), (XXII), (XXIII), or (XXIV), Z is N and W is O.

In some embodiments, in the compounds represented by formula (XIX), (XX), (XXI), (XXII), (XXIII), or (XXIV), Y is a covalent bond, O, S, N(R^{k}), or CH₂, and n is 0, 1, 2, 3, or 4.

In some embodiments, in the compounds represented by formula (XIX), (XX), (XXI), (XXII), (XXIII), or (XXIV), G is absent.

In some embodiments, in the compounds represented by formula (XIX), (XX), (XXI), (XXII), (XXIII), or (XXIV), G is >C═N—R, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k} C(NR)NR^{k}—, —NR^{k}C(O)NR^{k}—, —NR^{k}C(S)NR^{k}—, —NR^{k}S(O)₂NR^{k}—, —C(NR)NR^{k}—, or —NR^{k}CR^{g}R^{g}C(O)—.

In some embodiments, in the compounds represented by formula (XIX), (XX), (XXI), (XXII), (XXIII), or (XXIV), R₃ is an optionally substituted alkyl, an optionally substituted aryl, an optionally substituted heteroaryl, an optionally substituted cycloalkyl, an optionally substituted cyclyl, an optionally substituted heterocycloalkyl, an optionally substituted heterocyclyl, nitro, cyano, halo, OR^{k}, SR^{k}, or NR^{h}R^{j}.

In some embodiments, in the compounds represented by formula (XIX), (XX), (XXI), (XXII), (XXIII), or (XXIV), R₃ is optionally substituted aryl or optionally substituted heteroaryl.

In some embodiments, in the compounds represented by formula (XIX), (XX), (XXI), (XXII), (XXIII), or (XXIV), R₃ is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments, in the compounds represented by formula (XIX), (XX), (XXI), (XXII), (XXIII), or (XXIV), R₃ is an optionally substituted heterocycloalkyl.

In some embodiments, in the compounds represented by formula (XIX), (XX), (XXI), (XXII), (XXIII), or (XXIV), R₃ is an optionally substituted piperidinyl, an optionally substituted piperazinyl, an optionally substituted 2-oxopiperazinyl, an optionally substituted 2-oxopiperidinyl, an optionally substituted 2-oxopyrrolidinyl, an optionally substituted 4-piperidonyl, an optionally substituted tetrahydropyranyl, an optionally substituted oxazolidinyl, an optionally substituted 2-oxo-oxazolidinyl, an optionally substituted tetrahydrothiopyranyl, an optionally substituted tetrahydrothiopyranyl sulfone, an optionally substituted morpholinyl, an optionally substituted thiomorpholinyl, an optionally substituted thiomorpholinyl sulfoxide, an optionally substituted thiomorpholinyl sulfone, an optionally substituted 1,3-dioxolanyl, an optionally substituted [1,4]dioxanyl, an optionally substituted 2-oxo-imidazolidinyl, tetrahydrofuranyl, or an optionally substituted tetrahydrothienyl.

In some embodiments, in the compounds represented by formula (XIX), (XX), (XXI), (XXII), (XXIII), or (XXIV), R₃ is —OR^{k} or —NR^{h}R^{j}, and R^{f}, R^{h} and R^{j} are each, independently, H, an optionally substituted alkyl, an optionally substituted aryl, an optionally substituted heteroaryl, an optionally substituted cycloalkyl, an optionally substituted heterocycloalkyl, or —C(O)R^{c}.

In some embodiments, in the compounds represented by formula (XIX), (XX), (XXI), (XXII), (XXIII), or (XXIV), R₃ is —C(O)OR^{k}, —OC(O)R^{k}, —C(O)NR^{h}R^{j}, —NR^{k}C(O)R^{k}, —C(S)OR^{k}, —OC(S)R^{k}, —NR^{k}C(O)NR^{h}R^{j}, —NR^{k}C(S)NR^{h}R^{j}, —C(O)NRhRj, —S(O)₂R^{k}, —S(O)₂NR^{h}R^{j}, —OC(O)NR^{h}R^{j}, or —NR^{k}C(O)OR^{k}.

In some embodiments, in the compounds represented by formula (X), the compound is represented by one of the following structural formulas:

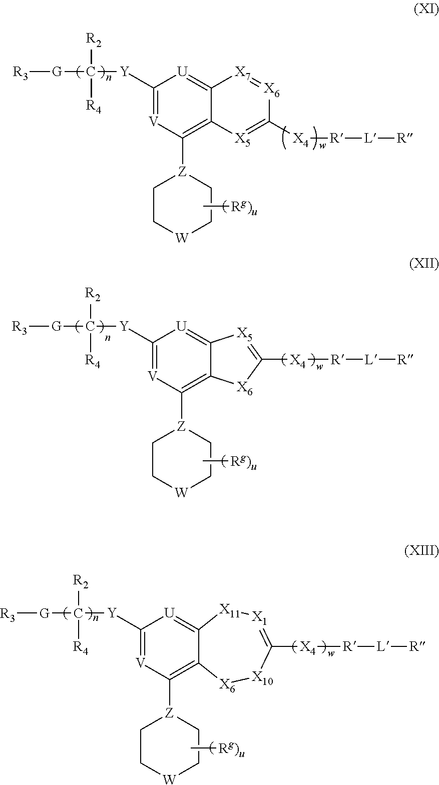

or a pharmaceutically acceptable salt, solvate, clathrate, hydrate, polymorph, or prodrug thereof, wherein:

G, Y, R₂, R₃, R₄, R^{g} and n are defined as for formula (I);

R′, R″, L′, X₄, U, V, W, Z, and u are defined as for formula (IV);

w is defined as for formula (X);

X₅, X₆ and X₇ are each, independently, N or CR^{g}; and

X₈, X₁₀, and X₁₁ are each, independently, CR^{g}R^{g}, O, S(O)ₚ, or NR^{k}, wherein R^{k} is defined as for formula (I).

In some embodiments, in the compounds represented by formula (XI), U and V are N; and X₅ and X₆ are CR^{g}.

In some embodiments, in the compounds represented by formula (XI), U and V are N; X₅ and X₆ are CR^{g}; and X₇ is N.

In some embodiments, in the compounds represented by formula (XI), U and V are N; X₅ and X₆ are CR^{g}; and X₇ is CR^{g}.

In some embodiments, in the compounds represented by formula (XI), (XII), or (XIII), w is 0, and R′ and L′ are absent.

In some embodiments, in the compounds represented by formula (XI), (XII), or (XIII), R″ is an optionally substituted cycloalkyl, an optionally substituted cyclyl, an optionally substituted aryl, an optionally substituted heterocycloalkyl, an optionally substituted heterocyclyl, or an optionally substituted heteroaryl.

In some embodiments, in the compounds represented by formula (XI), (XII), or (XIII), R″ is an optionally substituted aryl or an optionally substituted heteroaryl.

In some embodiments, in the compounds represented by formula (XI), (XII), or (XIII), R″ is substituted with one or more substituent selected from the group consisting of a lower alkyl, cyano, halo, nitro, —NH₂, a lower alkylamino, a lower dialkylamino, a lower alkoxy a lower haloalkyl, —S(O)ₚR^{c}, and —C(O)R^{c}.

In some embodiments, in the compounds represented by formula (XI), (XII), or (XIII), Z is N and W is O.

In some embodiments; in the compounds represented by formula (XI), (XII), or (XIII), Y is a covalent bond, O, S, N(R^{k}), or CH₂, and n is 0, 1, 2, 3, or 4.

In some embodiments, in the compounds represented by formula (XI), (XII), or (XIII), G is absent.

In some embodiments, in the compounds represented by formula (XI), (XII), or (XIII), G is >C═N—R, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}C(NR)NR^{k}—, —NR^{k}C(O)NR^{k}—, —NR^{k}C(S)NR^{k}—, —NR^{k}S(O)₂NR^{k}—, —C(NR)NR^{k}—, or —NR^{k}CR^{g}R^{g}C(O)—.

In some embodiments, in the compounds represented by formula (XI), (XII), or (XIII), R₃ is an optionally substituted alkyl, an optionally substituted aryl, an optionally substituted heteroaryl, an optionally substituted cycloalkyl, an optionally substituted cyclyl, an optionally substituted heterocycloalkyl, an optionally substituted heterocyclyl, nitro, cyano, halo, OR^{k}, SR^{k}, or NR^{h}R^{j}.

In some embodiments, in the compounds represented by formula (XI), (XII), or (XIII), R₃ is optionally substituted aryl or optionally substituted heteroaryl.

In some embodiments, in the compounds represented by formula (XI), (XII), or (XIII), R₃ is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments, in the compounds represented by formula (XI), (XII), or (XIII), R₃ is an optionally substituted heterocycloalkyl.

In some embodiments, in the compounds represented by formula (XI), (XII), or (XIII), R₃ is an optionally substituted piperidinyl, an optionally substituted piperazinyl, an optionally substituted 2-oxopiperazinyl, an optionally substituted 2-oxopiperidinyl, an optionally substituted 2-oxopyrrolidinyl, an optionally substituted 4-piperidonyl, an optionally substituted tetrahydropyranyl, an optionally substituted oxazolidinyl, an optionally substituted 2-oxo-oxazolidinyl, an optionally substituted tetrahydrothiopyranyl, an optionally substituted tetrahydrothiopyranyl sulfone, an optionally substituted morpholinyl, an optionally substituted thiomorpholinyl, an optionally substituted thiomorpholinyl sulfoxide, an optionally substituted thiomorpholinyl sulfone, an optionally substituted 1,3-dioxolanyl, an optionally substituted [1,4]dioxanyl, an optionally substituted 2-oxo-imidazolidinyl, tetrahydrofuranyl, or an optionally substituted tetrahydrothienyl.

In some embodiments, in the compounds represented by formula (XI), (XII), or (XIII), R₃ is —OR^{k} or —NR^{h}R^{j}, and R^{f}, R^{h} and R^{j} are each, independently, H, an optionally substituted alkyl, an optionally substituted aryl, an optionally substituted heteroaryl, an optionally substituted cycloalkyl, an optionally substituted heterocycloalkyl, or —C(O)R^{c}.

In some embodiments, in the compounds represented by formula (XI), (XII), or (XIII), R₃ is —C(O)OR^{k}, —OC(O)R^{k}, —C(O)NR^{h}R^{j}, —NR^{k}C(O)R^{k}, —C(S)OR^{k}, —OC(S)R^{k}, —NR^{k}C(O)NR^{h}R^{j}, —NR^{k}C(S)NR^{h}R^{j}, —C(O)NRhRj, —S(O)₂R^{k}, —S(O)₂NR^{h}R^{j}, —OC(O)NR^{h}R^{j}, or —NR^{k}C(O)OR^{k}.

In some embodiments, in the compounds represented by formula (XI), (XII), or (XIII), w is 1; X₄ is O, S, or NR^{k}; and R′ and L′ are absent.

In some embodiments, in the compounds represented by formula (XIV), the compound is represented by formula (XV):

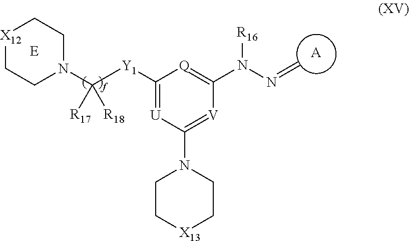

or a pharmaceutically acceptable salt, solvate, clathrate, hydrate, polymorph, or prodrug thereof, wherein:

Q, U, and V are defined as for formula (I);

R₁₆ is defined as for formula (XIV);

ring E is optionally substituted with one to four substituents selected from a lower alkyl, a halo, an amino, a lower alkyl amino, a lower dialkyl amino, a cyano, a nitro, a lower haloalkyl, a hydroxyl, and a lower hydroxyalkyl;

X₁₂ is O, S, S(O), S(O)₂, or CR^{g}R^{g};

X₁₃ is O, S, S(O), S(O)₂, or CH₂;

Y₁ is O, S, NR^{k}, or CH₂;

R₁₇ and R₁₈, for each occurrence, are independently, H or a lower alkyl; or R₁₇ and R₁₈ taken together with the carbon to which they are attached form a cycloalkyl; and

f is 0, 1, 2, or 3.

In some embodiments, in the compounds represented by formula (XIV), the compound is represented by formula (XVI):

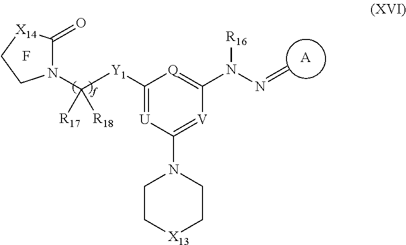

or a pharmaceutically acceptable salt, solvate, clathrate, hydrate, polymorph, or prodrug thereof, wherein:

Q, U, and V are defined as for formula (I);

R₁₆ is defined as for formula (XIV);

Y₁, R₁₇, R₁₈, X₁₃, and f are defined as for formula (XV);

ring F is optionally substituted with one or two substituents selected from a lower alkyl, a halo, an amino, a lower alkyl amino, a lower dialkyl amino, a cyano, a nitro, a lower haloalkyl, a hydroxyl, and a lower hydroxyalkyl; and

X₁₄ is O, NR^{k}, or CR^{g}R^{g}.

In some embodiments, in the compounds represented by formula (XIV), the compound is represented by formula (XVII):

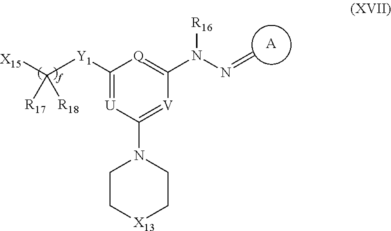

or a pharmaceutically acceptable salt, solvate, clathrate, hydrate, polymorph, or prodrug thereof; wherein:

Q, U, and V are defined as for formula (I);

R₁₆ is defined as for formula (XIV);

Y₁, R₁₇, R₁₈, X₁₃, and f are defined as for formula (XV); and

X₁₅ is —OH, —NH₂ or —SH.

In some embodiments, in the compounds represented by formula (XV), (XVI), or (XVII), Q, U, and V are N.

In some embodiments, in the compounds represented by formula (XV), (XVI), or (XVII), one of Q, U, or V is CR^{g}, and the other two are N.

In some embodiments, in the compounds represented by formula (XV), (XVI), or (XVII), V is CR^{g}, Q and U are N.

In some embodiments, in the compounds represented by formula (XV), (XVI), or (XVII), Q is CR^{g}, V and U are N.

In some embodiments, in the compounds represented by formula (XV), (XVI), or (XVII), U is CR^{g}, V and Q are N.

In some embodiments, in the compounds represented by formula (XV), (XVI), or (XVII), one of Q, U, or V is N, and the other two are CR^{g}.

In some embodiments, in the compounds represented by formula (XV), (XVI), or (XVII), V is N, and Q and U are CR^{g}.

In some embodiments, in the compounds represented by formula (XV), (XVI), or (XVII), Q is N, and V and U are CR^{g}.

In some embodiments, in the compounds represented by formula (XV), (XVI), or (XVII), U is N and Q, and V are CR^{g}.

In some embodiments, in the compounds represented by formula (XV), (XVI), or (XVII), —NR₅R₆ is an optionally substituted morpholino, an optionally substituted thiomorpholino, an optionally substituted 1-oxo-thiomorpholino, an optionally substituted 1,1-dioxo-thiomorpholino, an optionally substituted piperidinyl, or an optionally substituted piperazinyl.

In some embodiments, in the compounds represented by formula (XIV), (XV), (XVI), or (XVII), ring A is a ring system selected from the group consisting of:

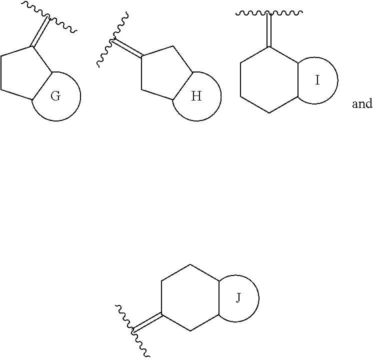

wherein:

“ [symbol] ” represents the point of attachment;

rings G, H, I, and J are each, independently, an aryl or a heteroaryl; and

each ring system is optionally substituted with one or more substituents.

In some embodiments, in the compounds represented by formula (XIV), (XV), (XVI), or (XVII), ring A is a ring system selected from the group consisting of:

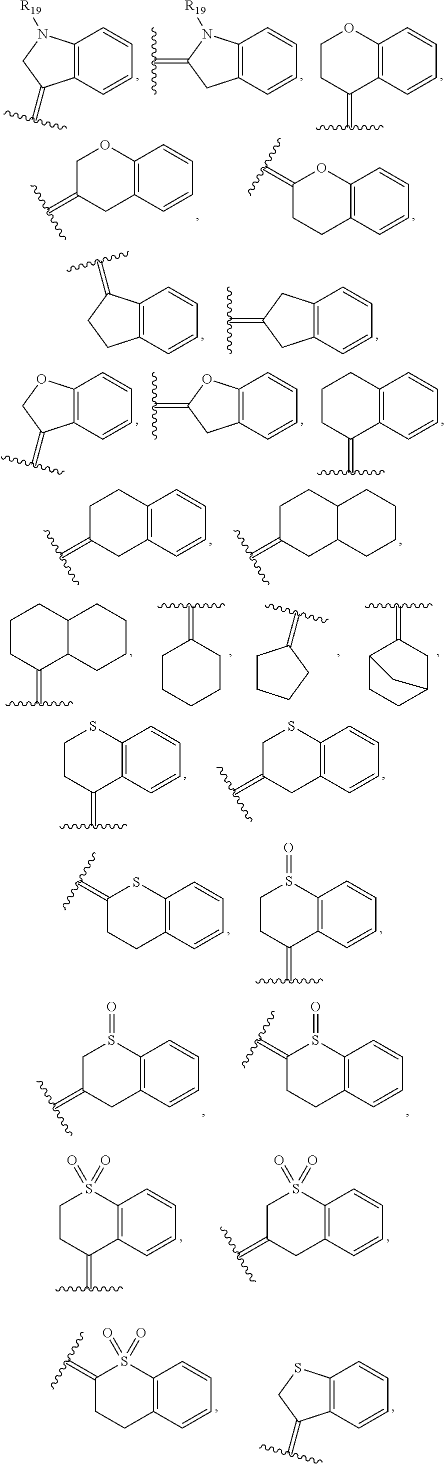

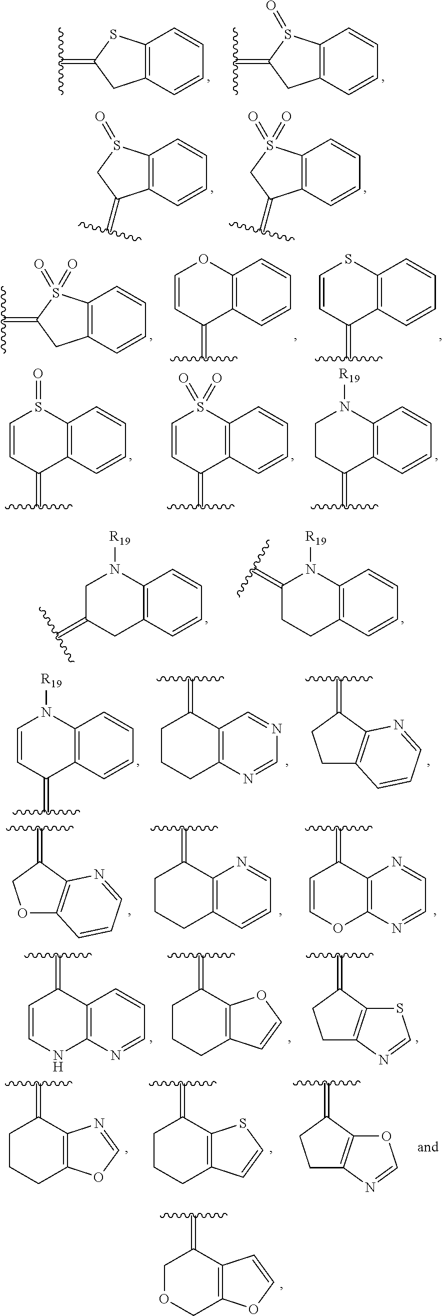

wherein:

each ring system is optionally substituted with one or more substituents;

“ [symbol] ” represents the point of attachment; and

R₁₉ is H, an alkyl, an aralkyl, or an alkylcarbonyl.

In some embodiments, in the compounds represented by formula (XIV), (XV), (XVI), or (XVII), ring A is a ring system selected from the group consisting of:

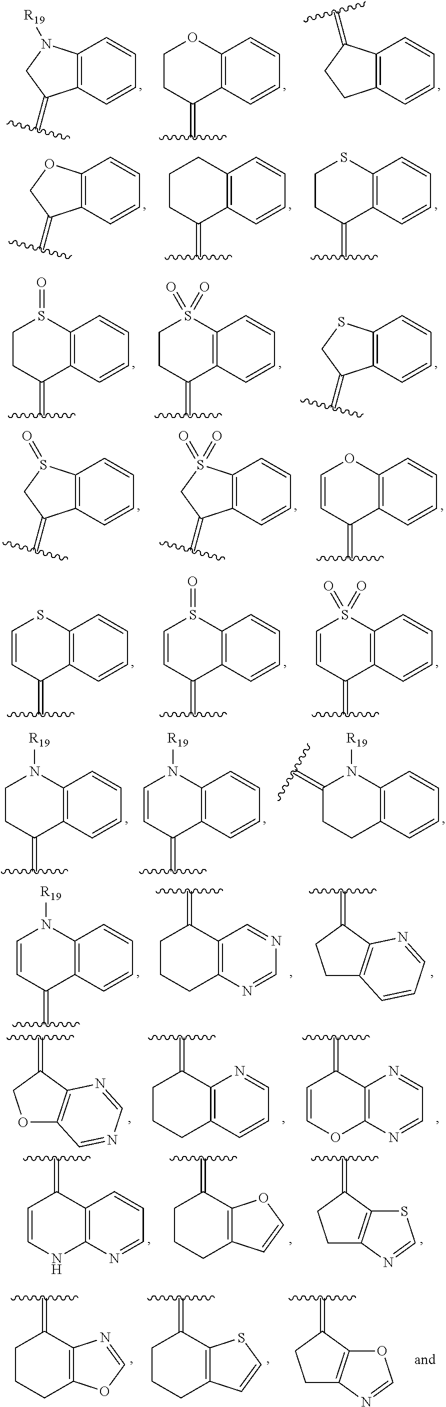

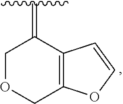 [SMILES: *=C1COCc2occc21]

wherein:

each ring system is optionally substituted with one or more substituents.

In some embodiments, in the compounds represented by formula (XIV), (XV), (XVI), or (XVII), ring A is optionally substituted with one or more substituents selected from the group consisting of an optionally substituted alkyl, an optionally substituted alkoxy, an optionally substituted alkyl sulfanyl, an optionally substituted alkenyl, an optionally substituted alkynyl, an optionally substituted cycloalkyl, an optionally substituted cyclyl, an optionally substituted heterocyclyl, an optionally substituted heterocycloalkyl, an optionally substituted aryl, an optionally substituted heteroaryl, an optionally substituted aralkyl, an optionally substituted heteroaralkyl, a haloalkyl, halo, cyano, nitro, haloalkoxy, ═O, ═S, ═NR, —OR^{k}, —NR^{h}R^{j}, —SR^{k}, —C(O)R^{k}, —C(O)NR^{h}R^{j}, —NR^{k}C(O)R^{k}, —C(O)OR^{k}, —OC(O)R^{k}, —NR^{k}C(O)NR^{h}R^{j}, —OC(O)NR^{h}R^{j}, —NR^{k}C(O)OR^{k}, —C(NR)R^{k}, —C(NR)NR^{h}R^{j}, —NR^{k}C(NR)R^{k}, —C(NR)OR^{k}, —OC(NR)R^{k}, —NR^{k}C(NR)NR^{h}R^{j}, —OC(NR)NR^{h}R^{j}, —NR^{k}C(NR)OR^{k}, —C(S)R^{k}, —C(S)NR^{h}R^{j}, —NR^{k}C(S)R^{k}, —C(S)OR^{k}, —OC(S)R^{k}, —NR^{k}C(S)NR^{h}R^{j}, —OC(S)NR^{h}R^{j}, —NR^{k}C(S)OR^{k}, —C(O)SR^{k}, —SC(O)R^{k}, —S(O)ₚR^{k}, —S(O)ₚNR^{h}R^{j}, —OS(O)ₚR^{k}, —S(O)ₚOR^{k}, —OS(O)ₚOR^{k}, —P(O)(OR^{k})₂, —OP(O)(OR^{k})₂, —P(S)(OR^{k})₂, —SP(O)(OR^{k})₂, —P(O)ₚOR^{k}, —OP(O)(SR^{k})(OR^{k}), —P(O)(SR^{k})₂, or —OP(O)(SR^{k})₂, wherein p is 1 or 2.

In some embodiments, in the compounds represented by formula (XIV), (XV), (XVI), or (XVII), ring A is optionally substituted with from one to three substituents selected from the group consisting of a lower alkyl, a lower alkoxy, ═O, nitro, cyano, hydroxy, amino, lower alkyl amino, lower dialkyl amino, mercapto, lower alkyl sulfanyl, halo, or haloalkyl.

In some embodiments, in the compounds represented by formula (XV), (XVI), or (XVII), Y₁ is O.

In some embodiments, in the compounds represented by formula (XV), (XVI), or (XVII), Y₁ is a covalent bond.

In some embodiments, in the compounds represented by formula (XIV), Y is O or CH₂; G is absent; and n is 0, 1, 2, 3 or 4.

In some embodiments, in the compounds represented by formula (XIV), Y is absent, O, S, NR^{k}, or CH₂; and n is 0, 1, 2, 3, or 4.

In some embodiments, in the compounds represented by formula (XV), X₁₂, X₁₃, Y₁ is O; and R₁₇ and R₁₈ are each, independently, H or a lower alkyl.

In some embodiments, in the compounds represented by formula (XVI), X₁₃, X₁₄, and Y₁ are O; and R₁₇ and R₁₈ are each, independently, H or a lower alkyl.

In some embodiments, in the compounds represented by formula (XVII), X₁₃ and Y₁ are O; X₁₅ is —OH; and R₁₇ and R₁₈ are each, independently, H or a lower alkyl.

Specific examples of compounds of the invention are set forth below in Table 1:

TABLE 1

| No. | Structure | Name |
|---|---|---|
| 1 | 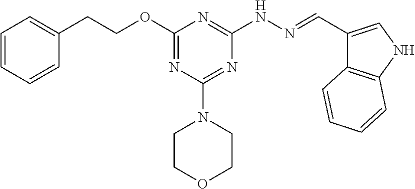 [SMILES: C(=N\Nc1nc(OCCc2ccccc2)nc(N2CCOCC2)n1)/c1c[nH]c2ccccc12] | N-(1H-indol-3-ylmethylene)- N′-(4-morpholin-4-yl-6- phenethyloxy-[1,3,5]triazin-2- yl)-hydrazine |
| 2 | 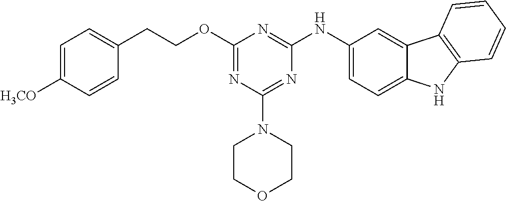 [SMILES: COc1ccc(CCOc2nc(Nc3ccc4[nH]c5ccccc5c4c3)nc(N3CCOCC3)n2)cc1] | N-(9H-carbazol-3-yl)-{4-[2-(4- methoxy-phenyl)-ethoxy]-6- morpholin-4-yl-[1,3,5]triazin- 2-yl}-amine |
| 3 | 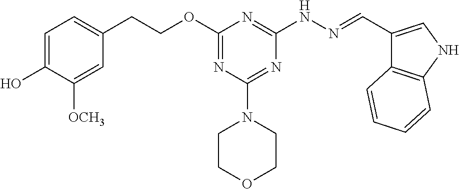 [SMILES: COc1cc(CCOc2nc(N/N=C/c3c[nH]c4ccccc34)nc(N3CCOCC3)n2)ccc1O] | N-(1H-Indol-3-ylmethylene)- N′-(4-morpholin-4-yl-6-(3- methoxy-4-hydroxy- phenethyloxy-[1,3,5]triazin-2- yl)-hydrazine |
| 4 | 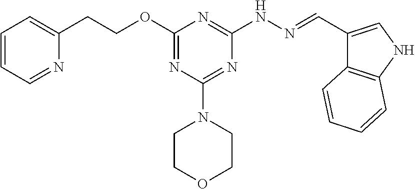 [SMILES: C(=N\Nc1nc(OCCc2ccccn2)nc(N2CCOCC2)n1)/c1c[nH]c2ccccc12] | N-(1H-Indol-3-ylmethylene)- N′-(4-morpholin-4-yl-6-(2- pyridin-2-yl-ethyloxy- [1,3,5]triazin-2-yl)-hydrazine |
| 5 | 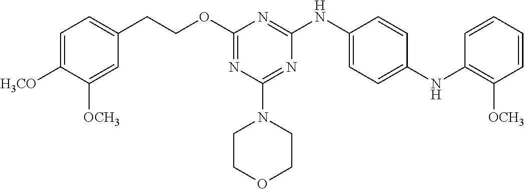 [SMILES: COc1ccccc1Nc1ccc(Nc2nc(OCCc3ccc(OC)c(OC)c3)nc(N3CCOCC3)n2)cc1] | N-[4-(2-methoxy- phenylamino)-phenyl]-{4-[2- (3,4-dimethoxy-phenethyloxy]- 6-morpholin-4-yl- [1,3,5]triazin-2-yl}-amine |
| 6 | 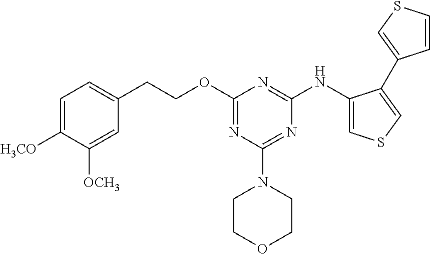 [SMILES: COc1ccc(CCOc2nc(Nc3cscc3-c3ccsc3)nc(N3CCOCC3)n2)cc1OC] | [3,3′]Bithiophen-4-yl-{4-[2- (3,4-dimethoxy-phenethyloxy]- 6-morpholin-4-yl- [1,3,5]triazin-2-yl}-amine |
| 7 | 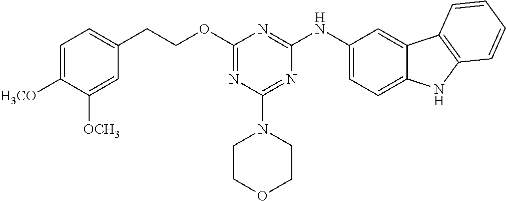 [SMILES: COc1ccc(CCOc2nc(Nc3ccc4[nH]c5ccccc5c4c3)nc(N3CCOCC3)n2)cc1OC] | N-(9H-carbazol-3-yl)-{4-[2- (3,4-dimethoxy-phenyl)- ethoxy]-6-morpholin-4-yl- [1,3,5]triazin-2-yl}-amine |
| 8 | 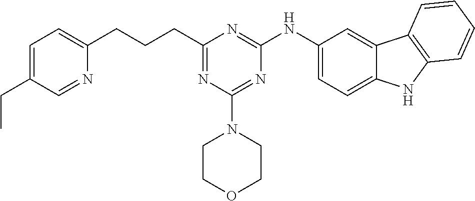 [SMILES: CCc1ccc(CCCc2nc(Nc3ccc4[nH]c5ccccc5c4c3)nc(N3CCOCC3)n2)nc1] | N-(9H-carbazol-3-yl)-{4-[3-(5- ethyl-phenyl)-propyl]-6- morpholin-4-yl-[1,3,5]triazin- 2-yl}-amine |
| 9 | 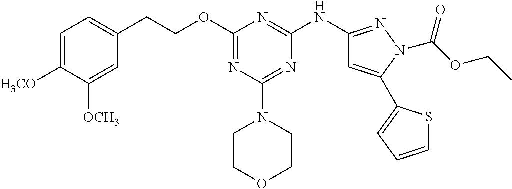 [SMILES: CCOC(=O)n1nc(Nc2nc(OCCc3ccc(OC)c(OC)c3)nc(N3CCOCC3)n2)cc1-c1cccs1] | 3-{4-[2-(3,4-Dimethoxy- phenyl)-ethoxy]-6-morpholin-4-yl- [1,3,5]triazin-2-ylamino}- 5-thiophen-2-yl-pyrazole- 1-carboxylic acid ethyl ester |
| 10 | 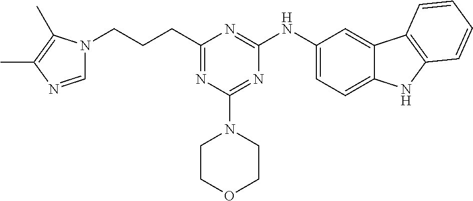 [SMILES: Cc1ncn(CCCc2nc(Nc3ccc4[nH]c5ccccc5c4c3)nc(N3CCOCC3)n2)c1C] | (9H-Carbazol-3-yl)-{4-[3- (4,5-dimethyl-imidazol- 1-yl)-propyl]-6-morpholin- 4-yl-[1,3,5]triazin-2-yl}- amine |
| 11 | 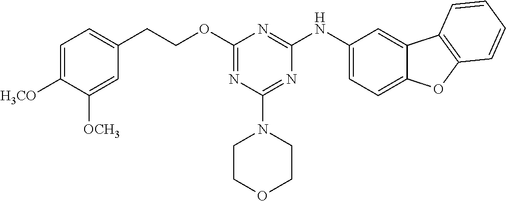 [SMILES: COc1ccc(CCOc2nc(Nc3ccc4oc5ccccc5c4c3)nc(N3CCOCC3)n2)cc1OC] | Dibenzofuran-2-yl-{4-[2- (3,4-dimethoxy-phenyl)- ethoxy]-6-morpholin- 4-yl-[1,3,5]triazin-2-yl}- amine |
| 12 | 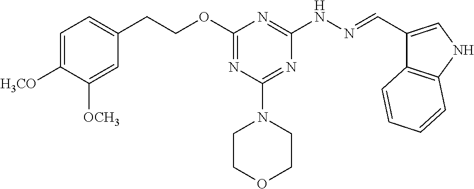 [SMILES: COc1ccc(CCOc2nc(N/N=C/c3c[nH]c4ccccc34)nc(N3CCOCC3)n2)cc1OC] | N-{4-[2-(3,4-Dimethoxy- phenyl)-ethoxy]-6-morpholin- 4-yl-[1,3,5-]triazin-2-yl}- N′-(1H-indol-3-ylmethylene)- hydrazine |
| 13 | 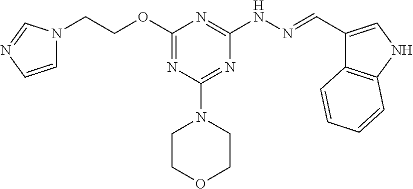 [SMILES: C(=N\Nc1nc(OCCn2ccnc2)nc(N2CCOCC2)n1)/c1c[nH]c2ccccc12] | N-[4-(2-Imidazol-1-yl-ethoxy)- 6-morpholin-4-yl- [1,3,5]triazin-2-yl]-N′-(1H- indol-3-ylmethylene)-hydrazine |
| 14 | 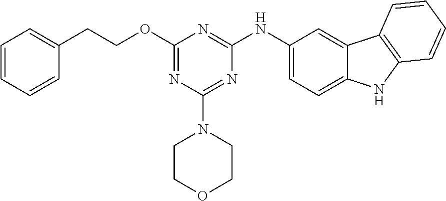 [SMILES: c1ccc(CCOc2nc(Nc3ccc4[nH]c5ccccc5c4c3)nc(N3CCOCC3)n2)cc1] | (9H-Carbazol-3-yl)-(4-morpholin- 4-yl-6-phenethyloxy- [1,3,5]triazin-2-yl)-amine |
| 15 | 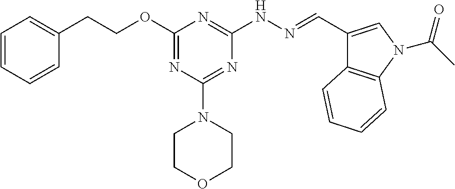 [SMILES: CC(=O)n1cc(/C=N/Nc2nc(OCCc3ccccc3)nc(N3CCOCC3)n2)c2ccccc21] | 1-{3-[(4-Morpholin-4- yl-6-phenethyloxy- [1,3,5]triazin-2-yl)- hydrazonomethyl]-indol- 1-yl}-ethanone |
| 16 | 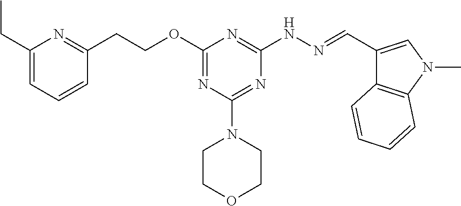 [SMILES: CCc1cccc(CCOc2nc(N/N=C/c3cn(C)c4ccccc34)nc(N3CCOCC3)n2)n1] | N-{4-[2-(6-Ethyl-pyridin-2-yl)- ethoxy]-6-morpholin-4-yl- [1,3,5]triazin-2-yl}-N′-(1-methyl- 1H-indol-3-ylmethylene)-hydrazine |
| 17 | 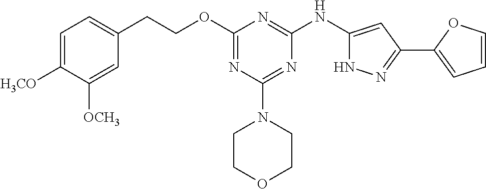 [SMILES: COc1ccc(CCOc2nc(Nc3cc(-c4ccco4)n[nH]3)nc(N3CCOCC3)n2)cc1OC] | {4-[2-(3,4-Dimethoxy-phenyl)- ethoxy]-6-morpholin-4-yl- [1,3,5]triazin-2-yl}-(5- furan-2-yl-2H-pyrazol-3- yl)-amine |
| 18 | 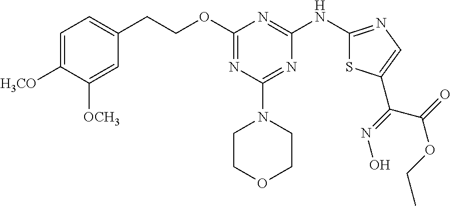 [SMILES: CCOC(=O)/C(=N\O)c1cnc(Nc2nc(OCCc3ccc(OC)c(OC)c3)nc(N3CCOCC3)n2)s1] | (2-{4-[2-(3,4-Dimethoxy-phenyl)- ethoxy]-6-morpholin-4-yl- [1,3,5]triazin-2-ylamino}-thiazol- 5-yl)-hydroxyimino-acetic acid ethyl ester |
| 19 | 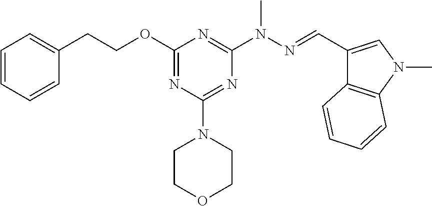 [SMILES: CN(/N=C/c1cn(C)c2ccccc12)c1nc(OCCc2ccccc2)nc(N2CCOCC2)n1] | N-Methyl-N′-(1-methyl-1H- indol-3-ylmethylene)-N- (4-morpholin-4-yl-6- phenethyloxy-[1,3,5]triazin- 2-yl)-hydrazine |
| 20 | 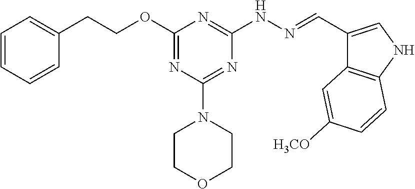 [SMILES: COc1ccc2[nH]cc(/C=N/Nc3nc(OCCc4ccccc4)nc(N4CCOCC4)n3)c2c1] | N-(5-Methoxy-1H-indol-3- ylmethylene)-N′-(4-morpholin- 4-yl-6-phenethyloxy- [1,3,5]triazin-2-yl)-hydrazine |
| 21 | 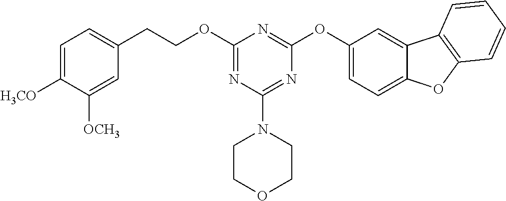 [SMILES: COc1ccc(CCOc2nc(Oc3ccc4oc5ccccc5c4c3)nc(N3CCOCC3)n2)cc1OC] | 2-(Dibenzofuran-2-yloxy)-4- [2-(3,4-dimethoxy-phenyl)- ethoxy]-6-morpholin-4-yl- [1,3,5]triazine |
| 22 | 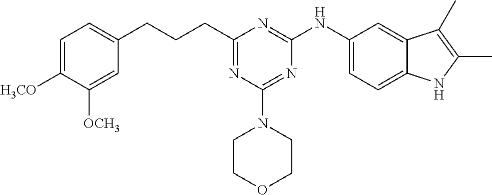 [SMILES: COc1ccc(CCCc2nc(Nc3ccc4[nH]c(C)c(C)c4c3)nc(N3CCOCC3)n2)cc1OC] | {4-[3-(3,4-Dimethoxy-phenyl)- propyl]-6-morpholin-4-yl- [1,3,5]triazin-2-yl}-(2,3- dimethyl-1H-indol-5-yl)-amine |
| 23 | 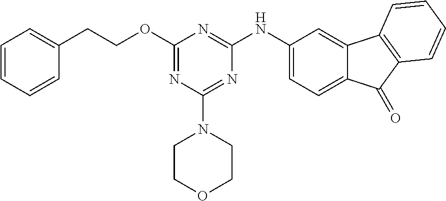 [SMILES: O=C1c2ccccc2-c2cc(Nc3nc(OCCc4ccccc4)nc(N4CCOCC4)n3)ccc21] | 3-(4-Morpholin-4-yl-6- phenethyloxy-[1,3,5]triazin- 2-ylamino)-fluoren-9-one |
| 24 | 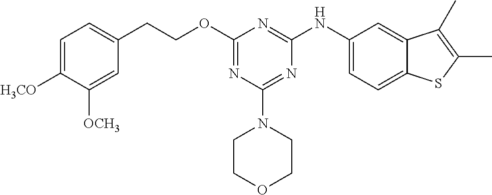 [SMILES: COc1ccc(CCOc2nc(Nc3ccc4sc(C)c(C)c4c3)nc(N3CCOCC3)n2)cc1OC] | {4-[2-(3,4-Dimethoxy-phenyl)- ethoxy]-6-morpholin-4-yl- [1,3,5]triazin-2-yl}-(2,3- dimethyl-benzo[b]thiophen-5- yl)-amine |
| 25 | 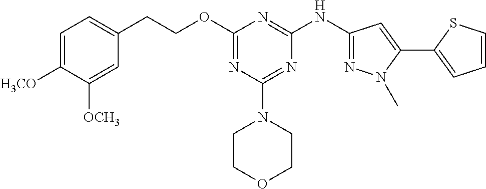 [SMILES: COc1ccc(CCOc2nc(Nc3cc(-c4cccs4)n(C)n3)nc(N3CCOCC3)n2)cc1OC] | {4-[2-(3,4-Dimethoxy-phenyl)- ethoxy]-6-morpholin-4-yl- [1,3,5]triazin-2-yl}-(1- methyl-5-thiophen-2-yl- 1H-pyrazol-3-yl)-amine |
| 26 | 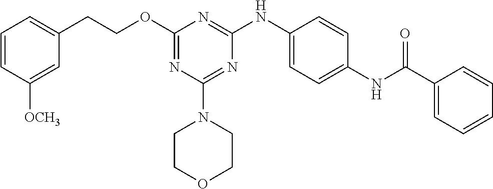 [SMILES: COc1cccc(CCOc2nc(Nc3ccc(NC(=O)c4ccccc4)cc3)nc(N3CCOCC3)n2)c1] | N-(4-{4-[2-(3-Methoxy-phenyl)- ethoxy]-6-morpholin-4-yl- [1,3,5]triazin-2-ylamino}- phenyl)-benzamide |
| 27 | 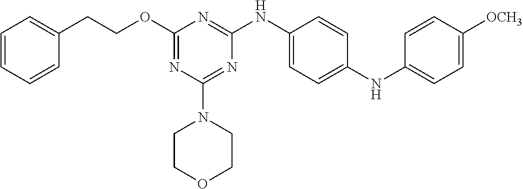 [SMILES: COc1ccc(Nc2ccc(Nc3nc(OCCc4ccccc4)nc(N4CCOCC4)n3)cc2)cc1] | N-(4-Methoxy-phenyl)-N′-(4- morpholin-4-yl-6-phenethyloxy- [1,3,5]triazin-2-yl)-benzene- 1,4-diamine |
| 28 | 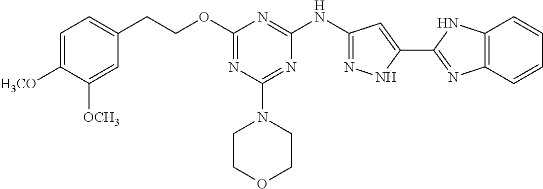 [SMILES: COc1ccc(CCOc2nc(Nc3cc(-c4nc5ccccc5[nH]4)[nH]n3)nc(N3CCOCC3)n2)cc1OC] | [5-(1H-Benzoimidazol-2-yl)- 1H-pyrazol-3-yl]-{4-[2-(3,4- dimethoxy-phenyl)-ethoxy]-6- morpholin-4-yl-[1,3,5]triazin- 2-yl}-amine |
| 29 | 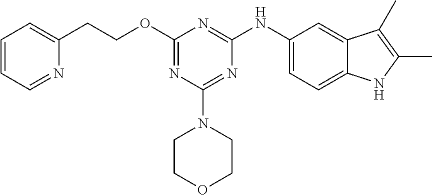 [SMILES: Cc1[nH]c2ccc(Nc3nc(OCCc4ccccn4)nc(N4CCOCC4)n3)cc2c1C] | (2,3-Dimethyl-1H-indol-5-yl)- [4-morpholin-4-yl-6-(2-pyridin- 2-yl-ethoxy)-[1,3,5]triazin-2-yl]- amine |
| 30 | 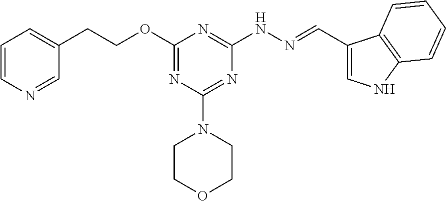 [SMILES: C(=N\Nc1nc(OCCc2cccnc2)nc(N2CCOCC2)n1)/c1c[nH]c2ccccc12] | N-(1H-Indol-3-ylmethylene)-N′- [4-morpholin-4-yl-6-(2-pyridin- 3-yl-ethoxy)-[1,3,5]triazin-2- yl]-hydrazine |
| 31 | 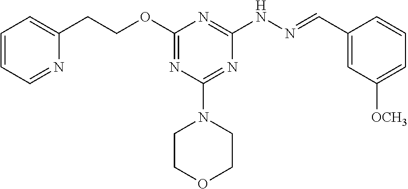 [SMILES: COc1cccc(/C=N/Nc2nc(OCCc3ccccn3)nc(N3CCOCC3)n2)c1] | N-(3-Methoxy-benzylidene)-N′- [4-morpholin-4-yl-6-(2- pyridin-2-yl-ethoxy)- [1,3,5]triazin-2-yl]-hydrazine |
| 32 | 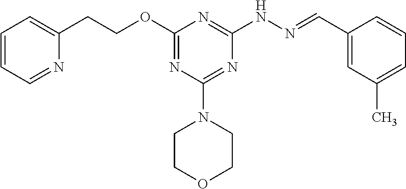 [SMILES: Cc1cccc(/C=N/Nc2nc(OCCc3ccccn3)nc(N3CCOCC3)n2)c1] | N-(3-Methyl-benzylidene)-N′- [4-morpholin-4-yl-6-(2- pyridin-2-yl-ethoxy)- [1,3,5]triazin-2-yl]-hydrazine |
| 33 | 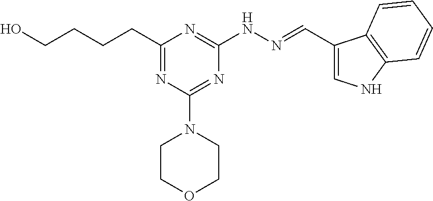 [SMILES: OCCCCc1nc(N/N=C/c2c[nH]c3ccccc23)nc(N2CCOCC2)n1] | 4-{4-[N′-(1H-Indol-3- ylmethylene)-hydrazino]-6- morpholin-4-yl-[1,3,5]triazin- 2-yl}-butan-1-ol |
| 34 | 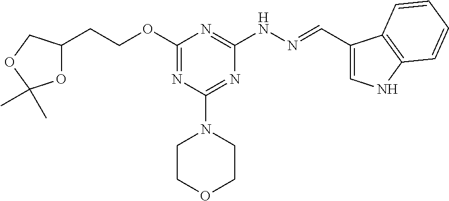 [SMILES: CC1(C)OCC(CCOc2nc(N/N=C/c3c[nH]c4ccccc34)nc(N3CCOCC3)n2)O1] | N-{4-[2-(2,2-Dimethyl- [1,3]dioxolan-4-yl)-ethoxy]- 6-morpholin-4-yl- [1,3,5]triazin-2-yl}-N′-(1H-indol- 3-ylmethylene)-hydrazine |
| 35 | 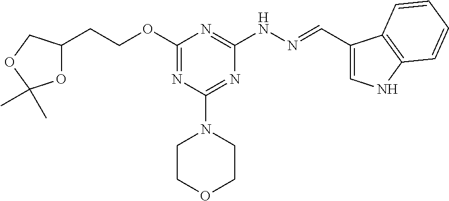 [SMILES: CC1(C)OCC(CCOc2nc(N/N=C/c3c[nH]c4ccccc34)nc(N3CCOCC3)n2)O1] | N-{4-[2-(2,2-Dimethyl- [1,3]dioxolan-4-yl)-ethoxy]- 6-morpholin-4-yl-[1,3,5]triazin- 2-yl}-N′-(1H-indol-3- ylmethylene)-hydrazine |
| 36 | 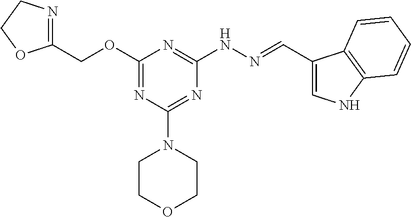 [SMILES: C(=N\Nc1nc(OCC2=NCCO2)nc(N2CCOCC2)n1)/c1c[nH]c2ccccc12] | N-[4-(4,5-Dihydro-oxazol-2- ylmethoxy)-6-morpholin-4-yl- [1,3,5]triazin-2-yl]-N′-(1H-indol- 3-ylmethylene)-hydrazine |
| 37 | 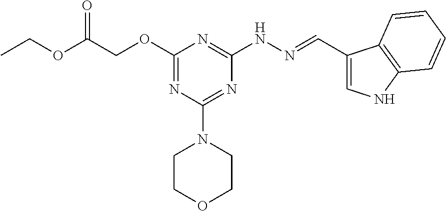 [SMILES: CCOC(=O)COc1nc(N/N=C/c2c[nH]c3ccccc23)nc(N2CCOCC2)n1] | {4-[N′-(1H-Indol- 3-ylmethylene)-hydrazino]- 6-morpholin-4-yl-[1,3,5]triazin- 2-yloxy}-acetic acid ethyl ester |
| 38 | 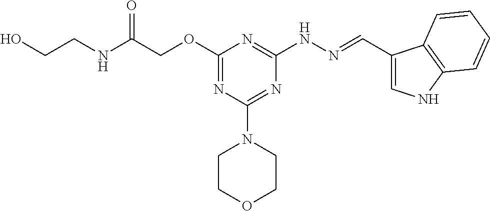 [SMILES: O=C(COc1nc(N/N=C/c2c[nH]c3ccccc23)nc(N2CCOCC2)n1)NCCO] | N-(2-Hydroxy-ethyl)-2-{4-[N′- (1H-indol-3-ylmethylene)- hydrazino]-6-morpholin-4-yl- [1,3,5]triazin-2-yloxy}-acetamide |
| 39 | 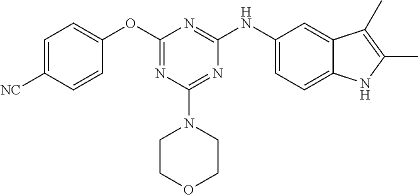 [SMILES: Cc1[nH]c2ccc(Nc3nc(Oc4ccc(C#N)cc4)nc(N4CCOCC4)n3)cc2c1C] | 4-[4-(2,3-Dimethyl-1H- indol-5-ylamino)-6- morpholin-4-yl-[1,3,5]triazin- 2-yloxy]-benzonitrile |
| 40 | 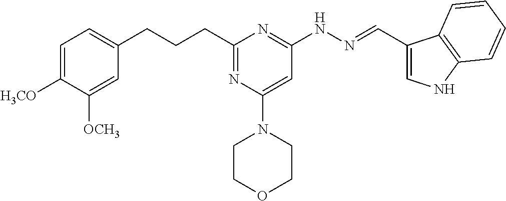 [SMILES: COc1ccc(CCCc2nc(N/N=C/c3c[nH]c4ccccc34)cc(N3CCOCC3)n2)cc1OC] | N-{2-[3-(3,4-Dimethoxy- phenyl)-propyl]-6- morpholin-4-yl-pyrimidin-4-yl}- N′-(1H-indol-3-ylmethylene)- hydrazine |
| 41 | 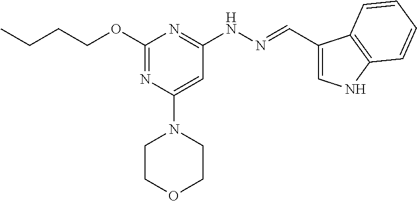 [SMILES: CCCCOc1nc(N/N=C/c2c[nH]c3ccccc23)cc(N2CCOCC2)n1] | N-(2-Butoxy-6-morpholin- 4-yl-pyrimidin-4-yl)- N′-(1H-indol-3-ylmethylene)- hydrazine |
| 42 | 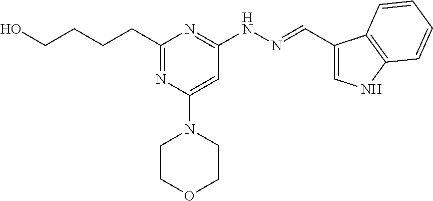 [SMILES: OCCCCc1nc(N/N=C/c2c[nH]c3ccccc23)cc(N2CCOCC2)n1] | 4-{4-[N′-(1H-indol- 3-ylmethylene)-hydrazino]- 6-morpholin-4-yl-pyrimidin- 2-yl}-butan-1-ol |
| 43 | 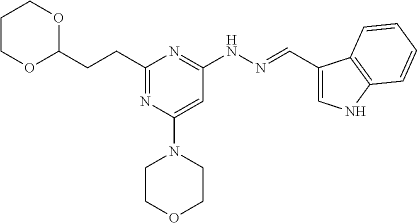 [SMILES: C(=N\Nc1cc(N2CCOCC2)nc(CCC2OCCCO2)n1)/c1c[nH]c2ccccc12] | N-[2-(2-[1,3]Dioxan-2-yl-ethyl)- 6-morpholin-4-yl-pyrimidin-4-yl]- N′-(1H-indol-3-ylmethylene)- hydrazine |
| 44 | 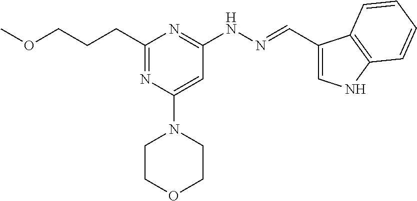 [SMILES: COCCCc1nc(N/N=C/c2c[nH]c3ccccc23)cc(N2CCOCC2)n1] | N-(1H-Indol-3-ylmethylene)- N′-[2-(3-methoxy-propyl)- 6-morpholin-4-yl-pyrimidin- 4-yl]-hydrazine |
| 45 | 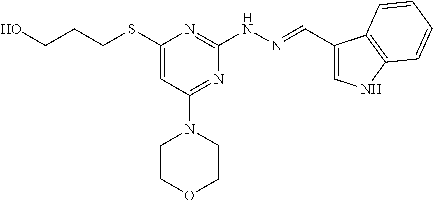 [SMILES: OCCCSc1cc(N2CCOCC2)nc(N/N=C/c2c[nH]c3ccccc23)n1] | 3-{2-[N′-(1H-Indol-3- ylmethylene)-hydrazino]-6- morpholin-4-yl-pyrimidin-4- ylsulfanyl}-propan-1-ol |
| 46 | 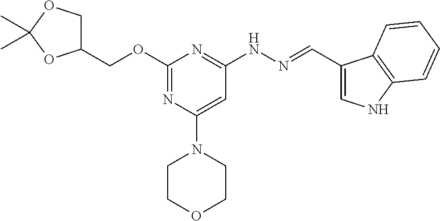 [SMILES: CC1(C)OCC(COc2nc(N/N=C/c3c[nH]c4ccccc34)cc(N3CCOCC3)n2)O1] | N-[2-(2,2-Dimethyl- [1,3]dioxolan-4-ylmethoxy)- 6-morpholin-4-yl-pyrimidin- 4-yl]-N′-(1H-indol-3- ylmethylene)-hydrazine |
| 47 | 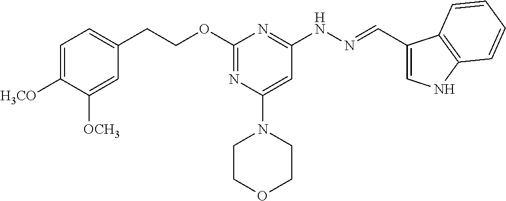 [SMILES: COc1ccc(CCOc2nc(N/N=C/c3c[nH]c4ccccc34)cc(N3CCOCC3)n2)cc1OC] | N-{2-[2-(3,4-Dimethoxy- phenyl)-ethoxy]-6- morpholin-4-yl-pyrimidin-4-yl}- N′-(1H-indol-3-ylmethylene)- hydrazine |
| 48 | 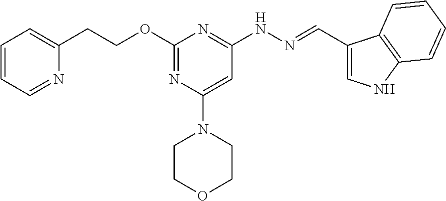 [SMILES: C(=N\Nc1cc(N2CCOCC2)nc(OCCc2ccccn2)n1)/c1c[nH]c2ccccc12] | N-(1H-Indol-3-ylmethylene)- N′-[6-morpholin-4-yl-2-(2- pyridin-2-yl-ethoxy)- pyrimidin-4-yl]-hydrazine |
| 49 | 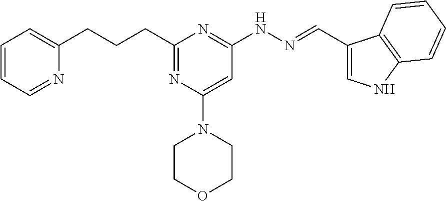 [SMILES: C(=N\Nc1cc(N2CCOCC2)nc(CCCc2ccccn2)n1)/c1c[nH]c2ccccc12] | N-(1H-Indol-3-ylmethylene)- N′-[6-morpholin-4-yl-2-(3- pyridin-2-yl-propyl)- pyrimidin-4-yl]-hydrazine |
| 50 | 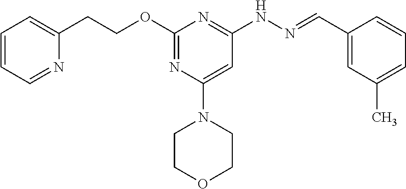 [SMILES: Cc1cccc(/C=N/Nc2cc(N3CCOCC3)nc(OCCc3ccccn3)n2)c1] | N-(3-Methyl-benzylidene)- N′-[6-morpholin-4-yl-2-(2- pyridin-2-yl-ethoxy)- pyrimidin-4-yl]-hydrazine |
| 60 | 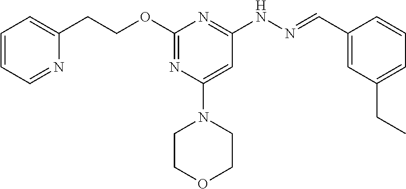 [SMILES: CCc1cccc(/C=N/Nc2cc(N3CCOCC3)nc(OCCc3ccccn3)n2)c1] | N-(3-Ethyl-benzylidene)- N′-[6-morpholin-4-yl-2-(2- pyridin-2-yl-ethoxy)- pyrimidin-4-yl]-hydrazine |
| 61 | 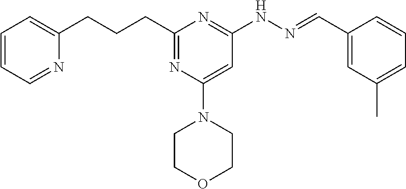 [SMILES: Cc1cccc(/C=N/Nc2cc(N3CCOCC3)nc(CCCc3ccccn3)n2)c1] | N-(3-Methyl-benzylidene)- N′-[6-morpholin-4-yl-2-(3- pyridin-2-yl-propyl)- pyrimidin-4-yl]-hydrazine |
| 62 | 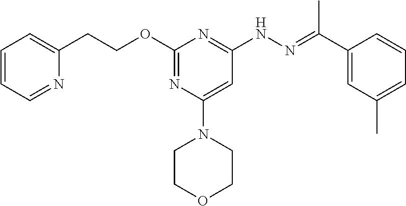 [SMILES: C/C(=N\Nc1cc(N2CCOCC2)nc(OCCc2ccccn2)n1)c1cccc(C)c1] | N-[6-Morpholin-4-yl-2-(2- pyridin-2-yl-ethoxy)- pyrimidin-4-yl]-N′-(1-m- tolyl-ethylidene)-hydrazine |
| 63 | 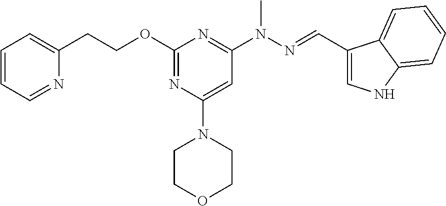 [SMILES: CN(/N=C/c1c[nH]c2ccccc12)c1cc(N2CCOCC2)nc(OCCc2ccccn2)n1] | N′-(1H-Indol-3-ylmethylene)- N-methyl-N-[6-morpholin-4- yl-2-(2-pyridin-2-yl-ethoxy)- pyrimidin-4-yl]-hydrazine |
| 64 | 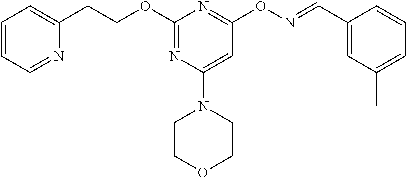 [SMILES: Cc1cccc(/C=N/Oc2cc(N3CCOCC3)nc(OCCc3ccccn3)n2)c1] | 3-Methyl-benzaldehyde O-[6-morpholin-4-yl-2-(2- pyridin-2-yl-ethoxy)- pyrimidin-4-yl]-oxime |
| 65 | 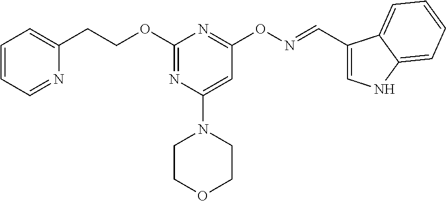 [SMILES: C(=N\Oc1cc(N2CCOCC2)nc(OCCc2ccccn2)n1)/c1c[nH]c2ccccc12] | 1H-Indole-3-carbaldehyde O-[6-morpholin-4-yl-2-(2- pyridin-2-yl-ethoxy)- pyrimidin-4-yl]-oxime |
| 66 | 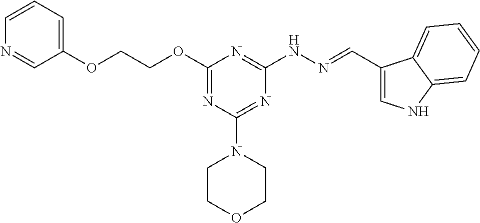 [SMILES: C(=N\Nc1nc(OCCOc2cccnc2)nc(N2CCOCC2)n1)/c1c[nH]c2ccccc12] | N-(1H-Indol-3-ylmethylene)- N′-{6-morpholin-4-yl-2-[2- (pyridin-3-yloxy)-ethoxy]- pyrimidin-4-yl}-hydrazine |
| 67 | 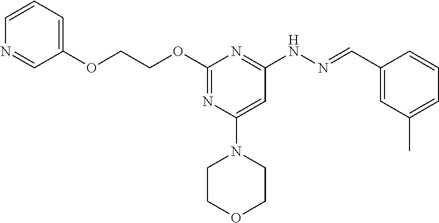 [SMILES: Cc1cccc(/C=N/Nc2cc(N3CCOCC3)nc(OCCOc3cccnc3)n2)c1] | N-(3-Methyl-benzylidene)- N′-{6-morpholin-4-yl-2-[2- (pyridin-3-yloxy)-ethoxy]- pyrimidin-4-yl}-hydrazine |
| 68 | 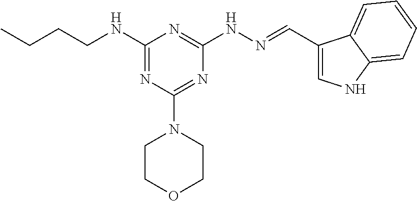 [SMILES: CCCCNc1nc(N/N=C/c2c[nH]c3ccccc23)nc(N2CCOCC2)n1] | Butyl-{4-[N′-(1H-indol-3- ylmethylene)-hydrazino]- 6-morpholin-4-yl-pyrimidin- 2-yl}-amine |
| 69 | 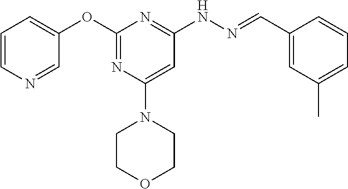 [SMILES: Cc1cccc(/C=N/Nc2cc(N3CCOCC3)nc(Oc3cccnc3)n2)c1] | N-(3-Methyl-benzylidene)- N′-[6-morpholin-4-yl-2- (pyridin-3-yloxy)- pyrimidin-4-yl]-hydrazine |
| 70 | 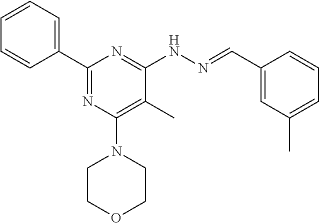 [SMILES: Cc1cccc(/C=N/Nc2nc(-c3ccccc3)nc(N3CCOCC3)c2C)c1] | N-(3-Methyl-benzylidene)- N′-(5-methyl-6-morpholin- 4-yl-2-phenyl-pyrimidin- 4-yl)-hydrazine |
| 71 | 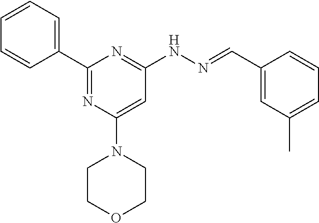 [SMILES: Cc1cccc(/C=N/Nc2cc(N3CCOCC3)nc(-c3ccccc3)n2)c1] | N-(3-Methyl-benzylidene)- N′-(6-morpholin-4-yl-2- phenyl-pyrimidin-4-yl)- hydrazine |
| 72 | 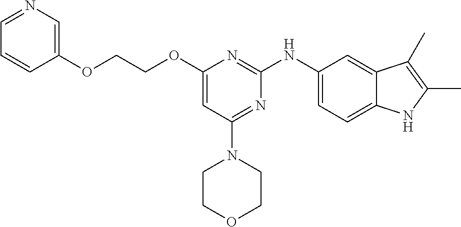 [SMILES: Cc1[nH]c2ccc(Nc3nc(OCCOc4cccnc4)cc(N4CCOCC4)n3)cc2c1C] | (2,3-Dimethyl-1H-indol- 5-yl)-{4-morpholin-4-yl-6- [2-(pyridin-3-yloxy)-ethoxy]- pyrimidin-2-yl}-amine |
| 73 | 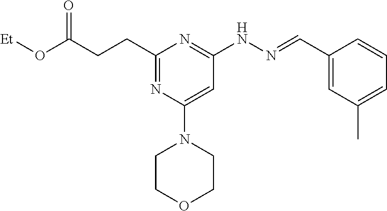 [SMILES: CCOC(=O)CCc1nc(N/N=C/c2cccc(C)c2)cc(N2CCOCC2)n1] | 3-{4-[N′-(3-Methyl- benzylidene)-hydrazino]-6- morpholin-4-yl-pyrimidin-2- yl}-propionic acid ethyl ester |
| 74 | 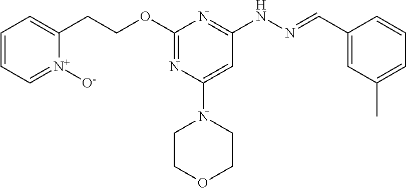 [SMILES: Cc1cccc(/C=N/Nc2cc(N3CCOCC3)nc(OCCc3cccc[n+]3[O-])n2)c1] | N-(3-Methyl-benzylidene)- N′-{6-morpholin-4-yl-2-[2- (1-oxy-pyridin-2-yl)-ethoxy]- pyrimidin-4-yl}-hydrazine |
| 75 | 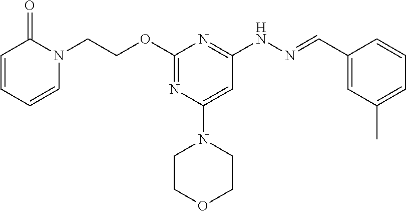 [SMILES: Cc1cccc(/C=N/Nc2cc(N3CCOCC3)nc(OCCn3ccccc3=O)n2)c1] | 1-(2-{4-[N′-(3-Methyl- benzylidene)-hydrazino]-6- morpholin-4-yl-pyrimidin- 2-yloxy}-ethyl)-1H- pyridin-2-one |
| 76 | 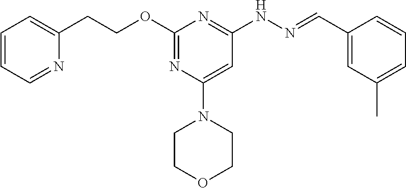 [SMILES: Cc1cccc(/C=N/Nc2cc(N3CCOCC3)nc(OCCc3ccccn3)n2)c1] | N-(3-Iodo-benzylidene)- N′-[6-morpholin-4-yl-2- (2-pyridin-2-yl-ethoxy)- pyrimidin-4-yl]-hydrazine |
| 77 | 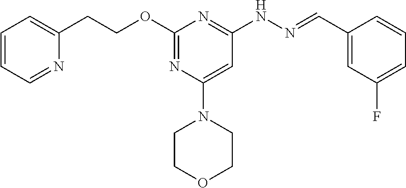 [SMILES: Fc1cccc(/C=N/Nc2cc(N3CCOCC3)nc(OCCc3ccccn3)n2)c1] | N-(3-Fluoro-benzylidene)- N′-[6-morpholin-4-yl-2-(2- pyridin-2-yl-ethoxy)- pyrimidin-4-yl]-hydrazine |
| 78 | 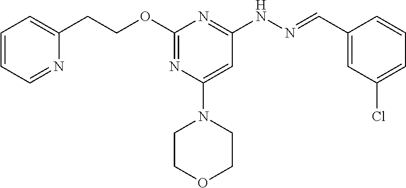 [SMILES: Clc1cccc(/C=N/Nc2cc(N3CCOCC3)nc(OCCc3ccccn3)n2)c1] | N-(3-Chloro-benzylidene)- N′-[6-morpholin-4-yl-2-(2- pyridin-2-yl-ethoxy)- pyrimidin-4-yl]-hydrazine |
| 79 | 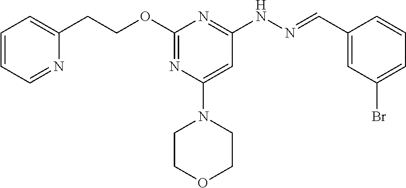 [SMILES: Brc1cccc(/C=N/Nc2cc(N3CCOCC3)nc(OCCc3ccccn3)n2)c1] | N-(3-Bromo-benzylidene)- N′-[6-morpholin-4-yl-2-(2- pyridin-2-yl-ethoxy)- pyrimidin-4-yl]-hydrazine |
| 80 | 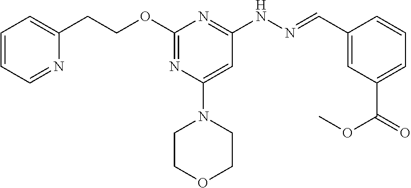 [SMILES: COC(=O)c1cccc(/C=N/Nc2cc(N3CCOCC3)nc(OCCc3ccccn3)n2)c1] | 3-{[6-Morpholin-4-yl-2- (2-pyridin-2-yl-ethoxy)- pyrimidin-4-yl]- hydrazonomethyl}-benzoic acid methyl ester |
| 81 | 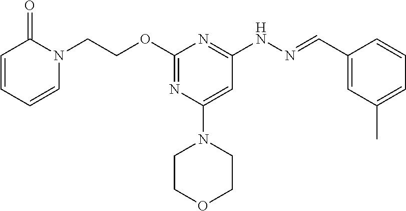 [SMILES: Cc1cccc(/C=N/Nc2cc(N3CCOCC3)nc(OCCn3ccccc3=O)n2)c1] | 1-(2-{4-[N′-(3-Iodo- benzylidene)-hydrazino]- 6-morpholin-4-yl- pyrimidin-2-yloxy}-ethyl)- 1H-pyridin-2-one |
| 82 | 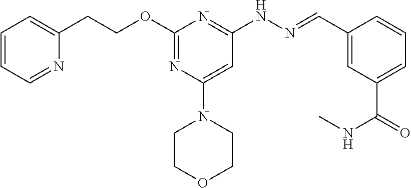 [SMILES: CNC(=O)c1cccc(/C=N/Nc2cc(N3CCOCC3)nc(OCCc3ccccn3)n2)c1] | N-Methyl-3-{[6-morpholin- 4-yl-2-(2-pyridin-2-yl- ethoxy)-pyrimidin-4-yl]- hydrazonomethyl}-benzamide |
| 83 | 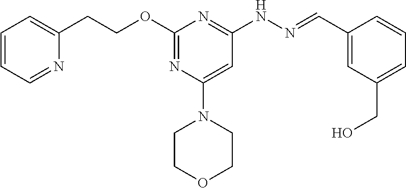 [SMILES: OCc1cccc(/C=N/Nc2cc(N3CCOCC3)nc(OCCc3ccccn3)n2)c1] | (3-{[6-Morpholin-4-yl-2- (2-pyridin-2-yl-ethoxy)- pyrimidin-4-yl]- hydrazonomethyl}-phenyl)- methanol |
| 84 | 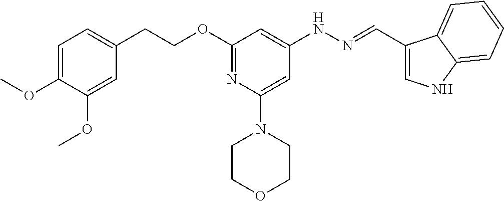 [SMILES: COc1ccc(CCOc2cc(N/N=C/c3c[nH]c4ccccc34)cc(N3CCOCC3)n2)cc1OC] | N-{2-[2-(3,4-Dimethoxy- phenyl)-ethoxy]-6- morpholin-4-yl-pyridin-4- yl}-N′-(1H-indol-3- ylmethylene)-hydrazine |
| 85 | 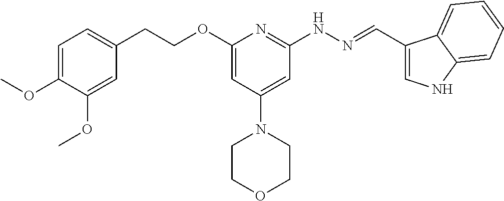 [SMILES: COc1ccc(CCOc2cc(N3CCOCC3)cc(N/N=C/c3c[nH]c4ccccc34)n2)cc1OC] | N-{6-[2-(3,4-Dimethoxy- phenyl)-ethoxy]-4- morpholin-4-yl-pyridin- 2-yl}-N′-(1H-indol-3- ylmethylene)-hydrazine |
| 86 | 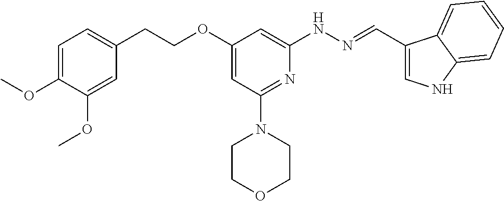 [SMILES: COc1ccc(CCOc2cc(N/N=C/c3c[nH]c4ccccc34)nc(N3CCOCC3)c2)cc1OC] | N-{4-[2-(3,4-Dimethoxy- phenyl)-ethoxy]-6- morpholin-4-yl-pyridin- 2-yl}-N′-(1H-indol-3- ylmethylene)-hydrazine |
| 87 | 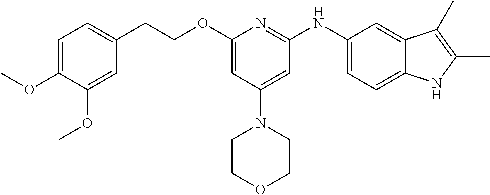 | {6-[2-(3,4-Dimethoxy- phenyl)-ethoxy]-4- morpholin-4-yl-pyridin- 2-yl}-(2,3-dimethyl- 1H-indol-5-yl)-amine |
| 88 | 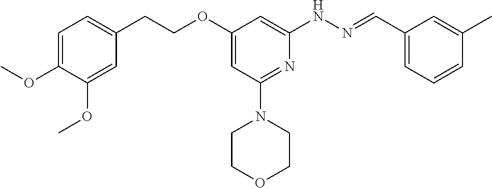 | N-{4-[2-(3,4-Dimethoxy- phenyl)-ethoxy]-6- morpholin-4-yl-pyridin- 2-yl}-N′-(3-methyl- benzylidene)-hydrazine |
| 89 | 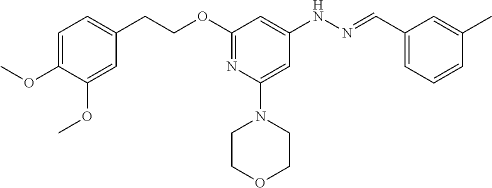 | N-{2-[2-(3,4-Dimethoxy- phenyl)-ethoxy]-6- morpholin-4-yl-pyridin- 4-yl}-N′-(3-methyl- benzylidene)-hydrazine |
| 90 | 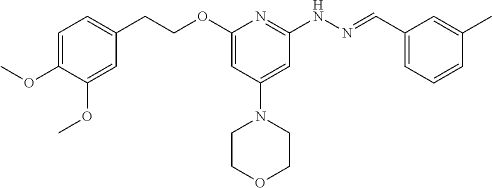 | N-{6-[2-(3,4-Dimethoxy- phenyl)-ethoxy]-4- morpholin-4-yl-pyridin- 2-yl}-N′-(3-methyl- benzylidene)-hydrazine |
| 91 | 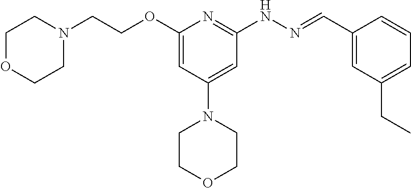 [SMILES: CCc1cccc(/C=N/Nc2cc(N3CCOCC3)cc(OCCN3CCOCC3)n2)c1] | N-(3-Ethyl-benzylidene)-N′-[4- morpholin-4-yl-6-(2- morpholin-4-yl-ethoxy)- pyridin-2-yl]-hydrazine |
| 92 | 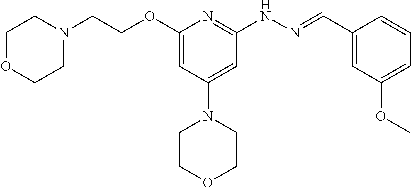 [SMILES: COc1cccc(/C=N/Nc2cc(N3CCOCC3)cc(OCCN3CCOCC3)n2)c1] | N-(3-Methoxy-benzylidene)- N′-[4-morpholin-4-yl-6-(2- morpholin-4-yl-ethoxy)- pyridin-2-yl]-hydrazine |
| 93 | 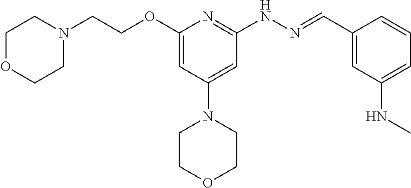 [SMILES: CNc1cccc(/C=N/Nc2cc(N3CCOCC3)cc(OCCN3CCOCC3)n2)c1] | Methyl-(3-{[4-morpholin-4-yl- 6-(2-morpholin-4-yl-ethoxy)- pyridin-2-yl]- hydrazonomethyl}-phenyl)- amine |
| 94 | 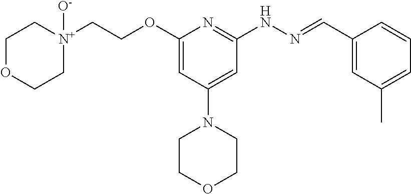 [SMILES: Cc1cccc(/C=N/Nc2cc(N3CCOCC3)cc(OCC[N+]3([O-])CCOCC3)n2)c1] | N-(3-Methyl-benzylidene)-N′- {4-morpholin-4-yl-6-[2-(4-oxy- morpholin-4-yl)-ethoxy]- pyridin-2-yl}-hydrazine |
| 95 | 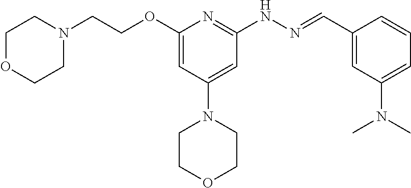 [SMILES: CN(C)c1cccc(/C=N/Nc2cc(N3CCOCC3)cc(OCCN3CCOCC3)n2)c1] | Dimethyl-(3-{[4-morpholin-4- yl-6-(2-morpholin-4-yl- ethoxy)-pyridin-2-yl]- hydrazonomethyl}-phenyl)- amine |
| 96 | 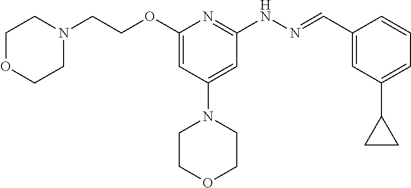 [SMILES: C(=N\Nc1cc(N2CCOCC2)cc(OCCN2CCOCC2)n1)/c1cccc(C2CC2)c1] | N-(3-Cyclopropyl- benzylidene)-N′-[4-morpholin- 4-yl-6-(2-morpholin-4-yl- ethoxy)-pyridin-2-yl]- hydrazine |
| 97 | 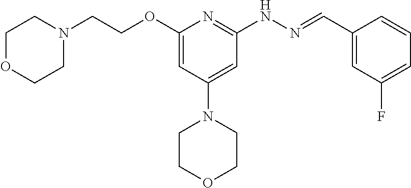 [SMILES: Fc1cccc(/C=N/Nc2cc(N3CCOCC3)cc(OCCN3CCOCC3)n2)c1] | N-(3-Fluoro-benzylidene)-N′- [4-morpholin-4-yl-6-(2- morpholin-4-yl-ethoxy)- pyridin-2-yl]-hydrazine |
| 98 | 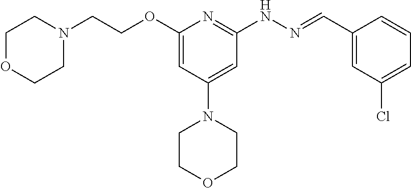 [SMILES: Clc1cccc(/C=N/Nc2cc(N3CCOCC3)cc(OCCN3CCOCC3)n2)c1] | N-(3-Chloro-benzylidene)-N′- [4-morpholin-4-yl-6-(2- morpholin-4-yl-ethoxy)- pyridin-2-yl]-hydrazine |
| 99 | 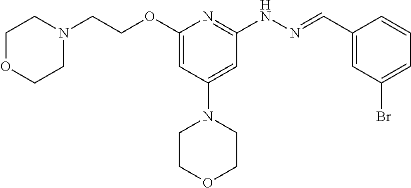 [SMILES: Brc1cccc(/C=N/Nc2cc(N3CCOCC3)cc(OCCN3CCOCC3)n2)c1] | N-(3-Bromo-benzylidene)-N′- [4-morpholin-4-yl-6-(2- morpholin-4-yl-ethoxy)- pyridin-2-yl]-hydrazine |
| 100 | 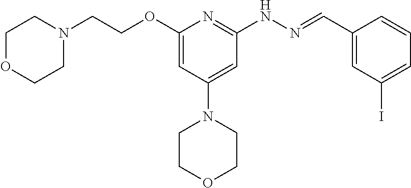 [SMILES: Ic1cccc(/C=N/Nc2cc(N3CCOCC3)cc(OCCN3CCOCC3)n2)c1] | N-(3-Iodo-benzylidene)-N′-[4- morpholin-4-yl-6-(2- morpholin-4-yl-ethoxy)- pyridin-2-yl]-hydrazine |
| 101 | 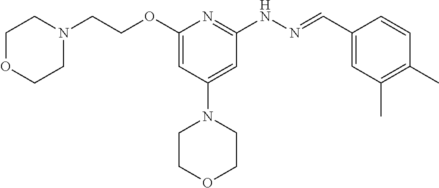 [SMILES: Cc1ccc(/C=N/Nc2cc(N3CCOCC3)cc(OCCN3CCOCC3)n2)cc1C] | N-(3,4-Dimethyl-benzylidene)- N′-[4-morpholin-4-yl-6-(2- morpholin-4-yl-ethoxy)- pyridin-2-yl]-hydrazine |
| 102 | 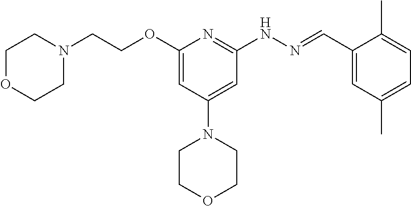 [SMILES: Cc1ccc(C)c(/C=N/Nc2cc(N3CCOCC3)cc(OCCN3CCOCC3)n2)c1] | N-(2,5-Dimethyl-benzylidene)- N′-[4-morpholin-4-yl-6-(2- morpholin-4-yl-ethoxy)- pyridin-2-yl]-hydrazine |
| 103 | 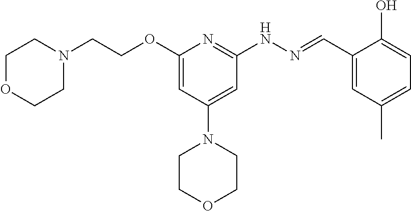 [SMILES: Cc1ccc(O)c(/C=N/Nc2cc(N3CCOCC3)cc(OCCN3CCOCC3)n2)c1] | 4-Methyl-2-{[4-morpholin-4- yl-6-(2-morpholin-4-yl- ethoxy)-pyridin-2-yl]- hydrazonomethyl}-phenol |
| 104 | 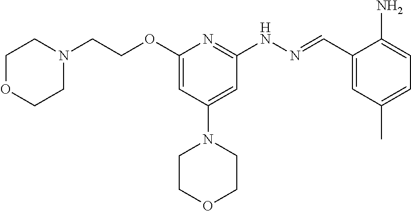 [SMILES: Cc1ccc(N)c(/C=N/Nc2cc(N3CCOCC3)cc(OCCN3CCOCC3)n2)c1] | 4-Methyl-2-{[4-morpholin-4- yl-6-(2-morpholin-4-yl- ethoxy)-pyridin-2-yl]- hydrazonomethyl}- phenylamine |
| 105 | 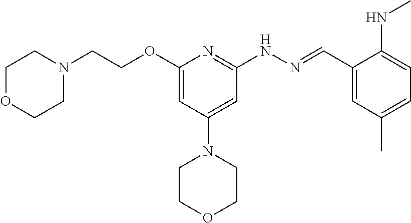 [SMILES: CNc1ccc(C)cc1/C=N/Nc1cc(N2CCOCC2)cc(OCCN2CCOCC2)n1] | Methyl-(4-methyl-2-{[4- morpholin-4-yl-6-(2- morpholin-4-yl-ethoxy)- pyridin-2-yl]- hydrazonomethyl}-phenyl)- amine |
| 106 | 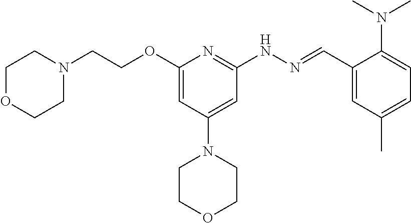 [SMILES: Cc1ccc(N(C)C)c(/C=N/Nc2cc(N3CCOCC3)cc(OCCN3CCOCC3)n2)c1] | Dimethyl-(4-methyl-2-{[4- morpholin-4-yl-6-(2- morpholin-4-yl-ethoxy)- pyridin-2-yl]- hydrazonomethyl}-phenyl)- amine |
| 107 | 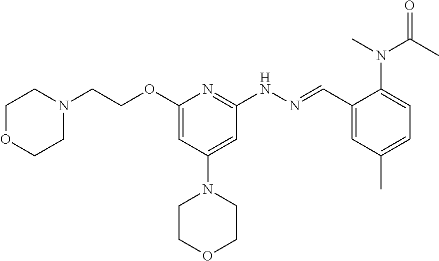 [SMILES: CC(=O)N(C)c1ccc(C)cc1/C=N/Nc1cc(N2CCOCC2)cc(OCCN2CCOCC2)n1] | N-Methyl-N-(4-methyl-2-{[4- morpholin-4-yl-6-(2- morpholin-4-yl-ethoxy)- pyridin-2-yl]- hydrazonomethyl}-phenyl)- acetamide |
| 108 | 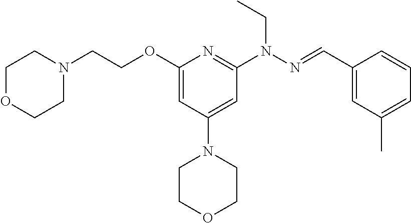 [SMILES: CCN(/N=C/c1cccc(C)c1)c1cc(N2CCOCC2)cc(OCCN2CCOCC2)n1] | N-Ethyl-N′-(3-methyl- benzylidene)-N-[4-morpholin- 4-yl-6-(2-morpholin-4-yl- ethoxy)-pyridin-2-yl]- hydrazine |
| 109 | 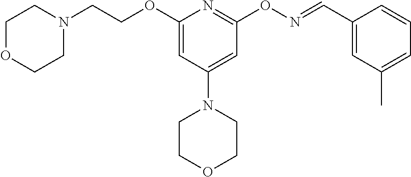 [SMILES: Cc1cccc(/C=N/Oc2cc(N3CCOCC3)cc(OCCN3CCOCC3)n2)c1] | 3-Methyl-benzaldehyde O-[4- morpholin-4-yl-6-(2- morpholin-4-yl-ethoxy)- pyridin-2-yl]-oxime |
| 110 | 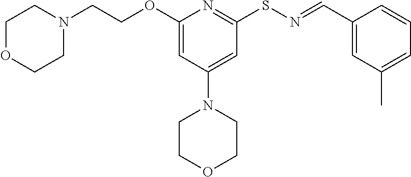 [SMILES: Cc1cccc(/C=N/Sc2cc(N3CCOCC3)cc(OCCN3CCOCC3)n2)c1] | 3-Methyl-benzaldehyde O-[4- morpholin-4-yl-6-(2- morpholin-4-yl-ethoxy)- pyridin-2-yl]-thiooxime |
| 111 | 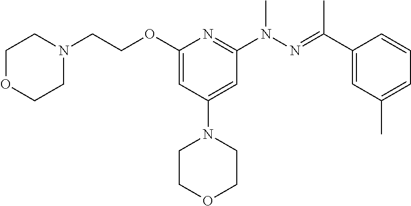 [SMILES: C/C(=N\N(C)c1cc(N2CCOCC2)cc(OCCN2CCOCC2)n1)c1cccc(C)c1] | N-Methyl-N-[4-morpholin-4- yl-6-(2-morpholin-4-yl- ethoxy)-pyridin-2-yl]-N′-(1-m- tolyl-ethylidene)-hydrazine |
| 112 | 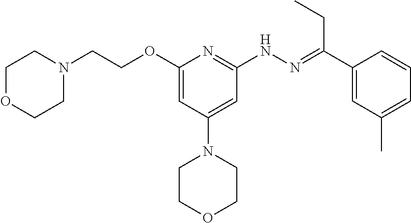 [SMILES: CC/C(=N\Nc1cc(N2CCOCC2)cc(OCCN2CCOCC2)n1)c1cccc(C)c1] | N-[4-Morpholin-4-yl-6-(2- morpholin-4-yl-ethoxy)- pyridin-2-yl]-N′-(1-m-tolyl- propylidene)-hydrazine |
| 113 | 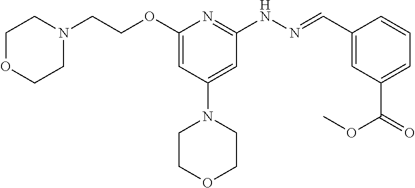 [SMILES: COC(=O)c1cccc(/C=N/Nc2cc(N3CCOCC3)cc(OCCN3CCOCC3)n2)c1] | 3-{[4-Morpholin-4-yl-6-(2- morpholin-4-yl-ethoxy)- pyridin-2-yl]- hydrazonomethyl}-benzoic acid methyl ester |
| 114 | 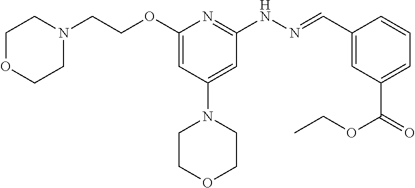 [SMILES: CCOC(=O)c1cccc(/C=N/Nc2cc(N3CCOCC3)cc(OCCN3CCOCC3)n2)c1] | 3-{[4-Morpholin-4-yl-6-(2- morpholin-4-yl-ethoxy)- pyridin-2-yl]- hydrazonomethyl}-benzoic acid ethyl ester |
| 115 | 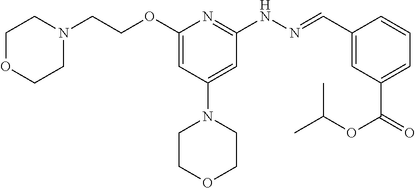 [SMILES: CC(C)OC(=O)c1cccc(/C=N/Nc2cc(N3CCOCC3)cc(OCCN3CCOCC3)n2)c1] | 3-{[4-Morpholin-4-yl-6-(2- morpholin-4-yl-ethoxy)- pyridin-2-yl]- hydrazonomethyl}-benzoic acid isopropyl ester |
| 116 | 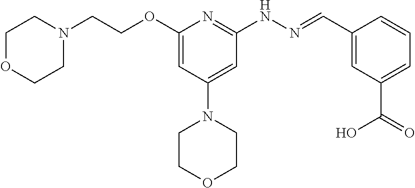 [SMILES: O=C(O)c1cccc(/C=N/Nc2cc(N3CCOCC3)cc(OCCN3CCOCC3)n2)c1] | 3-{[4-Morpholin-4-yl-6-(2- morpholin-4-yl-ethoxy)- pyridin-2-yl]- hydrazonomethyl}-benzoic acid |
| 117 | 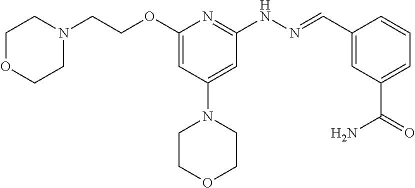 [SMILES: NC(=O)c1cccc(/C=N/Nc2cc(N3CCOCC3)cc(OCCN3CCOCC3)n2)c1] | 3-{[4-Morpholin-4-yl-6-(2- morpholin-4-yl-ethoxy)- pyridin-2-yl]- hydrazonomethyl}-benzamide |
| 118 | 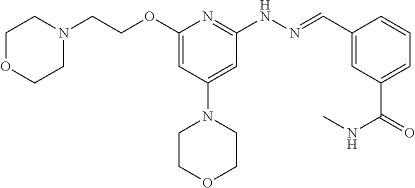 [SMILES: CNC(=O)c1cccc(/C=N/Nc2cc(N3CCOCC3)cc(OCCN3CCOCC3)n2)c1] | N-Methyl-3-{[4-morpholin-4- yl-6-(2-morpholin-4-yl- ethoxy)-pyridin-2-yl]- hydrazonomethyl}-benzamide |
| 119 | 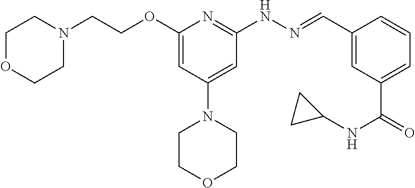 [SMILES: O=C(NC1CC1)c1cccc(/C=N/Nc2cc(N3CCOCC3)cc(OCCN3CCOCC3)n2)c1] | N-Cyclopropyl-3-{[4- morpholin-4-yl-6-(2- morpholin-4-yl-ethoxy)- pyridin-2-yl]- hydrazonomethyl}-benzamide |
| 120 | 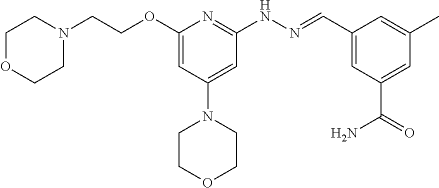 [SMILES: Cc1cc(/C=N/Nc2cc(N3CCOCC3)cc(OCCN3CCOCC3)n2)cc(C(N)=O)c1] | 3-Methyl-5-{[4-morpholin-4- yl-6-(2-morpholin-4-yl- ethoxy)-pyridin-2-yl]- hydrazonomethyl}-benzamide |
| 121 | 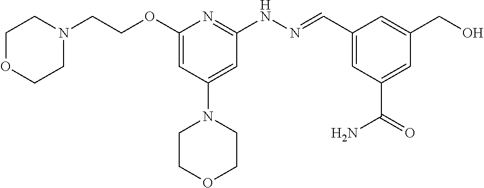 [SMILES: NC(=O)c1cc(/C=N/Nc2cc(N3CCOCC3)cc(OCCN3CCOCC3)n2)cc(CO)c1] | 3-Hydroxymethyl-5-{[4- morpholin-4-yl-6-(2- morpholin-4-yl-ethoxy)- pyridin-2-yl]- hydrazonomethyl}-benzamide |
| 122 | 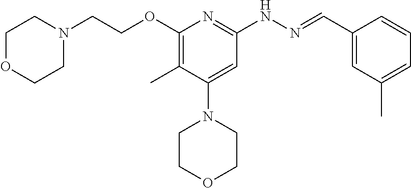 [SMILES: Cc1cccc(/C=N/Nc2cc(N3CCOCC3)c(C)c(OCCN3CCOCC3)n2)c1] | N-(3-Methyl-benzylidene)-N′- [5-methyl-4-morpholin-4-yl-6- (2-morpholin-4-yl-ethoxy)- pyridin-2-yl]-hydrazine |
| 123 | 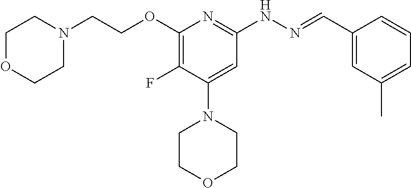 [SMILES: Cc1cccc(/C=N/Nc2cc(N3CCOCC3)c(F)c(OCCN3CCOCC3)n2)c1] | N-[5-Fluoro-4-morpholin-4-yl- 6-(2-morpholin-4-yl-ethoxy)- pyridin-2-yl]-N′-(3-methyl- benzylidene)-hydrazine |
| 124 | 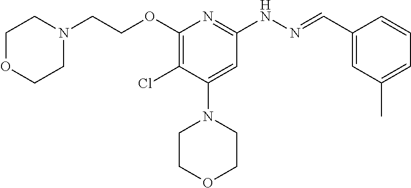 [SMILES: Cc1cccc(/C=N/Nc2cc(N3CCOCC3)c(Cl)c(OCCN3CCOCC3)n2)c1] | N-[5-Chloro-4-morpholin-4-yl- 6-(2-morpholin-4-yl-ethoxy)- pyridin-2-yl]-N′-(3-methyl- benzylidene)-hydrazine |
| 125 | 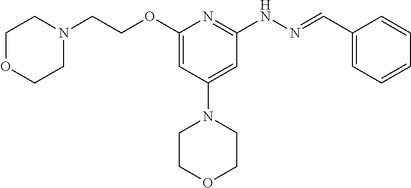 [SMILES: C(=N\Nc1cc(N2CCOCC2)cc(OCCN2CCOCC2)n1)/c1ccccc1] | N-Benzylidene-N′-[4- morpholin-4-yl-6-(2- morpholin-4-yl-ethoxy)- pyridin-2-yl]-hydrazine |
| 126 | 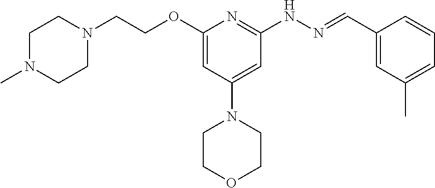 [SMILES: Cc1cccc(/C=N/Nc2cc(N3CCOCC3)cc(OCCN3CCN(C)CC3)n2)c1] | N-(3-Methyl-benzylidene-N′- {6-[2-(4-methyl-piperazin-1- yl)-ethoxy]-4-morpholin-4-yl- pyridin-2-yl}-hydrazine |
| 127 | 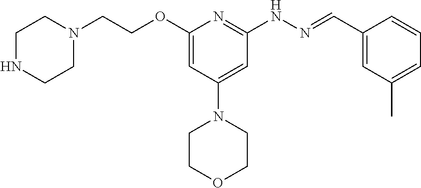 [SMILES: Cc1cccc(/C=N/Nc2cc(N3CCOCC3)cc(OCCN3CCNCC3)n2)c1] | N-(3-Methyl-benzylidene)-N′- [4-morpholin-4-yl-6-(2- piperazin-1-yl-ethoxy)-pyridin- 2-yl]-hydrazine |
| 128 | 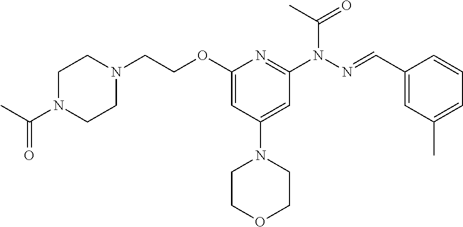 [SMILES: CC(=O)N1CCN(CCOc2cc(N3CCOCC3)cc(N(/N=C/c3cccc(C)c3)C(C)=O)n2)CC1] | Acetic acid N-{6-[2-(4-acetyl- piperazin-1-yl)-ethoxy]-4- morpholin-4-yl-pyridin-2-yl}- N′-(3-methyl-benzylidene)- hydrazide |
| 129 | 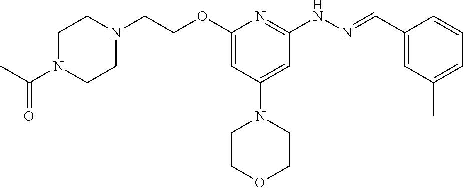 [SMILES: CC(=O)N1CCN(CCOc2cc(N3CCOCC3)cc(N/N=C/c3cccc(C)c3)n2)CC1] | 1-[4-(2-{6-[N′-(3-Methyl- benzylidene)-hydrazino]-4- morpholin-4-yl-pyridin-2- yloxy}-ethyl)-piperazin-1-yl]- ethanone |
| 130 | 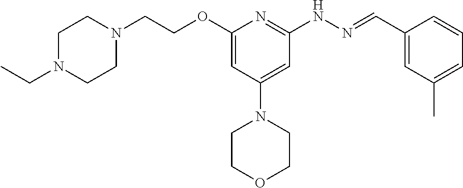 [SMILES: CCN1CCN(CCOc2cc(N3CCOCC3)cc(N/N=C/c3cccc(C)c3)n2)CC1] | N-{6-[2-(4-Ethyl-piperazin-1- yl)-ethoxy]-4-morpholin-4-yl- pyridin-2-yl}-N′-(3-methyl- benzylidene)-hydrazine |
| 131 | 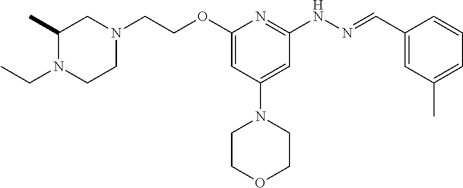 [SMILES: CCN1CCN(CCOc2cc(N3CCOCC3)cc(N/N=C/c3cccc(C)c3)n2)C[C@@H]1C] | N-{6-[2-(4-Ethyl-3-methyl- piperazin-1-yl)-ethoxy]-4- morpholin-4-yl-pyridin-2-yl}- N′-(3-methyl-benzylidene)- hydrazine |
| 132 | 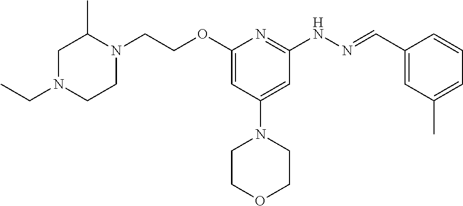 [SMILES: CCN1CCN(CCOc2cc(N3CCOCC3)cc(N/N=C/c3cccc(C)c3)n2)C(C)C1] | N-{6-[2-(4-Ethyl-2-methyl- piperazin-1-yl)-ethoxy]-4- morpholin-4-yl-pyridin-2-yl}- N′-(3-methyl-benzylidene)- hydrazine |
| 133 | 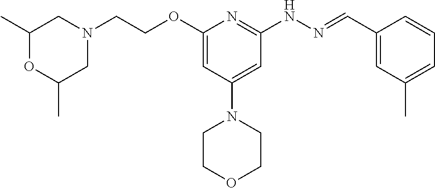 [SMILES: Cc1cccc(/C=N/Nc2cc(N3CCOCC3)cc(OCCN3CC(C)OC(C)C3)n2)c1] | N-{6-[2-(2,6-Dimethyl- morpholin-4-yl)-ethoxy]-4- morpholin-4-yl-pyridin-2-yl}- N′-(3-methyl-benzylidene)- hydrazine |
| 134 | 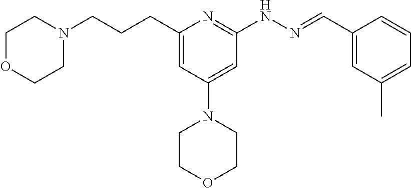 [SMILES: Cc1cccc(/C=N/Nc2cc(N3CCOCC3)cc(CCCN3CCOCC3)n2)c1] | N-(3-Methyl-benzylidene)-N′- [4-morpholin-4-yl-6-(3- morpholin-4-yl-propyl)- pyridin-2-yl]-hydrazine |
| 135 | 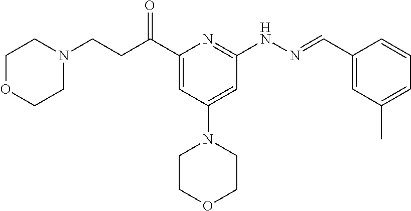 [SMILES: Cc1cccc(/C=N/Nc2cc(N3CCOCC3)cc(C(=O)CCN3CCOCC3)n2)c1] | 1-{6-[N′-(3-Methyl- benzylidene)-hydrazino]-4- morpholin-4-yl-pyridin-2-yl}- 3-morpholin-4-yl-propan-1-one |
| 136 | 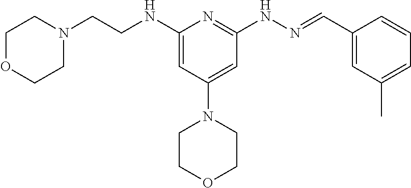 [SMILES: Cc1cccc(/C=N/Nc2cc(N3CCOCC3)cc(NCCN3CCOCC3)n2)c1] | {6-[N′-(3-Methyl-benzylidene)- hydrazino]-4-morpholin-4-yl- pyridin-2-yl}-(2-morpholin-4- yl-ethyl)-amine |
| 137 | 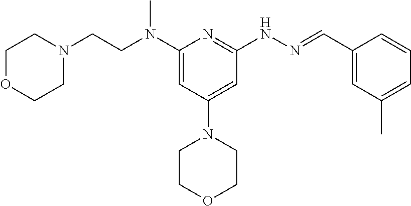 [SMILES: Cc1cccc(/C=N/Nc2cc(N3CCOCC3)cc(N(C)CCN3CCOCC3)n2)c1] | Methyl-{6-[N′-(3-methyl- benzylidene)-hydrazino]-4- morpholin-4-yl-pyridin-2-yl}- (2-morpholin-4-yl-ethyl)-amine |
| 138 | 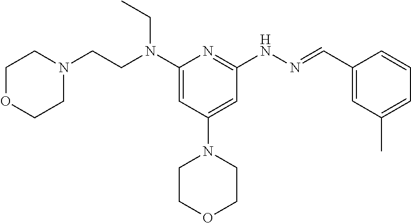 [SMILES: CCN(CCN1CCOCC1)c1cc(N2CCOCC2)cc(N/N=C/c2cccc(C)c2)n1] | Ethyl-{6-[N′-(3-methyl- benzylidene)-hydrazino]-4- morpholin-4-yl-pyridin-2-yl}- (2-morpholin-4-yl-ethyl)-amine |
| 139 | 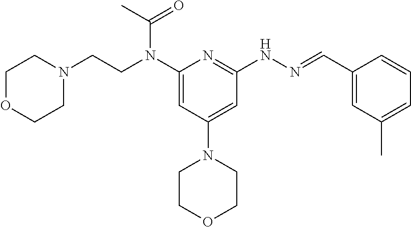 [SMILES: CC(=O)N(CCN1CCOCC1)c1cc(N2CCOCC2)cc(N/N=C/c2cccc(C)c2)n1] | N-{6-[N′-(3-Methyl- benzylidene)-hydrazino]-4- morpholin-4-yl-pyridin-2-yl}- N-(2-morpholin-4-yl-ethyl)- acetamide |
| 140 | 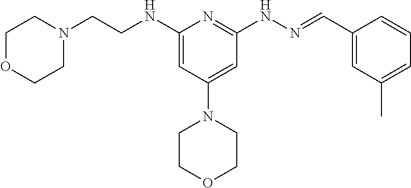 [SMILES: Cc1cccc(/C=N/Nc2cc(N3CCOCC3)cc(NCCN3CCOCC3)n2)c1] | N-{6-[N′-(3-Methyl- benzylidene)-hydrazino]-4- morpholin-4-yl-pyridin-2-yl}- 2-morpholin-4-yl-acetamide |
| 141 | 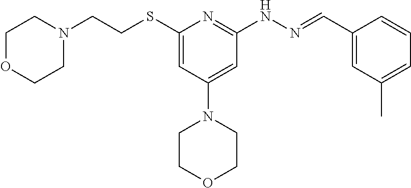 [SMILES: Cc1cccc(/C=N/Nc2cc(N3CCOCC3)cc(SCCN3CCOCC3)n2)c1] | N-(3-Methyl-benzylidene)-N′- [4-morpholin-4-yl-6-(2- morpholin-4-yl-ethylsulfanyl)- pyridin-2-yl]-hydrazine |
| 142 | 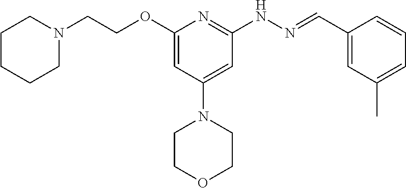 [SMILES: Cc1cccc(/C=N/Nc2cc(N3CCOCC3)cc(OCCN3CCCCC3)n2)c1] | N-(3-Methyl-benzylidene)-N′- [4-morpholin-4-yl-6-(2- piperidin-1-yl-ethoxy)-pyridin- 2-yl]-hydrazine |
| 143 | 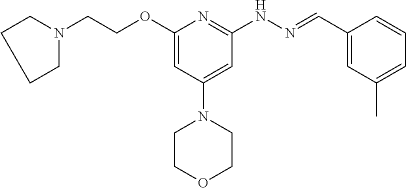 [SMILES: Cc1cccc(/C=N/Nc2cc(N3CCOCC3)cc(OCCN3CCCC3)n2)c1] | N-(3-Methyl-benzylidene)-N′- [4-morpholin-4-yl-6-(2- pyrrolidin-1-yl-ethoxy)- pyridin-2-yl]-hydrazine |
| 144 | 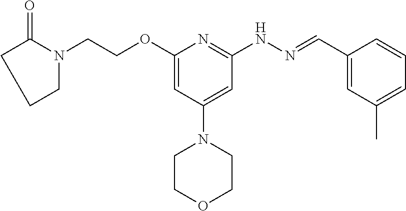 [SMILES: Cc1cccc(/C=N/Nc2cc(N3CCOCC3)cc(OCCN3CCCC3=O)n2)c1] | 1-(2-{6-[N′-(3-Methyl- benzylidene)-hydrazino]-4- morpholin-4-yl-pyridin-2- yloxy}-ethyl)-pyrrolidin-2-one |
| 145 | 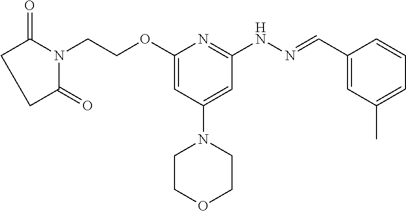 [SMILES: Cc1cccc(/C=N/Nc2cc(N3CCOCC3)cc(OCCN3C(=O)CCC3=O)n2)c1] | 1-(2-{6-[N′-(3-Methyl- benzylidene)-hydrazino]-4- morpholin-4-yl-pyridin-2- yloxy}-ethyl)-pyrrolidine-2,4- dione |
| 146 | 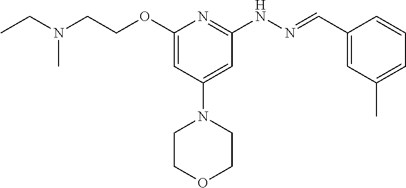 [SMILES: CCN(C)CCOc1cc(N2CCOCC2)cc(N/N=C/c2cccc(C)c2)n1] | Ethyl-methyl-(2-{6-[N′-(3- methyl-benzylidene)- hydrazino]-4-morpholin-4-yl- pyridin-2-yloxy}-ethyl)-amine |
| 147 | 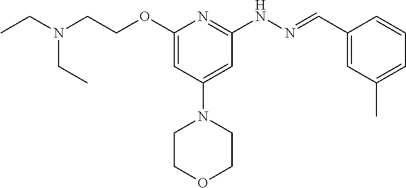 [SMILES: CCN(CC)CCOc1cc(N2CCOCC2)cc(N/N=C/c2cccc(C)c2)n1] | Diethyl-(2-{6-[N′-(3-methyl- benzylidene)-hydrazino]-4- morpholin-4-yl-pyridin-2- yloxy}-ethyl)-amine |
| 148 | 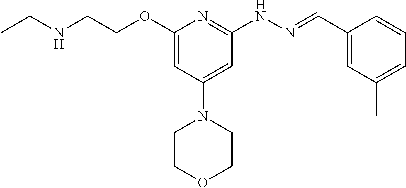 [SMILES: CCNCCOc1cc(N2CCOCC2)cc(N/N=C/c2cccc(C)c2)n1] | Ethyl-(2-{6-[N′-(3-methyl- benzylidene)-hydrazino]-4- morpholin-4-yl-pyridin-2- yloxy}-ethyl)-amine |
| 149 | 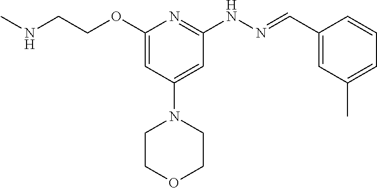 [SMILES: CNCCOc1cc(N2CCOCC2)cc(N/N=C/c2cccc(C)c2)n1] | Methyl-(2-{6-[N′-(3-methyl- benzylidene)-hydrazino]-4- morpholin-4-yl-pyridin-2- yloxy}-ethyl)-amine |
| 150 | 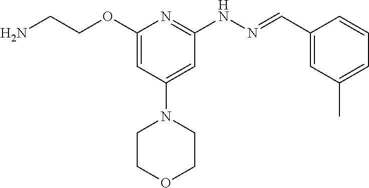 [SMILES: Cc1cccc(/C=N/Nc2cc(N3CCOCC3)cc(OCCN)n2)c1] | 2-{6-[N′-(3-Methyl- benzylidene)-hydrazino]-4- morpholin-4-yl-pyridin-2- yloxy}-ethylamine |
| 151 | 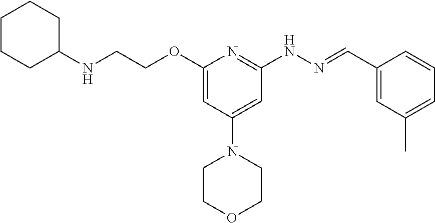 [SMILES: Cc1cccc(/C=N/Nc2cc(N3CCOCC3)cc(OCCNC3CCCCC3)n2)c1] | Cyclohexyl-(2-{6-[N′-(3- methyl-benzylidene)- hydrazino]-4-morpholin-4-yl- pyridin-2-yloxy}-ethyl)-amine |
| 152 | 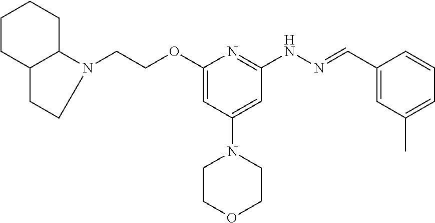 [SMILES: Cc1cccc(/C=N/Nc2cc(N3CCOCC3)cc(OCCN3CCC4CCCCC43)n2)c1] | N-(3-Methyl-benzylidene)-N′- {4-morpholin-4-yl-6-[2- (octahydro-indol-1-yl)-ethoxy]- pyridin-2-yl}-hydrazine |
| 153 | 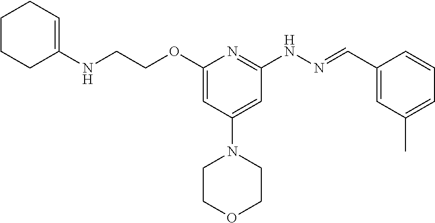 [SMILES: Cc1cccc(/C=N/Nc2cc(N3CCOCC3)cc(OCCNC3=CCCCC3)n2)c1] | Cyclohex-1-enyl-(2-{6-[N′-(3- methyl-benzylidene)- hydrazino]-4-morpholin-4-yl- pyridin-2-yloxy}-ethyl)-amine |
| 154 | 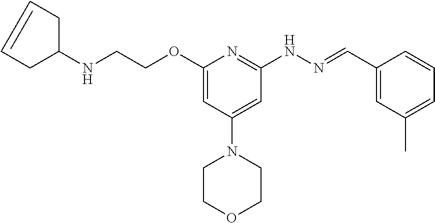 [SMILES: Cc1cccc(/C=N/Nc2cc(N3CCOCC3)cc(OCCNC3CC=CC3)n2)c1] | Cyclopent-3-enyl-(2-{6-[N′-(3- methyl-benzylidene)- hydrazino]-4-morpholin-4-yl- pyridin-2-yloxy}-ethyl)-amine |
| 155 | 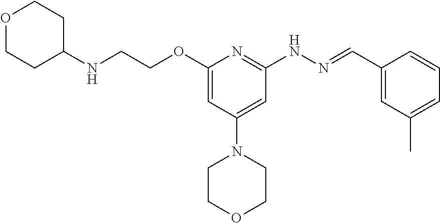 [SMILES: Cc1cccc(/C=N/Nc2cc(N3CCOCC3)cc(OCCNC3CCOCC3)n2)c1] | (2-{6-[N′-(3-Methyl- benzylidene)-hydrazino]-4- morpholin-4-yl-pyridin-2- yloxy}-ethyl)-(tetrahydro- pyran-4-yl)-amine |
| 156 | 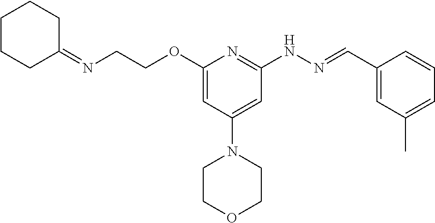 [SMILES: Cc1cccc(/C=N/Nc2cc(N3CCOCC3)cc(OCCN=C3CCCCC3)n2)c1] | Cyclohexylidene-(2-{6-[N′-(3- methyl-benzylidene)- hydrazino]-4-morpholin-4-yl- pyridin-2-yloxy}-ethyl)-amine |
| 157 | 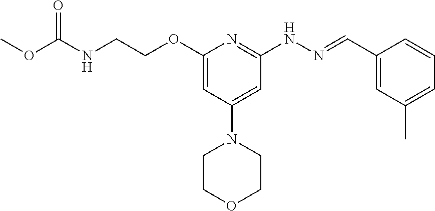 [SMILES: COC(=O)NCCOc1cc(N2CCOCC2)cc(N/N=C/c2cccc(C)c2)n1] | (2-{6-[N′-(3-Methyl- benzylidene)-hydrazino]-4- morpholin-4-yl-pyridin-2- yloxy}-ethyl)-carbamic acid methyl ester |
| 158 | 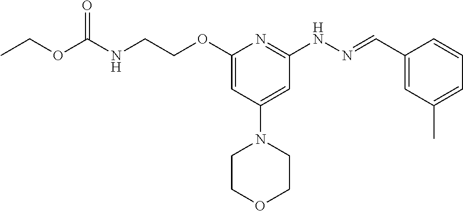 [SMILES: CCOC(=O)NCCOc1cc(N2CCOCC2)cc(N/N=C/c2cccc(C)c2)n1] | (2-{6-[N′-(3-Methyl- benzylidene)-hydrazino]-4- morpholin-4-yl-pyridin-2- yloxy}-ethyl)-carbamic acid ethyl ester |
| 159 | 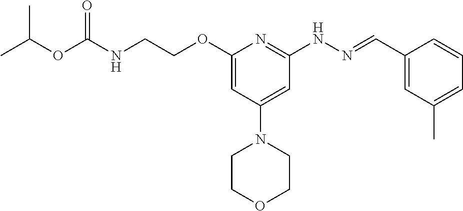 [SMILES: Cc1cccc(/C=N/Nc2cc(N3CCOCC3)cc(OCCNC(=O)OC(C)C)n2)c1] | (2-{6-[N′-(3-Methyl- benzylidene)-hydrazino]-4- morpholin-4-yl-pyridin-2- yloxy}-ethyl)-carbamic acid isopropyl ester |
| 160 | 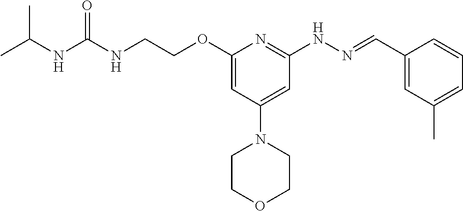 [SMILES: Cc1cccc(/C=N/Nc2cc(N3CCOCC3)cc(OCCNC(=O)NC(C)C)n2)c1] | 1-Isopropyl-3-(2-{6-[N′-(3- methyl-benzylidene)- hydrazino]-4-morpholin-4-yl- pyridin-2-yloxy}-ethyl)-urea |
| 161 | 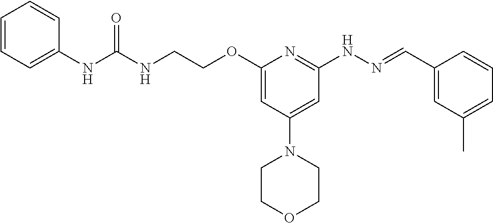 [SMILES: Cc1cccc(/C=N/Nc2cc(N3CCOCC3)cc(OCCNC(=O)Nc3ccccc3)n2)c1] | 1-(2-{6-[N′-(3-Methyl- benzylidene)-hydrazino]-4- morpholin-4-yl-pyridin-2- yloxy}-ethyl)-3-phenyl-urea |
| 162 | 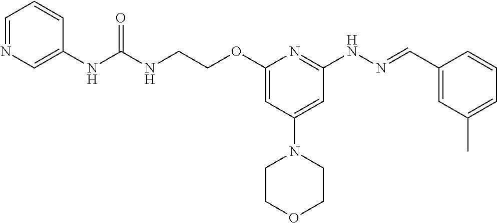 [SMILES: Cc1cccc(/C=N/Nc2cc(N3CCOCC3)cc(OCCNC(=O)Nc3cccnc3)n2)c1] | 1-(2-{6-[N′-(3-Methyl- benzylidene)-hydrazino]-4- morpholin-4-yl-pyridin-2- yloxy}-ethyl)-3-pyridin-3-yl- urea |
| 163 | 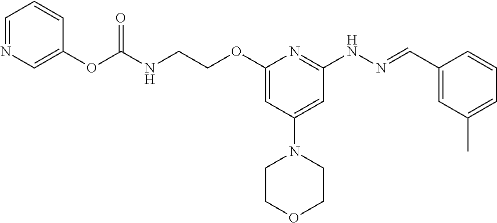 [SMILES: Cc1cccc(/C=N/Nc2cc(N3CCOCC3)cc(OCCNC(=O)Oc3cccnc3)n2)c1] | (2-{6-[N′-(3-Methyl- benzylidene)-hydrazino]-4- morpholin-4-yl-pyridin-2- yloxy}-ethyl)-carbamic acid pyridin-3-yl ester |
| 164 | 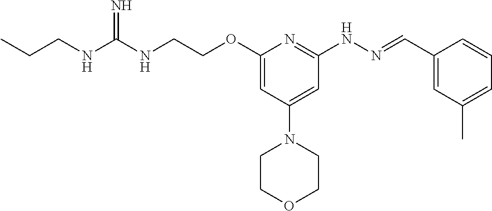 [SMILES: CCCNC(=N)NCCOc1cc(N2CCOCC2)cc(N/N=C/c2cccc(C)c2)n1] | N-(2-{6-[N′-(3-Methyl- benzylidene)-hydrazino]-4- morpholin-4-yl-pyridin-2- yloxy}-ethyl)-N′-propyl- guanidine |
| 165 | 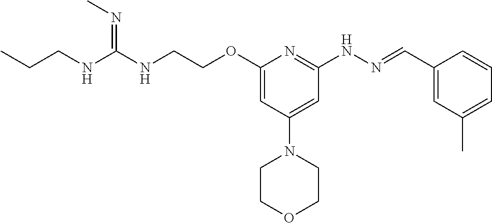 [SMILES: CCCN/C(=N\C)NCCOc1cc(N2CCOCC2)cc(N/N=C/c2cccc(C)c2)n1] | N-Methyl-N′-(2-{6-[N′-(3- methyl-benzylidene)- hydrazino]-4-morpholin-4-yl- pyridin-2-yloxy}-ethyl)-N″- propyl-guanidine |
| 166 | 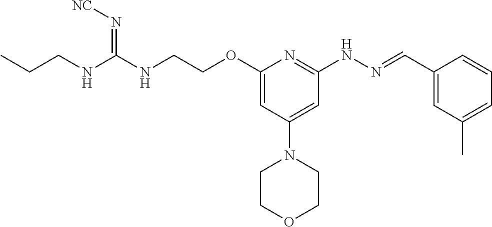 [SMILES: CCCN/C(=N\C#N)NCCOc1cc(N2CCOCC2)cc(N/N=C/c2cccc(C)c2)n1] | N-Cyano-N′-(2-{6-[N′-(3- methyl-benzylidene)- hydrazino]-4-morpholin-4-yl- pyridin-2-yloxy}-ethyl)-N″- propyl-guanidine |
| 167 | 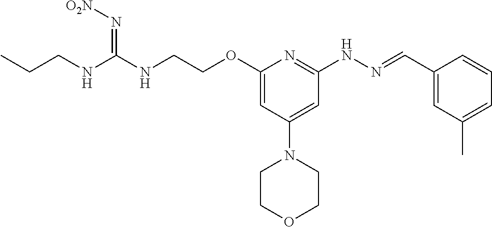 [SMILES: CCCN/C(=N\[N+](=O)[O-])NCCOc1cc(N2CCOCC2)cc(N/N=C/c2cccc(C)c2)n1] | N-Nitro-N′-(2-{6-[N′-(3- methyl-benzylidene)- hydrazino]-4-morpholin-4-yl- pyridin-2-yloxy}-ethyl)-N″- propyl-guanidine |
| 168 | 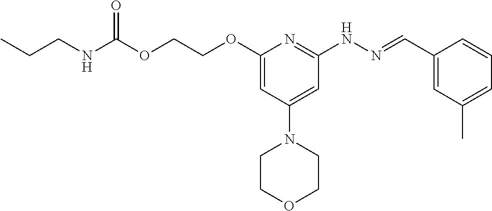 [SMILES: CCCNC(=O)OCCOc1cc(N2CCOCC2)cc(N/N=C/c2cccc(C)c2)n1] | Propyl-carbamic acid 2-{6-[N′- (3-methyl-benzylidene)- hydrazino]-4-morpholin-4-yl- pyridin-2-yloxy}-ethyl ester |
| 169 | 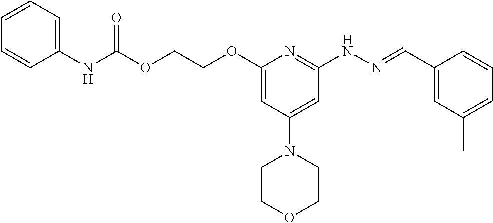 [SMILES: Cc1cccc(/C=N/Nc2cc(N3CCOCC3)cc(OCCOC(=O)Nc3ccccc3)n2)c1] | Phenyl-carbamic acid 2-{6-[N′- (3-methyl-benzylidene)- hydrazino]-4-morpholin-4-yl- pyridin-2-yloxy}-ethyl ester |
| 170 | 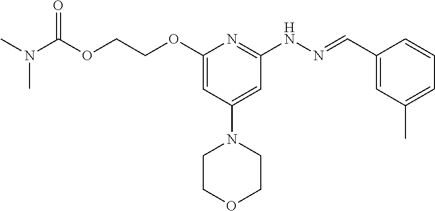 [SMILES: Cc1cccc(/C=N/Nc2cc(N3CCOCC3)cc(OCCOC(=O)N(C)C)n2)c1] | Dimethyl-carbamic acid 2-{6- [N′-(3-methyl-benzylidene)- hydrazino]-4-morpholin-4-yl- pyridin-2-yloxy}-ethyl ester |
| 171 | 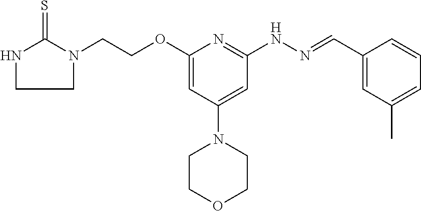 [SMILES: Cc1cccc(/C=N/Nc2cc(N3CCOCC3)cc(OCCN3CCNC3=S)n2)c1] | 1-(2-{6-[N′-(3-Methyl- benzylidene)-hydrazino]-4- morpholin-4-yl-pyridin-2- yloxy}-ethyl)-imidazolidine-2- thione |
| 172 | 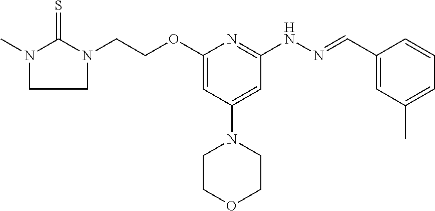 [SMILES: Cc1cccc(/C=N/Nc2cc(N3CCOCC3)cc(OCCN3CCN(C)C3=S)n2)c1] | 1-Methyl-3-(2-{6-[N′-(3- methyl-benzylidene)- hydrazino]-4-morpholino-4-yl- pyridin-2-yloxy}-ethyl)- imidazolidine-2-thione |
| 173 | 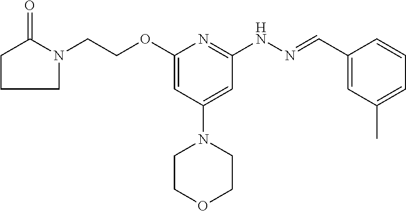 [SMILES: Cc1cccc(/C=N/Nc2cc(N3CCOCC3)cc(OCCN3CCCC3=O)n2)c1] | 1-(2-{6-[N′-(3-Methyl- benzylidene)-hydrazino]-4- morpholin-4-yl-pyridin-2- yloxy}-ethyl)-pyrrolidin-2-one |
| 174 | 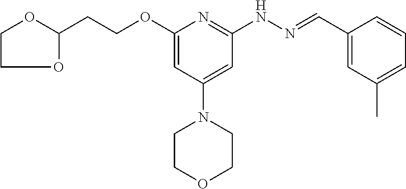 [SMILES: Cc1cccc(/C=N/Nc2cc(N3CCOCC3)cc(OCCC3OCCO3)n2)c1] | N-[6-(2-[1,3]Dioxolan-2-yl- ethoxy)-4-morpholin-4-yl- pyridin-2-yl]-N′-(3-methyl- benzylidene)-hydrazine |
| 175 | 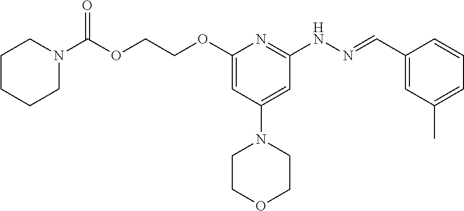 [SMILES: Cc1cccc(/C=N/Nc2cc(N3CCOCC3)cc(OCCOC(=O)N3CCCCC3)n2)c1] | Piperidine-1-carboxylic acid 2- {6-[N′-(3-methyl-benzylidene)- hydrazino]-4-morpholin-4-yl- pyridin-2-yloxy}-ethyl ester |
| 176 | 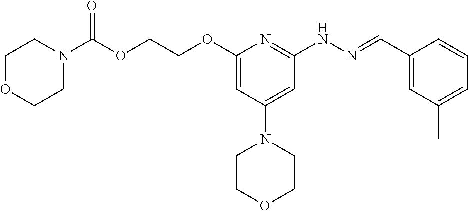 [SMILES: Cc1cccc(/C=N/Nc2cc(N3CCOCC3)cc(OCCOC(=O)N3CCOCC3)n2)c1] | Morpholine-4-carboylic acid 2-{6-[N′-(3-methyl- benzylidene)-hydrazino]-4- morpholin-4-yl-pyridin-2- yloxy}-ethyl ester |
| 177 | 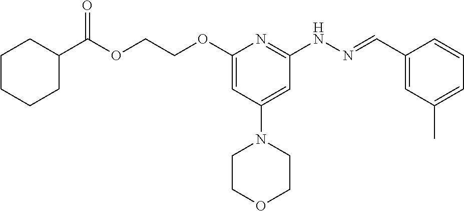 [SMILES: Cc1cccc(/C=N/Nc2cc(N3CCOCC3)cc(OCCOC(=O)C3CCCCC3)n2)c1] | Cyclohexanecarboxylic acid 2- {6-[N′-(3-methyl-benzylidene)- hydrazino]-4-morpholin-4-yl- pyridin-2-yloxy}-ethyl ester |
| 178 | 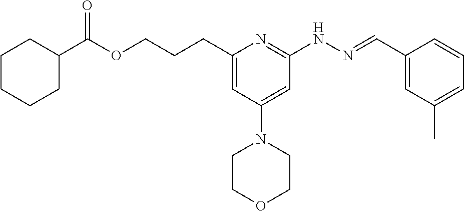 [SMILES: Cc1cccc(/C=N/Nc2cc(N3CCOCC3)cc(CCCOC(=O)C3CCCCC3)n2)c1] | Cyclohexanecarboxylic acid 3- {6-[N′-(3-methyl-benzylidene)- hydrazino]-4-morpholin-4-yl- pyridin-2-yl}-propyl ester |
| 179 | 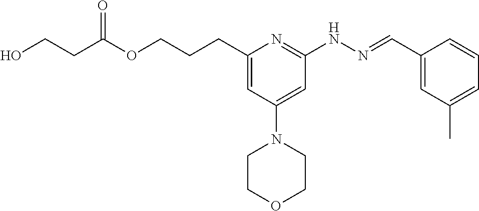 [SMILES: Cc1cccc(/C=N/Nc2cc(N3CCOCC3)cc(CCCOC(=O)CCO)n2)c1] | 3-Hydroxy-propionic acid 3- {6-[N′-(3-methyl-benzylidene)- hydrazino]-4-morpholin-4-yl- pyridin-2-yl}-propyl ester |
| 180 | 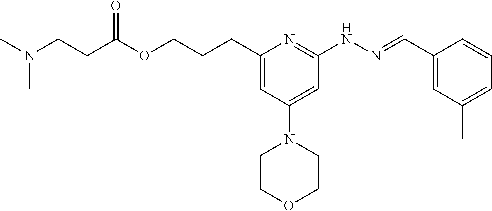 [SMILES: Cc1cccc(/C=N/Nc2cc(N3CCOCC3)cc(CCCOC(=O)CCN(C)C)n2)c1] | 3-Dimethylamino-propionic acid 3-{6-[N′-(3-methyl- benzylidene)-hydrazino]-4- morpholin-4-yl-pyridin-2-yl}- propyl ester |
| 181 | 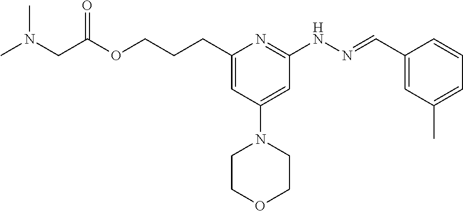 [SMILES: Cc1cccc(/C=N/Nc2cc(N3CCOCC3)cc(CCCOC(=O)CN(C)C)n2)c1] | Dimethylamino-acetic acid 3- {6-[N′-(3-methyl-benzylidene)- hydrazino]-4-morpholin-4-yl- pyridin-2-yl}-propyl ester |
| 182 | 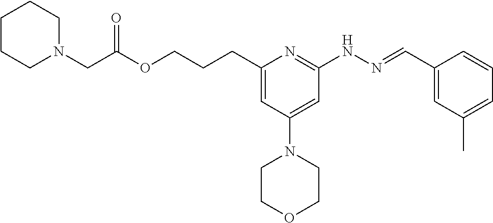 [SMILES: Cc1cccc(/C=N/Nc2cc(N3CCOCC3)cc(CCCOC(=O)CN3CCCCC3)n2)c1] | Piperidin-1-yl-acetic acid 3-{6- [N′-(3-methyl-benzylidene)- hydrazino]-4-morpholin-4-yl- pyridin-2-yl}-propyl ester |
| 183 | 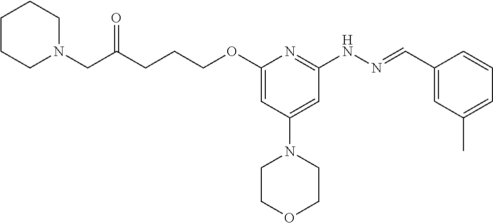 [SMILES: Cc1cccc(/C=N/Nc2cc(N3CCOCC3)cc(OCCCC(=O)CN3CCCCC3)n2)c1] | 5-{6-[N′-(3-Methyl- benzylidene)-hydrazino]-4- morpholin-4-yl-pyridin-2- yloxy}-1-piperidin-1-yl- pentan-2-one |
| 184 | 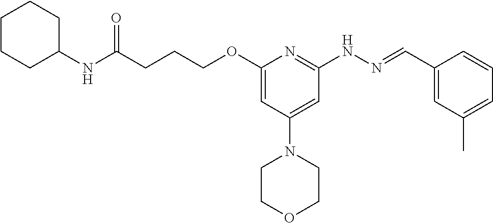 [SMILES: Cc1cccc(/C=N/Nc2cc(N3CCOCC3)cc(OCCCC(=O)NC3CCCCC3)n2)c1] | N-Cyclohexyl-4-{6-[N′-(3- methyl-benzylidene)- hydrazino]-4-morpholin-4-yl- pyridin-2-yloxy}-butyramide |
| 185 | 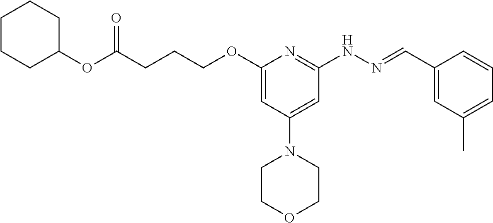 [SMILES: Cc1cccc(/C=N/Nc2cc(N3CCOCC3)cc(OCCCC(=O)OC3CCCCC3)n2)c1] | 4-{6-[N′-(3-Methyl- benzylidene)-hydrazino]-4- morpholin-4-yl-pyridin-2- yloxy}-butyric acid cyclohexyl ester |
| 186 | 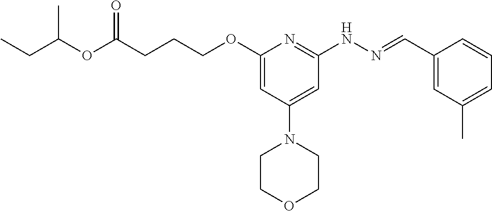 [SMILES: CCC(C)OC(=O)CCCOc1cc(N2CCOCC2)cc(N/N=C/c2cccc(C)c2)n1] | 4-{6-[N′-(3-Methyl- benzylidene)-hydrazino]-4- morpholin-4-yl-pyridin-2- yloxy}-butyric acid sec-butyl ester |
| 187 | 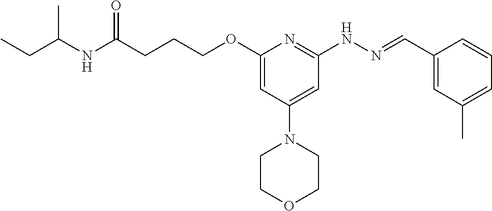 [SMILES: CCC(C)NC(=O)CCCOc1cc(N2CCOCC2)cc(N/N=C/c2cccc(C)c2)n1] | N-sec-Butyl-4-{6-[N′-(3- methyl-benzylidene)- hydrazino]-4-morpholin-4-yl- pyridin-2-yloxy}-butyramide |
| 188 | 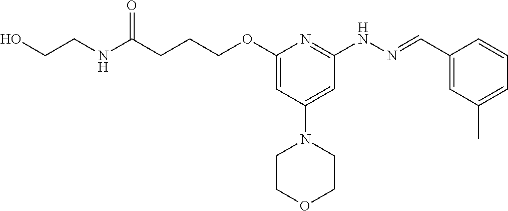 [SMILES: Cc1cccc(/C=N/Nc2cc(N3CCOCC3)cc(OCCCC(=O)NCCO)n2)c1] | N-(2-Hydroxy-ethyl)-4-{6-[N′- (3-methyl-benzylidene)- hydrazino]-4-morpholin-4-yl- pyridin-2-yloxy}-butyramide |
| 189 | 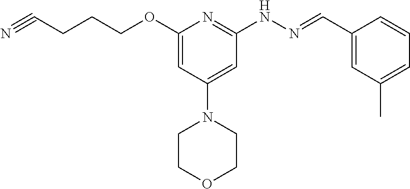 [SMILES: Cc1cccc(/C=N/Nc2cc(N3CCOCC3)cc(OCCCC#N)n2)c1] | 4-{6-[N′-(3-Methyl- benzylidene)-hydrazino]-4- morpholin-4-yl-pyridin-2- yloxy}-butyronitrile |
| 190 | 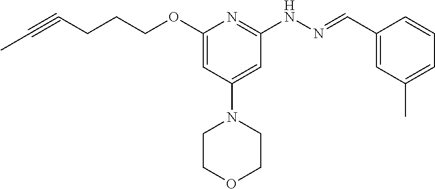 [SMILES: CC#CCCCOc1cc(N2CCOCC2)cc(N/N=C/c2cccc(C)c2)n1] | N-(6-Hex-4-ynyloxy-4- morpholin-4-yl-pyridin-2-yl)- N′-(3-methyl-benzylidene)- hydrazine |
| 191 | 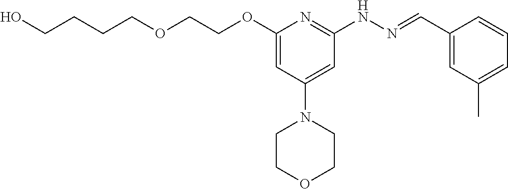 [SMILES: Cc1cccc(/C=N/Nc2cc(N3CCOCC3)cc(OCCOCCCCO)n2)c1] | 4-(2-{6-[N′-(3-Methyl- benzylidene)-hydrazino]-4- morpholin-4-yl-pyridin-2- yloxy}-ethoxy)-butan-1-ol |
| 192 | 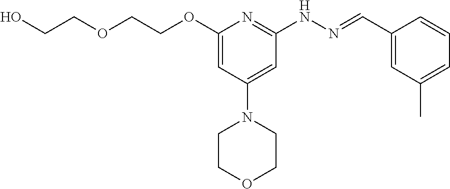 [SMILES: Cc1cccc(/C=N/Nc2cc(N3CCOCC3)cc(OCCOCCO)n2)c1] | 2-(2-{6-[N′-(3-Methyl- benzylidene)-hydrazino]-4- morpholin-4-yl-pyridin-2- yloxy}-ethoxy)-ethanol |
| 193 | 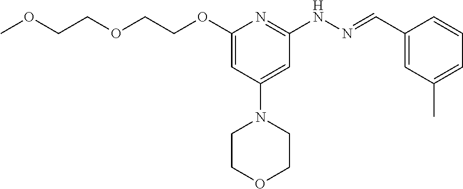 [SMILES: COCCOCCOc1cc(N2CCOCC2)cc(N/N=C/c2cccc(C)c2)n1] | N-{6-[2-(2-Methoxy-ethoxy)- ethoxy]-4-morpholin-4-yl- pyridin-2-yl}-N′-(3-methyl- benzylidene)-hydrazine |
| 194 | 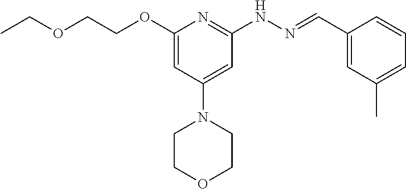 [SMILES: CCOCCOc1cc(N2CCOCC2)cc(N/N=C/c2cccc(C)c2)n1] | N-[6-(2-Ethoxy-ethoxy)-4- morpholin-4-yl-pyridin-2-yl]- N′-(3-methyl-benzylidene)- hydrazine |
| 195 | 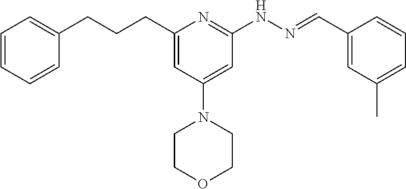 [SMILES: Cc1cccc(/C=N/Nc2cc(N3CCOCC3)cc(CCCc3ccccc3)n2)c1] | N-(3-Methyl-benzylidene)-N′- [4-morpholin-4-yl-6-(3-phenyl- propyl)-pyridin-2-yl]-hydrazine |
| 196 | 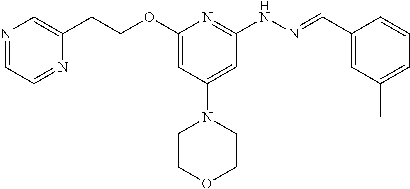 [SMILES: Cc1cccc(/C=N/Nc2cc(N3CCOCC3)cc(OCCc3cnccn3)n2)c1] | N-(3-Methyl-benzylidene)-N′- [4-morpholin-4-yl-6-(2- pyrazin-2-yl-ethoxy)-pyridin-2- yl]-hydrazine |
| 197 | 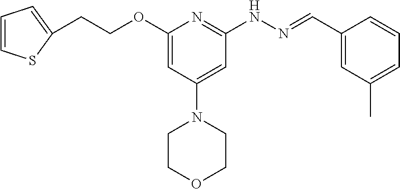 [SMILES: Cc1cccc(/C=N/Nc2cc(N3CCOCC3)cc(OCCc3cccs3)n2)c1] | N-(3-Methyl-benzylidene)-N′- [4-morpholin-4-yl-6-(2- thiophen-2-yl-ethoxy)-pyridin- 2-yl]-hydrazine |
| 198 | 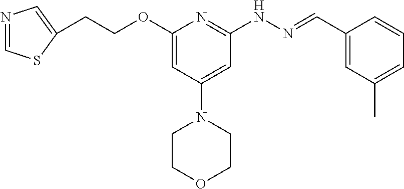 [SMILES: Cc1cccc(/C=N/Nc2cc(N3CCOCC3)cc(OCCc3cncs3)n2)c1] | N-(3-Methyl-benzylidene)-N′- [4-morpholin-4-yl-6-(2-thiazol- 5-yl-ethoxy)-pyridin-2-yl]- hydrazine |
| 199 | 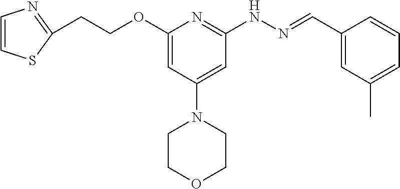 [SMILES: Cc1cccc(/C=N/Nc2cc(N3CCOCC3)cc(OCCc3nccs3)n2)c1] | N-(3-Methyl-benzylidene)-N′- [4-morpholin-4-yl-6-(2-thiazol- 2-yl-ethoxy)-pyridin-2-yl]- hydrazine |
| 200 | 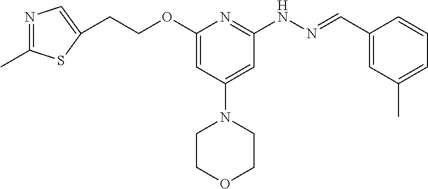 [SMILES: Cc1cccc(/C=N/Nc2cc(N3CCOCC3)cc(OCCc3cnc(C)s3)n2)c1] | N-(3-Methyl-benzylidene)-N′- {6-[2-(2-methyl-thiazol-5-yl)- ethoxy]-4-morpholin-4-yl- pyridin-2-yl}-hydrazine |
| 201 | 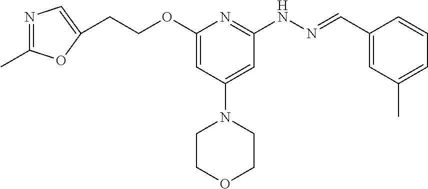 [SMILES: Cc1cccc(/C=N/Nc2cc(N3CCOCC3)cc(OCCc3cnc(C)o3)n2)c1] | N-(3-Methyl-benzylidene)-N′- {6-[2-(2-methyl-oxazol-5-l)- ethoxy]-4-morpholin-4-yl- pyridin-2-yl}-hydrazine |
| 202 | 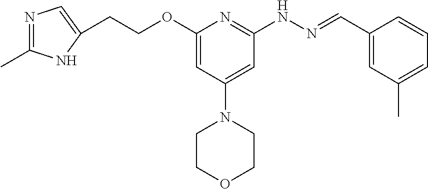 [SMILES: Cc1cccc(/C=N/Nc2cc(N3CCOCC3)cc(OCCc3cnc(C)[nH]3)n2)c1] | N-(3-Methyl-benzylidene)-N′- {6-[2-(2-methyl-3H-imidazol- 4-yl)-ethoxy]-4-morpholin-4- yl-pyridin-2-yl}-hydrazine |
| 203 | 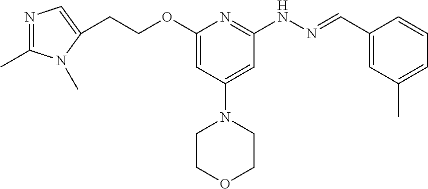 [SMILES: Cc1cccc(/C=N/Nc2cc(N3CCOCC3)cc(OCCc3cnc(C)n3C)n2)c1] | N-{6-[2-(2,3-Dimethyl-3H- imidazol-4-yl)-ethoxy]-4- morpholin-4-yl-pyridin-2-yl}- N′-(3-methyl-benzylidene)- hydrazine |
| 204 | 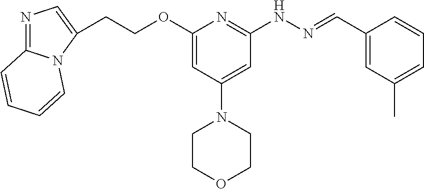 [SMILES: Cc1cccc(/C=N/Nc2cc(N3CCOCC3)cc(OCCc3cnc4ccccn34)n2)c1] | N-[6-(2-Imidazol[1,2-a]pyridin- 3-yl-ethoxy)-4-morpholin-4-yl- pyridin-2-yl]-N′-(3-methyl- benzylidene)-hydrazine |
| 205 | 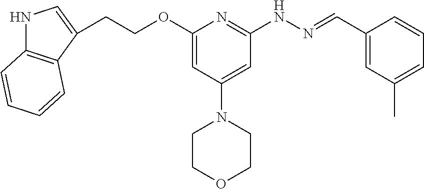 [SMILES: Cc1cccc(/C=N/Nc2cc(N3CCOCC3)cc(OCCc3c[nH]c4ccccc34)n2)c1] | N-{6-[2-(1H-Indol-3-yl)- ethoxy]-4-morpholin-4-yl- pyridin-2-yl}-N′-(3-methyl- benzylidene)-hydrazine |
| 206 | 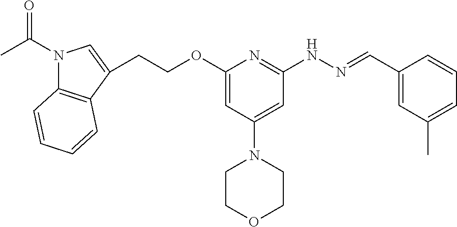 [SMILES: CC(=O)n1cc(CCOc2cc(N3CCOCC3)cc(N/N=C/c3cccc(C)c3)n2)c2ccccc21] | 1-[3-(2-{6-[N′-(3-Methyl- benzylidene)-hydrazino]-4- morpholin-4-yl-pyridin-2- yloxy}-ethyl)-indol-1-yl]- ethanone |
| 207 | 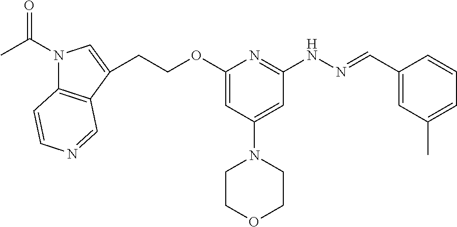 [SMILES: CC(=O)n1cc(CCOc2cc(N3CCOCC3)cc(N/N=C/c3cccc(C)c3)n2)c2cnccc21] | 1-[3-(2-{6-[N′-(3-Methyl- benzylidene)-hydrazino]-4- morpholin-4-yl-pyridin-2- yloxy}-ethyl)-pyrrolo[3,2- c]pyridin-1-yl]-ethanone |
| 208 | 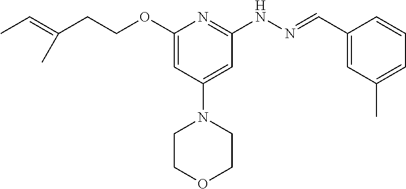 [SMILES: C/C=C(\C)CCOc1cc(N2CCOCC2)cc(N/N=C/c2cccc(C)c2)n1] | N-(3-Methyl-benzylidene)-N′- [6-(3-methyl-pent-3-enyloxy)- 4-morpholin-4-yl-pyridin-2-yl]- hydrazine |
| 209 | 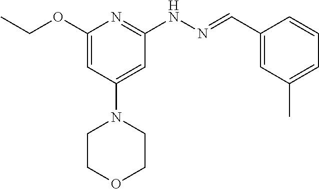 [SMILES: CCOc1cc(N2CCOCC2)cc(N/N=C/c2cccc(C)c2)n1] | N-(6-Ethoxy-4-morpholin-4-yl- pyridin-2-yl)-N′-(3-methyl- benzylidene)-hydrazine |
| 210 | 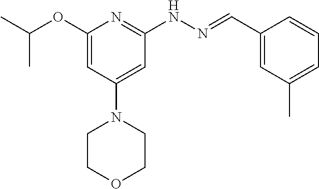 [SMILES: Cc1cccc(/C=N/Nc2cc(N3CCOCC3)cc(OC(C)C)n2)c1] | N-(6-Isopropoxy-4-morpholin- 4-yl-pyridin-2-yl)-N′-(3- methyl-benzylidene)-hydrazine |
| 211 | 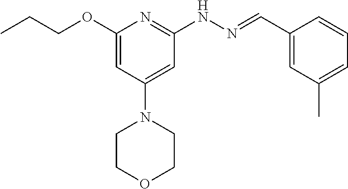 [SMILES: CCCOc1cc(N2CCOCC2)cc(N/N=C/c2cccc(C)c2)n1] | N-(3-Methyl-benzylidene)-N′- (4-morpholin-4-yl-6-propoxy- pyridin-2-yl)-hydrazine |
| 212 | 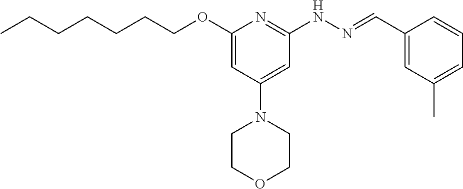 [SMILES: CCCCCCCOc1cc(N2CCOCC2)cc(N/N=C/c2cccc(C)c2)n1] | N-(6-Heptyloxy-4-morpholin- 4-yl-pyridin-2-yl)-N′-(3- methyl-benzylidene)-hydrazine |
| 213 | 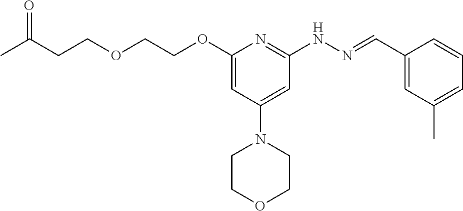 [SMILES: CC(=O)CCOCCOc1cc(N2CCOCC2)cc(N/N=C/c2cccc(C)c2)n1] | 4-(2-{6-[N′-(3-Methyl- benzylidene)-hydrazino]-4- morpholin-4-yl-pyridin-2- yloxy}-ethoxy)-butan-2-one |
| 214 | 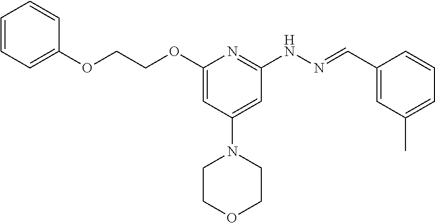 [SMILES: Cc1cccc(/C=N/Nc2cc(N3CCOCC3)cc(OCCOc3ccccc3)n2)c1] | N-(3-Methyl-benzylidene)-N′- [4-morpholin-4-yl-6-(2- phenoxy-ethoxy)-pyridin-2-yl]- hydrazine |
| 215 | 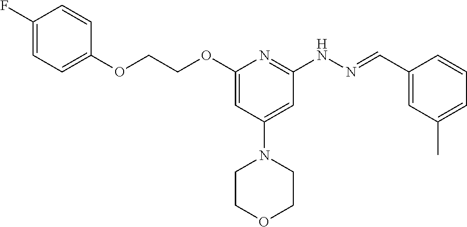 [SMILES: Cc1cccc(/C=N/Nc2cc(N3CCOCC3)cc(OCCOc3ccc(F)cc3)n2)c1] | N-{6-[2-(4-Fluoro-phenoxy)- ethoxy]-4-morpholin-4-yl- pyridin-2-yl}-N′-(3-methyl- benzylidene)-hydrazine |
| 216 | 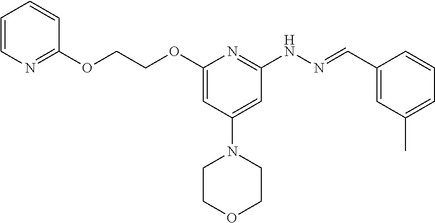 [SMILES: Cc1cccc(/C=N/Nc2cc(N3CCOCC3)cc(OCCOc3ccccn3)n2)c1] | N-(3-Methyl-benzylidene)-N′- {4-morpholin-4-yl-6-[2- (pyridin-2-yloxy)-ethoxy]- pyridin-2-yl}-hydrazine |
| 217 | 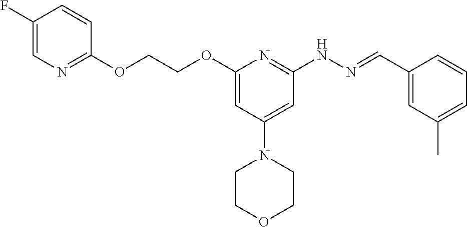 [SMILES: Cc1cccc(/C=N/Nc2cc(N3CCOCC3)cc(OCCOc3ccc(F)cn3)n2)c1] | N-{6-[2-(5-Fluoro-pyridin-2- yloxy)-ethoxy]-4-morpholin-4- yl-pyridin-2-yl}-N′-(3-methyl- benzylidene)-hydrazine |
| 218 | 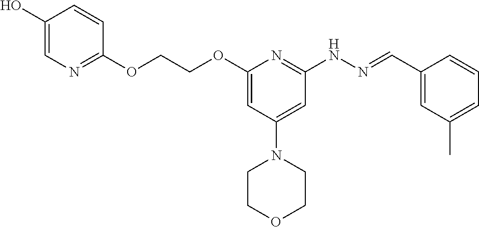 [SMILES: Cc1cccc(/C=N/Nc2cc(N3CCOCC3)cc(OCCOc3ccc(O)cn3)n2)c1] | 6-(2-{6-[N′-(3-Methyl- benzylidene)-hydrazino]-4- morpholin-4-yl-pyridin-2- yloxy}-ethoxy)-pyridin-3-ol |
| 219 | 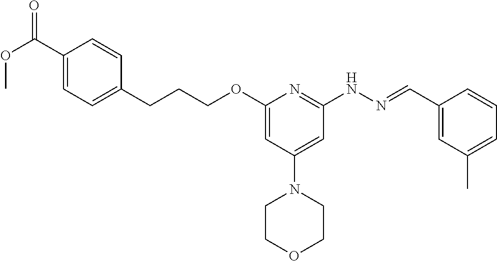 [SMILES: COC(=O)c1ccc(CCCOc2cc(N3CCOCC3)cc(N/N=C/c3cccc(C)c3)n2)cc1] | 4-(3-{6-[N′-(3-Methyl- benzylidene)-hydrazino]-4- morpholin-4-yl-pyridin-2- yloxy}-propyl)-benzoic acid methyl ester |
| 220 | 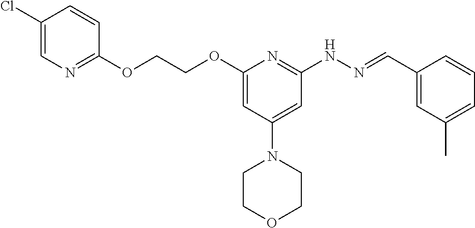 [SMILES: Cc1cccc(/C=N/Nc2cc(N3CCOCC3)cc(OCCOc3ccc(Cl)cn3)n2)c1] | N-{6-[2-(5-Chloro-pyridin-2- yloxy)-ethoxy]-4-morpholin-4- yl-pyridin-2-yl}-N′-(3-methyl- benzylidene)-hydrazine |
| 221 | 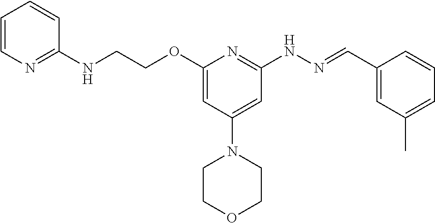 [SMILES: Cc1cccc(/C=N/Nc2cc(N3CCOCC3)cc(OCCNc3ccccn3)n2)c1] | (2-{6-[N′-(3-Methyl- benzylidene)-hydrazino]-4- morpholin-4-yl-pyridin-2- yloxy}-ethyl)-pyridin-2-yl- amine |
| 222 | 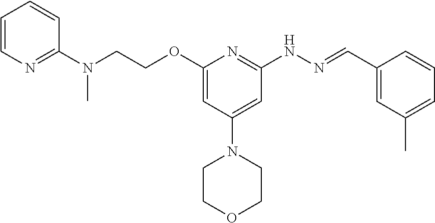 [SMILES: Cc1cccc(/C=N/Nc2cc(N3CCOCC3)cc(OCCN(C)c3ccccn3)n2)c1] | Methyl-(2-{6-[N′-(3-methyl- benzylidene)-hydrazino]-4- morpholin-4-yl-pyridin-2- yloxy}-ethyl)-pyridin-2-yl- amine |
| 223 | 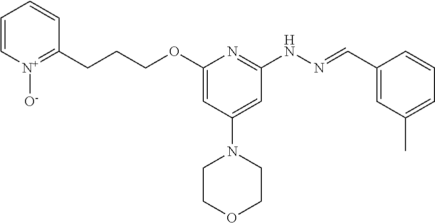 [SMILES: Cc1cccc(/C=N/Nc2cc(N3CCOCC3)cc(OCCCc3cccc[n+]3[O-])n2)c1] | N-(3-Methyl-benzylidene)-N′- {4-morpholin-4-yl-6-[3-(1-oxy- pyridin-2-yl)-propoxy]-pyridin- 2-yl}-hydrazine |
| 224 | 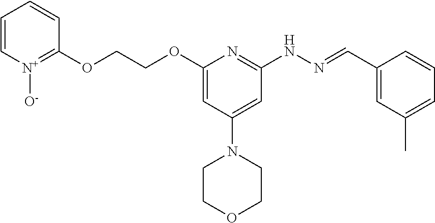 [SMILES: Cc1cccc(/C=N/Nc2cc(N3CCOCC3)cc(OCCOc3cccc[n+]3[O-])n2)c1] | N-(3-Methyl-benzylidene)-N′- {4-morpholin-4-yl-6-[2-(1-oxy- pyridin-2-yloxy)-ethoxy]- pyridin-2-yl}-hydrazine |
| 225 | 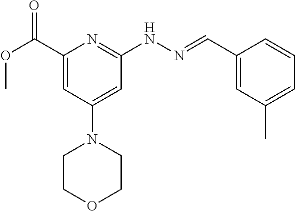 [SMILES: COC(=O)c1cc(N2CCOCC2)cc(N/N=C/c2cccc(C)c2)n1] | 6-[N′-(3-Methyl-benzylidene)- hydrazino]-4-morpholin-4-yl- pyridine-2-carboxylic acid methyl ester |
| 226 | 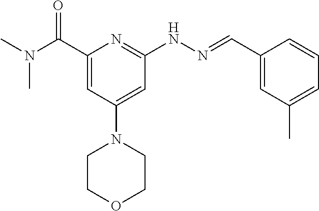 [SMILES: Cc1cccc(/C=N/Nc2cc(N3CCOCC3)cc(C(=O)N(C)C)n2)c1] | 6-[N′-(3-Methyl-benzylidene)- hydrazino]-4-morpholin-4-yl- pyridine-2-carboxylic acid dimethylamide |
| 227 | 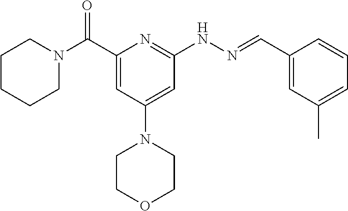 [SMILES: Cc1cccc(/C=N/Nc2cc(N3CCOCC3)cc(C(=O)N3CCCCC3)n2)c1] | {6-[N′-(3-Methyl-benzylidene)- hydrazino]-4-morpholin-4-yl- pyridin-2-yl}-piperidin-1-yl- methanone |
| 228 | 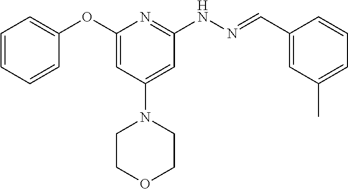 [SMILES: Cc1cccc(/C=N/Nc2cc(N3CCOCC3)cc(Oc3ccccc3)n2)c1] | N-(3-Methyl-benzylidene)-N′- (4-morpholin-4-yl-6-phenoxy- pyridin-2-yl)-hydrazine |
| 229 | 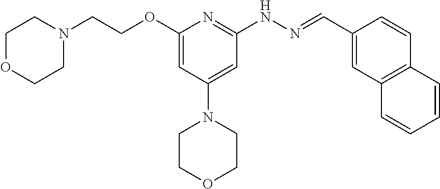 [SMILES: C(=N\Nc1cc(N2CCOCC2)cc(OCCN2CCOCC2)n1)/c1ccc2ccccc2c1] | N-[4-Morpholin-4-yl-6-(2- morpholin-4-yl-ethoxy)- pyridin-2-yl]-N′-naphthalen-2- ylmethylene-hydrazine |
| 230 | 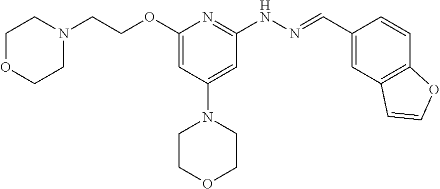 [SMILES: C(=N\Nc1cc(N2CCOCC2)cc(OCCN2CCOCC2)n1)/c1ccc2occc2c1] | N-Benzofuran-5-ylmethylene- N′-[4-morpholin-4-yl-6-(2- morpholin-4-yl-ethoxy)- pyridin-2-yl]-hydrazine |
| 231 | 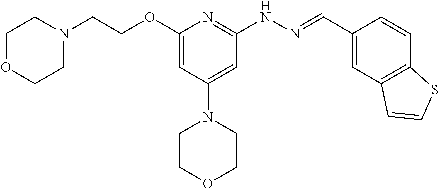 [SMILES: C(=N\Nc1cc(N2CCOCC2)cc(OCCN2CCOCC2)n1)/c1ccc2sccc2c1] | N-Benzo[b]thiophen-5- ylmethylene-N′-[4-morpholin- 4-yl-6-(2-morpholin-4-yl- ethoxy)-pyridin-2-yl]- hydrazine |
| 232 | 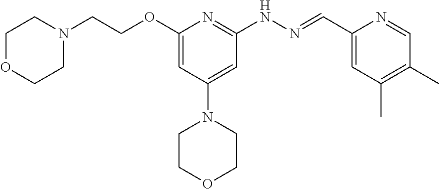 [SMILES: Cc1cnc(/C=N/Nc2cc(N3CCOCC3)cc(OCCN3CCOCC3)n2)cc1C] | N-(4,5-Dimethyl-pyridin-2- ylmethylene)-N′-[4-morpholin- 4-yl-6-(2-morpholin-4-yl- ethoxy)-pyridin-2-yl]- hydrazine |
| 233 | 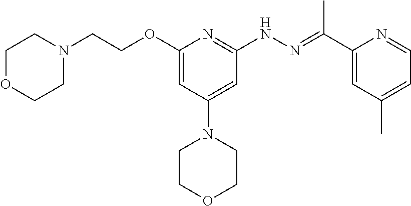 [SMILES: C/C(=N\Nc1cc(N2CCOCC2)cc(OCCN2CCOCC2)n1)c1cc(C)ccn1] | N-[1-(4-Methyl-pyridin-2-yl)- ethylidene]-N′-[4-morpholin-4- yl-6-(2-morpholin-4-yl- ethoxy)-pyridin-2-yl]- hydrazine |
| 234 | 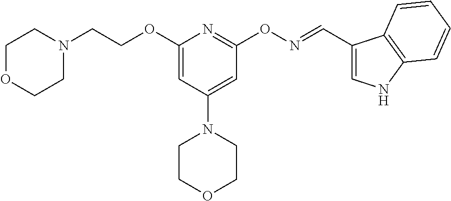 [SMILES: C(=N\Oc1cc(N2CCOCC2)cc(OCCN2CCOCC2)n1)/c1c[nH]c2ccccc12] | 1H-Indole-3-carbaldehyde O- [4-morpholin-4-yl-6-(2- morpholin-4-yl-ethoxy)- pyridin-2-yl]-oxime |
| 235 | 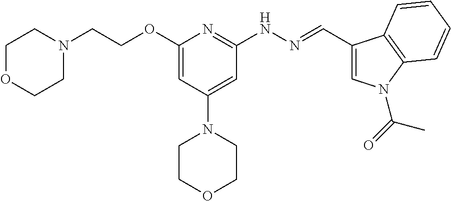 [SMILES: CC(=O)n1cc(/C=N/Nc2cc(N3CCOCC3)cc(OCCN3CCOCC3)n2)c2ccccc21] | 1-(3-{[4-Morpholin-4-yl-6-(2- morpholin-4-yl-ethoxy)- pyridin-2-yl]- hydrazonomethyl}-indol-1-yl)- ethanone |
| 236 | 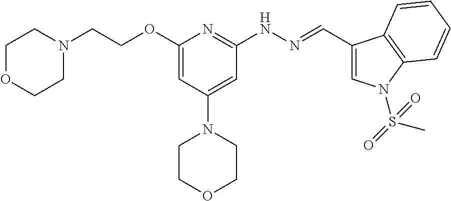 [SMILES: CS(=O)(=O)n1cc(/C=N/Nc2cc(N3CCOCC3)cc(OCCN3CCOCC3)n2)c2ccccc21] | N-(1-Methanesulfonyl-1H- indol-3-ylmethylene)-N′-[4- morpholin-4-yl-6-(2- morpholin-4-yl-ethoxy)- pyridin-2-yl]-hydrazine |
| 237 | 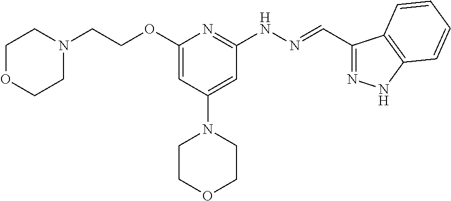 [SMILES: C(=N\Nc1cc(N2CCOCC2)cc(OCCN2CCOCC2)n1)/c1n[nH]c2ccccc12] | N-(1H-Indazol-3-ylmethylene)- N′-[4-morpholin-4-yl-6-(2- morpholin-4-yl-ethoxy)- pyridin-2-yl]-hydrazine |
| 238 | 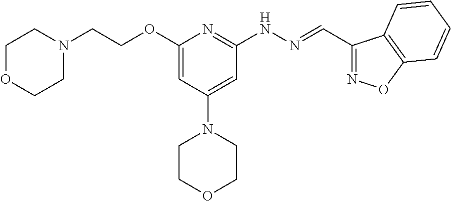 [SMILES: C(=N\Nc1cc(N2CCOCC2)cc(OCCN2CCOCC2)n1)/c1noc2ccccc12] | N-Benzo[d]isoxazol-3- ylmethylene-N′-[4-morpholin- 4-yl-6-(2-morpholin-4-yl- ethoxy)-pyridin-2-yl]- hydrazine |
| 239 | 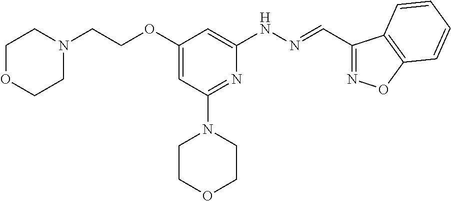 [SMILES: C(=N\Nc1cc(OCCN2CCOCC2)cc(N2CCOCC2)n1)/c1noc2ccccc12] | N-Benzo[d]isoxazol-3- ylmethylene-N′-[6-morpholin- 4-yl-4-(2-morpholin-4-yl- ethoxy)-pyridin-2-yl]- hydrazine |
| 240 | 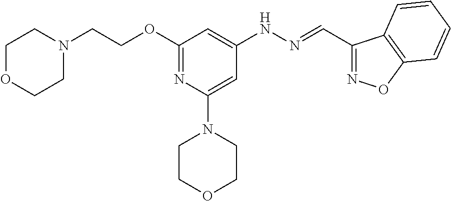 [SMILES: C(=N\Nc1cc(OCCN2CCOCC2)nc(N2CCOCC2)c1)/c1noc2ccccc12] | N-Benzo[d]isoxazol-3- ylmethylene-N′-[2-morpholin- 4-yl-6-(2-morpholin-4-yl- ethoxy)-pyridin-4-yl]- hydrazine |
| 241 | 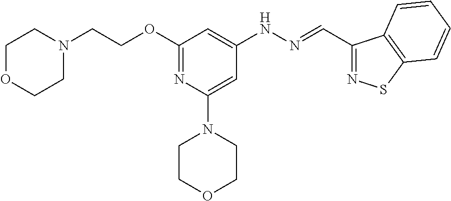 [SMILES: C(=N\Nc1cc(OCCN2CCOCC2)nc(N2CCOCC2)c1)/c1nsc2ccccc12] | N-Benzo[d]isothiazol-3- ylmethylene-N′-[2-morpholin- 4-yl-6-(2-morpholin-4-yl- ethoxy)-pyridin-4-yl]- hydrazine |
| 242 | 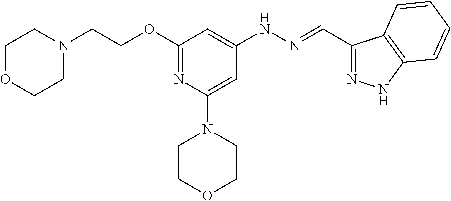 [SMILES: C(=N\Nc1cc(OCCN2CCOCC2)nc(N2CCOCC2)c1)/c1n[nH]c2ccccc12] | N-(1H-Indazol-3-ylmethylene)- N′-[2-morpholin-4-yl-6-(2- morpholin-4-yl-ethoxy)- pyridin-4-yl]-hydrazine |
| 243 | 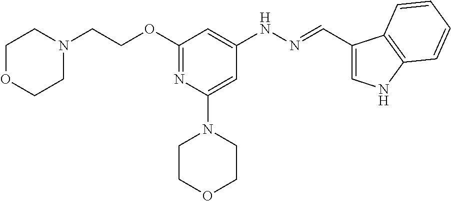 [SMILES: C(=N\Nc1cc(OCCN2CCOCC2)nc(N2CCOCC2)c1)/c1c[nH]c2ccccc12] | N-(1H-Indol-3-ylmethylene)- N′-[2-morpholin-4-yl-6-(2- morpholin-4-yl-ethoxy)- pyridin-4-yl]-hydrazine |
| 244 | 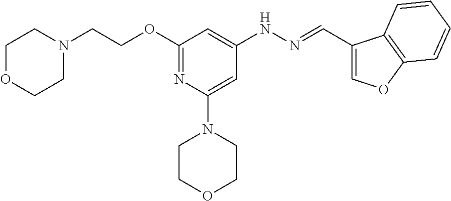 [SMILES: C(=N\Nc1cc(OCCN2CCOCC2)nc(N2CCOCC2)c1)/c1coc2ccccc12] | N-Benzofuran-3-ylmethylene- N′-[2-morpholin-4-yl-6-(2- morpholin-4-yl-ethoxy)- pyridin-4-yl]-hydrazine |
| 245 | 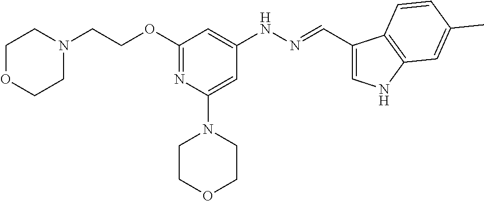 [SMILES: Cc1ccc2c(/C=N/Nc3cc(OCCN4CCOCC4)nc(N4CCOCC4)c3)c[nH]c2c1] | N-(6-Methyl-1H-indol-3- ylmethylene)-N′-[2-morpholin- 4-yl-6-(2-morpholin-4- ethoxy)-pyridin-4-yl]- hydrazine |
| 246 | 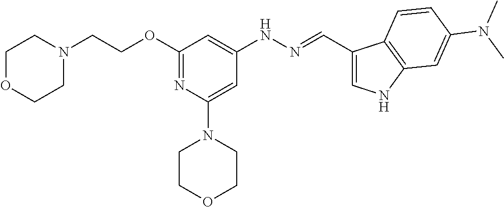 [SMILES: CN(C)c1ccc2c(/C=N/Nc3cc(OCCN4CCOCC4)nc(N4CCOCC4)c3)c[nH]c2c1] | Dimethyl-(3-{[2-morpholin-4- yl-6-(2-morpholin-4-yl- ethoxy)-pyridin-4-yl]- hydrazonomethyl}-1H-indol-6- yl)-amine |
| 247 | 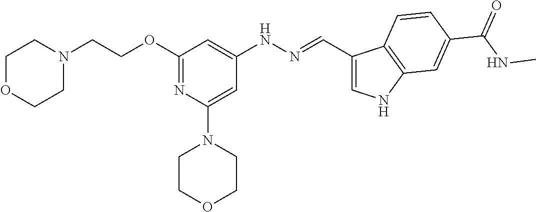 [SMILES: CNC(=O)c1ccc2c(/C=N/Nc3cc(OCCN4CCOCC4)nc(N4CCOCC4)c3)c[nH]c2c1] | 3-{[2-Morpholin-4-yl-6-(2- morpholin-4-yl-ethoxy)- pyridin-4-yl]- hydrazonomethyl}-1H-indole- 6-carboxylic acid methylamide |
| 248 | 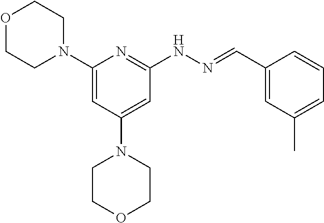 [SMILES: Cc1cccc(/C=N/Nc2cc(N3CCOCC3)cc(N3CCOCC3)n2)c1] | N-(4,6-Di-morpholin-4-yl- pyridin-2-yl)-N′-(3-methyl- benzylidene)-hydrazine |
| 249 | 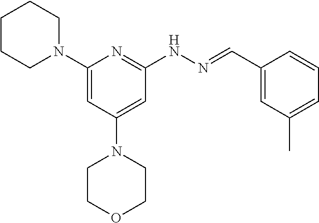 [SMILES: Cc1cccc(/C=N/Nc2cc(N3CCOCC3)cc(N3CCCCC3)n2)c1] | N-(3-Methyl-benzylidene)-N′- (4′-morpholin-4-yl-3,4,5,6- tetrahydro-2H- [1,2′]pyridinyl-6′-yl)- hydrazine |
| 250 | 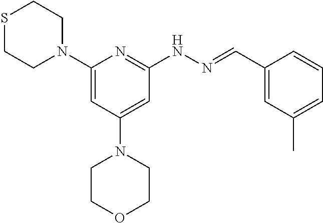 [SMILES: Cc1cccc(/C=N/Nc2cc(N3CCOCC3)cc(N3CCSCC3)n2)c1] | N-(3-Methyl-benzylidene)-N′- (4-morpholin-4-yl-6- thiomorpholin-4-yl-pyridin-2- yl)-hydrazine |
| 251 | 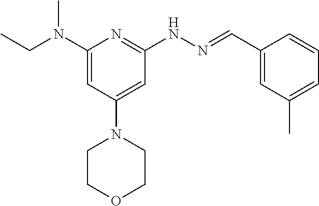 [SMILES: CCN(C)c1cc(N2CCOCC2)cc(N/N=C/c2cccc(C)c2)n1] | Ethyl-methyl-{6-[N′-(3-methyl- benzylidene)-hydrazino]-4- morpholin-4-yl-pyridin-2-yl}- amine |
| 252 | 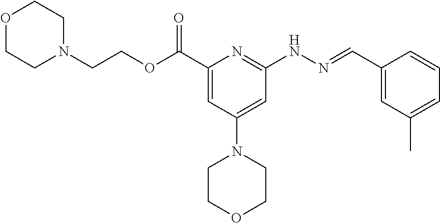 [SMILES: Cc1cccc(/C=N/Nc2cc(N3CCOCC3)cc(C(=O)OCCN3CCOCC3)n2)c1] | 6-[N′-(3-Methyl-benzylidene)- hydrazino]-4-morpholin-4-yl- pyridine-2-carboxylic acid 2- morpholin-4-yl-ethyl ester |
| 253 | 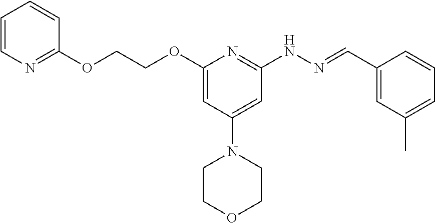 [SMILES: Cc1cccc(/C=N/Nc2cc(N3CCOCC3)cc(OCCOc3ccccn3)n2)c1] | N-(3-Methyl-benzylidene)-N′- {4-morpholin-4-yl-6-[2- (pyridin-2-yloxy)-ethoxy]- pyridin-2-yl}-hydrazine |
| 254 | 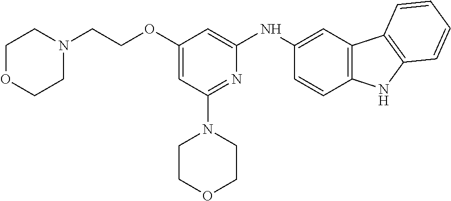 [SMILES: c1ccc2c(c1)[nH]c1ccc(Nc3cc(OCCN4CCOCC4)cc(N4CCOCC4)n3)cc12] | (9H-Carbazol-3-yl)-[6- morpholin-4-yl-4-(2- morpholin-4-yl-ethoxy)- pyridin-2-yl]-amine |
| 255 | 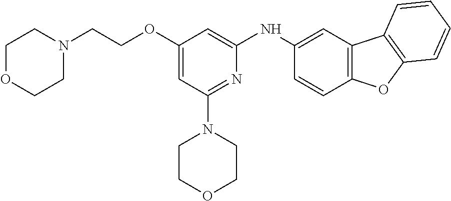 [SMILES: c1ccc2c(c1)oc1ccc(Nc3cc(OCCN4CCOCC4)cc(N4CCOCC4)n3)cc12] | Dibenzofuran-2-yl-[6- morpholin-4-yl-4-(2- morpholin-4-yl-ethoxy)- pyridin-2-yl]-amine |
| 256 | 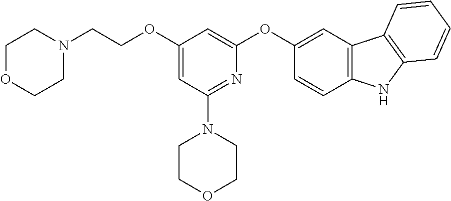 [SMILES: c1ccc2c(c1)[nH]c1ccc(Oc3cc(OCCN4CCOCC4)cc(N4CCOCC4)n3)cc12] | 3-[6-Morpholin-4-yl-4-(2- morpholin-4-yl-ethoxy)- pyridin-2-yloxy]-9H-carbazole |
| 257 | 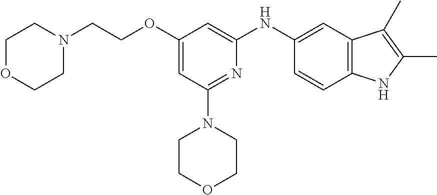 [SMILES: Cc1[nH]c2ccc(Nc3cc(OCCN4CCOCC4)cc(N4CCOCC4)n3)cc2c1C] | (2,3-Dimethyl-1H-indol-5-yl)- [6-morpholin-4-yl-4-(2- morpholin-4-yl-ethoxy)- pyridin-2-yl]-amine |
| 258 | 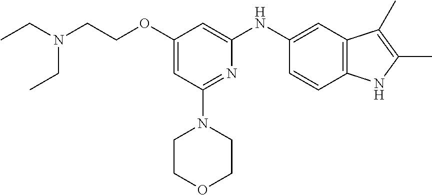 [SMILES: CCN(CC)CCOc1cc(Nc2ccc3[nH]c(C)c(C)c3c2)nc(N2CCOCC2)c1] | [4-(2-Diethylamino-ethoxy)-6- morpholin-4-yl-pyridin-2-yl]- (2,3-dimethyl-1H-indol-5-yl)- amine |
| 259 | 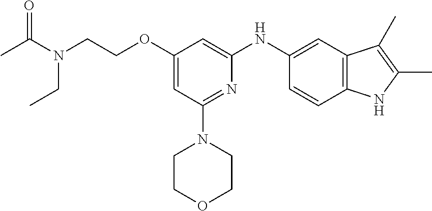 [SMILES: CCN(CCOc1cc(Nc2ccc3[nH]c(C)c(C)c3c2)nc(N2CCOCC2)c1)C(C)=O] | N-{2-[2-(2,3-Dimethyl-1H- indol-5-ylamino)-6-morpholin- 4-yl-pyridin-4-yloxy]-ethyl}- N-ethyl-acetamide |
| 260 | 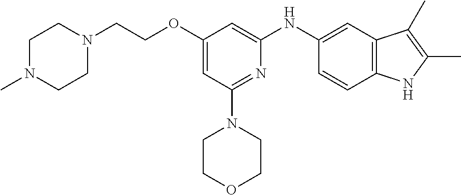 | (2,3-Dimethyl-1H-indol-5-yl)- {4-[2-(4-methyl-piperazin-1- yl)-ethoxy]-6-morpholin-4-yl- pyridin-2-yl}-amine |
| 261 | 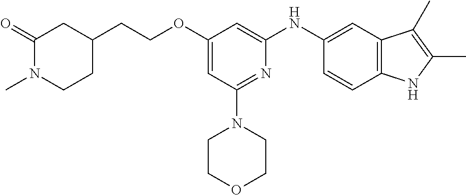 | 4-{2-[2-(3,4-Dimethyl-1H- indol-5-ylamino)-6-morpholin- 4-yl-pyridin-4-yloxy]-ethyl}-1- methyl-piperidin-2-one |
| 262 | 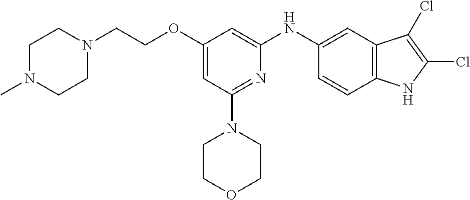 [SMILES: CN1CCN(CCOc2cc(Nc3ccc4[nH]c(Cl)c(Cl)c4c3)nc(N3CCOCC3)c2)CC1] | (2,3-Dichloro-1H-indol-5-yl)- {4-[2-(4-methyl-piperazin-1- yl)-ethoxy]-6-morpholin-4-yl- pyridin-2-yl}-amine |
| 263 | 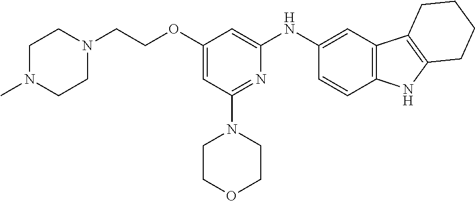 [SMILES: CN1CCN(CCOc2cc(Nc3ccc4[nH]c5c(c4c3)CCCC5)nc(N3CCOCC3)c2)CC1] | {4-[2-(4-Methyl-piperazin-1- yl)-ethoxy]-6-morpholin-4-yl- pyridin-2-yl}-(6,7,8,9- tetrahydro-5H-carbazol-3-yl)- amine |
| 264 | 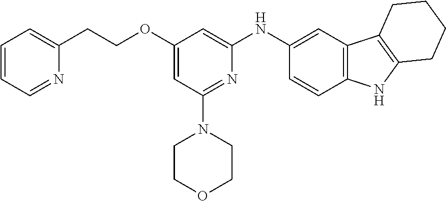 [SMILES: c1ccc(CCOc2cc(Nc3ccc4[nH]c5c(c4c3)CCCC5)nc(N3CCOCC3)c2)nc1] | [6-Morpholin-4-yl-4-(2- pyridin-2-yl-ethoxy)-pyridin-2- yl]-(6,7,8,9-tetrahydro-5H- carbazol-3-yl)-amine |
| 265 | 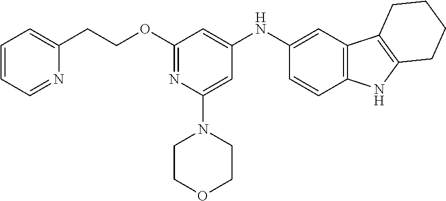 [SMILES: c1ccc(CCOc2cc(Nc3ccc4[nH]c5c(c4c3)CCCC5)cc(N3CCOCC3)n2)nc1] | [2-Morpholin-4-yl-6-(2- pyridin-2-yl-ethoxy)-pyridin-4- yl]-(6,7,8,9-tetrahydro-5H- carbazol-3-yl)-amine |
| 266 | 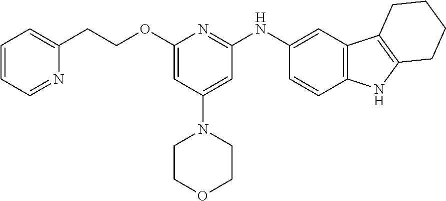 [SMILES: c1ccc(CCOc2cc(N3CCOCC3)cc(Nc3ccc4[nH]c5c(c4c3)CCCC5)n2)nc1] | [4-Morpholin-4-yl-6-(2- pyridin-2-yl-ethoxy)-pyridin-2- yl]-(6,7,8,9-tetrahydro-5H- carbazol-3-yl)-amine |
| 267 | 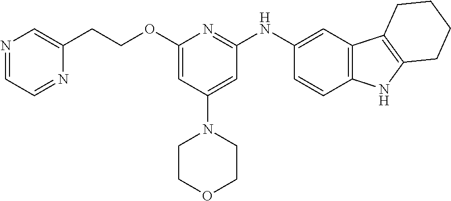 [SMILES: c1cnc(CCOc2cc(N3CCOCC3)cc(Nc3ccc4[nH]c5c(c4c3)CCCC5)n2)cn1] | [4-Morpholin-4-yl-6-(2- pyrazin-2-yl-ethoxy)-pyridin-2- yl]-(6,7,8,9-tetrahydro-5H- carbazol-3-yl)-amine |
| 268 | 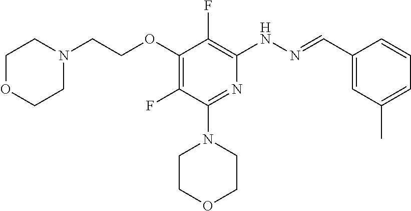 [SMILES: Cc1cccc(/C=N/Nc2nc(N3CCOCC3)c(F)c(OCCN3CCOCC3)c2F)c1] | N-[3,5-Difluoro-6-morpholin- 4-yl-4-(2-morpholin-4-yl- ethoxy)-pyridin-2-yl]-N′-(3- methyl-benzylidene)-hydrazine |
| 269 | 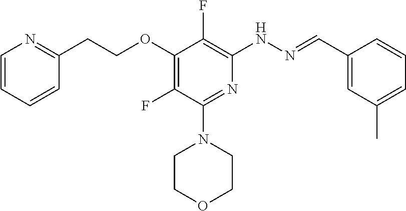 [SMILES: Cc1cccc(/C=N/Nc2nc(N3CCOCC3)c(F)c(OCCc3ccccn3)c2F)c1] | N-[3,5-Dichloro-6-morpholin- 4-yl-4-(2-pyridin-2-yl-ethoxy)- pyridin-2-yl]-N′-(3-methyl- benzylidene)-hydrazine |
| 270 | 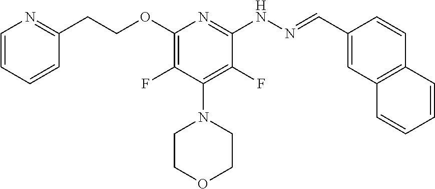 [SMILES: Fc1c(N/N=C/c2ccc3ccccc3c2)nc(OCCc2ccccn2)c(F)c1N1CCOCC1] | N-[3,5-Difluoro-4-morpholin- 4-yl-6-(2-pyridin-2-yl-ethoxy)- pyridin-2-yl]-N′-naphthalen-2- ylmethylene-hydrazine |
| 271 | 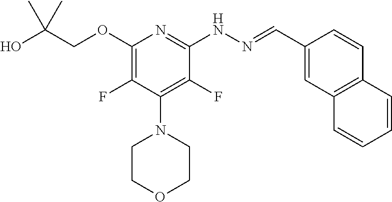 [SMILES: CC(C)(O)COc1nc(N/N=C/c2ccc3ccccc3c2)c(F)c(N2CCOCC2)c1F] | 1-[3,5-Difluoro-4-morpholin-4- yl-6-(N′-naphthalen-2- ylmethylene-hydrazino)- pyridin-2-yloxy]-2-methyl- propan-2-ol |
| 272 | 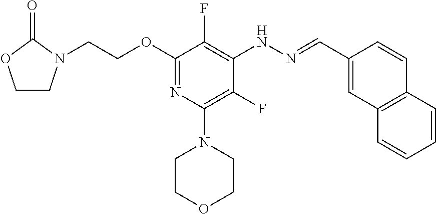 [SMILES: O=C1OCCN1CCOc1nc(N2CCOCC2)c(F)c(N/N=C/c2ccc3ccccc3c2)c1F] | 3-{2-[3,5-Difluoro-6- morpholin-4-yl-4-(N′- naphthalen-2-ylmethylene- hydrazino)-pyridin-2-yloxy]- ethyl}-oxazolidin-2-one |
| 273 | 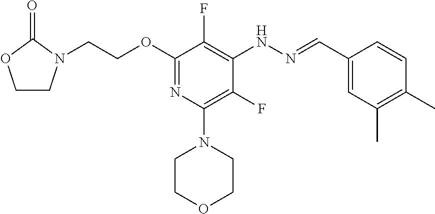 [SMILES: Cc1ccc(/C=N/Nc2c(F)c(OCCN3CCOC3=O)nc(N3CCOCC3)c2F)cc1C] | 3-(2-{4-[N′-(3,4-Dimethyl- benzylidene)-hydrazino]-3,5- difluoro-6-morpholin-4-yl- pyridin-2-yloxy}-ethyl)- oxazolidin-2-one |
| 274 | 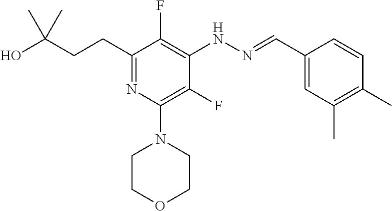 [SMILES: Cc1ccc(/C=N/Nc2c(F)c(CCC(C)(C)O)nc(N3CCOCC3)c2F)cc1C] | 4-{4-[N′-(3,4-Dimethyl- benzylidene)-hydrazino]-3,5- difluoro-6-morpholin-4-yl- pyridin-2-yl}-2-methyl-butan- 2-ol |
| 275 | 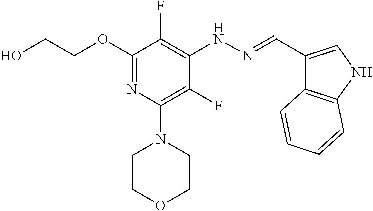 [SMILES: OCCOc1nc(N2CCOCC2)c(F)c(N/N=C/c2c[nH]c3ccccc23)c1F] | 2-{3,5-Difluoro-4-[N′-(1H- indol-3-ylmethylene)- hydrazino]-6-morpholin-4-yl- pyridin-2-yloxy}-ethanol |
| 276 | 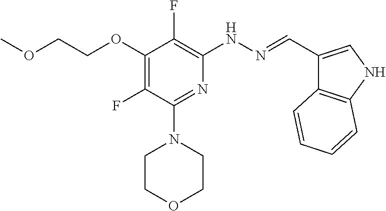 [SMILES: COCCOc1c(F)c(N/N=C/c2c[nH]c3ccccc23)nc(N2CCOCC2)c1F] | N-[3,5-Difluoro-4-(2-methoxy- ethoxy)-6-morpholin-4-yl- pyridin-2-yl]-N′-(1H-indol-3- ylmethylene)-hydrazine |
| 277 | 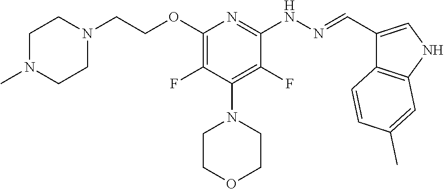 [SMILES: Cc1ccc2c(/C=N/Nc3nc(OCCN4CCN(C)CC4)c(F)c(N4CCOCC4)c3F)c[nH]c2c1] | N-{3,5-Difluoro-6-[2-(4- methyl-piperazin-1-yl)- ethoxy]-4-morpholin-4-yl- pyridin-2-yl}-N′-(6-methyl-1H- indol-3-ylmethylene)-hydrazine |
| 278 | 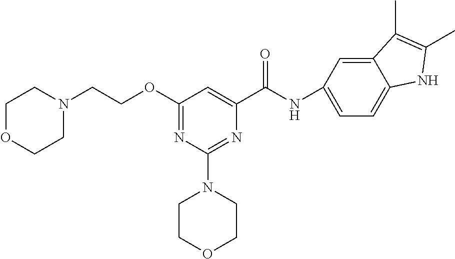 [SMILES: Cc1[nH]c2ccc(NC(=O)c3cc(OCCN4CCOCC4)nc(N4CCOCC4)n3)cc2c1C] | 2-Morpholin-4-yl-6-(2- morpholin-4-yl-ethoxy)- pyrimidine-4-carboxylic acid (2,3-dimethyl-1H-indol-5-yl)- amide |
| 279 | 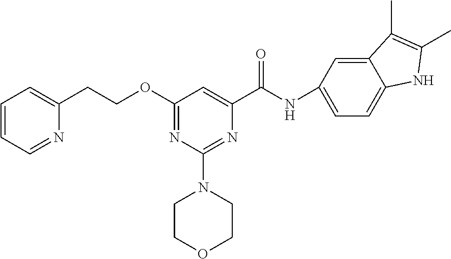 [SMILES: Cc1[nH]c2ccc(NC(=O)c3cc(OCCc4ccccn4)nc(N4CCOCC4)n3)cc2c1C] | 2-Morpholin-4-yl-6-(2-pyridin- 2-yl-ethoxy)-pyrimidine-4- carboxylic acid (2,3-dimethyl- 1H-indol-5-yl)-amide |
| 280 | 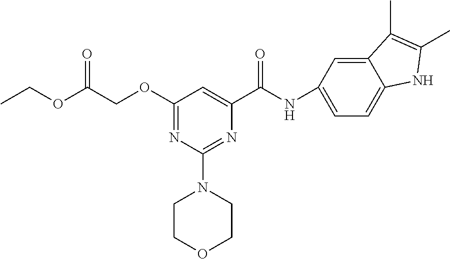 [SMILES: CCOC(=O)COc1cc(C(=O)Nc2ccc3[nH]c(C)c(C)c3c2)nc(N2CCOCC2)n1] | [6-(2,3-Dimethyl-1H-indol-5- ylcarbamoyl)-2-morpholin-4-yl- pyrimidin-4-yloxy]-acetic acid ethyl ester |
| 281 | 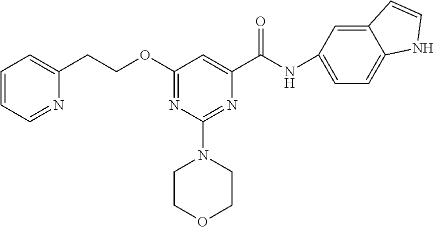 [SMILES: O=C(Nc1ccc2[nH]ccc2c1)c1cc(OCCc2ccccn2)nc(N2CCOCC2)n1] | 2-Morpholin-4-yl-6-(2-pyidin- 2-yl-ethoxy)-pyrimidine-4- carboxylic acid (1H-indol-5- yl)-amide |
| 282 | 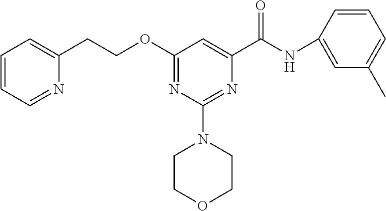 [SMILES: Cc1cccc(NC(=O)c2cc(OCCc3ccccn3)nc(N3CCOCC3)n2)c1] | 2-Morpholin-4-yl-6-(2-pyridin- 2-yl-ethoxy)-pyrimidine-4- carboxylic acid m-tolylamide |
| 283 | 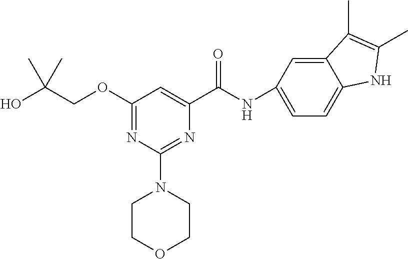 [SMILES: Cc1[nH]c2ccc(NC(=O)c3cc(OCC(C)(C)O)nc(N4CCOCC4)n3)cc2c1C] | 6-(2-hydroxy-2-methyl- propoxy)-2-morpholin-4-yl- pyrimidin-4-carboxylic acid (2,3-dimethyl-1H-indol-5-yl)- amide |
| 284 | 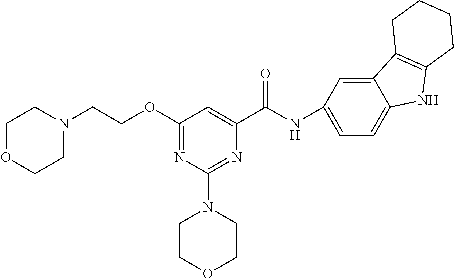 [SMILES: O=C(Nc1ccc2[nH]c3c(c2c1)CCCC3)c1cc(OCCN2CCOCC2)nc(N2CCOCC2)n1] | 2-Morpholin-4-yl-6-(2- morpholin-4-yl-ethoxy)- pyrimidine-4-carboxylic acid (6,7,8,9-tetrahydro-5H- carbazol-3-yl)-amide |
| 285 | 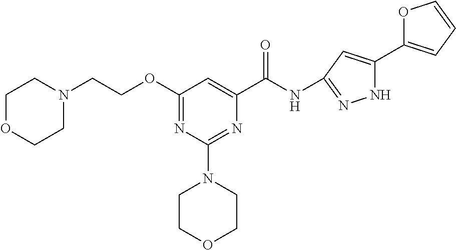 [SMILES: O=C(Nc1cc(-c2ccco2)[nH]n1)c1cc(OCCN2CCOCC2)nc(N2CCOCC2)n1] | 2-Morpholin-4-yl-6-(2- morpholin-4-yl-ethoxy)- pyrimidine-4-carboxylic acid (5-furan-2-yl-1H-pyrazol-3-yl)- amide |
| 286 | 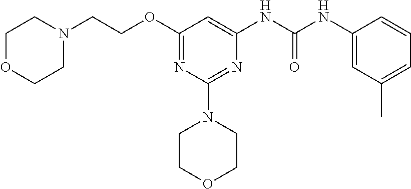 [SMILES: Cc1cccc(NC(=O)Nc2cc(OCCN3CCOCC3)nc(N3CCOCC3)n2)c1] | 1-[2-Morpholin-4-yl-6-(2- morpholin-4-yl-ethoxy)- pyrimidin-4-yl]-3-m-tolyl-urea |
| 287 | 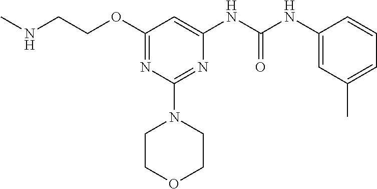 [SMILES: CNCCOc1cc(NC(=O)Nc2cccc(C)c2)nc(N2CCOCC2)n1] | 1-[6-(2-Methylamino-ethoxy)- 2-morpholin-4-yl-pyrimidin-4- yl]-3-m-tolyl-urea |
| 288 | 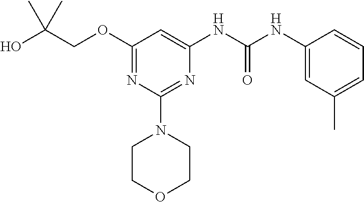 [SMILES: Cc1cccc(NC(=O)Nc2cc(OCC(C)(C)O)nc(N3CCOCC3)n2)c1] | 1-[6-(2-Hydroxy-2-methyl- propoxy)-2-morpholin-4-yl- pyrimidin-4-yl]-3-m-tolyl-urea |
| 289 | 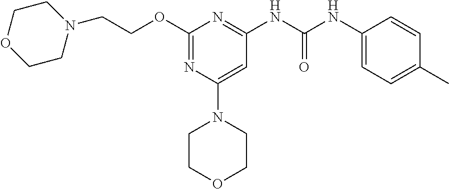 [SMILES: Cc1ccc(NC(=O)Nc2cc(N3CCOCC3)nc(OCCN3CCOCC3)n2)cc1] | 1-[6-Morpholin-4-yl-2-(2- morpholin-4-yl-ethoxy)- pyrimidin-4-yl]-3-p-tolyl- thiourea |
| 290 | 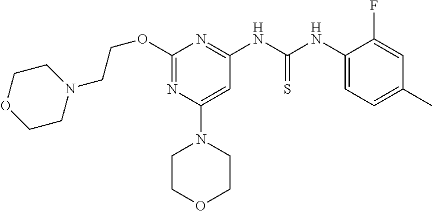 [SMILES: Cc1ccc(NC(=S)Nc2cc(N3CCOCC3)nc(OCCN3CCOCC3)n2)c(F)c1] | 1-(2-Bromo-4-methyl-phenyl)- 3-[6-morpholin-4-yl-2-(2- morpholin-4-yl-ethoxy)- pyrimidin-4-yl]-thiourea |
| 291 | 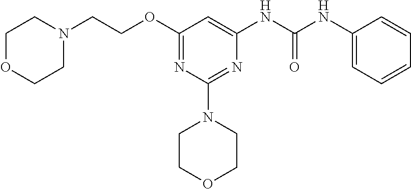 [SMILES: O=C(Nc1ccccc1)Nc1cc(OCCN2CCOCC2)nc(N2CCOCC2)n1] | 1-[2-Morpholin-4-yl-6-(2- morpholin-4-yl-ethoxy)- pyrimidin-4-yl]-3-phenyl-urea |
| 293 | 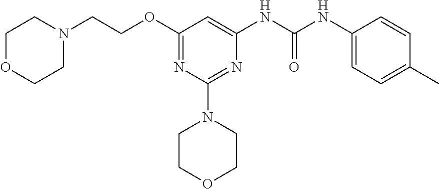 [SMILES: Cc1ccc(NC(=O)Nc2cc(OCCN3CCOCC3)nc(N3CCOCC3)n2)cc1] | 1-[2-Morpholin-4-yl-6-(2- morpholin-4-yl-ethoxy)- pyrimidin-4-yl]-3-p-tolyl-urea |
| 294 | 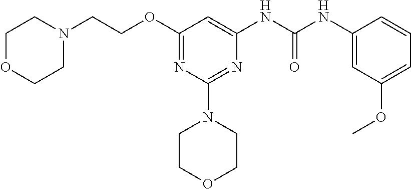 [SMILES: COc1cccc(NC(=O)Nc2cc(OCCN3CCOCC3)nc(N3CCOCC3)n2)c1] | 1-(3-Methoxy-phenyl)-3-[2- morpholin-4-yl-6-(2- morpholin-4-yl-ethoxy)- pyrimidin-4-yl]-urea |
| 295 | 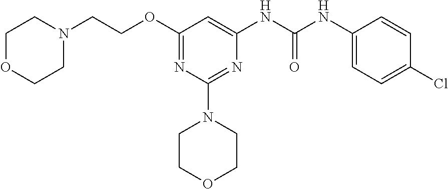 [SMILES: O=C(Nc1ccc(Cl)cc1)Nc1cc(OCCN2CCOCC2)nc(N2CCOCC2)n1] | 1-(4-Chloro-phenyl)-3-[2- morpholin-4-yl-6-(2- morpholin-4-yl-ethoxy)- pyrimidin-4-yl]-urea |
| 296 | 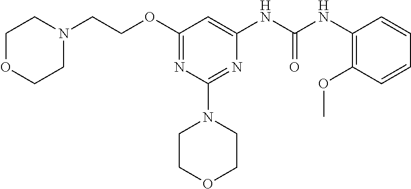 [SMILES: COc1ccccc1NC(=O)Nc1cc(OCCN2CCOCC2)nc(N2CCOCC2)n1] | 1-(2-Methoxy-phenyl)-3-[2- morpholin-4-yl-6-(2- morpholin-4-yl-ethoxy)- pyrimidin-4-yl]-urea |
| 297 | 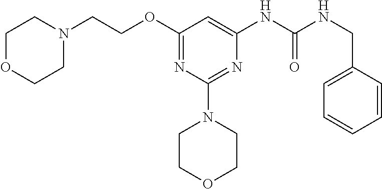 [SMILES: O=C(NCc1ccccc1)Nc1cc(OCCN2CCOCC2)nc(N2CCOCC2)n1] | 1-Benzyl-3-[2-morpholin-4-yl- 6-(2-morpholin-4-yl-ethoxy)- pyrimidin-4-yl]-urea |
| 298 | 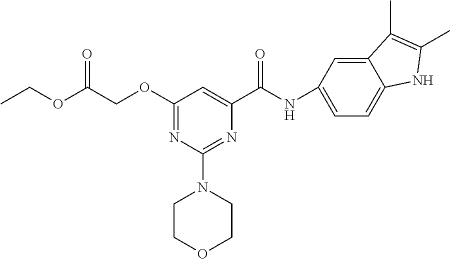 [SMILES: CCOC(=O)COc1cc(C(=O)Nc2ccc3[nH]c(C)c(C)c3c2)nc(N2CCOCC2)n1] | [6-(2,3-Dimethyl-1H-indol-5- ylcarbamoyl)-2-morpholin-4- yl-pyrimidin-4-yloxy]-acetic acid ethyl ester |
| 299 | 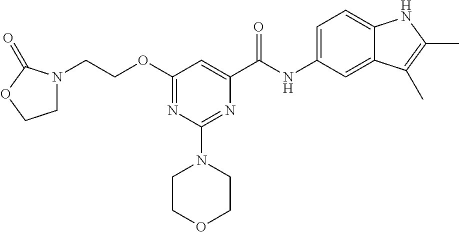 [SMILES: Cc1[nH]c2ccc(NC(=O)c3cc(OCCN4CCOC4=O)nc(N4CCOCC4)n3)cc2c1C] | 2-Morpholin-4-yl-6-[2-(2-oxo- oxazolidin-3-yl)-ethoxy]- pyrimidine-4-carboxylic acid (2,3-dimethyl-1H-indol-5-yl)- amide |
| 300 | 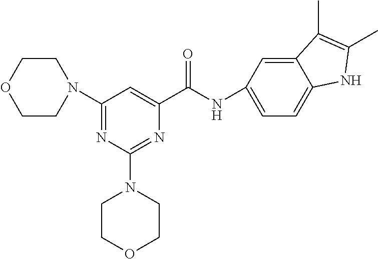 [SMILES: Cc1[nH]c2ccc(NC(=O)c3cc(N4CCOCC4)nc(N4CCOCC4)n3)cc2c1C] | 2,6-Di-morpholin-4-yl- pyrimidine-4-carboxylic acid (2,3-dimethyl-1H-indol-5-yl)- amide |
| 301 | 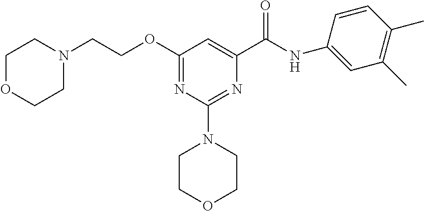 [SMILES: Cc1ccc(NC(=O)c2cc(OCCN3CCOCC3)nc(N3CCOCC3)n2)cc1C] | 2-Morpholin-4-yl-6-(2- morpholin-4-yl-ethoxy)- pyrimidine-4-carboxylic acid (3,4-dimethyl-phenyl)-amide |
| 302 | 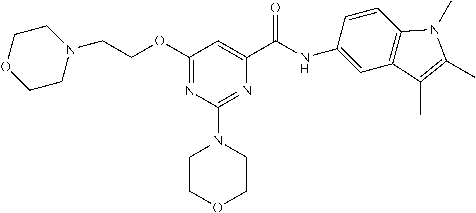 [SMILES: Cc1c(C)n(C)c2ccc(NC(=O)c3cc(OCCN4CCOCC4)nc(N4CCOCC4)n3)cc12] | 2-Morpholin-4-yl-6-(2- morpholin-4-yl-ethoxy)- pyrimidine-4-carboxylic acid (1,2,3-trimethyl-1H-indol-5- yl)-amide |
| 303 | 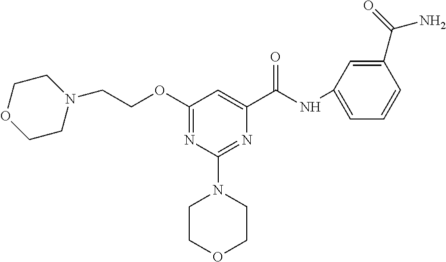 [SMILES: NC(=O)c1cccc(NC(=O)c2cc(OCCN3CCOCC3)nc(N3CCOCC3)n2)c1] | 2-Morpholin-4-yl-6-(2- morpholin-4-yl-ethoxy)- pyrimidine-4-carboxylic acid (3-carbamoyl-phenyl)-amide |
| 304 | 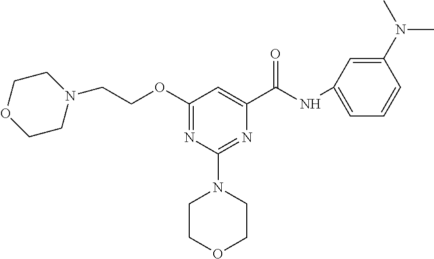 [SMILES: CN(C)c1cccc(NC(=O)c2cc(OCCN3CCOCC3)nc(N3CCOCC3)n2)c1] | 2-Morpholin-4-yl-6-(2- morpholin-4-yl-ethoxy)- pyrimidine-4-carboxylic acid (3-dimethylamino-phenyl)- amide |
| 305 | 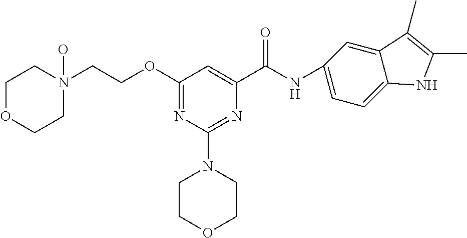 | 2-Morpholin-4-yl-6-[2-(4-oxy- morpholin-4-yl)-ethoxy]-pyrimidine- 4-carboxylic acid (2,3-dimethyl-1H- indol-5-yl)-amide |
| 306 | 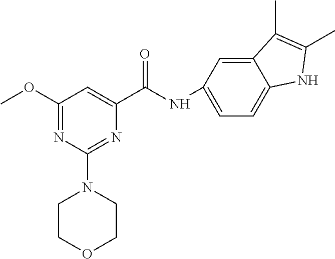 | 6-Methoxy-2-morpholin-4-yl- pyrimidine-4-carboxylic acid (2,3-dimethyl-1H-indol-5-yl)- amide |
| 307 | 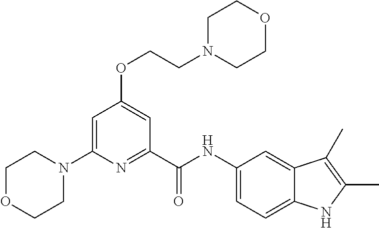 [SMILES: Cc1[nH]c2ccc(NC(=O)c3cc(OCCN4CCOCC4)cc(N4CCOCC4)n3)cc2c1C] | 6-Morpholin-4-yl-4-(2- morpholin-4-yl-ethoxy)- pyridine-2-carboxylic acid (2,3-dimethyl-1H-indol-5-yl)- amide |
| 307 | 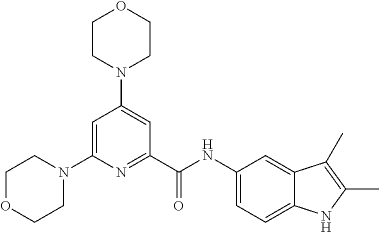 [SMILES: Cc1[nH]c2ccc(NC(=O)c3cc(N4CCOCC4)cc(N4CCOCC4)n3)cc2c1C] | 4,6-Di-morpholin-4-yl- pyridine-2-carboxylic aci (2,3- dimethyl-1H-indol-5-yl)-amide |
| 308 | 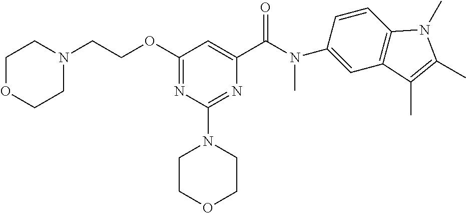 [SMILES: Cc1c(C)n(C)c2ccc(N(C)C(=O)c3cc(OCCN4CCOCC4)nc(N4CCOCC4)n3)cc12] | 2-Morpholin-4-yl-6-(2- morpholin-4-yl-ethoxy)- pyrimidine-4-carboxylic acid methyl-(1,2,3-trimethyl-1H- indol-5-yl)-amide |
| 309 | 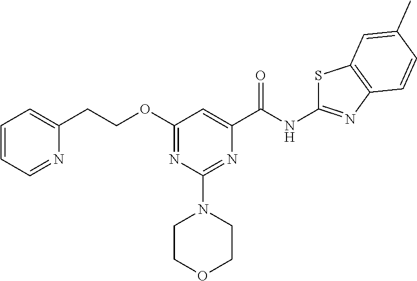 [SMILES: Cc1ccc2nc(NC(=O)c3cc(OCCc4ccccn4)nc(N4CCOCC4)n3)sc2c1] | 2-Morpholin-4-yl-6-(2-pyridin- 2-yl-ethoxy)-pyrimidine-4- carboxylic acid (9-ethyl-9H- carbazol-2-yl)-amide |
| 310 | 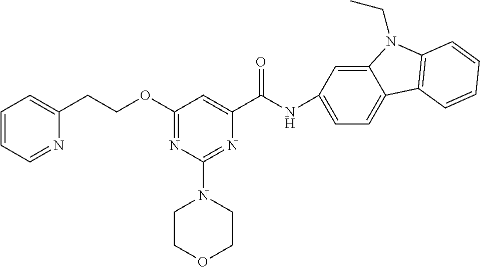 [SMILES: CCn1c2ccccc2c2ccc(NC(=O)c3cc(OCCc4ccccn4)nc(N4CCOCC4)n3)cc21] | 2-Morpholin-4-yl-6-(2-pyridin- 2-yl-ethoxy)-pyrimidine-4- carboxylic acid (9-ethyl-9H- carbazol-2-yl)-amide |
| 311 | 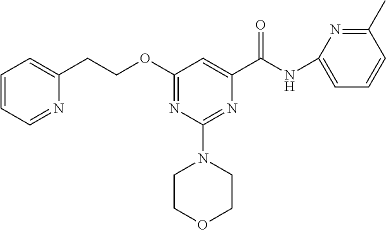 [SMILES: Cc1cccc(NC(=O)c2cc(OCCc3ccccn3)nc(N3CCOCC3)n2)n1] | 2-Morpholin-4-yl-6-(2-pyridin- 2-yl-ethoxy)-pyrimidine-4- carboxylic acid (6-methyl- pyridin-2-yl)-amide |
| 312 | 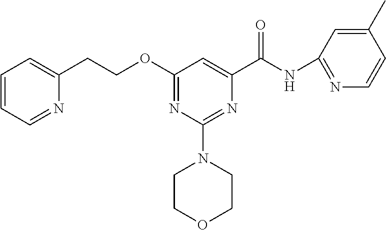 [SMILES: Cc1ccnc(NC(=O)c2cc(OCCc3ccccn3)nc(N3CCOCC3)n2)c1] | 2-Morpholin-4-yl-6-(2-pyridin- 2-yl-ethoxy)-pyrimidine-4- carboxylic acid (4-methyl- pyridin-2-yl)-amide |
| 313 | 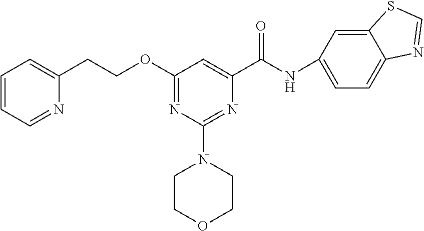 [SMILES: O=C(Nc1ccc2ncsc2c1)c1cc(OCCc2ccccn2)nc(N2CCOCC2)n1] | 2-Morpholin-4-yl-6-(2-pyridin- 2-yl-ethoxy)-pyrimidine-4- carboxylic acid benzothiazol-6- ylamide |
| 314 | 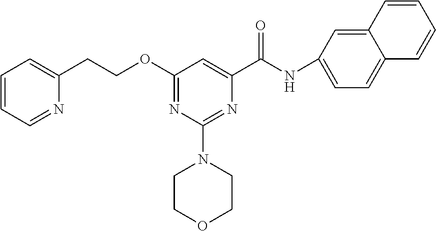 [SMILES: O=C(Nc1ccc2ccccc2c1)c1cc(OCCc2ccccn2)nc(N2CCOCC2)n1] | 2-Morpholin-4-yl-6-(2-pyridin- 2-yl-ethoxy)-pyrimidine-4- carboxylic acid naphthalen-2- ylamide |
| 315 | 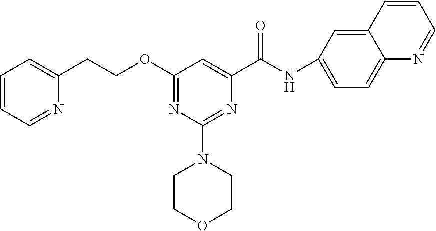 [SMILES: O=C(Nc1ccc2ncccc2c1)c1cc(OCCc2ccccn2)nc(N2CCOCC2)n1] | 2-Morpholin-4-yl-6-(2-pyridin- 2-yl-ethoxy)-pyrimidine-4- carboxylic acid quinolin-6- ylamide |
| 316 | 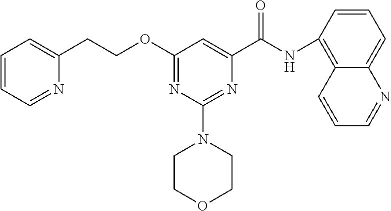 [SMILES: O=C(Nc1cccc2ncccc12)c1cc(OCCc2ccccn2)nc(N2CCOCC2)n1] | 2-Morpholin-4-yl-6-(2-pyridin- 2-yl-ethoxy)-pyrimidine-4- carboxylic acid quinolin-5- ylamide |
| 317 | 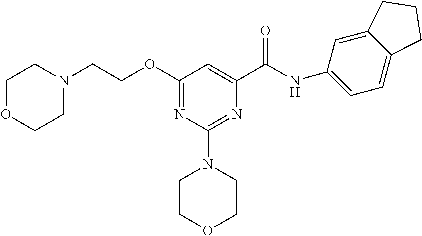 [SMILES: O=C(Nc1ccc2c(c1)CCC2)c1cc(OCCN2CCOCC2)nc(N2CCOCC2)n1] | 2-Morpholin-4-yl-6-(2- morpholin-4-yl-ethoxy)- pyrimidine-4-carboxylic acid indan-5-ylamide |
| 318 | 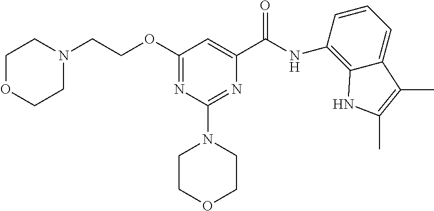 [SMILES: Cc1[nH]c2c(NC(=O)c3cc(OCCN4CCOCC4)nc(N4CCOCC4)n3)cccc2c1C] | 2-Morpholin-4-yl-6-(2- morpholin-4-yl-ethoxy)- pyrimidine-4-carboxylic acid (2,3-dimethyl-1H-indol-7-yl)- amide |
| 319 | 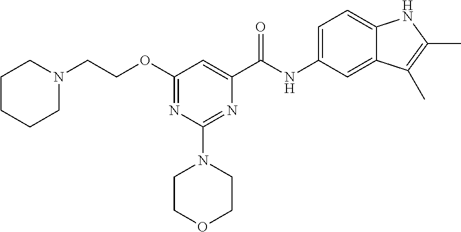 [SMILES: Cc1[nH]c2ccc(NC(=O)c3cc(OCCN4CCCCC4)nc(N4CCOCC4)n3)cc2c1C] | 2-Morpholin-4-yl-6-(2- piperidin-1-yl-ethoxy)- pyrimidine-4-carboxylic acid (2,3-dimethyl-1H-indol-5-yl)- amide |
| 320 | 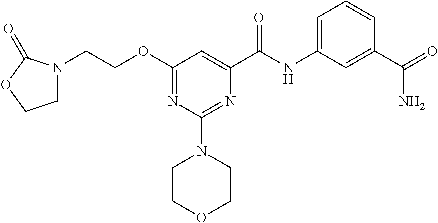 [SMILES: NC(=O)c1cccc(NC(=O)c2cc(OCCN3CCOC3=O)nc(N3CCOCC3)n2)c1] | 2-Morpholin-4-yl-6-[2-(2-oxo- oxazolidin-3-yl)-ethoxy]- pyrimidine-4-carboxylic acid (3-carbamoyl-phenyl)-amide |
| 321 | 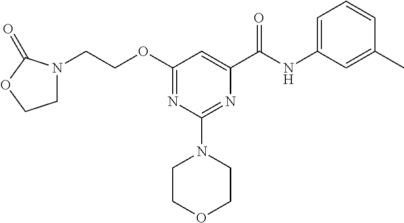 [SMILES: Cc1cccc(NC(=O)c2cc(OCCN3CCOC3=O)nc(N3CCOCC3)n2)c1] | 2-Morpholin-4-yl-6-[2-(2-oxo- oxazolidin-3-yl)-ethoxy]- pyrimidine-4-carboxylic acid m-tolylamide |
| 322 | 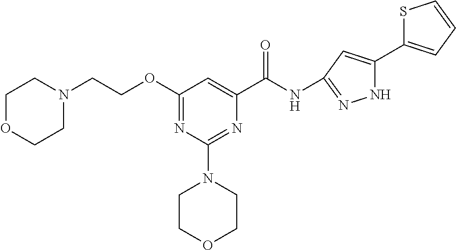 [SMILES: O=C(Nc1cc(-c2cccs2)[nH]n1)c1cc(OCCN2CCOCC2)nc(N2CCOCC2)n1] | 2-Morpholin-4-yl-6-(2- morpholin-4-yl-ethoxy)- pyrimidine-4-carboxylic acid (5-thiophen-2-yl-1H-pyrazol-3- yl)-amide |
| 323 | 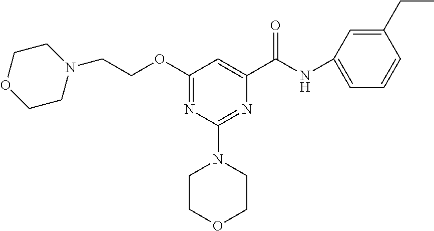 [SMILES: CCc1cccc(NC(=O)c2cc(OCCN3CCOCC3)nc(N3CCOCC3)n2)c1] | 2-Morpholin-4-yl-6-(2- morpholin-4-yl-ethoxy)- pyrimidine-4-carboxylic acid (3-ethyl-phenyl)-amide |
| 324 | 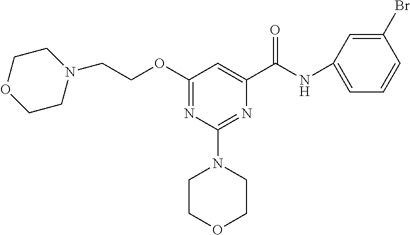 [SMILES: O=C(Nc1cccc(Br)c1)c1cc(OCCN2CCOCC2)nc(N2CCOCC2)n1] | 2-Morpholin-4-yl-6-(2- morpholin-4-yl-ethoxy)- pyrimidine-4-carboxylic acid (3-bromo-phenyl)-amide |
| 325 | 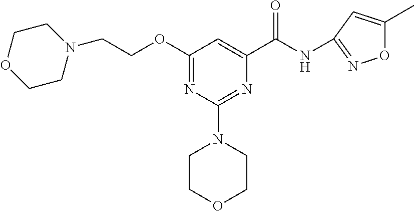 [SMILES: Cc1cc(NC(=O)c2cc(OCCN3CCOCC3)nc(N3CCOCC3)n2)no1] | 2-Morpholin-4-yl-6-(2- morpholin-4-yl-ethoxy)- pyrimidine-4-carboxylic acid (5-methyl-isoxazol-3-yl)-amide |
| 326 | 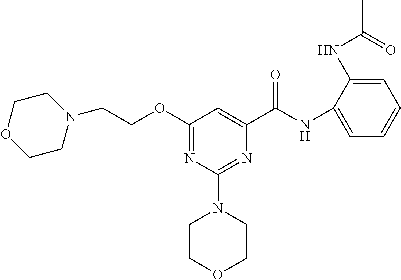 [SMILES: CC(=O)Nc1ccccc1NC(=O)c1cc(OCCN2CCOCC2)nc(N2CCOCC2)n1] | 2-Morpholin-4-yl-6-(2- morpholin-4-yl-ethoxy)- pyrimidine-4-carboxylic acid (2-acetylamino-phenyl)-amide |
| 327 | 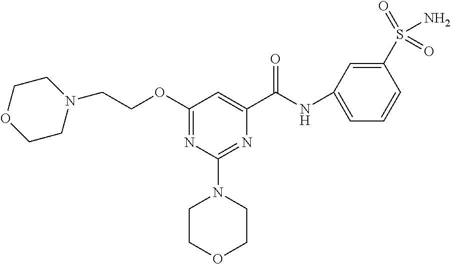 [SMILES: NS(=O)(=O)c1cccc(NC(=O)c2cc(OCCN3CCOCC3)nc(N3CCOCC3)n2)c1] | 2-Morpholin-4-yl-6-(2- morpholin-4-yl-ethoxy)- pyrimidine-4-carboxylic acid (3-sulfamoyl-phenyl)-amide |
| 328 | 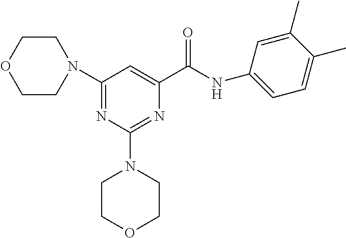 [SMILES: Cc1ccc(NC(=O)c2cc(N3CCOCC3)nc(N3CCOCC3)n2)cc1C] | 2,6-Di-morpholin-4-yl- pyrimidine-4-carboxylic acid (3,4-dimethyl-phenyl)-amide |
| 329 | 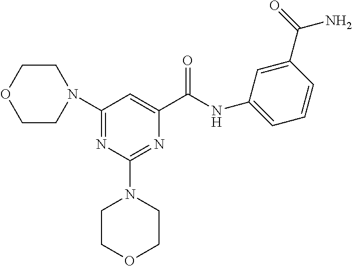 [SMILES: NC(=O)c1cccc(NC(=O)c2cc(N3CCOCC3)nc(N3CCOCC3)n2)c1] | 2,6-Di-morpholin-4-yl- pyrimidine-4-carboxylic acid (3-carbamoyl-phenyl)-amide |
| 330 | 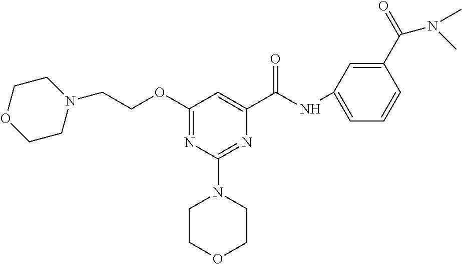 [SMILES: CN(C)C(=O)c1cccc(NC(=O)c2cc(OCCN3CCOCC3)nc(N3CCOCC3)n2)c1] | 2-Morpholin-4-yl-6-(2- morpholin-4-yl-ethoxy)- pyrimidine-4-carboxylic acid (3-dimethylcarbamoyl-phenyl)- amide |
| 331 | 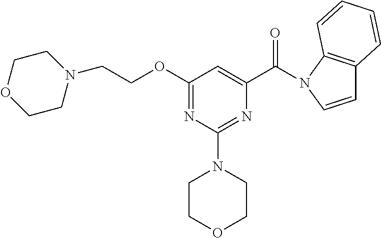 [SMILES: O=C(c1cc(OCCN2CCOCC2)nc(N2CCOCC2)n1)n1ccc2ccccc21] | Indol-1-yl-[2-morpholin-4-yl- 6-(2-morpholin-4-yl-ethoxy)- pyrimidin-4-yl]-methanone |
| 332 | 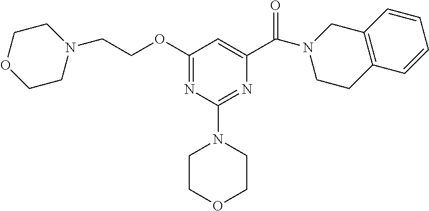 [SMILES: O=C(c1cc(OCCN2CCOCC2)nc(N2CCOCC2)n1)N1CCc2ccccc2C1] | (3,4-Dihydro-1H-isoquinolin- 2-yl)-[2-morpholin-4-yl-6-(2- morpholin-4-yl-ethoxy)- pyrimidin-4-yl]-methanone |
| 333 | 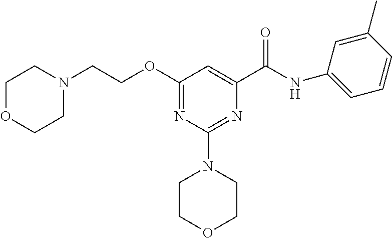 [SMILES: Cc1cccc(NC(=O)c2cc(OCCN3CCOCC3)nc(N3CCOCC3)n2)c1] | 2-Morpholin-4-yl-6-(2- morpholin-4-yl-ethoxy)- pyrimidine-4-carboxylic acid m-tolylamide |
| 334 | 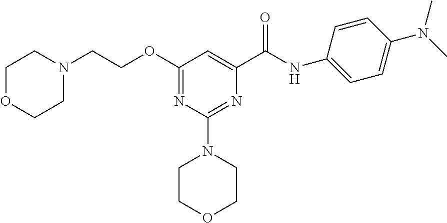 [SMILES: CN(C)c1ccc(NC(=O)c2cc(OCCN3CCOCC3)nc(N3CCOCC3)n2)cc1] | 2-Morpholin-4-yl-6-(2- morpholin-4-yl-ethoxy)- pyrimidine-4-carboxylic acid (4-dimethylamino-phenyl)- amide |
| 335 | 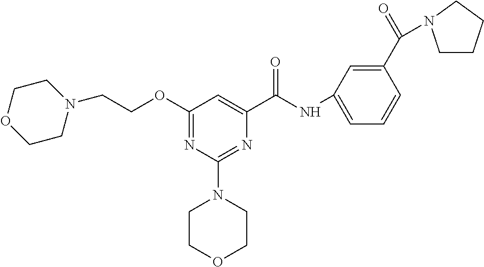 [SMILES: O=C(Nc1cccc(C(=O)N2CCCC2)c1)c1cc(OCCN2CCOCC2)nc(N2CCOCC2)n1] | 2-Morpholin-4-yl-6-(2- morpholin-4-yl-ethoxy)- pyrimidine-4-carboxylic acid [3-(pyrrolidine-1-carbonyl)- phenyl]-amide |
| 336 | 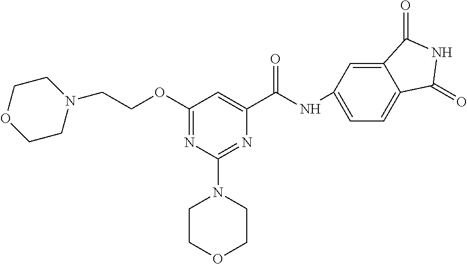 [SMILES: O=C(Nc1ccc2c(c1)C(=O)NC2=O)c1cc(OCCN2CCOCC2)nc(N2CCOCC2)n1] | 2-Morpholin-4-yl-6-(2- morpholin-4-yl-ethoxy)- pyrimidine-4-carboxylic acid (1,3-dioxo-2,3-dihydro-1H- isoindol-5-yl)-amide |
| 337 | 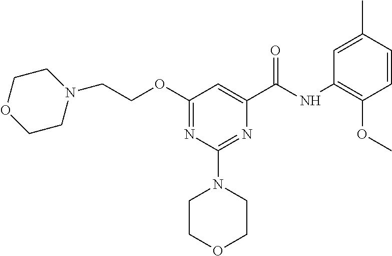 [SMILES: COc1ccc(C)cc1NC(=O)c1cc(OCCN2CCOCC2)nc(N2CCOCC2)n1] | 2-Morpholin-4-yl-6-(2- morpholin-4-yl-ethoxy)- pyrimidine-4-carboxylic acid (2-methoxy-5-methyl-phenyl)- amide |
| 338 | 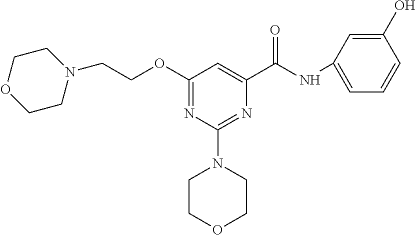 [SMILES: O=C(Nc1cccc(O)c1)c1cc(OCCN2CCOCC2)nc(N2CCOCC2)n1] | 2-Morpholin-4-yl-6-(2- morpholin-4-yl-ethoxy)- pyrimidine-4-carboxylic acid (3-hydroxy-phenyl)-amide |
| 339 | 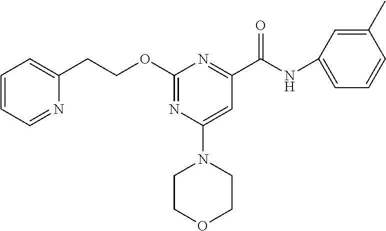 [SMILES: Cc1cccc(NC(=O)c2cc(N3CCOCC3)nc(OCCc3ccccn3)n2)c1] | 6-Morpholin-4-yl-2-(2-pyridin- 2-yl-ethoxy)-pyrimidine-4- carboxylic acid m-tolylamide |
| 340 | 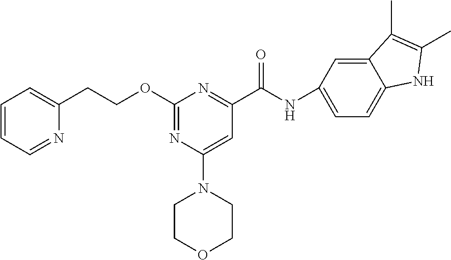 [SMILES: Cc1[nH]c2ccc(NC(=O)c3cc(N4CCOCC4)nc(OCCc4ccccn4)n3)cc2c1C] | 6-Morpholin-4-yl-2-(2-pyridin- 2-yl-ethoxy)-pyrimidine-4- carboxylic acid (2,3-dimethyl- 1H-indol-5-yl)-amide |
| 341 | 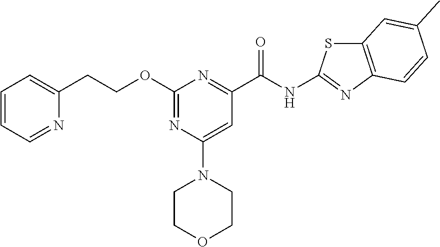 [SMILES: Cc1ccc2nc(NC(=O)c3cc(N4CCOCC4)nc(OCCc4ccccn4)n3)sc2c1] | 6-Morpholin-4-yl-2-(2-pyridin- 2-yl-ethoxy)-pyrimidine-4- carboxylic acid (6-methyl- benzothiazol-2-yl)-amide |
| 342 | 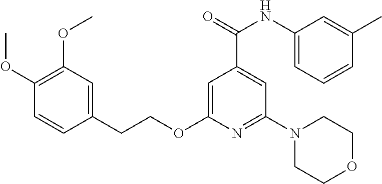 [SMILES: COc1ccc(CCOc2cc(C(=O)Nc3cccc(C)c3)cc(N3CCOCC3)n2)cc1OC] | 2-[2-(3,4-Dimethoxy-phenyl)- ethoxy]-6-morpholin-4-yl-N-m- tolyl-isonicotinamide |
| 343 | 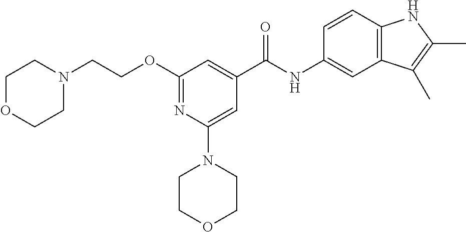 [SMILES: Cc1[nH]c2ccc(NC(=O)c3cc(OCCN4CCOCC4)nc(N4CCOCC4)c3)cc2c1C] | N-(2,3-Dimethyl-1H-indol-5- yl)-2-morpholin-4-yl-6-(2- morpholin-4-yl-ethoxy)- isonicotinamide |
| 344 | 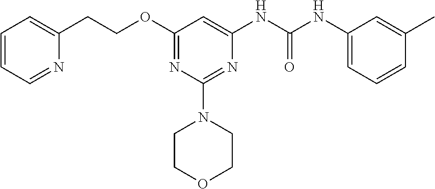 [SMILES: Cc1cccc(NC(=O)Nc2cc(OCCc3ccccn3)nc(N3CCOCC3)n2)c1] | 1-[2-Morpholin-4-yl-6-(2- pyridin-2-yl-ethoxy)- pyrimidin-4-yl]-3-m-tolyl-urea |
| 345 | 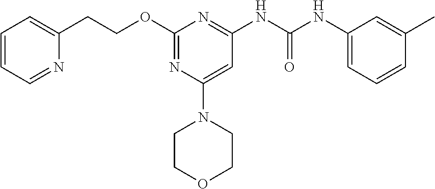 [SMILES: Cc1cccc(NC(=O)Nc2cc(N3CCOCC3)nc(OCCc3ccccn3)n2)c1] | 1-[6-Morpholin-4-yl-2-(2- pyridin-2-yl-ethoxy)- pyrimidin-4-yl]-3-m-tolyl-urea |
| 346 | 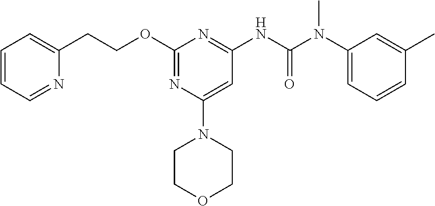 [SMILES: Cc1cccc(N(C)C(=O)Nc2cc(N3CCOCC3)nc(OCCc3ccccn3)n2)c1] | 1-Methyl-3-[6-morpholin-4-yl- 2-(2-pyridin-2-yl-ethoxy)- pyrimidin-4-yl]-1-m-tolyl-urea |
| 347 | 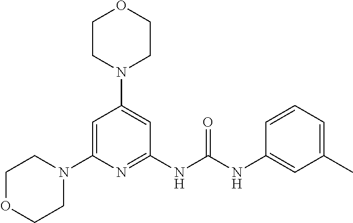 [SMILES: Cc1cccc(NC(=O)Nc2cc(N3CCOCC3)cc(N3CCOCC3)n2)c1] | 1-(4,6-Di-morpholin-4-yl- pyridin-2-yl)-3-m-tolyl-urea |
| 348 | 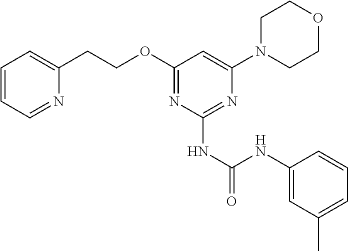 [SMILES: Cc1cccc(NC(=O)Nc2nc(OCCc3ccccn3)cc(N3CCOCC3)n2)c1] | 1-[(4-Morpholin-4-yl-6-(2- pyridin-2-yl-ethoxy)- pyrimidin-2-yl]-3-m-tolyl-urea |
| 349 | 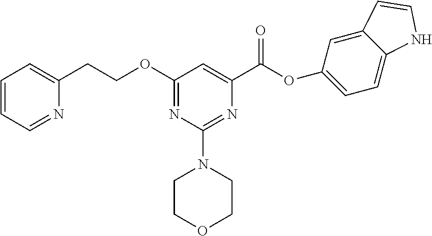 [SMILES: O=C(Oc1ccc2[nH]ccc2c1)c1cc(OCCc2ccccn2)nc(N2CCOCC2)n1] | 2-Morpholin-4-yl-6-(2-pyridin- 2-yl-ethoxy)-pyrimidine-4- carboxylic acid 1H-indol-5-yl ester |
| 350 | 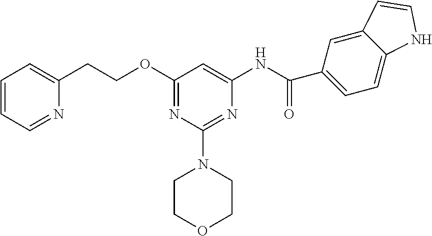 [SMILES: O=C(Nc1cc(OCCc2ccccn2)nc(N2CCOCC2)n1)c1ccc2[nH]ccc2c1] | 1H-Indole-5-carboxylic acid [2-morpholin-4-yl-6-(2- pyridin-2-yl-ethoxy)- pyrimidin-4-yl]-amide |
| 351 | 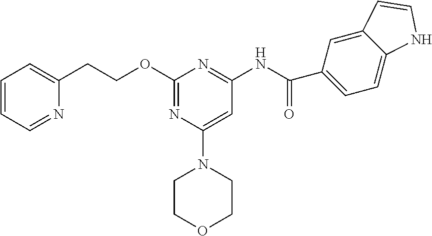 [SMILES: O=C(Nc1cc(N2CCOCC2)nc(OCCc2ccccn2)n1)c1ccc2[nH]ccc2c1] | 1H-Indole-5-carboxylic acid [6-morpholin-4-yl-2-(2- pyridin-2-yl-ethoxy)- pyrimidin-4-yl]-amide |
| 352 | 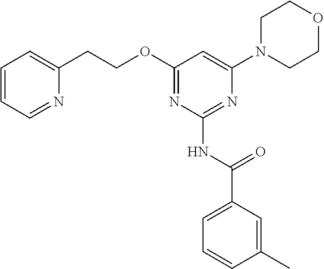 [SMILES: Cc1cccc(C(=O)Nc2nc(OCCc3ccccn3)cc(N3CCOCC3)n2)c1] | 3-Methyl-N-[4-morpholin-4-yl- 6-(2-pyridin-2-yl-ethoxy)- pyrimidin-2-yl]-benzamide |
| 353 | 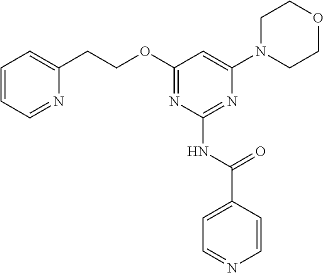 [SMILES: O=C(Nc1nc(OCCc2ccccn2)cc(N2CCOCC2)n1)c1ccncc1] | N-[4-Morpholin-4-yl-6-(2- pyridin-2-yl-ethoxy)- pyrimidin-2-yl]- isonicotinamide |
| 354 | 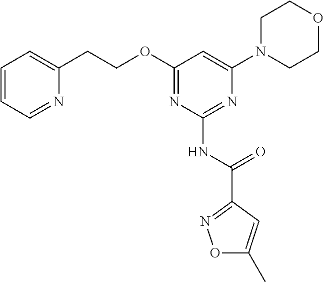 [SMILES: Cc1cc(C(=O)Nc2nc(OCCc3ccccn3)cc(N3CCOCC3)n2)no1] | 5-Methyl-isoxazole-3- carboxylic acid-[4-morpholin- 4-yl-6-(2-pyridin-2-yl-ethoxy)- pyrimidin-2-yl]-amide |
| 355 | 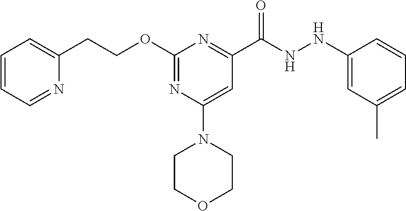 [SMILES: Cc1cccc(NNC(=O)c2cc(N3CCOCC3)nc(OCCc3ccccn3)n2)c1] | 6-Morpholin-4-yl-2-(2-pyridin- 2-yl-ethoxy)-pyrimidine-4- carboxylic acid N′-m-tolyl- hydrazide |
| 356 | 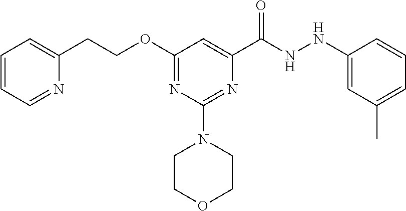 [SMILES: Cc1cccc(NNC(=O)c2cc(OCCc3ccccn3)nc(N3CCOCC3)n2)c1] | 2-Morpholin-4-yl-6-(2-pyridin- 2-yl-ethoxy)-pyrimidine-4- carboxylic acid N′-m-tolyl- hydrazide |
| 357 | 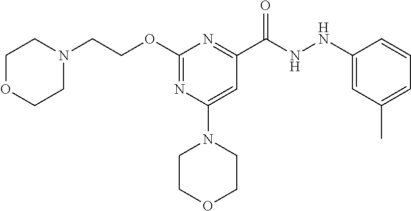 [SMILES: Cc1cccc(NNC(=O)c2cc(N3CCOCC3)nc(OCCN3CCOCC3)n2)c1] | 6-Morpholin-4-yl-2-(2- morpholin-4-yl-ethoxy)- pyrimidine-4-carboxylic acid N′-m-tolyl-hydrazide |
| 358 | 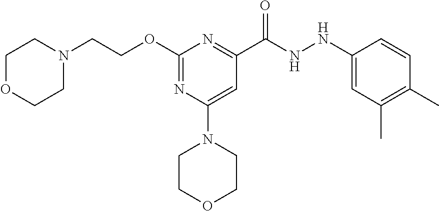 [SMILES: Cc1ccc(NNC(=O)c2cc(N3CCOCC3)nc(OCCN3CCOCC3)n2)cc1C] | 6-Morpholin-4-yl-2-(2- morpholin-4-yl-ethoxy)- pyrimidine-4-carboxylic acid N′-(3,4-dimethyl-phenyl)- hydrazide |
| 359 | 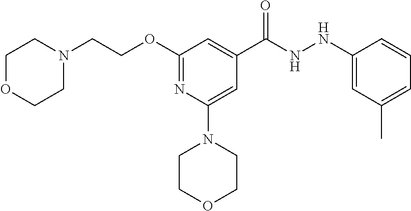 [SMILES: Cc1cccc(NNC(=O)c2cc(OCCN3CCOCC3)nc(N3CCOCC3)c2)c1] | 2-Morpholin-4-yl-6-(2- morpholin-4-yl-ethoxy)- isonicotinic acid N′-m-tolyl- hydrazide |
| 360 | 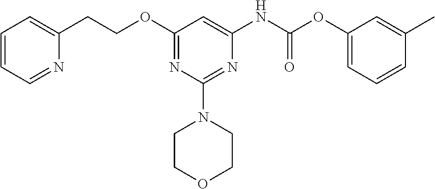 [SMILES: Cc1cccc(OC(=O)Nc2cc(OCCc3ccccn3)nc(N3CCOCC3)n2)c1] | [2-Morpholin-4-yl-6-(2- pyridin-2-yl-ethoxy)- pyrimidin-4-yl]-carbamic acid m-tolyl ester |
| 361 | 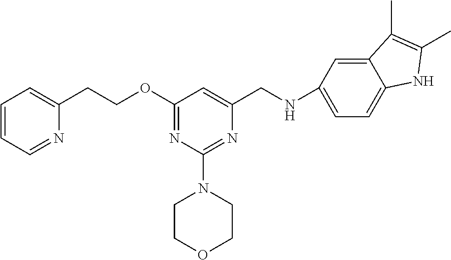 [SMILES: Cc1[nH]c2ccc(NCc3cc(OCCc4ccccn4)nc(N4CCOCC4)n3)cc2c1C] | (2,3-Dimethyl-1H-indol-5-yl)- [2-morpholin-4-yl-6-(2- pyridin-2-yl-ethoxy)- pyrimidin-4-ylmethyl]-amine |
| 362 | 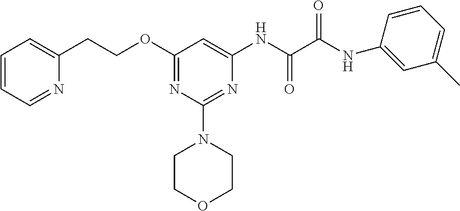 [SMILES: Cc1cccc(NC(=O)C(=O)Nc2cc(OCCc3ccccn3)nc(N3CCOCC3)n2)c1] | N-[2-Morpholin-4-yl-6-(2- pyridin-2-yl-ethoxy)- pyrimidin-4-yl]-N′-m-tolyl- oxalamide |
| 363 | 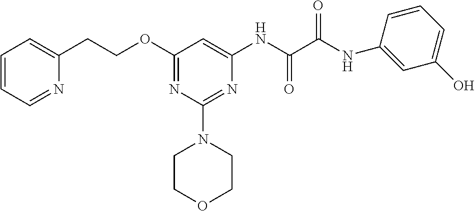 [SMILES: O=C(Nc1cccc(O)c1)C(=O)Nc1cc(OCCc2ccccn2)nc(N2CCOCC2)n1] | N-(3-Hydroxy-phenyl)-N′-[2- morpholin-4-yl-6-(2-pyridin-2- yl-ethoxy)-pyrimidin-4-yl]- oxalamide |
| 364 | 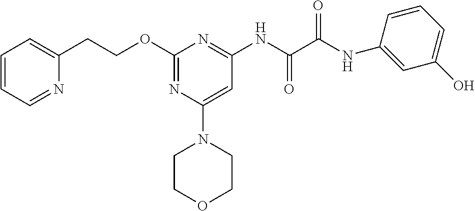 [SMILES: O=C(Nc1cccc(O)c1)C(=O)Nc1cc(N2CCOCC2)nc(OCCc2ccccn2)n1] | N-(3-Hydroxy-phenyl)-N′-[6- morpholin-4-yl-2-(2-pyridin-2- yl-ethoxy)-pyrimidin-4-yl]- oxalamide |
| 365 | 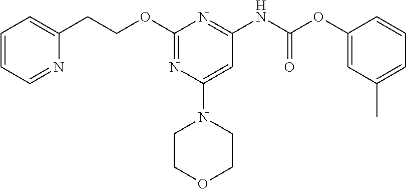 [SMILES: Cc1cccc(OC(=O)Nc2cc(N3CCOCC3)nc(OCCc3ccccn3)n2)c1] | [6-Morpholin-4-yl-2-(2- pyridin-2-yl-ethoxy)- pyrimidin-4-yl]-carbamic acid m-tolyl ester |
| 366 | 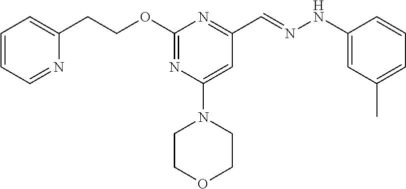 [SMILES: Cc1cccc(N/N=C/c2cc(N3CCOCC3)nc(OCCc3ccccn3)n2)c1] | N-[6-Morpholin-4-yl-2-(2- pyridin-2-yl-ethoxy)- pyrimidin-4-ylmethylene]-N′- m-tolyl-hydrazine |
| 367 | 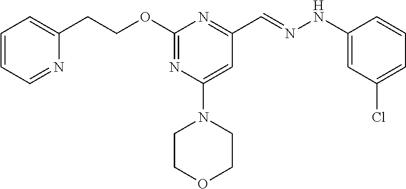 [SMILES: Clc1cccc(N/N=C/c2cc(N3CCOCC3)nc(OCCc3ccccn3)n2)c1] | N-(3-Chloro-phenyl)-N′-[6- morpholin-4-yl-2-(2-pyridin-2- yl-ethoxy)-pyrimidin-4- ylmethylene]-hydrazine |
| 368 | 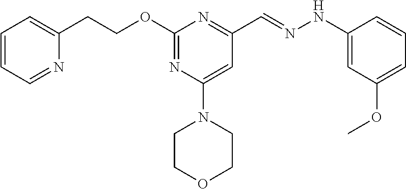 [SMILES: COc1cccc(N/N=C/c2cc(N3CCOCC3)nc(OCCc3ccccn3)n2)c1] | N-(3-Methoxy-phenyl)-N′-[6- morpholin-4-yl-2-(2-pyridin-2- yl-ethoxy)-pyrimidin-4- ylmethylene]-hydrazine |
| 369 | 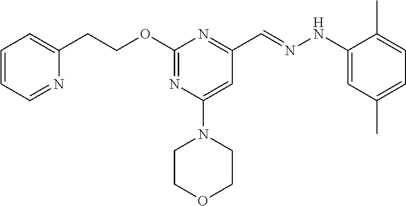 [SMILES: Cc1ccc(C)c(N/N=C/c2cc(N3CCOCC3)nc(OCCc3ccccn3)n2)c1] | N-(2,5-Dimethyl-phenyl)-N′- [6-morpholin-4-yl-2-(2- pyridin-2-yl-ethoxy)- pyrimidin-4-ylmethylene]- hydrazine |
| 370 | 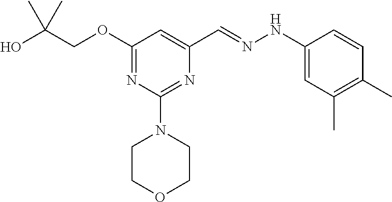 [SMILES: Cc1ccc(N/N=C/c2cc(OCC(C)(C)O)nc(N3CCOCC3)n2)cc1C] | 1-{6-[(3,4-Dimethyl-phenyl)- hydrazonomethyl]-2- morpholin-4-yl-pyrimidin-4- yloxy}-2-methyl-propan-2-ol |
| 371 | 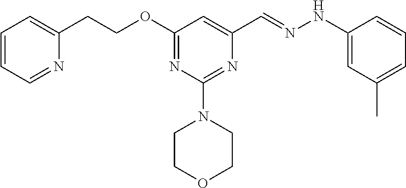 [SMILES: Cc1cccc(N/N=C/c2cc(OCCc3ccccn3)nc(N3CCOCC3)n2)c1] | N-[2-Morpholin-4-yl-6-(2- pyridin-2-yl-ethoxy)- pyrimidin-4-ylmethylene]-N′- m-tolyl-hydrazine |
| 372 | 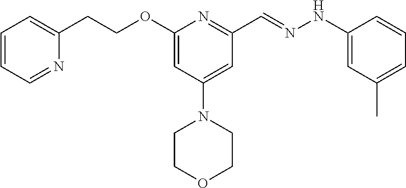 [SMILES: Cc1cccc(N/N=C/c2cc(N3CCOCC3)cc(OCCc3ccccn3)n2)c1] | N-[4-Morpholin-4-yl-6-(2- pyridin-2-yl-ethoxy)-pyridin-2- ylmethylene]-N′-m-tolyl- hydrazine |
| 373 | 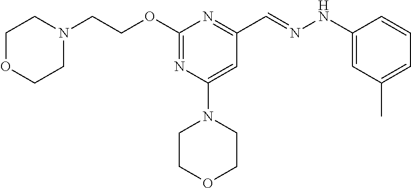 [SMILES: Cc1cccc(N/N=C/c2cc(N3CCOCC3)nc(OCCN3CCOCC3)n2)c1] | N-[6-Morpholin-4-yl-2-(2- morpholin-4-yl-ethoxy)- pyrimidin-4-ylmethylene]-N′- m-tolyl-hydrazine |
| 374 | 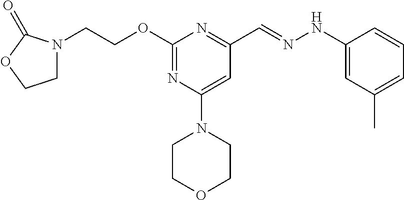 [SMILES: Cc1cccc(N/N=C/c2cc(N3CCOCC3)nc(OCCN3CCOC3=O)n2)c1] | 3-{2-[4-Morphpolin-4-yl-6-(m- tolyl-hydrazonomethyl)- pyrimidin-2-yloxy]-ethyl}- oxazolidin-2-one |
| 375 | 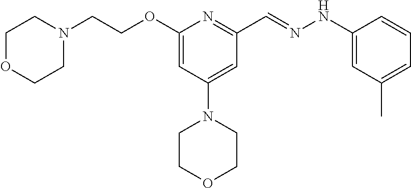 [SMILES: Cc1cccc(N/N=C/c2cc(N3CCOCC3)cc(OCCN3CCOCC3)n2)c1] | N-[4-Morpholin-4-yl-6-(2- morpholin-4-yl-ethoxy)- pyridin-2-ylmethylene]-N′-m- tolyl-hydrazine |
| 376 | 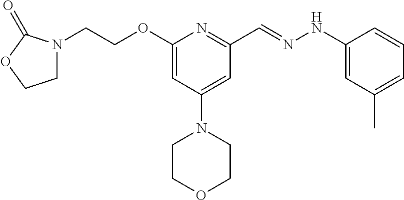 [SMILES: Cc1cccc(N/N=C/c2cc(N3CCOCC3)cc(OCCN3CCOC3=O)n2)c1] | 3-{2-[4-Morpholin-4-yl-6-(m- tolyl-hydrazonomethyl)- pyridin-2-yloxy]-ethyl}- oxazolidin-2-one |
| 377 | 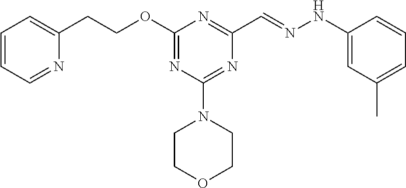 [SMILES: Cc1cccc(N/N=C/c2nc(OCCc3ccccn3)nc(N3CCOCC3)n2)c1] | N-[4-Morpholin-4-yl-6-(2- pyridin-2-yl-ethoxy)- [1,3,5]triazin-2-ylmethylene]- N′-m-tolyl-hydrazine |
| 378 | 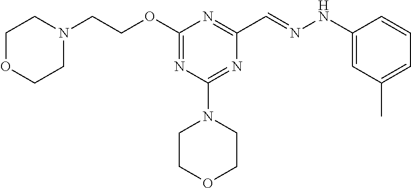 [SMILES: Cc1cccc(N/N=C/c2nc(OCCN3CCOCC3)nc(N3CCOCC3)n2)c1] | N-[4-Morpholin-4-yl-6-(2- morpholin-4-yl-ethoxy)- [1,3,5]triazin-2-ylmethylene]- N′-m-tolyl-hydrazine |
| 379 | 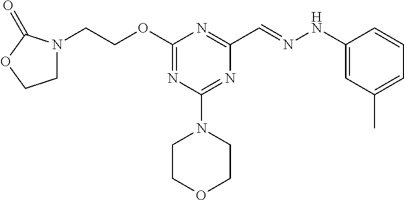 [SMILES: Cc1cccc(N/N=C/c2nc(OCCN3CCOC3=O)nc(N3CCOCC3)n2)c1] | 3-{2-[4-Morpholin-4-yl-6-(m- tolyl-hydrazonomethyl)- [1,3,5]triazin-2-yloxy]-ethyl}- oxazolidin-2-one |
| 380 | 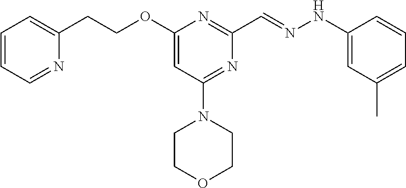 [SMILES: Cc1cccc(N/N=C/c2nc(OCCc3ccccn3)cc(N3CCOCC3)n2)c1] | N-[4-Morpholin-4-yl-6-(2- pyridin-2-yl-ethoxy)- pyrimidin-2-ylmethylene]-N′- m-tolyl-hydrazine |
| 381 | 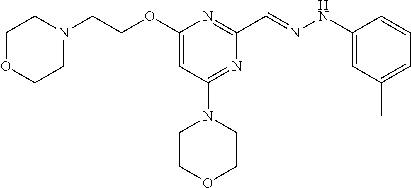 [SMILES: Cc1cccc(N/N=C/c2nc(OCCN3CCOCC3)cc(N3CCOCC3)n2)c1] | N-[4-Morpholin-4-yl-6-(2- morpholin-4-yl-ethoxy)- pyrimidin-2-ylmethylene]-N′- m-tolyl-hydrazine |
| 382 | 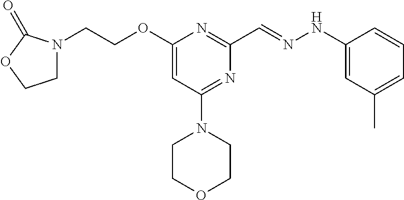 [SMILES: Cc1cccc(N/N=C/c2nc(OCCN3CCOC3=O)cc(N3CCOCC3)n2)c1] | 3-{2-[6-Morpholin-4-yl-2-(m- tolyl-hydrazonomethyl)- pyrimidin-4-yloxy]-ethyl}- oxazolidin-2-one |
| 383 | 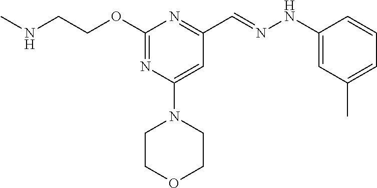 [SMILES: CNCCOc1nc(/C=N/Nc2cccc(C)c2)cc(N2CCOCC2)n1] | Methyl-{2-[4-morpholin-4-yl- 6-(m-tolyl-hydrazonomethyl)- pyrimidin-2-yloxy]-ethyl}- amine |
| 384 | 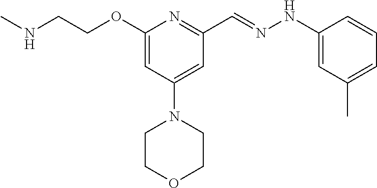 [SMILES: CNCCOc1cc(N2CCOCC2)cc(/C=N/Nc2cccc(C)c2)n1] | Methyl-{2-[4-morpholin-4-yl- 6-(m-tolyl-hydrazonomethyl)- pyridin-2-yloxy]-ethyl}-amine |
| 385 | 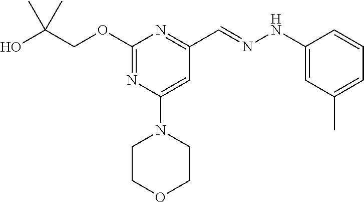 [SMILES: Cc1cccc(N/N=C/c2cc(N3CCOCC3)nc(OCC(C)(C)O)n2)c1] | 2-Methyl-1-[4-morpholin-4-yl- 6-(m-tolyl-hydrazonomethyl)- pyrimidin-2-yloxy]-propan-2-ol |
| 386 | 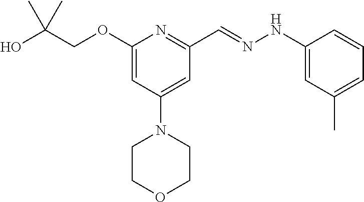 [SMILES: Cc1cccc(N/N=C/c2cc(N3CCOCC3)cc(OCC(C)(C)O)n2)c1] | 2-Methyl-1-[4-morpholin-4-yl- 6-(m-tolyl-hydrazonomethyl)- pyridin-2-yloxy]-propan-2-ol |
| 387 | 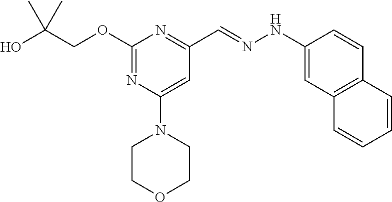 [SMILES: CC(C)(O)COc1nc(/C=N/Nc2ccc3ccccc3c2)cc(N2CCOCC2)n1] | 2-Methyl-1-[4-morpholin-4-yl- 6-(naphthalen-2-yl- hydrazonomethyl)-pyrimidin-2- yloxy]-propan-2-ol |
| 388 | 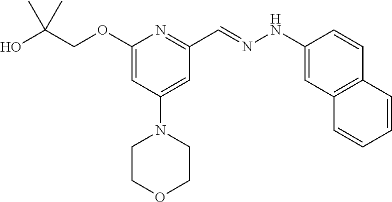 [SMILES: CC(C)(O)COc1cc(N2CCOCC2)cc(/C=N/Nc2ccc3ccccc3c2)n1] | 2-Methyl-1-[4-morpholin-4-yl- 6-(naphthalen-2-yl- hydrazonomethyl)-pyridin-2- yloxy]-propan-2-ol |
| 389 | 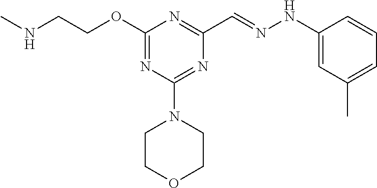 [SMILES: CNCCOc1nc(/C=N/Nc2cccc(C)c2)nc(N2CCOCC2)n1] | Methyl-{2-[4-morpholin-4-yl- 6-(m-tolyl-hydrazonomethyl)- [1,3,5]triazin-2-yloxy]-ethyl}- amine |
| 390 | 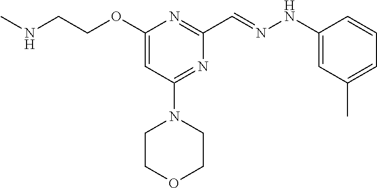 [SMILES: CNCCOc1cc(N2CCOCC2)nc(/C=N/Nc2cccc(C)c2)n1] | Methyl-{2-[6-morpholin-4-yl- 2-(m-tolyl-hydrazonomethyl)- pyrimidin-4-yloxy]-ethyl}- amine |
| 391 | 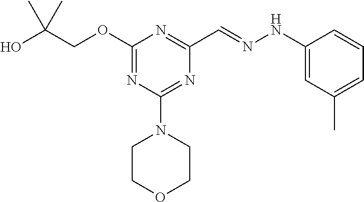 [SMILES: Cc1cccc(N/N=C/c2nc(OCC(C)(C)O)nc(N3CCOCC3)n2)c1] | 2-Methyl-1-[4-morpholin-4-yl- 6-(m-tolyl-hydrazonomethyl)- [1,3,5]triazin-2-yloxy]-propan- 2-ol |
| 392 | 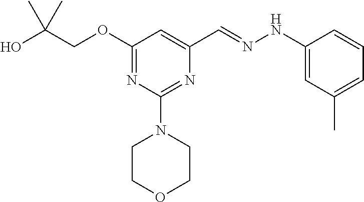 [SMILES: Cc1cccc(N/N=C/c2cc(OCC(C)(C)O)nc(N3CCOCC3)n2)c1] | 2-Methyl-1-[2-morpholin-4-yl- 6-(m-tolyl-hydrazonomethyl)- pyrimidin-4-yloxy]-propan-2-ol |
| 393 | 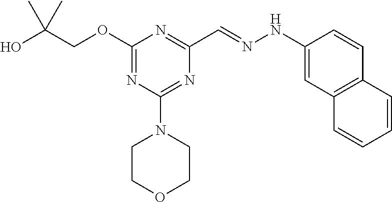 [SMILES: CC(C)(O)COc1nc(/C=N/Nc2ccc3ccccc3c2)nc(N2CCOCC2)n1] | 2-Methyl-1-[4-morpholin-4-yl- 6-(naphthalen-2-yl- hydrazonomethyl)- [1,3,5]triazin-2-yloxy]-propan- 2-ol |
| 394 | 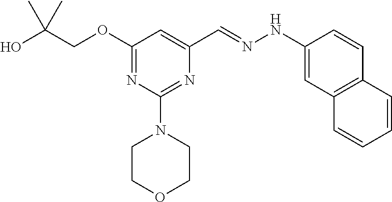 [SMILES: CC(C)(O)COc1cc(/C=N/Nc2ccc3ccccc3c2)nc(N2CCOCC2)n1] | 2-Methyl-1-[2-morpholin-4-yl- 6-(naphthalen-2-yl- hydrazonomethyl)-pyrimidin-4- yloxy]-propan-2-ol |
| 395 | 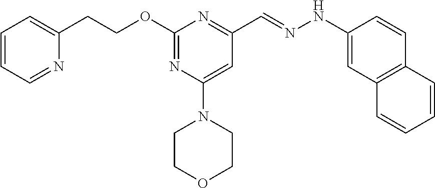 [SMILES: C(=N\Nc1ccc2ccccc2c1)/c1cc(N2CCOCC2)nc(OCCc2ccccn2)n1] | N-[6-Morpholin-4-yl-2-(2- pyridin-2-yl-ethoxy)- pyrimidin-4-ylmethylene]-N′- naphthalen-2-yl-hydrazine |
| 396 | 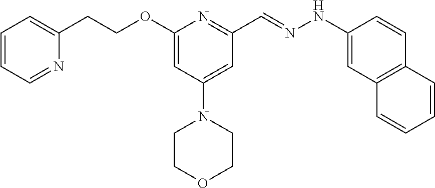 [SMILES: C(=N\Nc1ccc2ccccc2c1)/c1cc(N2CCOCC2)cc(OCCc2ccccn2)n1] | N-[4-Morpholin-4-yl-6-(2- pyridin-2-yl-ethoxy)-pyridin-2- ylmethylene]-N′-naphthalen-2- yl-hydrazine |
| 397 | 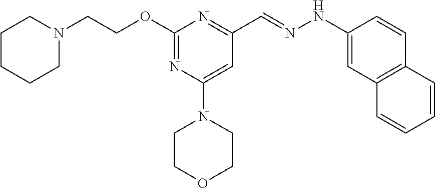 [SMILES: C(=N\Nc1ccc2ccccc2c1)/c1cc(N2CCOCC2)nc(OCCN2CCCCC2)n1] | N-[6-Morpholin-4-yl-2-(2- piperidin-1-yl-ethoxy)- pyrimidin-4-ylmethylene]-N′- naphthalen-2-yl-hydrazine |
| 398 | 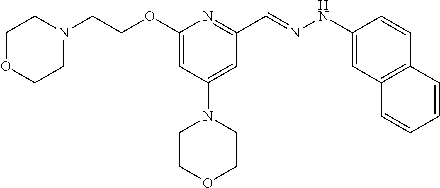 [SMILES: C(=N\Nc1ccc2ccccc2c1)/c1cc(N2CCOCC2)cc(OCCN2CCOCC2)n1] | N-[4-Morpholin-4-yl-6-(2- morpholin-4-yl-ethoxy)- pyridin-2-ylmethylene]-N′- naphthalen-2-yl-hydrazine |
| 399 | 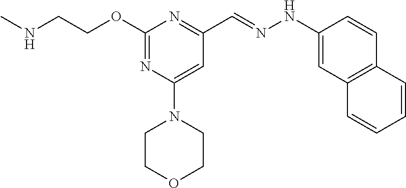 [SMILES: CNCCOc1nc(/C=N/Nc2ccc3ccccc3c2)cc(N2CCOCC2)n1] | Methyl-{2-[4-morpholin-4-yl- 6-(naphthalen-2-yl- hydrazonomethyl)-pyrimidin-2- yloxy]-ethyl}-amine |
| 400 | 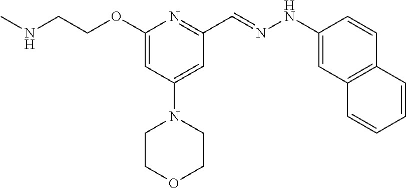 [SMILES: CNCCOc1cc(N2CCOCC2)cc(/C=N/Nc2ccc3ccccc3c2)n1] | Methyl-{2-[4-morpholin-4-yl- 6-(naphthalen-2-yl- hydrazonomethyl)-pyridin-2- yloxy]-ethyl}-amine |
| 401 | 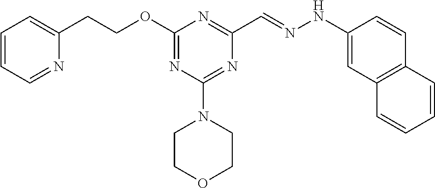 [SMILES: C(=N\Nc1ccc2ccccc2c1)/c1nc(OCCc2ccccn2)nc(N2CCOCC2)n1] | N-[4-Morpholin-4-yl-6-(2- pyridin-2-yl-ethoxy)- [1,3,5]triazin-2-ylmethylene]- N′-naphthalen-2-yl-hydrazine |
| 402 | 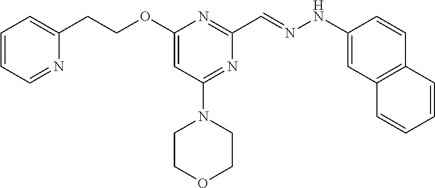 [SMILES: C(=N\Nc1ccc2ccccc2c1)/c1nc(OCCc2ccccn2)cc(N2CCOCC2)n1] | N-[4-Morpholin-4-yl-6-(2- pyridin-2-yl-ethoxy)- pyrimidin-2-ylmethylene]-N′- naphthalen-2-yl-hydrazine |
| 403 | 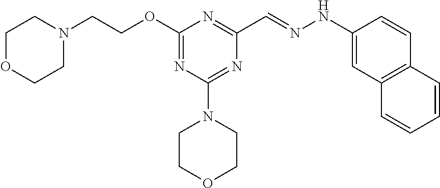 [SMILES: C(=N\Nc1ccc2ccccc2c1)/c1nc(OCCN2CCOCC2)nc(N2CCOCC2)n1] | N-[4-Morpholin-4-yl-6-(2- morpholin-4-yl-ethoxy)- [1,3,5]triazin-2-ylmethylene]- N′-naphthalen-2-yl-hydrazine |
| 404 | 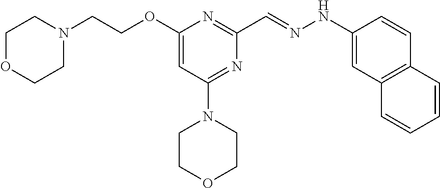 [SMILES: C(=N\Nc1ccc2ccccc2c1)/c1nc(OCCN2CCOCC2)cc(N2CCOCC2)n1] | N-[4-Morpholin-4-yl-6-(2- morpholin-4-yl-ethoxy)- pyrimidin-2-ylmethylene]-N′- naphthalen-2-yl-hydrazine |
| 405 | 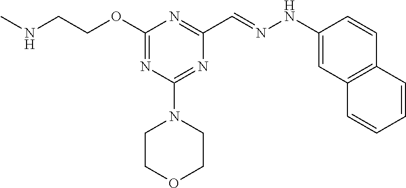 [SMILES: CNCCOc1nc(/C=N/Nc2ccc3ccccc3c2)nc(N2CCOCC2)n1] | Methyl-{2-[4-morpholin-4-yl- 6-(naphthalen-2-yl- hydrazonomethyl)- [1,3,5]triazin-2-yloxy]-ethyl}- amine |
| 406 | 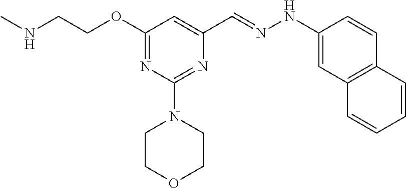 [SMILES: CNCCOc1cc(/C=N/Nc2ccc3ccccc3c2)nc(N2CCOCC2)n1] | Methyl-{2-[2-morpholin-4-yl- 6-(naphthalen-2-yl- hydrazonomethyl)-pyrimidin-4- yloxy]-ethyl}-amine |
| 407 | 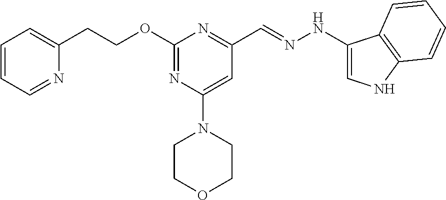 [SMILES: C(=N\Nc1c[nH]c2ccccc12)/c1cc(N2CCOCC2)nc(OCCc2ccccn2)n1] | N-(1H-Indol-3-yl)-N′-[6- morpholin-4-yl-2-(2-pyridin-2- yl-ethoxy)-pyrimidin-4- ylmethylene]-hydrazine |
| 408 | 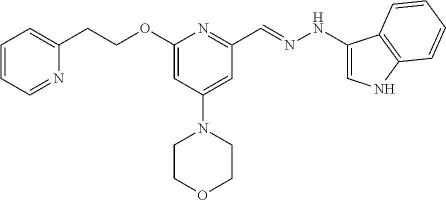 [SMILES: C(=N\Nc1c[nH]c2ccccc12)/c1cc(N2CCOCC2)cc(OCCc2ccccn2)n1] | N-(1H-Indol-3-yl)-N′-[4- morpholin-4-yl-6-(2-pyridin-2- yl-ethoxy)-pyridin-2- ylmethylene]-hydrazine |
| 409 | 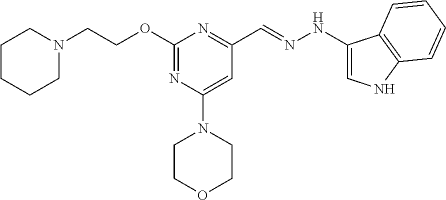 [SMILES: C(=N\Nc1c[nH]c2ccccc12)/c1cc(N2CCOCC2)nc(OCCN2CCCCC2)n1] | N-(1H-Indol-3-yl)-N′-[6- morpholin-4-yl-2-(2-piperidin- 1-yl-ethoxy)-pyrimidin-4- ylmethylene]-hydrazine |
| 410 | 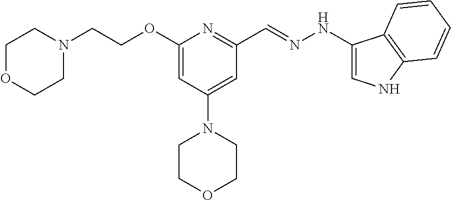 [SMILES: C(=N\Nc1c[nH]c2ccccc12)/c1cc(N2CCOCC2)cc(OCCN2CCOCC2)n1] | N-(1H-Indol-3-yl)-N′-[4- morpholin-4-yl-6-(2- morpholin-4-yl-ethoxy)- pyridin-2-ylmethylene]- hydrazine |
| 411 | 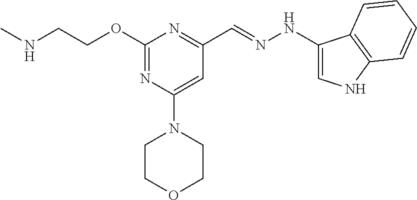 [SMILES: CNCCOc1nc(/C=N/Nc2c[nH]c3ccccc23)cc(N2CCOCC2)n1] | (2-{4-[(1H-Indol-3-yl)- hydrazonomethyl]-6- morpholin-4-yl-pyrimidin-2- yloxy}-ethyl)-methyl-amine |
| 412 | 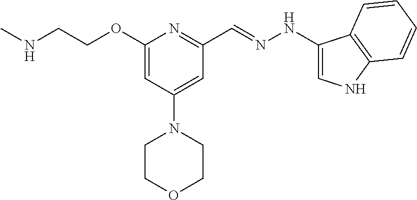 [SMILES: CNCCOc1cc(N2CCOCC2)cc(/C=N/Nc2c[nH]c3ccccc23)n1] | (2-{6-[(1H-Indol-3-yl)- hydrazonomethyl]-4- morpholin-4-yl-pyridin-2- yloxy}-ethyl)-methyl-amine |
| 413 | 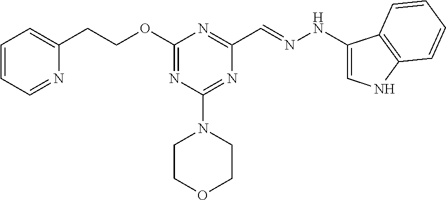 [SMILES: C(=N\Nc1c[nH]c2ccccc12)/c1nc(OCCc2ccccn2)nc(N2CCOCC2)n1] | N-(1H-Indol-3-yl)-N′-[4- morpholin-4-yl-6-(2-pyridin-2- yl-ethoxy)-[1,3,5]triazin-2- ylmethylene]-hydrazine |
| 414 | 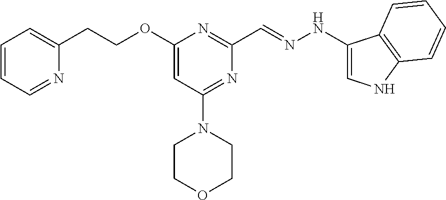 [SMILES: C(=N\Nc1c[nH]c2ccccc12)/c1nc(OCCc2ccccn2)cc(N2CCOCC2)n1] | N-(1H-Indol-3-yl)-N′-[4- morpholin-4-yl-6-(2-pyridin-2- yl-ethoxy)-pyrimidin-2- ylmethylene]-hydrazine |
| 415 | 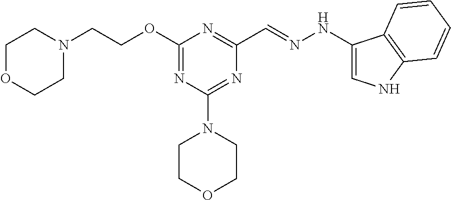 [SMILES: C(=N\Nc1c[nH]c2ccccc12)/c1nc(OCCN2CCOCC2)nc(N2CCOCC2)n1] | N-(1H-Indol-3-yl)-N′-[4- morpholin-4-yl-6-(2- morpholin-4-yl-ethoxy)- [1,3,5]triazin-2-ylmethylene]- hydrazine |
| 416 | 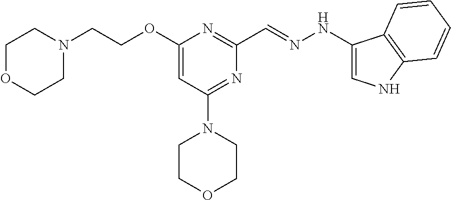 [SMILES: C(=N\Nc1c[nH]c2ccccc12)/c1nc(OCCN2CCOCC2)cc(N2CCOCC2)n1] | N-(1H-Indol-3-yl)-N′-[4- morpholin-4-yl-6-(2- morpholin-4-yl-ethoxy)- pyrimidin-2-ylmethylene]- hydrazine |
| 417 | 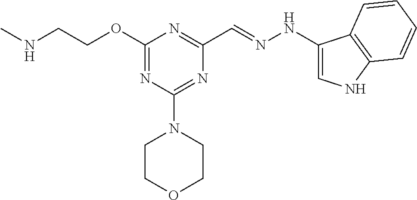 [SMILES: CNCCOc1nc(/C=N/Nc2c[nH]c3ccccc23)nc(N2CCOCC2)n1] | (2-{4-[(1H-Indol-3-yl)- hydrazonomethyl]-6- morpholin-4-yl[1,3,5]trazin- 2-yloxy}-ethyl)-methyl-amine |
| 418 | 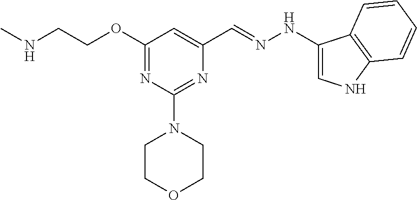 [SMILES: CNCCOc1cc(/C=N/Nc2c[nH]c3ccccc23)nc(N2CCOCC2)n1] | (2-{6-[(1H-Indol-3-yl)- hydrazonomethyl]-2- morpholin-4-yl-pyrimidin-4- yloxy}-ethyl)-methyl-amine |
| 419 | 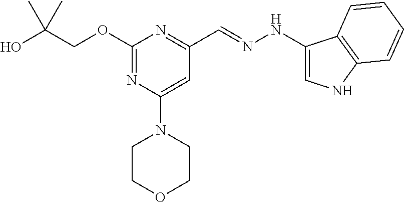 [SMILES: CC(C)(O)COc1nc(/C=N/Nc2c[nH]c3ccccc23)cc(N2CCOCC2)n1] | 1-{4-[(1H-Indol-3-yl)- hydrazonomethyl]-6- morpholin-4-yl-pyrimidin-2- yloxy}-2-methyl-propan-2-ol |
| 420 | 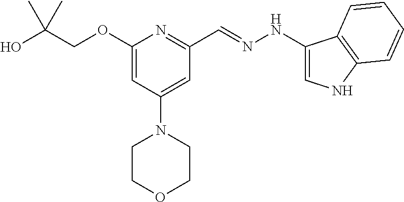 [SMILES: CC(C)(O)COc1cc(N2CCOCC2)cc(/C=N/Nc2c[nH]c3ccccc23)n1] | 1-{6-[(1H-Indol-3-yl)- hydrazonomethyl]-4- morpholin-4-yl-pyridin-2- yloxy}-2-methyl-propan-2-ol |
| 421 | 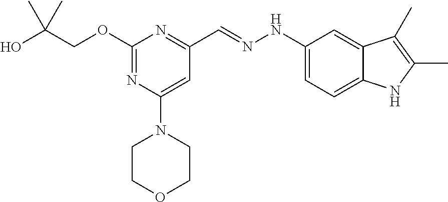 [SMILES: Cc1[nH]c2ccc(N/N=C/c3cc(N4CCOCC4)nc(OCC(C)(C)O)n3)cc2c1C] | 1-{4-[(2,3-Dimethyl-1H-indol- 5-yl)-hydrazonomethyl]-6- morpholin-4-yl-pyrimidin-2- yloxy}-2-methyl-propan-2-ol |
| 422 | 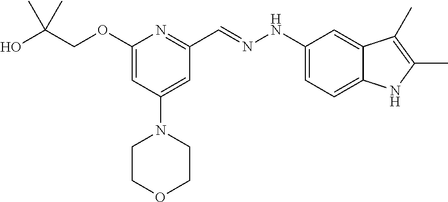 [SMILES: Cc1[nH]c2ccc(N/N=C/c3cc(N4CCOCC4)cc(OCC(C)(C)O)n3)cc2c1C] | 1-{6-[(2,3-Dimethyl-1H-indol- 5-yl)-hydrazonomethyl]-4- morpholin-4-yl-pyridin-2- yloxy}-2-methyl-propan-2-ol |
| 423 | 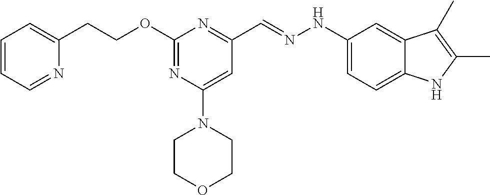 [SMILES: Cc1[nH]c2ccc(N/N=C/c3cc(N4CCOCC4)nc(OCCc4ccccn4)n3)cc2c1C] | N-(2,3-Dimethyl-1H-indol-5- yl)-N′-[6-morpholin-4-yl-2-(2- pyridin-2-yl-ethoxy)- pyrimidin-4-ylmethylene]- hydrazine |
| 424 | 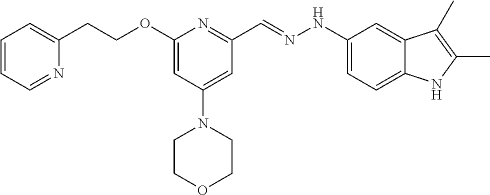 [SMILES: Cc1[nH]c2ccc(N/N=C/c3cc(N4CCOCC4)cc(OCCc4ccccn4)n3)cc2c1C] | N-(2,3-Dimethyl-1H-indol-5- yl)-N′-[4-morpholin-4-yl-6-(2- pyridin-2-yl-ethoxy)-pyridin-2- ylmethylene]-hydrazine |
| 425 | 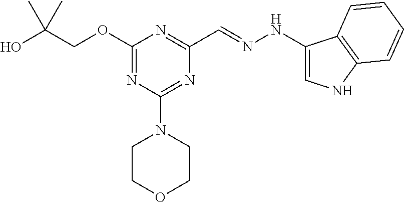 [SMILES: CC(C)(O)COc1nc(/C=N/Nc2c[nH]c3ccccc23)nc(N2CCOCC2)n1] | 1-{4-[(1H-Indol-3-yl)- hydrazonomethyl]-6- morpholin-4-yl-[1,3,5]triazin- 2-yloxy}-2-methyl-propan-2-ol |
| 426 | 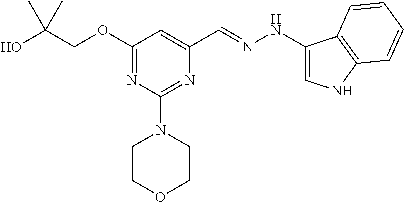 [SMILES: CC(C)(O)COc1cc(/C=N/Nc2c[nH]c3ccccc23)nc(N2CCOCC2)n1] | 1-{6-[(1H-Indol-3-yl)- hydrazonomethyl]-2- morpholin-4-yl-pyrimidin-4- yloxy}-2-methyl-propan-2-ol |
| 427 | 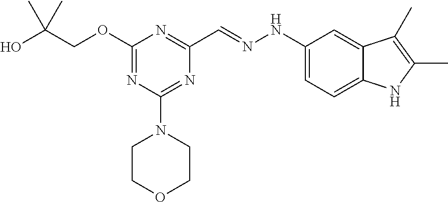 [SMILES: Cc1[nH]c2ccc(N/N=C/c3nc(OCC(C)(C)O)nc(N4CCOCC4)n3)cc2c1C] | 1-{4-[(2,3-Dimethyl-1H-indol- 5-yl)-hydrazonomethyl]-6- morpholin-4-yl-[1,3,5]triazin- 2-yloxy}-2-methyl-propan-2-ol |
| 428 | 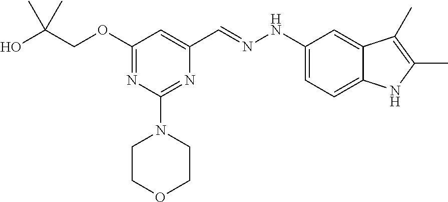 [SMILES: Cc1[nH]c2ccc(N/N=C/c3cc(OCC(C)(C)O)nc(N4CCOCC4)n3)cc2c1C] | 1-{6-[(2,3-Dimethyl-1H-indol- 5-yl)-hydrazonomethyl]-2- morpholin-4-yl-pyrimidin-4- yloxy}-2-methyl-propan-2-ol |
| 429 | 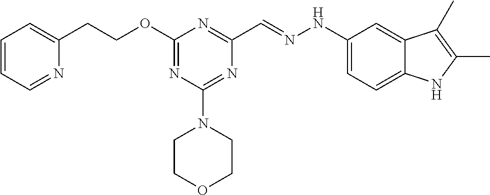 [SMILES: Cc1[nH]c2ccc(N/N=C/c3nc(OCCc4ccccn4)nc(N4CCOCC4)n3)cc2c1C] | N-(2,3-Dimethyl-1H-indol-5- yl)-N′-[4-morpholin-4-yl-6-(2- pyridin-2-yl-ethoxy)- [1,3,5]triazin-2-ylmethylene]- hydrazine |
| 430 | 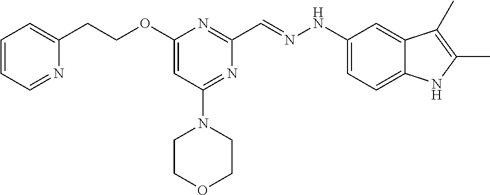 [SMILES: Cc1[nH]c2ccc(N/N=C/c3nc(OCCc4ccccn4)cc(N4CCOCC4)n3)cc2c1C] | N-(2,3-Dimethyl-1H-indol-5- yl)-N′-[4-morpholin-4-yl-6-(2- pyridin-2-yl-ethoxy)- pyrimidin-2-ylmethylene]- hydrazine |
| 431 | 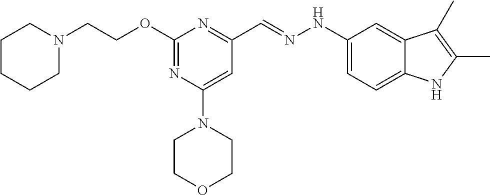 [SMILES: Cc1[nH]c2ccc(N/N=C/c3cc(N4CCOCC4)nc(OCCN4CCCCC4)n3)cc2c1C] | N-(2,3-Dimethyl-1H-indol-5- yl)-N′-[6-morpholin-4-yl-2-(2- piperidin-1-yl-ethoxy)- pyrimidin-4-ylmethylene]- hydrazine |
| 432 | 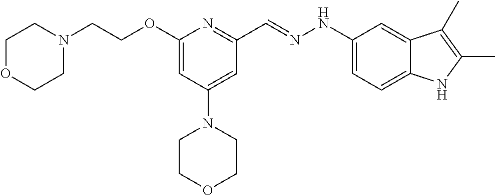 [SMILES: Cc1[nH]c2ccc(N/N=C/c3cc(N4CCOCC4)cc(OCCN4CCOCC4)n3)cc2c1C] | N-(2,3-Dimethyl-1H-indol-5- yl)-N′-[4-morpholin-4-yl-6-(2- morpholin-4-yl-ethoxy)- pyridin-2-ylmethylene]- hydrazine |
| 433 | 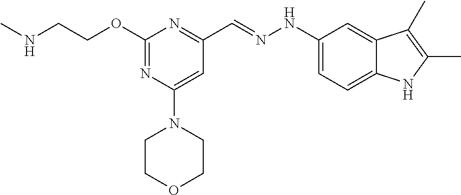 | (2-{4-[(2,3-Dimethyl-1H- indol-5-yl)-hydrazonomethyl]- 6-morpholin-4-yl-pyrimidin-2- yloxy}-ethyl)-methyl-amine |
| 434 | 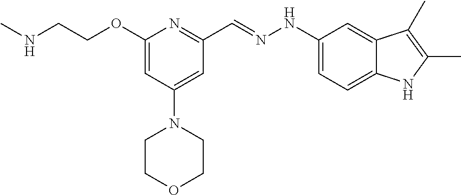 | (2-{6-[(2,3-Dimethyl-1H- indol-5-yl)-hydrazonomethyl]- 4-morpholin-4-yl-pyridin-2- yloxy}-ethyl)-methyl-amine |
| 435 | 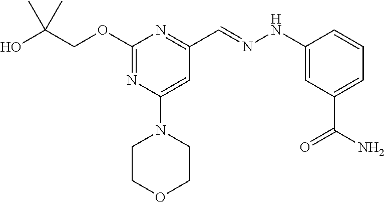 [SMILES: CC(C)(O)COc1nc(/C=N/Nc2cccc(C(N)=O)c2)cc(N2CCOCC2)n1] | 3-{N′-[2-(2-Hydroxy-2-methyl- propoxy)-6-morpholin-4-yl- pyrimidin-4-ylmethylene]- hydrazino}-benzamide |
| 436 | 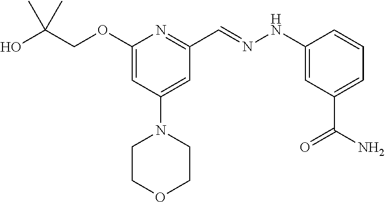 [SMILES: CC(C)(O)COc1cc(N2CCOCC2)cc(/C=N/Nc2cccc(C(N)=O)c2)n1] | 3-{N′-[6-(2-Hydroxy-2-methyl- propoxy)-4-morpholin-4-yl- pyridin-2-ylmethylene]- hydrazino}-benzamide |
| 437 | 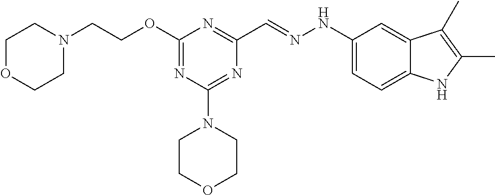 [SMILES: Cc1[nH]c2ccc(N/N=C/c3nc(OCCN4CCOCC4)nc(N4CCOCC4)n3)cc2c1C] | N-(2,3-Dimethyl-1H-indol-5- yl)-N′-[4-morpholin-4-yl-6-(2- morpholin-4-yl-ethoxy)- [1,3,5]triazin-2-ylmethylene]- hydrazine |
| 438 | 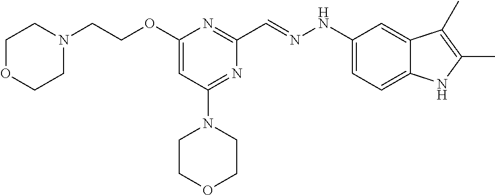 [SMILES: Cc1[nH]c2ccc(N/N=C/c3nc(OCCN4CCOCC4)cc(N4CCOCC4)n3)cc2c1C] | N-(2,3-Dimethyl-1H-indol-5- yl)-N′-[4-morpholin-4-yl-6-(2- morpholin-4-yl-ethoxy)- pyrimidin-2-ylmethylene]- hydrazine |
| 439 | 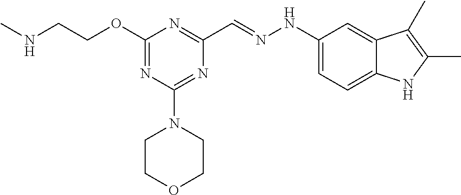 | (2-{4-[(2,3-Dimethyl-1H- indol-5-yl)-hydrazonomethyl]- 6-morpholin-4-yl- [1,3,5]triazin-2-yloxy}-ethyl)- methyl-amine |
| 440 | 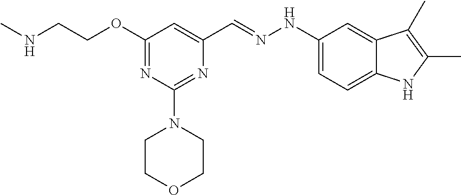 | (2-{6-[(2,3-Dimethyl-1H- indol-5-yl)-hydrazonomethyl]- 2-morpholin-4-yl-pyrimidin-4- yloxy}-ethyl)-methyl-amine |
| 441 | 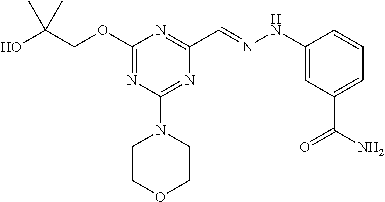 [SMILES: CC(C)(O)COc1nc(/C=N/Nc2cccc(C(N)=O)c2)nc(N2CCOCC2)n1] | 3-{N′-[4-(2-Hydroxy-2-methyl- propoxy)-6-morpholin-4-yl- [1,3,5]triazin-2-ylmethylene]- hydrazino}-benzamide |
| 442 | 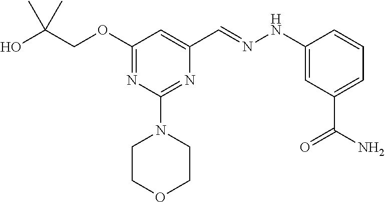 [SMILES: CC(C)(O)COc1cc(/C=N/Nc2cccc(C(N)=O)c2)nc(N2CCOCC2)n1] | 3-{N′-[6-(2-Hydroxy-2-methyl- propoxy)-2-morpholin-4-yl- pyrimidin-4-ylmethylene]- hydrazino}-benzamide |
| 443 | 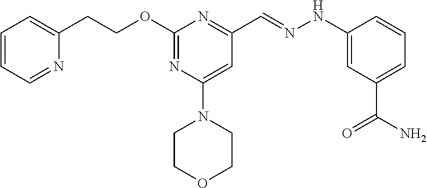 [SMILES: NC(=O)c1cccc(N/N=C/c2cc(N3CCOCC3)nc(OCCc3ccccn3)n2)c1] | 3-{N′-[6-Morpholin-4-yl-2-(2- pyridin-2-yl-ethoxy)- pyrimidin-4-ylmethylene]- hydrazino}-benzamide |
| 444 | 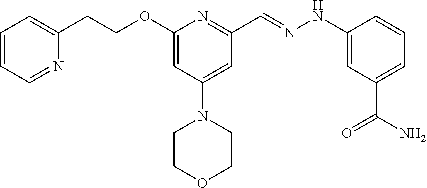 [SMILES: NC(=O)c1cccc(N/N=C/c2cc(N3CCOCC3)cc(OCCc3ccccn3)n2)c1] | 3-{N′-[4-Morpholin-4-yl-6-(2- pyridin-2-yl-ethoxy)-pyridin-2- ylmethylene]-hydrazino}- benzamide |
| 445 | 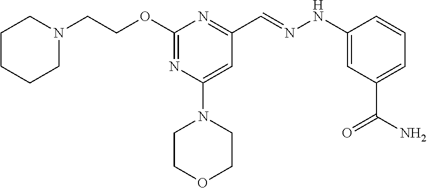 [SMILES: NC(=O)c1cccc(N/N=C/c2cc(N3CCOCC3)nc(OCCN3CCCCC3)n2)c1] | 3-{N′-[6-Morpholin-4-yl-2-(2- piperidin-1-yl-ethoxy)- pyrimidin-4-ylmethylene]- hydrazino}-benzamide |
| 446 | 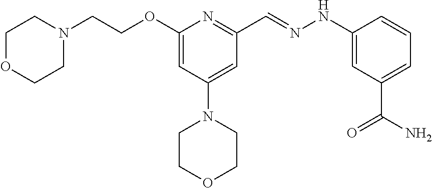 [SMILES: NC(=O)c1cccc(N/N=C/c2cc(N3CCOCC3)cc(OCCN3CCOCC3)n2)c1] | 3-{N′-[4-Morpholin-4-yl-6-(2- morpholin-4-yl-ethoxy)- pyridin-2-ylmethylene]- hydrazino}-benzamide |
| 447 | 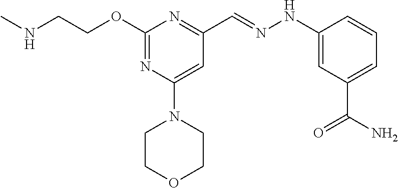 [SMILES: CNCCOc1nc(/C=N/Nc2cccc(C(N)=O)c2)cc(N2CCOCC2)n1] | 3-{N′-[2-(2-Methylamino- ethoxy)-6-morpholin-4-yl- pyrimidin-4-ylmethylene]- hydrazino}-benzamide |
| 448 | 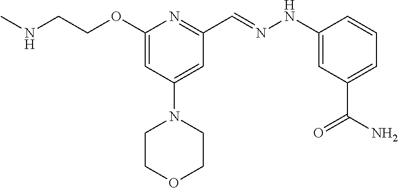 [SMILES: CNCCOc1cc(N2CCOCC2)cc(/C=N/Nc2cccc(C(N)=O)c2)n1] | 3-{N′-[6-(2-Methylamino- ethoxy)-4-morpholin-4-yl- pyridin-2-ylmethylene]- hydrazino}-benzamide |
| 449 | 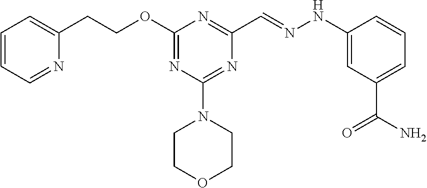 [SMILES: NC(=O)c1cccc(N/N=C/c2nc(OCCc3ccccn3)nc(N3CCOCC3)n2)c1] | 3-{N′-[4-Morpholin-4-yl-6-(2- pyridin-2-yl-ethoxy)- [1,3,5]triazin-2-ylmethylene]- hydrazino}-benzamide |
| 450 | 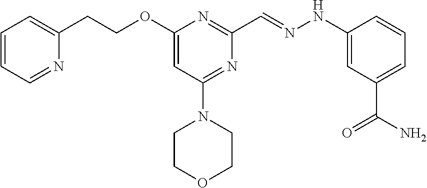 [SMILES: NC(=O)c1cccc(N/N=C/c2nc(OCCc3ccccn3)cc(N3CCOCC3)n2)c1] | 3-{N′-[4-Morpholin-4-yl-6-(2- pyridin-2-yl-ethoxy)- pyrimidin-2-ylmethylene]- hydrazino}-benzamide |
| 451 | 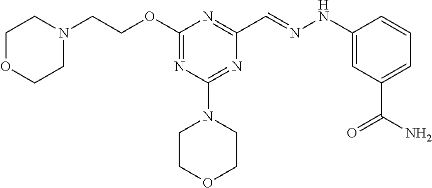 [SMILES: NC(=O)c1cccc(N/N=C/c2nc(OCCN3CCOCC3)nc(N3CCOCC3)n2)c1] | 3-{N′-[4-Morpholin-4-yl-6-(2- morpholin-4-yl-ethoxy)- [1,3,5]triazin-2-ylmethylene]- hydrazino}-benzamide |
| 452 | 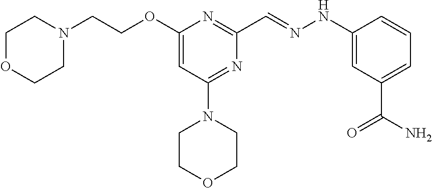 [SMILES: NC(=O)c1cccc(N/N=C/c2nc(OCCN3CCOCC3)cc(N3CCOCC3)n2)c1] | 3-{N′-[4-Morpholin-4-yl-6-(2- morpholin-4-yl-ethoxy)- pyrimidin-2-ylmethylene]- hydrazino}-benzamide |
| 453 | 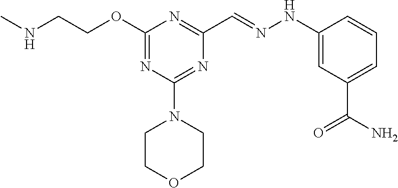 [SMILES: CNCCOc1nc(/C=N/Nc2cccc(C(N)=O)c2)nc(N2CCOCC2)n1] | 3-{N′-[4-(2-Methylamino- ethoxy)-6-morpholin-4-yl- [1,3,5]triazin-2-ylmethylene]- hydrazino}-benzamide |
| 454 | 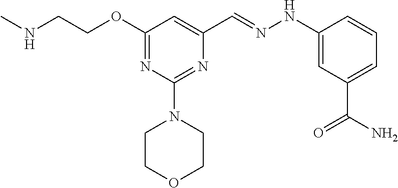 [SMILES: CNCCOc1cc(/C=N/Nc2cccc(C(N)=O)c2)nc(N2CCOCC2)n1] | 3-{N′-[6-(2-Methylamino- ethoxy)-2-morpholin-4-yl- pyrimidin-4-ylmethylene]- hydrazino}-benzamide |
| 455 | 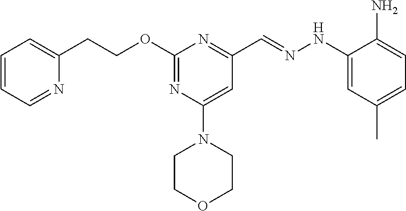 [SMILES: Cc1ccc(N)c(N/N=C/c2cc(N3CCOCC3)nc(OCCc3ccccn3)n2)c1] | 4-Methyl-2-{N′-[6-morpholin- 4-yl-2-(2-pyridin-2-yl-ethoxy)- pyrimidin-4-ylmethylene]- hydrazino}-phenylamine |
| 456 | 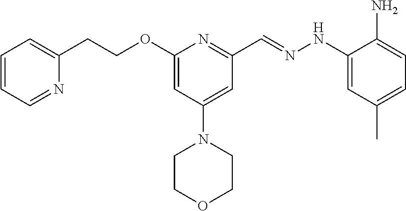 [SMILES: Cc1ccc(N)c(N/N=C/c2cc(N3CCOCC3)cc(OCCc3ccccn3)n2)c1] | 4-Methyl-2-{N′-[4-morpholin- 4-yl-6-(2-pyridin-2-yl-ethoxy)- pyridin-2-ylmethylene]- hydrazino}-benzamide |
| 457 | 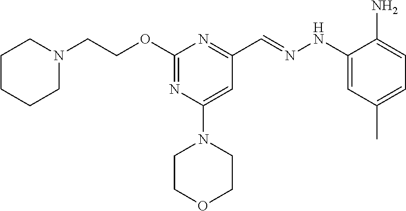 [SMILES: Cc1ccc(N)c(N/N=C/c2cc(N3CCOCC3)nc(OCCN3CCCCC3)n2)c1] | 4-Methyl-2-{N′-[6-morpholin- 4-yl-2-(2-piperidin-1-yl- ethoxy)-pyrimidin-4- ylmethylene]-hydrazino}- phenylamine |
| 458 | 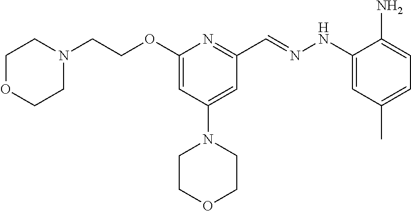 [SMILES: Cc1ccc(N)c(N/N=C/c2cc(N3CCOCC3)cc(OCCN3CCOCC3)n2)c1] | 4-Methyl-2-{N′-[4-morpholin- 4-yl-6-(2-morpholin-4-yl- ethoxy)-pyridin-2- ylmethylene]-hydrazino}- phenylamine |
| 459 | 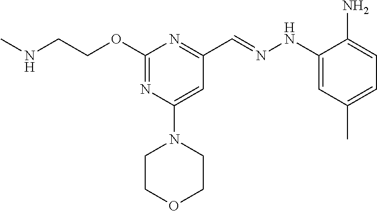 [SMILES: CNCCOc1nc(/C=N/Nc2cc(C)ccc2N)cc(N2CCOCC2)n1] | 4-Methyl-2-{N′-[2-(2- methylamino-ethoxy)-6- morpholin-4-yl-pyrimidin-4- ylmethylene]-hydrazino}- phenylamine |
| 460 | 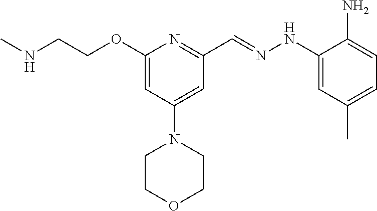 [SMILES: CNCCOc1cc(N2CCOCC2)cc(/C=N/Nc2cc(C)ccc2N)n1] | 4-Methyl-2-{N′-[6-(2- methylamino-ethoxy)-4- morpholin-4-yl-pyidin-2- ylmethylene]-hydrazino}- phenylamine |
| 461 | 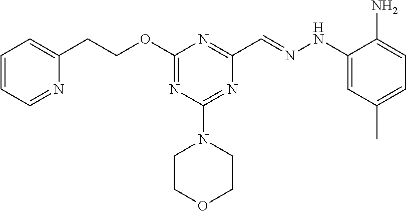 [SMILES: Cc1ccc(N)c(N/N=C/c2nc(OCCc3ccccn3)nc(N3CCOCC3)n2)c1] | 4-Methyl-2-{N′-[4-morpholin- 4-yl-6-(2-pyridin-2-yl-ethoxy)- [1,3,5]triazin-2-ylmethylene]- hydrazino}-phenylamine |
| 462 | 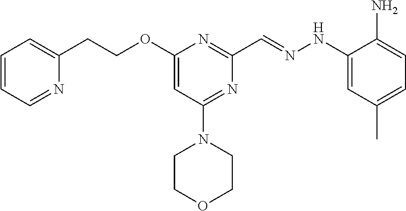 [SMILES: Cc1ccc(N)c(N/N=C/c2nc(OCCc3ccccn3)cc(N3CCOCC3)n2)c1] | 4-Methyl-2-{N′-[4-morpholin- 4-yl-6-(2-pyridin-2-yl-ethoxy)- pyrimidin-2-ylmethylene]- hydrazino}-phenylamine |
| 463 | 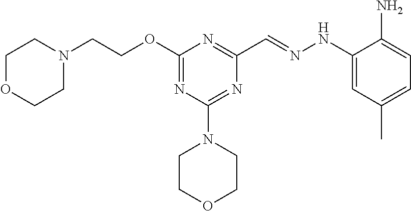 [SMILES: Cc1ccc(N)c(N/N=C/c2nc(OCCN3CCOCC3)nc(N3CCOCC3)n2)c1] | 4-Methyl-2-{N′-[4-morpholin- 4-yl-6-(2-morpholin-4-yl- ethoxy)-[1,3,5]triazin-2- ylmethylene]-hydrazino}- phenylamine |
| 464 | 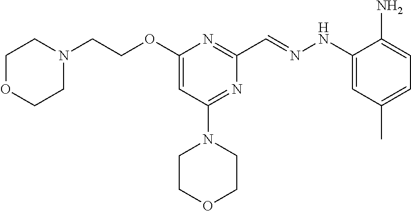 [SMILES: Cc1ccc(N)c(N/N=C/c2nc(OCCN3CCOCC3)cc(N3CCOCC3)n2)c1] | 4-Methyl-2-{N′-[4-morpholin- 4-yl-6-(2-morpholin-4-yl- ethoxy)-pyrimidin-2- ylmethylene]-hydrazino}- phenylamine |
| 465 | 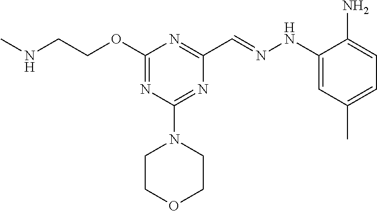 [SMILES: CNCCOc1nc(/C=N/Nc2cc(C)ccc2N)nc(N2CCOCC2)n1] | 4-Methyl-2-{N′-[4-(2- methylamino-ethoxy)-6- morpholin-4-yl-[1,3,5]triazin- 2-ylmethylene]-hydrazino}- phenylamine |
| 466 | 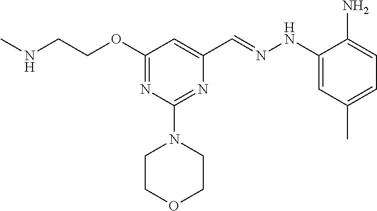 [SMILES: CNCCOc1cc(/C=N/Nc2cc(C)ccc2N)nc(N2CCOCC2)n1] | 4-Methyl-2-{N′-[6-(2- methylamino-ethoxy)-2- morpholin-4-yl-pyrimidin-4- ylmethylene]-hydrazino}- phenylamine |
| 467 | 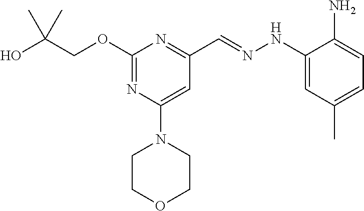 [SMILES: Cc1ccc(N)c(N/N=C/c2cc(N3CCOCC3)nc(OCC(C)(C)O)n2)c1] | 1-{4-[(2-Amino-5-methyl- phenyl)-hydrazonomethyl]-6- morpholin-4-yl-pyrimidin-2- yloxy}-2-methyl-propan-2-ol |
| 468 | 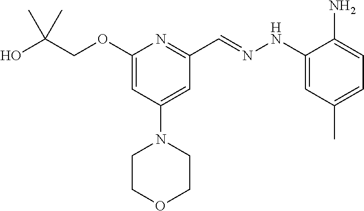 [SMILES: Cc1ccc(N)c(N/N=C/c2cc(N3CCOCC3)cc(OCC(C)(C)O)n2)c1] | 1-{6-[(2-Amino-5-methyl- phenyl)-hydrazonomethyl]-4- morpholin-4-yl-pyridin-2- yloxy}-2-methyl-propan-2-ol |
| 469 | 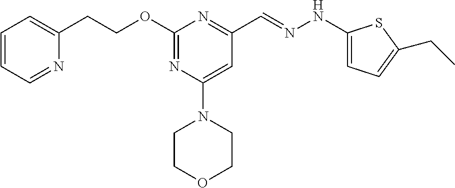 [SMILES: CCc1ccc(N/N=C/c2cc(N3CCOCC3)nc(OCCc3ccccn3)n2)s1] | N-(5-Ethyl-thiophen-2-yl)-N′- [6-morpholin-4-yl-2-(2- pyridin-2-yl-ethoxy)- pyrimidin-4-ylmethylene]- hydrazine |
| 470 | 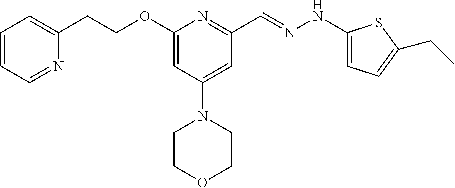 [SMILES: CCc1ccc(N/N=C/c2cc(N3CCOCC3)cc(OCCc3ccccn3)n2)s1] | N-(5-Ethyl-thiophen-2-yl)-N′- [4-morpholin-4-yl-6-(2- pyridin-2-yl-ethoxy)-pyridin-2- ylmethylene]-hydrazine |
| 471 | 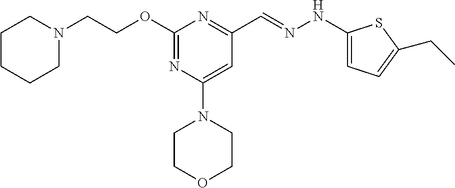 [SMILES: CCc1ccc(N/N=C/c2cc(N3CCOCC3)nc(OCCN3CCCCC3)n2)s1] | N-(5-Ethyl-thiophen-2-yl)-N′- [6-morpholin-4-yl-2-(2- piperidin-1-yl-ethoxy)- pyrimidin-4-ylmethylene]- hydrazine |
| 472 | 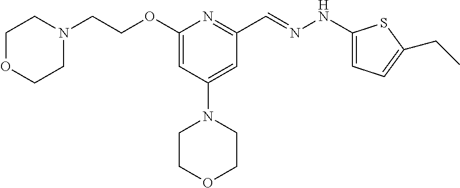 [SMILES: CCc1ccc(N/N=C/c2cc(N3CCOCC3)cc(OCCN3CCOCC3)n2)s1] | N-(5-Ethyl-thiophen-2-yl)-N′- [4-morpholin-4-yl-6-(2- morpholin-4-yl-ethoxy)- pyridin-2-ylmethylene]- hydrazine |
| 473 | 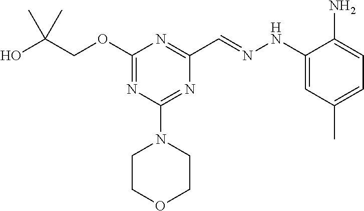 [SMILES: Cc1ccc(N)c(N/N=C/c2nc(OCC(C)(C)O)nc(N3CCOCC3)n2)c1] | 1-{4-[(2-Amino-5-methyl- phenyl)-hydrazonomethyl]-6- morpholin-4-yl-[1,3,5]triazin- 2-yloxy}-2-methyl-propan-2-ol |
| 474 | 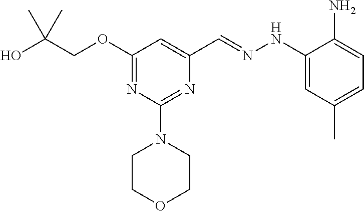 [SMILES: Cc1ccc(N)c(N/N=C/c2cc(OCC(C)(C)O)nc(N3CCOCC3)n2)c1] | 1-{6-[(2-Amino-5-methyl- phenyl)-hydrazonomethyl]-2- morpholin-4-yl-pyrimidin-4- yloxy}-2-methyl-propan-2-ol |
| 475 | 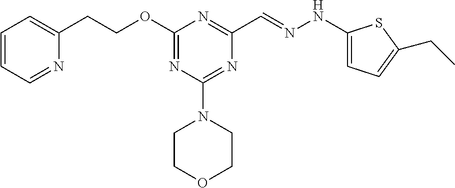 [SMILES: CCc1ccc(N/N=C/c2nc(OCCc3ccccn3)nc(N3CCOCC3)n2)s1] | N-(5-Ethyl-thiophen-2-yl)-N′- [4-morpholin-4-yl-6-(2- pyridin-2-yl-ethoxy)- [1,3,5]triazin-2-ylmethylene]- hydrazine |
| 476 | 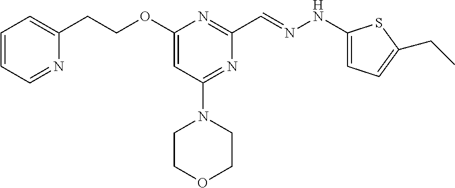 [SMILES: CCc1ccc(N/N=C/c2nc(OCCc3ccccn3)cc(N3CCOCC3)n2)s1] | N-(5-Ethyl-thiophen-2-yl)-N′- [4-morpholin-4-yl-6-(2- pyridin-2-yl-ethoxy)- pyrimidin-2-ylmethylene]- hydrazine |
| 477 | 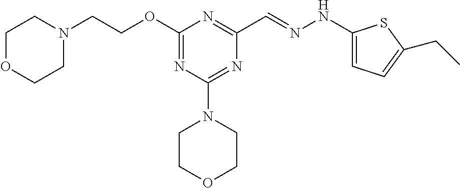 [SMILES: CCc1ccc(N/N=C/c2nc(OCCN3CCOCC3)nc(N3CCOCC3)n2)s1] | N-(5-Ethyl-thiophen-2-yl)-N′- [4-morpholin-4-yl-6-(2- morpholin-4-yl-ethoxy)- [1,3,5]triazin-2-ylmethylene]- hydrazine |
| 478 | 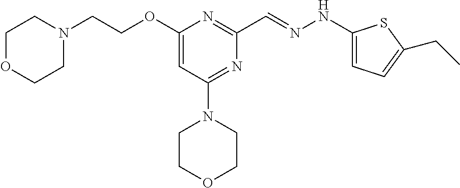 [SMILES: CCc1ccc(N/N=C/c2nc(OCCN3CCOCC3)cc(N3CCOCC3)n2)s1] | N-(5-Ethyl-thiophen-2-yl)-N′- [4-morpholin-4-yl-6-(2- morpholin-4-yl-ethoxy)- pyrimidin-2-ylmethylene]- hydrazine |
| 479 | 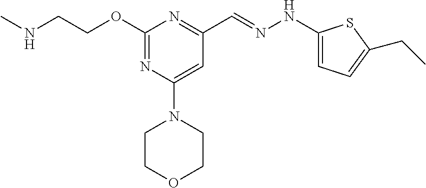 [SMILES: CCc1ccc(N/N=C/c2cc(N3CCOCC3)nc(OCCNC)n2)s1] | (2-{4-[(5-Ethyl-thiophen-2-yl)- hydrazonomethyl]-6- morpholin-4-yl-pyrimidin-2- yloxy}-ethyl)-methyl-amine |
| 480 | 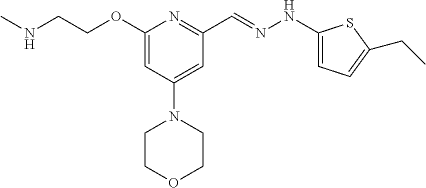 [SMILES: CCc1ccc(N/N=C/c2cc(N3CCOCC3)cc(OCCNC)n2)s1] | (2-{6-[(5-Ethyl-thiophen-2-yl)- hydrazonomethyl]-4- morpholin-4-yl-pyridin-2- yloxy}-ethyl)-methyl-amine |
| 481 | 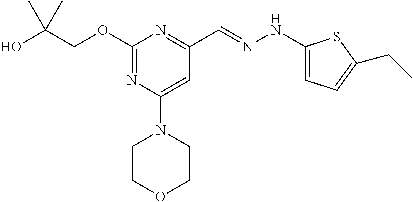 [SMILES: CCc1ccc(N/N=C/c2cc(N3CCOCC3)nc(OCC(C)(C)O)n2)s1] | 1-{4-[(5-Ethyl-thiophen-2-yl)- hydrazonomethyl]-6- morpholin-4-yl-pyrimidin-2- yloxy}-2-methyl-propan-2-ol |
| 482 | 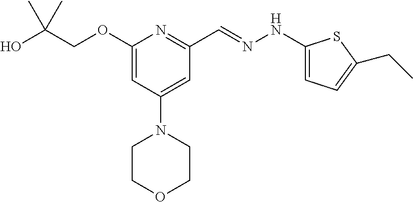 [SMILES: CCc1ccc(N/N=C/c2cc(N3CCOCC3)cc(OCC(C)(C)O)n2)s1] | 1-{6-[(5-Ethyl-thiophen-2-yl)- hydrazonomethyl]-4- morpholin-4-yl-pyridin-2- yloxy}-2-methyl-propan-2-ol |
| 483 | 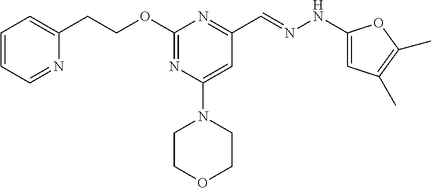 [SMILES: Cc1cc(N/N=C/c2cc(N3CCOCC3)nc(OCCc3ccccn3)n2)oc1C] | N-(4,5-Dimethyl-furan-2-yl)- N′-[6-morpholin-4-yl-2-(2- pyridin-2-yl-ethoxy)- pyrimidin-4-ylmethylene]- hydrazine |
| 484 | 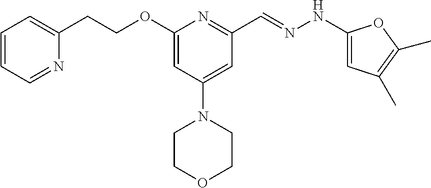 [SMILES: Cc1cc(N/N=C/c2cc(N3CCOCC3)cc(OCCc3ccccn3)n2)oc1C] | N-(4,5-Dimethyl-furan-2-yl)- N′-[4-morpholin-4-yl-6-(2- pyridin-2-yl-ethoxy)-pyridin-2- ylmethylene]-hydrazine |
| 485 | 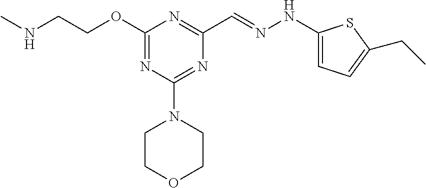 [SMILES: CCc1ccc(N/N=C/c2nc(OCCNC)nc(N3CCOCC3)n2)s1] | (2-{4-[(5-Ethyl-thiophen-2-yl)- hydrazonomethyl]-6- morpholin-4-yl-[1,3,5]triazin- 2-yloxy}-ethyl)-methyl-amine |
| 486 | 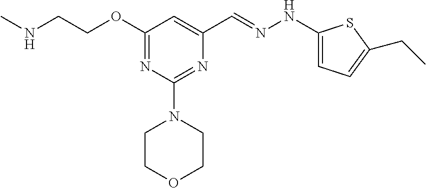 [SMILES: CCc1ccc(N/N=C/c2cc(OCCNC)nc(N3CCOCC3)n2)s1] | (2-{6-[(5-Ethyl-thiophen-2-yl)- hydrazonomethyl]-2- morpholin-4-yl-pyrimidin-4- yloxy}-ethyl)-methyl-amine |
| 487 | 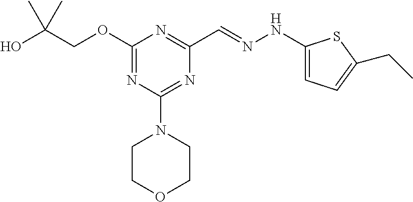 [SMILES: CCc1ccc(N/N=C/c2nc(OCC(C)(C)O)nc(N3CCOCC3)n2)s1] | 1-{4-[(5-Ethyl-thiophen-2-yl)- hydrazonomethyl]-6- morpholin-4-yl-[1,3,5]triazin- 2-yloxy}-2-methyl-propan-2-ol |
| 488 | 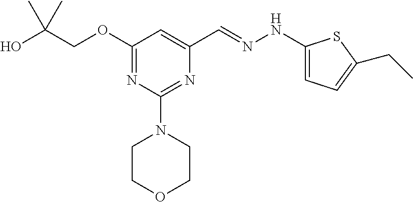 [SMILES: CCc1ccc(N/N=C/c2cc(OCC(C)(C)O)nc(N3CCOCC3)n2)s1] | 1-{6-[(5-Ethyl-thiophen-2-yl)- hydrazonomethyl]-2- morpholin-4-yl-pyrimidin-4- yloxy}-2-methyl-propan-2-ol |
| 489 | 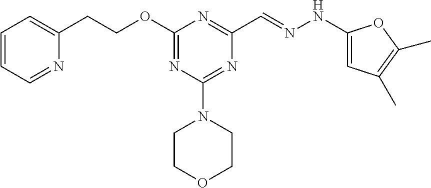 [SMILES: Cc1cc(N/N=C/c2nc(OCCc3ccccn3)nc(N3CCOCC3)n2)oc1C] | N-(4,5-Dimethyl-furan-2-yl)- N′-[4-morpholin-4-yl-6-(2- pyridin-2-yl-ethoxy)- [1,3,5]triazin-2-ylmethylene]- hydrazine |
| 490 | 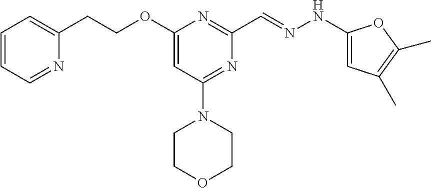 [SMILES: Cc1cc(N/N=C/c2nc(OCCc3ccccn3)cc(N3CCOCC3)n2)oc1C] | N-(4,5-Dimethyl-furan-2-yl)- N′-[4-morpholin-4-yl-6-(2- pyridin-2-yl-ethoxy)- pyrimidin-2-ylmethylene]- hydrazine |
| 491 | 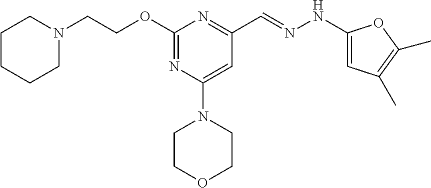 [SMILES: Cc1cc(N/N=C/c2cc(N3CCOCC3)nc(OCCN3CCCCC3)n2)oc1C] | N-(4,5-Dimethyl-furan-2-yl)- N′-[6-morpholin-4-yl-2-(2- piperidin-1-yl-thoxy)- pyrimidin-4-ylmethylene]- hydrazine |
| 492 | 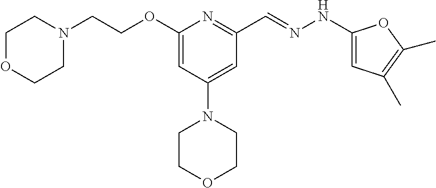 [SMILES: Cc1cc(N/N=C/c2cc(N3CCOCC3)cc(OCCN3CCOCC3)n2)oc1C] | N-(4,5-Dimethyl-furan-2-yl)- N′-[4-morpholin-4-yl-6-(2- morpholin-4-yl-ethoxy)- pyridin-2-ylmethylene]- hydrazine |
| 493 | 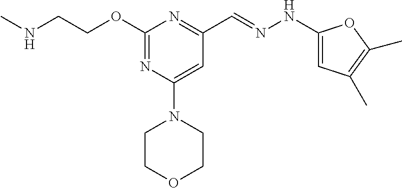 | (2-{4-[(4,5-Dimethyl-furan-2- yl)-hydrazonomethyl]-6- morpholin-4-yl-pyrimidin-2- yloxy}-ethyl)-methyl-amine |
| 494 | 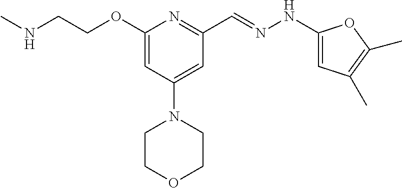 | (2-{6-[(4,5-Dimethyl-furan-2- yl)-hydrazonomethyl]-4- morpholin-4-yl-pyridin-2- yloxy}-ethyl)-methyl-amine |
| 495 | 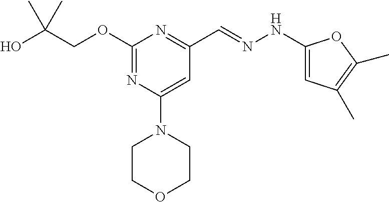 [SMILES: Cc1cc(N/N=C/c2cc(N3CCOCC3)nc(OCC(C)(C)O)n2)oc1C] | 1-{4-[(4,5-Dimethyl-furan-2- yl)-hydrazonomethyl]-6- morpholin-4-yl-pyrimidin-2- yloxy}-2-methyl-propan-2-ol |
| 496 | 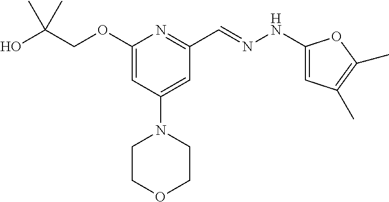 [SMILES: Cc1cc(N/N=C/c2cc(N3CCOCC3)cc(OCC(C)(C)O)n2)oc1C] | 1-{6-[(4,5-Dimethyl-furan-2- yl)-hydrazonomethyl]-4- morpholin-4-yl-pyridin-2- yloxy}-2-methyl-propan-2-ol |
| 497 | 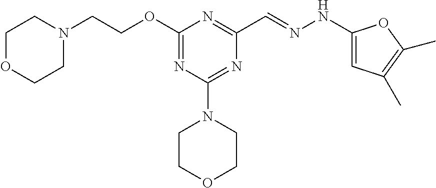 [SMILES: Cc1cc(N/N=C/c2nc(OCCN3CCOCC3)nc(N3CCOCC3)n2)oc1C] | N-(4,5-Dimethyl-furan-2-yl)- N′-[4-morpholin-4-yl-6-(2- morpholin-4-yl-ethoxy)- [1,3,5]triazin-2-ylmethylene]- hydrazine |
| 498 | 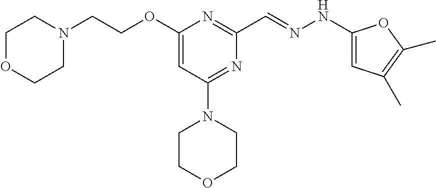 [SMILES: Cc1cc(N/N=C/c2nc(OCCN3CCOCC3)cc(N3CCOCC3)n2)oc1C] | N-(4,5-Dimethyl-furan-2-yl)- N′-[4-morpholin-4-yl-6-(2- morpholin-4-yl-ethoxy)- pyrimidin-2-ylmethylene]- hydrazine |
| 499 | 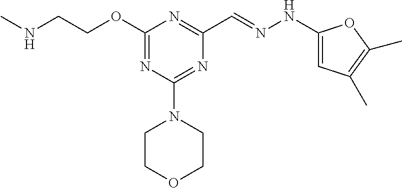 | (2-{4-[(4,5-Dimethyl-furan-2- yl)-hydrazonomethyl]-6- morpholin-4-yl-[1,3,5]triazin- 2-yloxy}-ethyl)-methyl-amine |
| 500 | 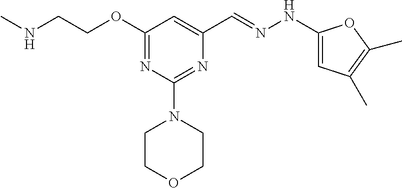 | (2-{6-[(4,5-Dimethyl-furan-2- yl)-hydrazonomethyl]-2- morpholin-4-yl-pyrimidin-4- yloxy}-ethyl)-methyl-amine |
| 501 | 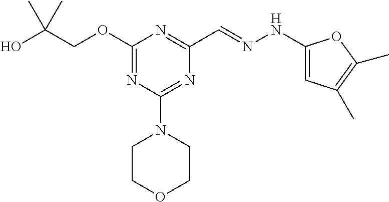 [SMILES: Cc1cc(N/N=C/c2nc(OCC(C)(C)O)nc(N3CCOCC3)n2)oc1C] | {4-[(4,5-Dimethyl-furan-2-yl)- hydrazonomethyl]-6- morpholin-4-yl-[1,3,5]triazin- 2-yloxy}-2-methyl-propan-2-ol |
| 502 | 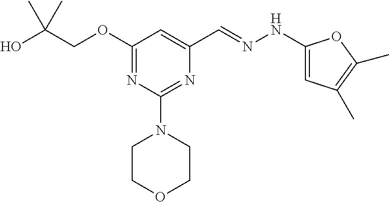 [SMILES: Cc1cc(N/N=C/c2cc(OCC(C)(C)O)nc(N3CCOCC3)n2)oc1C] | 1-{6-[(4,5-Dimethyl-furan-2- yl)-hydrazonomethyl]-2- morpholin-4-yl-pyrimidin-4- yloxy}-2-methyl-propan-2-ol |
| 503 | 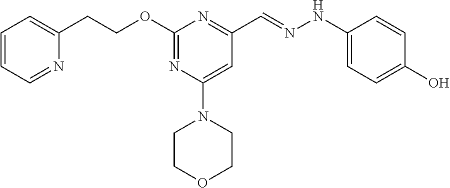 [SMILES: Oc1ccc(N/N=C/c2cc(N3CCOCC3)nc(OCCc3ccccn3)n2)cc1] | 4-{N′-[6-Morpholin-4-yl-2-(2- pyridin-2-yl-ethoxy)- pyrimidin-4-ylmethylene]- hydrazino}-phenol |
| 504 | 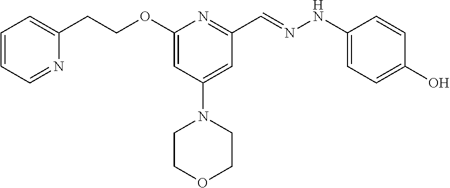 [SMILES: Oc1ccc(N/N=C/c2cc(N3CCOCC3)cc(OCCc3ccccn3)n2)cc1] | 4-{N′-[4-Morpholin-4-yl-6-(2- pyridin-2-yl-ethoxy)-pyridin-2- ylmethylene]-hydrazino}- phenol |
| 505 | 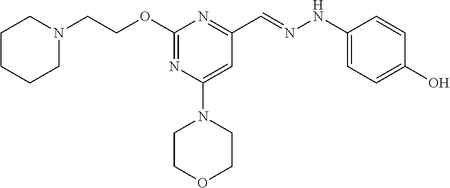 [SMILES: Oc1ccc(N/N=C/c2cc(N3CCOCC3)nc(OCCN3CCCCC3)n2)cc1] | 4-{N′-[6-Morpholin-4-yl-2-(2- piperidin-1-yl-ethoxy)- pyrimidin-4-ylmethylene]- hydrazino}-phenol |
| 506 | 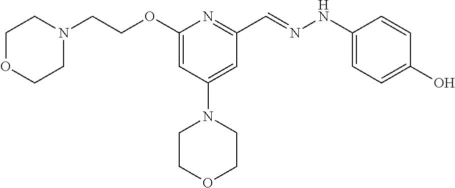 [SMILES: Oc1ccc(N/N=C/c2cc(N3CCOCC3)cc(OCCN3CCOCC3)n2)cc1] | 4-{N′-[4-Morpholin-4-yl-6-(2- morpholin-4-yl-ethoxy)- pyridin-2-ylmethylene]- hydrazino}-phenol |
| 507 | 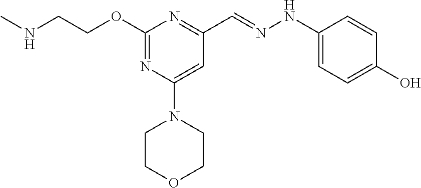 [SMILES: CNCCOc1nc(/C=N/Nc2ccc(O)cc2)cc(N2CCOCC2)n1] | 4-{N′-[2-(2-Methylamino- ethoxy)-6-morpholin-4-yl- pyrimidin-4-ylmethylene]- hydrazino}-phenol |
| 508 | 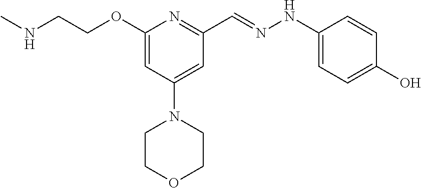 [SMILES: CNCCOc1cc(N2CCOCC2)cc(/C=N/Nc2ccc(O)cc2)n1] | 4-{N′-[6-(2-Methylamino- ethoxy)-4-morpholin-4-yl- pyridin-2-ylmethylene]- hydrazino}-phenol |
| 509 | 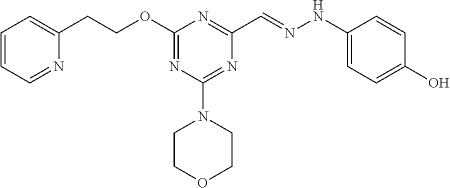 [SMILES: Oc1ccc(N/N=C/c2nc(OCCc3ccccn3)nc(N3CCOCC3)n2)cc1] | 4-{N′-[4-Morpholin-4-yl-6-(2- pyridin-2-yl-ethoxy)- [1,3,5]triazin-2-ylmethylene]- hydrazino}-phenol |
| 510 | 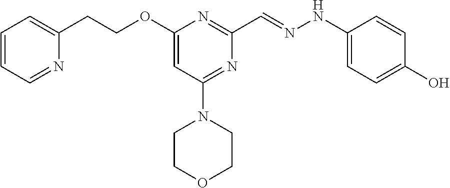 [SMILES: Oc1ccc(N/N=C/c2nc(OCCc3ccccn3)cc(N3CCOCC3)n2)cc1] | 4-{N′-[4-Morpholin-4-yl-6-(2- pyridin-2-yl-ethoxy)- pyrimidin-2-ylmethylene]- hydrazino}-phenol |
| 511 | 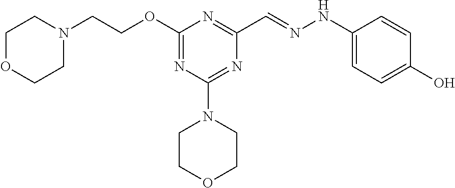 [SMILES: Oc1ccc(N/N=C/c2nc(OCCN3CCOCC3)nc(N3CCOCC3)n2)cc1] | 4-{N′-[4-Morpholin-4-yl-6-(2- morpholin-4-yl-ethoxy)- [1,3,5]triazin-2-ylmethylene]- hydrazino}-phenol |
| 512 | 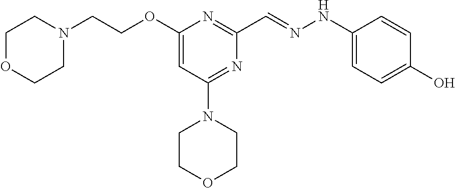 [SMILES: Oc1ccc(N/N=C/c2nc(OCCN3CCOCC3)cc(N3CCOCC3)n2)cc1] | 4-{N′-[4-Morpholin-4-yl-6-(2- morpholin-4-yl-ethoxy)- pyrimidin-2-ylmethylene]- hydrazino}-phenol |
| 513 | 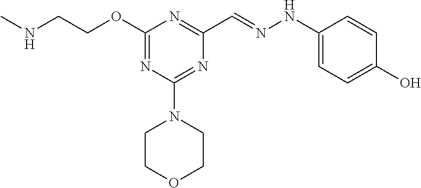 [SMILES: CNCCOc1nc(/C=N/Nc2ccc(O)cc2)nc(N2CCOCC2)n1] | 4-{N′-[4-(2-Methylamino- ethoxy)-6-morpholin-4-yl- [1,3,5]triazin-2-ylmethylene]- hydrazino}-phenol |
| 514 | 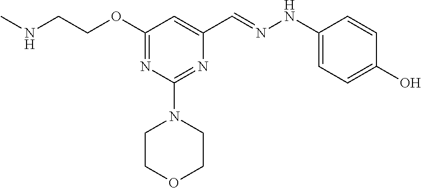 [SMILES: CNCCOc1cc(/C=N/Nc2ccc(O)cc2)nc(N2CCOCC2)n1] | 4-{N′-[6-(2-Methylamino- thoxy)-2-morpholin-4-yl- pyrimidin-4-ylmethylene]- hydrazino}-phenol |
| 515 | 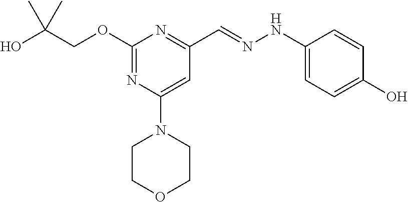 [SMILES: CC(C)(O)COc1nc(/C=N/Nc2ccc(O)cc2)cc(N2CCOCC2)n1] | 4-{N′-[2-(2-Hydroxy-2-methyl- propoxy)-6-morpholin-4-yl- pyrimidin-4-ylmethylene]- hydrazino}-phenol |
| 516 | 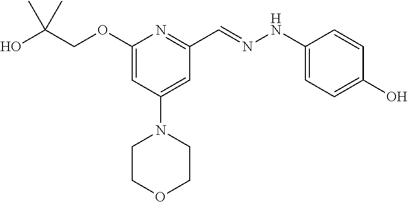 [SMILES: CC(C)(O)COc1cc(N2CCOCC2)cc(/C=N/Nc2ccc(O)cc2)n1] | 4-{N′-[6-(2-Hydroxy-2-methyl- propoxy)-4-morpholin-4-yl- pyridin-2-ylmethylene]- hydrazino}-phenol |
| 517 | 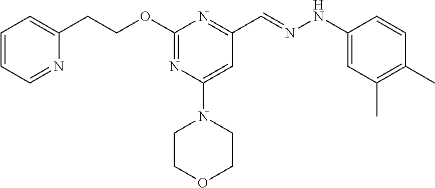 [SMILES: Cc1ccc(N/N=C/c2cc(N3CCOCC3)nc(OCCc3ccccn3)n2)cc1C] | N-(3,4-Dimethyl-phenyl)-N′- [6-morpholin-4-yl-2-(2- pyridin-2-yl-ethoxy)- pyrimidin-4-ylmethylene]- hydrazine |
| 518 | 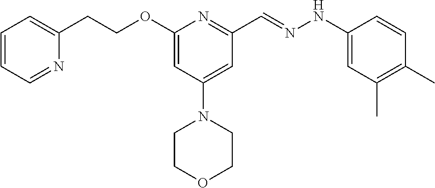 [SMILES: Cc1ccc(N/N=C/c2cc(N3CCOCC3)cc(OCCc3ccccn3)n2)cc1C] | N-(3,4-Dimethyl-phenyl)-N′- [4-morpholin-4-yl-6-(2- pyridin-2-yl-ethoxy)-pyridin-2- ylmethylene]-hydrazine |
| 519 | 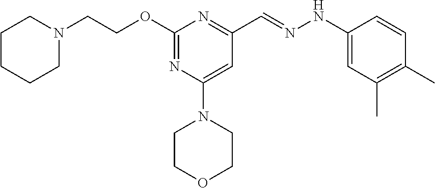 [SMILES: Cc1ccc(N/N=C/c2cc(N3CCOCC3)nc(OCCN3CCCCC3)n2)cc1C] | N-(3,4-Dimethyl-phenyl)-N′- [6-morpholin-4-yl-2-(2- piperidin-1-yl-ethoxy)- pyrimidin-4-ylmethylene]- hydrazine |
| 520 | 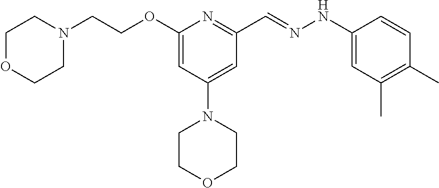 [SMILES: Cc1ccc(N/N=C/c2cc(N3CCOCC3)cc(OCCN3CCOCC3)n2)cc1C] | N-(3,4-Dimethyl-phenyl)-N′- [4-morpholin-4-yl-6-(2- morpholin-4-yl-ethoxy)- pyridin-2-ylmethylene]- hydrazine |
| 521 | 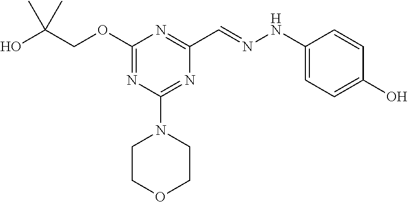 [SMILES: CC(C)(O)COc1nc(/C=N/Nc2ccc(O)cc2)nc(N2CCOCC2)n1] | 4-{N′-[4-(2-Hydroxy-2-methyl- propoxy)-6-morpholin-4-yl- [1,3,5]triazin-2-ylmethylene]- hydrazino}-phenol |
| 522 | 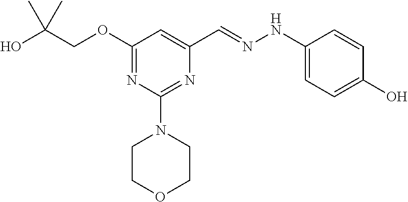 [SMILES: CC(C)(O)COc1cc(/C=N/Nc2ccc(O)cc2)nc(N2CCOCC2)n1] | 4-{N′-[6-(2-Hydroxy-2-methyl- propoxy)-2-morpholin-4-yl- pyrimidin-4-ylmethylene]- hydrazino}-phenol |
| 523 | 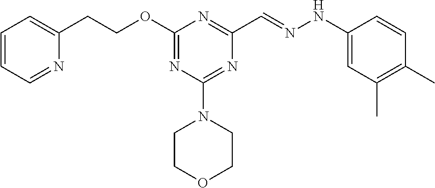 [SMILES: Cc1ccc(N/N=C/c2nc(OCCc3ccccn3)nc(N3CCOCC3)n2)cc1C] | N-(3,4-Dimethyl-phenyl)-N′- [4-morpholin-4-yl-6-(2- pyridin-2-yl-ethoxy)- [1,3,5]triazin-2-ylmethylene]- hydrazine |
| 524 | 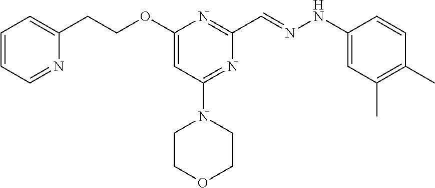 [SMILES: Cc1ccc(N/N=C/c2nc(OCCc3ccccn3)cc(N3CCOCC3)n2)cc1C] | N-(3,4-Dimethyl-phenyl)-N′- [4-morpholin-4-yl-6-(2- pyridin-2-yl-ethoxy)- pyrimidin-2-ylmethylene]- hydrazine |
| 525 | 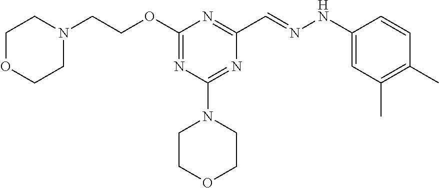 [SMILES: Cc1ccc(N/N=C/c2nc(OCCN3CCOCC3)nc(N3CCOCC3)n2)cc1C] | N-(3,4-Dimethyl-phenyl)-N′- [4-morpholin-4-yl-6-(2- morpholin-4-yl-ethoxy)- [1,3,5]triazin-2-ylmethylene]- hydrazine |
| 526 | 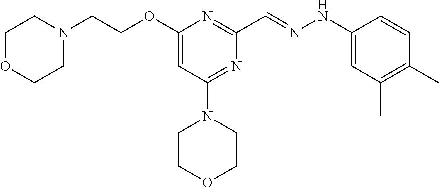 [SMILES: Cc1ccc(N/N=C/c2nc(OCCN3CCOCC3)cc(N3CCOCC3)n2)cc1C] | N-(3,4-Dimethyl-phenyl)-N′- [4-morpholin-4-yl-6-(2- morpholin-4-yl-ethoxy)- pyrimidin-2-ylmethylene]- hydrazine |
| 527 | 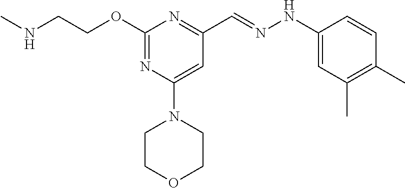 | (2-{4-[(3,4-Dimethyl-phenyl)- hydrazonomethyl]-6- morpholin-4-yl-pyrimidin-2- yloxy}-ethyl)-methyl-amine |
| 528 | 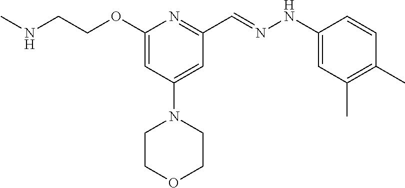 | (2-{6-[(3,4-Dimethyl-phenyl)- hydrazonomethyl]-4- morpholin-4-yl-pyridin-2- yloxy}-ethyl)-methyl-amine |
| 529 | 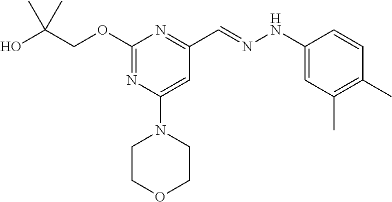 [SMILES: Cc1ccc(N/N=C/c2cc(N3CCOCC3)nc(OCC(C)(C)O)n2)cc1C] | 1-{4-[(3,4-Dimethyl-phenyl)- hydrazonomethyl]-6- morpholin-4-yl-pyrimidin-2- yloxy}-2-methyl-propan-2-ol |
| 530 | 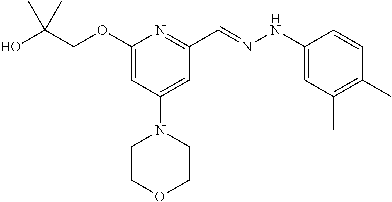 [SMILES: Cc1ccc(N/N=C/c2cc(N3CCOCC3)cc(OCC(C)(C)O)n2)cc1C] | 1-{6-[(3,4-Dimethyl-phenyl)- hydrazonomethyl]-4- morpholin-4-yl-pyridin-2- yloxy}-2-methyl-propan-2-ol |
| 531 | 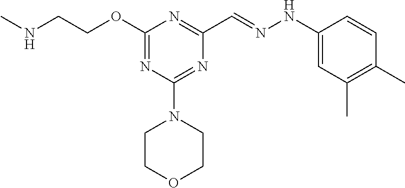 | (2-{4-[(3,4-Dimethyl-phenyl)- hydrazonomethyl]-6- morpholin-4-yl-[1,3,5]triazin- 2-yloxy}-ethyl)-methyl-amine |
| 532 | 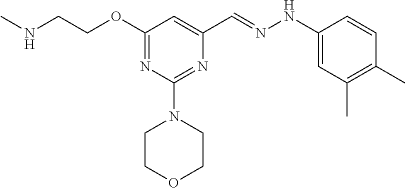 | (2-{6-[(3,4-Dimethyl-phenyl)- hydrazonomethyl]-2- morpholin-4-yl-pyrimidin-4- yloxy}-ethyl)-methyl-amine |
| 533 | 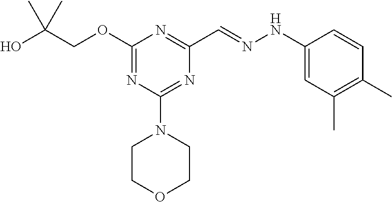 [SMILES: Cc1ccc(N/N=C/c2nc(OCC(C)(C)O)nc(N3CCOCC3)n2)cc1C] | 1-{4-[(3,4-Dimethyl-phenyl)- hydrazonomethyl]-6- morpholin-4-yl-[1,3,5]triazin- 2-yloxy}-2-methyl-propan-2-ol |
| 534 | 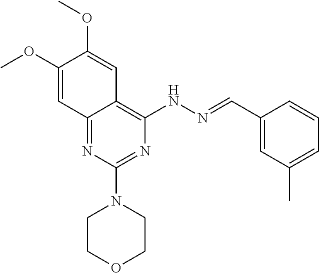 [SMILES: COc1cc2nc(N3CCOCC3)nc(N/N=C/c3cccc(C)c3)c2cc1OC] | N-(6,7-dimethoxy-2- morpholin-4-yl-quinazolin-4- yl)-N′-(3-methyl-benzylidene)- hydrazine |
| 535 | 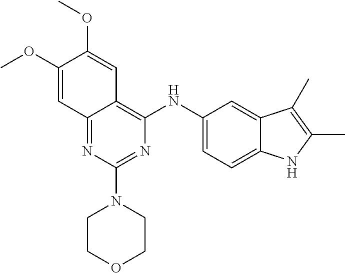 | (6,7-dimethoxy-2-morpholin-4- yl-quinazolin-4-yl)-(2,3- dimethyl-1H-indol-5-yl)-amine |
| 536 | 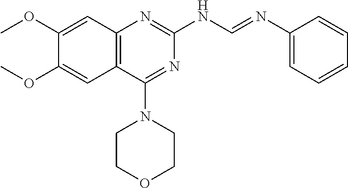 [SMILES: COc1cc2nc(N/C=N/c3ccccc3)nc(N3CCOCC3)c2cc1OC] | N-(6,7-dimethoxy-4- morpholin-4-yl-quinazolin-2- yl)-N′-(3-methyl-benzylidene)- hydrazine |
| 537 | 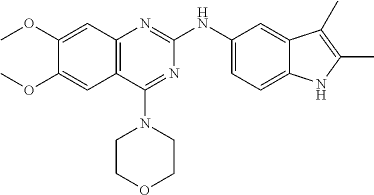 | (6,7-dimethoxy-4-morpholin-4- yl-quinazolin-2-yl)-(2,3- dimethyl-1H-indol-5-yl)-amine |
| 538 | 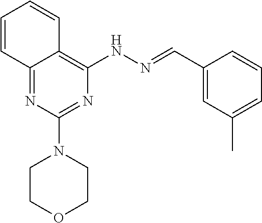 [SMILES: Cc1cccc(/C=N/Nc2nc(N3CCOCC3)nc3ccccc23)c1] | N-(3-methyl-benzylidene)-N′- (2-morpholin-4-yl-quinazolin- 4-yl)-hydrazine |
| 539 | 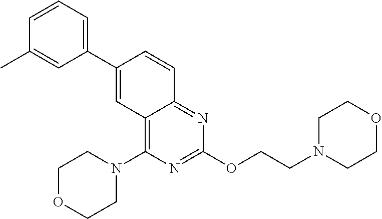 [SMILES: Cc1cccc(-c2ccc3nc(OCCN4CCOCC4)nc(N4CCOCC4)c3c2)c1] | 4-Morpholin-4-yl-2-(2- morpholin-4-yl-ethoxy)-6-m- tolyl-quinazoline |
| 540 | 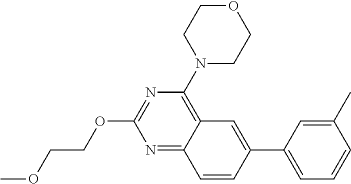 | 2-(2-Methoxy-ethoxy)-4- morpholin-4-yl-6-m-tolyl- quinazoline |
| 541 | 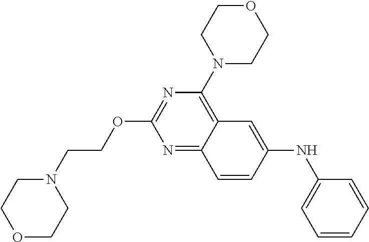 [SMILES: c1ccc(Nc2ccc3nc(OCCN4CCOCC4)nc(N4CCOCC4)c3c2)cc1] | [4-Morpholin-4-yl-2-(2- morpholin-4-yl-ethoxy)- quinazolin-6-yl]-phenyl-amine |
| 542 | 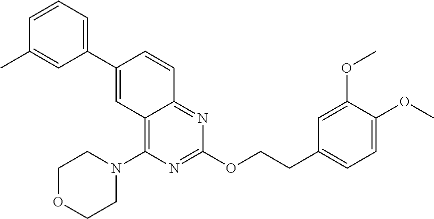 | 2-[2-(3,4-Dimethoxy-phenyl)- ethoxy]-4-morpholin-4-yl-6-m- tolyl-quinazoline |
| 543 | 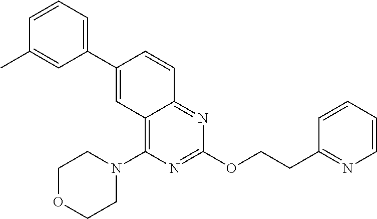 [SMILES: Cc1cccc(-c2ccc3nc(OCCc4ccccn4)nc(N4CCOCC4)c3c2)c1] | 4-Morpholin-4-yl-2-(2-pyridin- 2-yl-ethoxy)-6-m-tolyl- quinazoline |
| 544 | 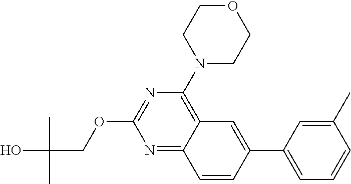 [SMILES: Cc1cccc(-c2ccc3nc(OCC(C)(C)O)nc(N4CCOCC4)c3c2)c1] | 2-Methyl-1-(4-morpholin-4-yl- 6-m-tolyl-quinazolin-2-yloxy)- propan-2-ol |
| 545 | 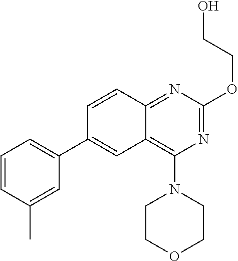 [SMILES: Cc1cccc(-c2ccc3nc(OCCO)nc(N4CCOCC4)c3c2)c1] | 2-(4-Morpholin-4-yl-6-m-tolyl- quinazolin-2-yloxy)-ethanol |
| 546 | 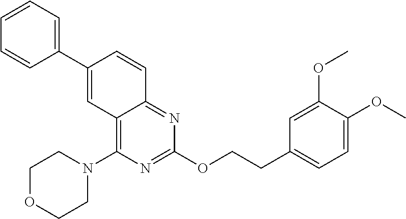 [SMILES: COc1ccc(CCOc2nc(N3CCOCC3)c3cc(-c4ccccc4)ccc3n2)cc1OC] | 2-[2-(3,4-Dimethoxy-phenyl)- ethoxy]-4-morpholin-4-yl-6- phenyl-quinazoline |
| 547 | 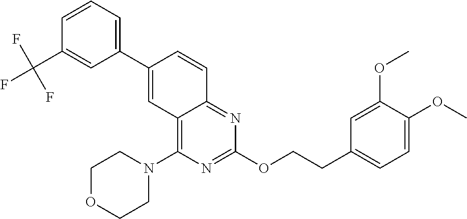 [SMILES: COc1ccc(CCOc2nc(N3CCOCC3)c3cc(-c4cccc(C(F)(F)F)c4)ccc3n2)cc1OC] | 2-[2-(3,4-Dimethoxy-phenyl)- ethoxy]-4-morpholin-4-yl-6-(3- trifluoromethyl-phenyl)- quinazoline |
| 548 | 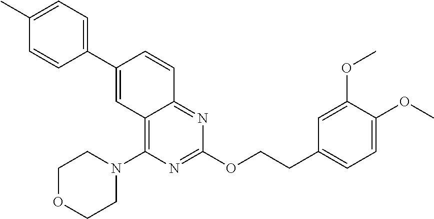 | 2-[2-(3,4-Dimethoxy-phenyl)- ethoxy]-4-morpholin-4-yl-6-p- tolyl-quinazoline |
| 549 | 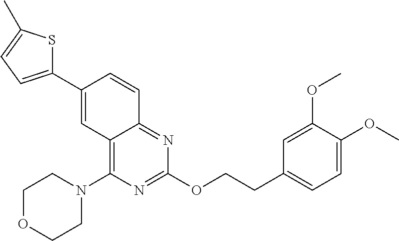 [SMILES: COc1ccc(CCOc2nc(N3CCOCC3)c3cc(-c4ccc(C)s4)ccc3n2)cc1OC] | 2-[2-(3,4-Dimethoxy-phenyl)- ethoxy]-6-(5-methyl-thiophen- 2-yl)-4-morpholin-4-yl- quinazoline |
| 550 | 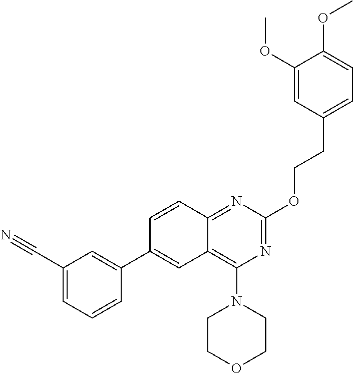 [SMILES: COc1ccc(CCOc2nc(N3CCOCC3)c3cc(-c4cccc(C#N)c4)ccc3n2)cc1OC] | 3-{2-[2-(3,4-Dimethoxy- phenyl)-ethoxy]-4-morpholin- 4-yl-quinazolin-6-yl}- benzonitrile |
| 551 | 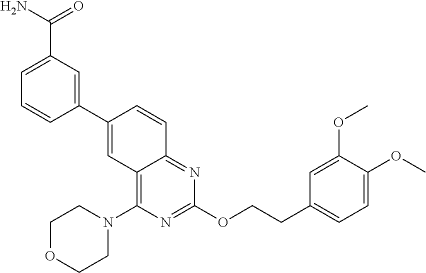 [SMILES: COc1ccc(CCOc2nc(N3CCOCC3)c3cc(-c4cccc(C(N)=O)c4)ccc3n2)cc1OC] | 3-{2-[2-(3,4-Dimethoxy- phenyl)-ethoxy]-4-morpholin- 4-yl-quinazolin-6-yl}- benzamide |
| 552 | 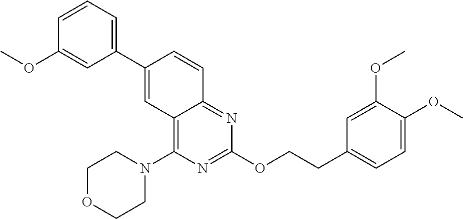 | 2-[2-(3,4-Dimethoxy-phenyl)- ethoxy]-6-(3-methoxy-phenyl)- 4-morpholin-4-yl-quinazoline |
| 553 | 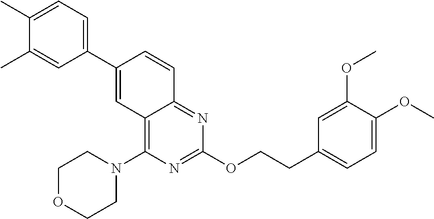 | 2-[2-(3,4-Dimethoxy-phenyl)- ethoxy]-6-(3,4-dimethyl- phenyl)-4-morpholin-4-yl- quinazoline |
| 554 | 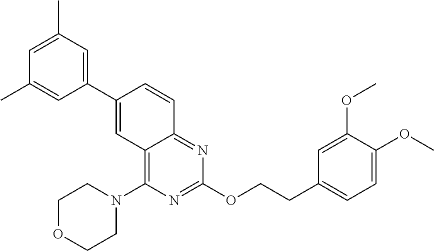 | 2-[2-(3,4-Dimethoxy-phenyl)- ethoxy]-6-(3,5-dimethyl- phenyl)-4-morpholin-4-yl- quinazoline |
| 555 | 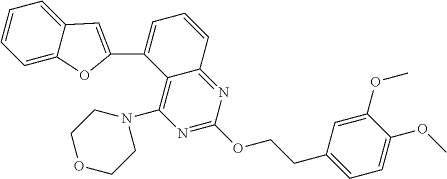 [SMILES: COc1ccc(CCOc2nc(N3CCOCC3)c3c(-c4cc5ccccc5o4)cccc3n2)cc1OC] | 6-Benzofuran-2-yl-2-[2-(3,4- dimethoxy-phenyl)-ethoxy]-4- morpholin-4-yl-quinazoline |
| 556 | 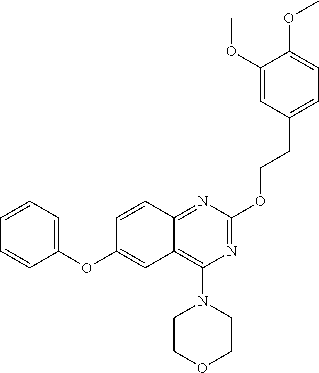 [SMILES: COc1ccc(CCOc2nc(N3CCOCC3)c3cc(Oc4ccccc4)ccc3n2)cc1OC] | 2-[2-(3,4-Dimethoxy-phenyl)- ethoxy]-4-morpholin-4-yl-6- phenoxy-quinazoline |
| 557 | 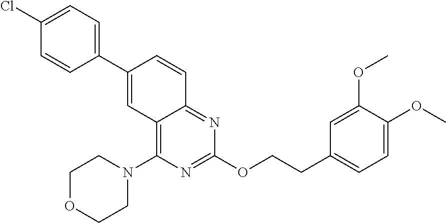 [SMILES: COc1ccc(CCOc2nc(N3CCOCC3)c3cc(-c4ccc(Cl)cc4)ccc3n2)cc1OC] | 6-(4-Chloro-phenyl)-2-[2-(3,4- dimethoxy-phenyl)-ethoxy]-4- morpholin-4-yl-quinazoline |
| 558 | 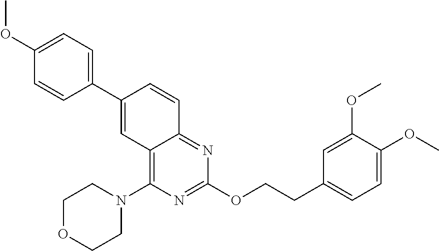 [SMILES: COc1ccc(-c2ccc3nc(OCCc4ccc(OC)c(OC)c4)nc(N4CCOCC4)c3c2)cc1] | 2-[2-(3,4-Dimethoxy-phenyl)- ethoxy]-6-(4-methoxy-phenyl)- 4-morpholin-4-yl-quinazoline |
| 559 | 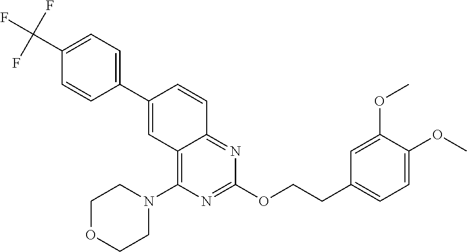 [SMILES: COc1ccc(CCOc2nc(N3CCOCC3)c3cc(-c4ccc(C(F)(F)F)cc4)ccc3n2)cc1OC] | 2-[2-(3,4-Dimethoxy-phenyl)- ethoxy]-4-morpholin-4-yl-6-(4- trifluoromethyl-phenyl)- quinazoline |
| 560 | 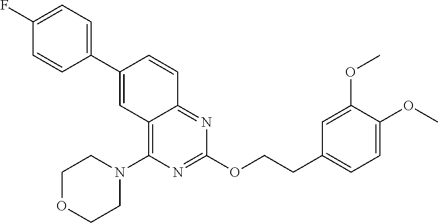 [SMILES: COc1ccc(CCOc2nc(N3CCOCC3)c3cc(-c4ccc(F)cc4)ccc3n2)cc1OC] | 6-(4-Fluoro-phenyl)-2-[2-(3,4- dimethoxy-phenyl)-ethoxy]-4- morpholin-4-yl-quinazoline |
| 561 | 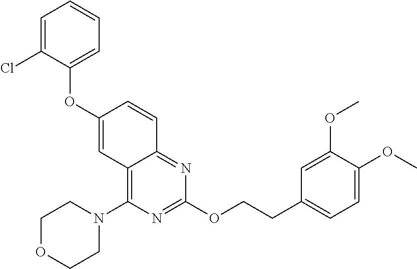 [SMILES: COc1ccc(CCOc2nc(N3CCOCC3)c3cc(Oc4ccccc4Cl)ccc3n2)cc1OC] | 6-(2-Chloro-phenoxy)-2-[2- (3,4-dimethoxy-phenyl)- ethoxy]-4-morpholin-4-yl- quinazoline |
| 562 | 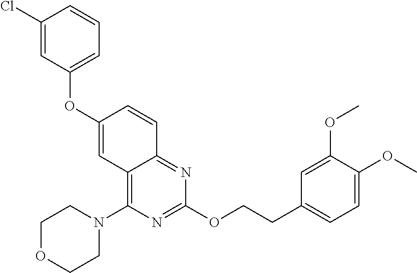 [SMILES: COc1ccc(CCOc2nc(N3CCOCC3)c3cc(Oc4cccc(Cl)c4)ccc3n2)cc1OC] | 6-(3-Chloro-phenoxy)-2-[2- (3,4-dimethoxy-phenyl)- ethoxy]-4-morpholin-4-yl quinazoline |
| 563 | 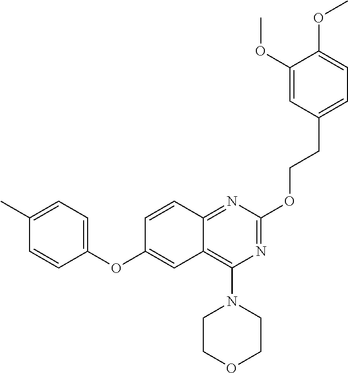 | 2-[2-(3,4-Dimethoxy-phenyl)- ethoxy]-4-morpholin-4-yl-6-p- tolyloxy-quinazoline |
| 564 | 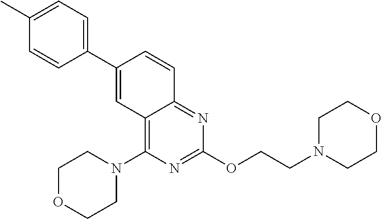 [SMILES: Cc1ccc(-c2ccc3nc(OCCN4CCOCC4)nc(N4CCOCC4)c3c2)cc1] | 4-Morpholin-4-yl-2-(2- morpholin-4-yl-ethoxy)-6-p- tolyl-quinazoline |
| 565 | 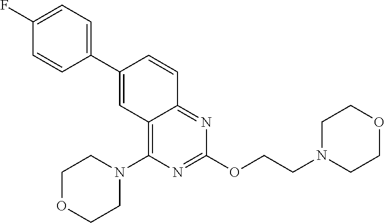 [SMILES: Fc1ccc(-c2ccc3nc(OCCN4CCOCC4)nc(N4CCOCC4)c3c2)cc1] | 6-(4-Fluoro-phenyl)-4- morpholin-4-yl-2-(2- morpholin-4-yl-ethoxy)- quinazoline |
| 566 | 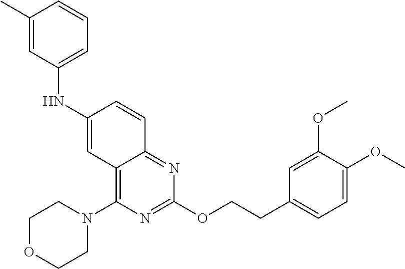 | {2-[2-(3,4-Dimethoxy-phenyl)- ethoxy]-4-morpholin-4-yl- quinazolin-6-yl}-m-tolyl-amine |
| 567 | 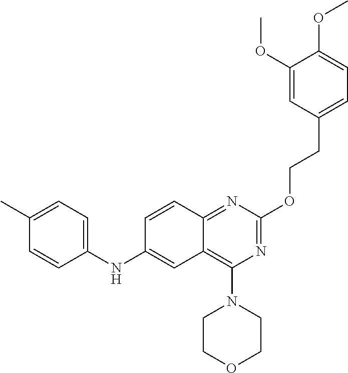 | {2-[2-(3,4-Dimethoxy-phenyl)- ethoxy]-4-morpholin-4-yl- quinazolin-6-yl}-p-tolyl-amine |
| 568 | 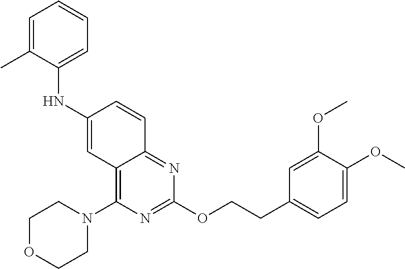 | {2-[2-(3,4-Dimethoxy-phenyl)- ethoxy]-4-morpholin-4-yl- quinazolin-6-yl}-o-tolyl-amine |
| 569 | 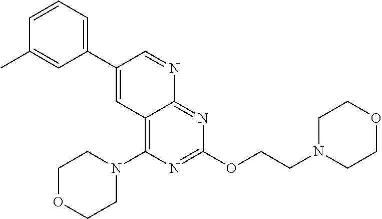 [SMILES: Cc1cccc(-c2cnc3nc(OCCN4CCOCC4)nc(N4CCOCC4)c3c2)c1] | 4-Morpholin-4-yl-2-(2- morpholin-4-yl-ethoxy)-6-m- tolyl-pyrido[2,3-d]pyrimidine |
| 570 | 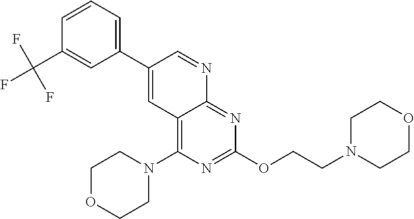 [SMILES: FC(F)(F)c1cccc(-c2cnc3nc(OCCN4CCOCC4)nc(N4CCOCC4)c3c2)c1] | 4-Morpholin-4-yl-2-(2- morpholin-4-yl-ethoxy)-6-(3- trifluoromethyl-phenyl)- pyrido[2,3-d]pyrimidine |
| 571 | 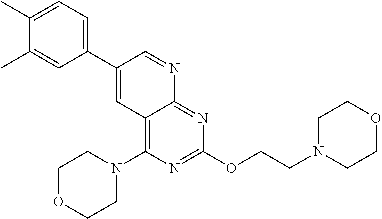 [SMILES: Cc1ccc(-c2cnc3nc(OCCN4CCOCC4)nc(N4CCOCC4)c3c2)cc1C] | 6-(3,4-Dimethyl-phenyl)-4- morpholin-4-yl-2-(2- morpholin-4-yl-ethoxy)- pyrido[2,3-d]pyrimidine |
| 572 | 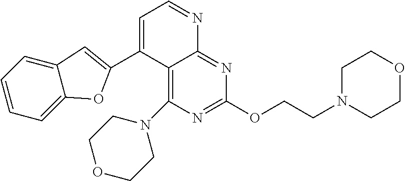 [SMILES: c1ccc2oc(-c3ccnc4nc(OCCN5CCOCC5)nc(N5CCOCC5)c34)cc2c1] | 6-Benzofuran-2-yl-4- morpholin-4-yl-2-(2- morpholin-4-yl-ethoxy)- pyrido[2,3-d]pyrimidine |
| 573 | 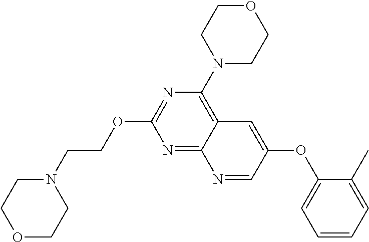 [SMILES: Cc1ccccc1Oc1cnc2nc(OCCN3CCOCC3)nc(N3CCOCC3)c2c1] | 4-Morpholin-4-yl-2-(2- morpholin-4-yl-ethoxy)-6-o- tolyloxy-pyrido[2,3- d]pyrimidine |
| 574 | 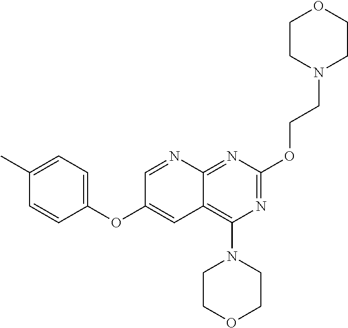 [SMILES: Cc1ccc(Oc2cnc3nc(OCCN4CCOCC4)nc(N4CCOCC4)c3c2)cc1] | 4-Morpholin-4-yl-2-(2- morpholin-4-yl-ethoxy)-6-p- tolyloxy-pyrido[2,3- d]pyrimidine |
| 575 | 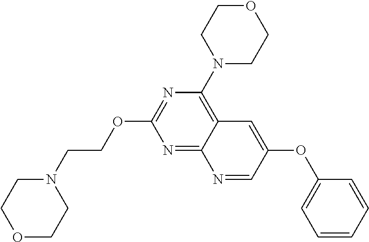 [SMILES: c1ccc(Oc2cnc3nc(OCCN4CCOCC4)nc(N4CCOCC4)c3c2)cc1] | 4-Morpholin-4-yl-2-(2- morpholin-4-yl-ethoxy)-6- phenoxy-pyrido[2,3- d]pyrimidine |
| 576 | 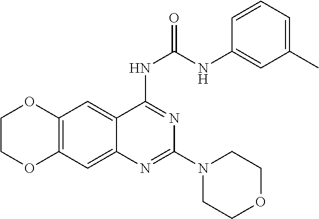 [SMILES: Cc1cccc(NC(=O)Nc2nc(N3CCOCC3)nc3cc4c(cc23)OCCO4)c1] | 1-(2-Morpholin-4-yl-7,8- dihydro-[1,4]dioxino[2,3- g]quinazolin-4-yl)-3-m-tolyl- urea |
| 577 | 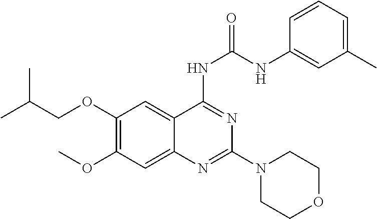 [SMILES: COc1cc2nc(N3CCOCC3)nc(NC(=O)Nc3cccc(C)c3)c2cc1OCC(C)C] | 1-(6-Isobutoxy-7-methoxy-2- morpholin-4-yl-quinazolin-4- yl)-3-m-tolyl-urea |
| 578 | 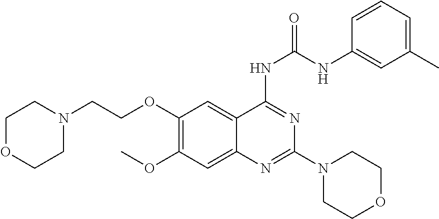 [SMILES: COc1cc2nc(N3CCOCC3)nc(NC(=O)Nc3cccc(C)c3)c2cc1OCCN1CCOCC1] | 1-[7-Methoxy-2-morpholin-2- yl-6-(2-morpholin-4-yl- ethoxy)-quinazolin-4-yl]-3-m- tolyl-urea |
| 579 | 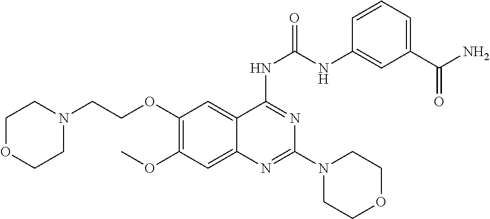 [SMILES: COc1cc2nc(N3CCOCC3)nc(NC(=O)Nc3cccc(C(N)=O)c3)c2cc1OCCN1CCOCC1] | 3-{3-[7-Methoxy-2-morpholin- 4-yl-6-(2-morpholin-4-yl- ethoxy)-quinazolin-4-yl]- ureido}-benzamide |
| 580 | 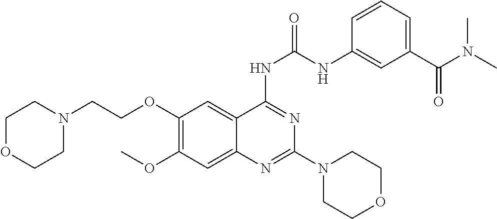 [SMILES: COc1cc2nc(N3CCOCC3)nc(NC(=O)Nc3cccc(C(=O)N(C)C)c3)c2cc1OCCN1CCOCC1] | 3-{3-[7-Methoxy-2-morpholin- 4-yl-6-(2-morpholin-4-yl- ethoxy)-quinazolin-4-yl]- ureido}-N,N-dimethyl- benzamide |
| 581 | 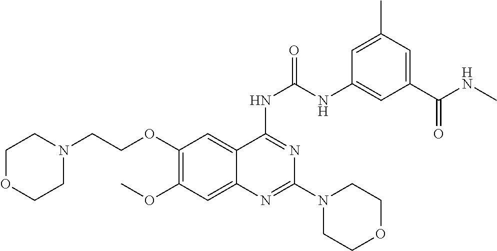 [SMILES: CNC(=O)c1cc(C)cc(NC(=O)Nc2nc(N3CCOCC3)nc3cc(OC)c(OCCN4CCOCC4)cc23)c1] | 3-{3-[7-Methoxy-2-morpholin- 4-yl-6-(2-morpholin-4-yl- ethoxy)-quinazolin-4-yl]- ureido}-5,N-dimethyl- benzamide |
| 582 | 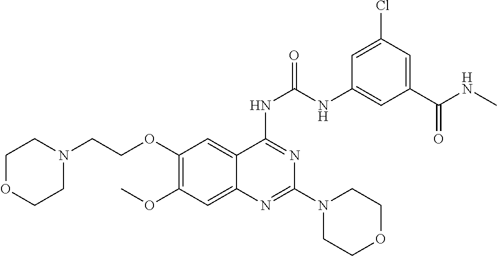 [SMILES: CNC(=O)c1cc(Cl)cc(NC(=O)Nc2nc(N3CCOCC3)nc3cc(OC)c(OCCN4CCOCC4)cc23)c1] | 3-Chloro-5-{3-[7-methoxy-2- morpholin-4-yl-6-(2- morpholin-4-yl-ethoxy)- quinazolin-4-yl]-ureido}-N- methyl-benzamide |
| 583 | 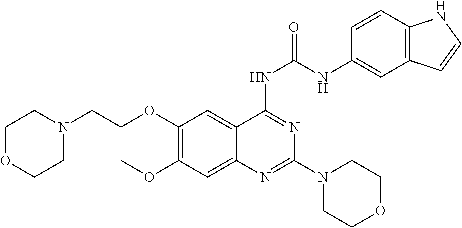 [SMILES: COc1cc2nc(N3CCOCC3)nc(NC(=O)Nc3ccc4[nH]ccc4c3)c2cc1OCCN1CCOCC1] | 1-(1H-Indol-5-yl)-3-[7- methoxy-2-morpholin-4-yl-6- (2-morpholin-4-yl-ethoxy)- quinazolin-4-yl]-urea |
| 584 | 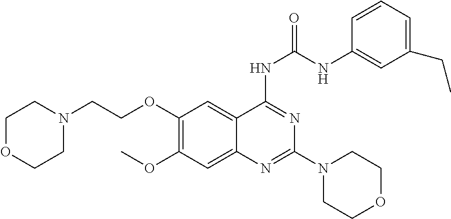 [SMILES: CCc1cccc(NC(=O)Nc2nc(N3CCOCC3)nc3cc(OC)c(OCCN4CCOCC4)cc23)c1] | 1-(3-Ethyl-phenyl)-3-[7- methoxy-2-morpholin-4-yl-6- (2-morpholin-4-yl-ethoxy)- quinazolin-4-yl]-urea |
| 585 | 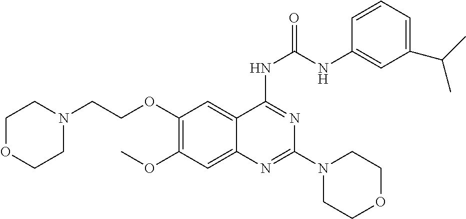 [SMILES: COc1cc2nc(N3CCOCC3)nc(NC(=O)Nc3cccc(C(C)C)c3)c2cc1OCCN1CCOCC1] | 1-(3-Isopropyl-phenyl)-3-[7- methoxy-2-morpholin-4-yl-6- (2-morpholin-4-yl-ethoxy)- quinazolin-4-yl]-urea |
| 586 | 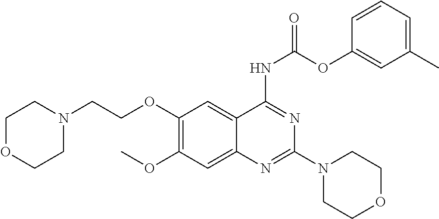 [SMILES: COc1cc2nc(N3CCOCC3)nc(NC(=O)Oc3cccc(C)c3)c2cc1OCCN1CCOCC1] | [7-Methoxy-2-morpholin-4-yl- 6-(2-morpholin-4-yl-ethoxy)- quinazolin-4-yl]-carbamic acid m-tolyl ester |
| 587 | 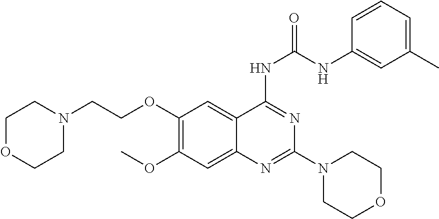 [SMILES: COc1cc2nc(N3CCOCC3)nc(NC(=O)Nc3cccc(C)c3)c2cc1OCCN1CCOCC1] | m-Tolyl-carbamic acid 7- methoxy-2-morpholin-4-yl-6- (2-morpholin-4-yl-ethoxy)- quinazolin-4-yl ester |
| 588 | 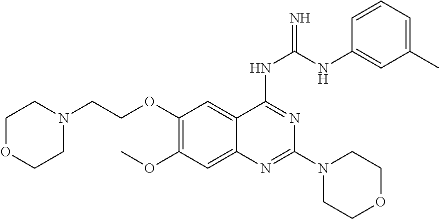 [SMILES: COc1cc2nc(N3CCOCC3)nc(NC(=N)Nc3cccc(C)c3)c2cc1OCCN1CCOCC1] | N-[7-Methoxy-2-morpholin-4- yl-6-(2-morpholin-4-yl- ethoxy)-quinazolin-4-yl]-N′-m- tolyl-guanidine |
| 589 | 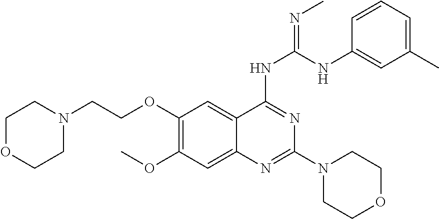 [SMILES: C/N=C(\Nc1cccc(C)c1)Nc1nc(N2CCOCC2)nc2cc(OC)c(OCCN3CCOCC3)cc12] | N-[7-Methoxy-2-morpholin-4- yl-6-(2-morpholin-4-yl- ethoxy)-quinazolin-4-yl]-N′- methyl-N″-m-tolyl-guanidine |
| 590 | 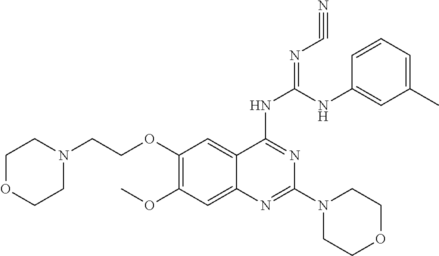 [SMILES: COc1cc2nc(N3CCOCC3)nc(N/C(=N/C#N)Nc3cccc(C)c3)c2cc1OCCN1CCOCC1] | N-[7-Methoxy-2-morpholin-4- yl-6-(2-morpholin-4-yl- ethoxy)quinazolin-4-yl]-N′- cyano-N″-m-tolyl-guanidine |
| 591 | 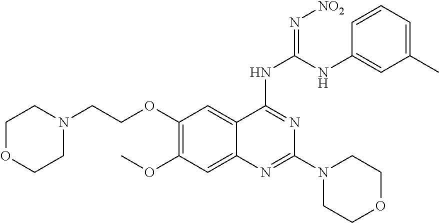 [SMILES: COc1cc2nc(N3CCOCC3)nc(N/C(=N/[N+](=O)[O-])Nc3cccc(C)c3)c2cc1OCCN1CCOCC1] | N-[7-Methoxy-2-morpholin-4- yl-6-(2-morpholin-4-yl- ethoxy)-quinazolin-4-yl]-N′- nitro-N″-m-tolyl-guanidine |
| 592 | 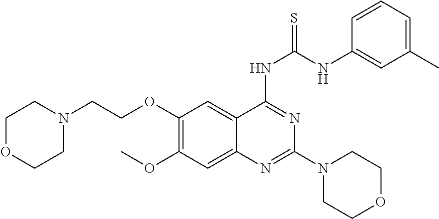 [SMILES: COc1cc2nc(N3CCOCC3)nc(NC(=S)Nc3cccc(C)c3)c2cc1OCCN1CCOCC1] | 1-[7-Methoxy-2-morpholin-4- yl-6-(2-morpholin-4-yl- ethoxy)-quinazolin-4-yl]-3-m- tolyl-thioure |
| 593 | 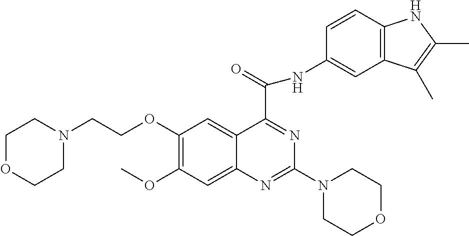 [SMILES: COc1cc2nc(N3CCOCC3)nc(C(=O)Nc3ccc4[nH]c(C)c(C)c4c3)c2cc1OCCN1CCOCC1] | 7-Methoxy-2-morpholin-4-yl- 6-(2-morpholin-4-yl-ethoxy)- quinazoline-4-carboxylic acid (2,3-dimethyl-1H-indol-5-yl)- amide |
| 594 | 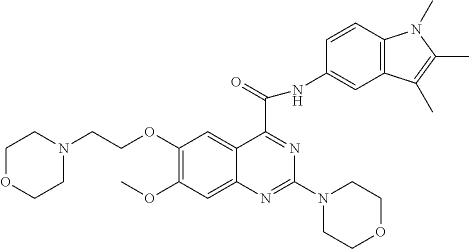 [SMILES: COc1cc2nc(N3CCOCC3)nc(C(=O)Nc3ccc4c(c3)c(C)c(C)n4C)c2cc1OCCN1CCOCC1] | 7-Methoxy-2-morpholin-4-yl- 6-(2-morpholin-4-yl-ethoxy)- quinazoline-4-carboxylic acid (1,2,3-trimethyl-1H-indol-5- yl)-amide |
| 595 | 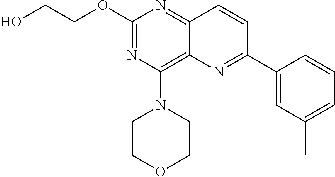 [SMILES: Cc1cccc(-c2ccc3nc(OCCO)nc(N4CCOCC4)c3n2)c1] | 2-(4-Morpholin-4-yl-6-m-tolyl- pyrido[3,2-d]pyrimidin-2- yloxy)-ethanol |
| 596 | 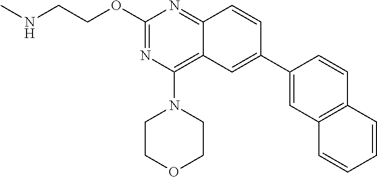 [SMILES: CNCCOc1nc(N2CCOCC2)c2cc(-c3ccc4ccccc4c3)ccc2n1] | Methyl-[2-(4-morpholin-4-yl- 6-naphthalen-2-yl-quinazolin- 2-yloxy)-ethyl]-amine |
| 597 | 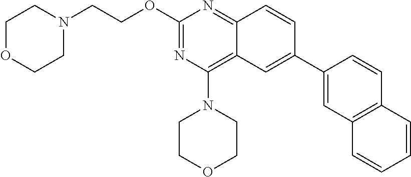 [SMILES: c1ccc2cc(-c3ccc4nc(OCCN5CCOCC5)nc(N5CCOCC5)c4c3)ccc2c1] | 4-Morpholin-4-yl-2-(2- morpholin-4-yl-ethoxy)-6- naphthalen-2-yl-quinazoline |
| 598 | 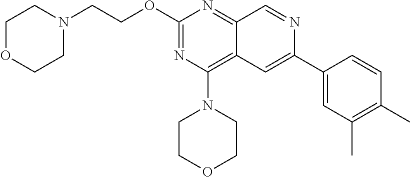 [SMILES: Cc1ccc(-c2cc3c(N4CCOCC4)nc(OCCN4CCOCC4)nc3cn2)cc1C] | 6-(3,4-Dimethyl-phenyl)-4- morpholin-4-yl-2-(2- morpholin-4-yl-ethoxy)- pyrido[3,4-d]pyrimidine |
| 599 | 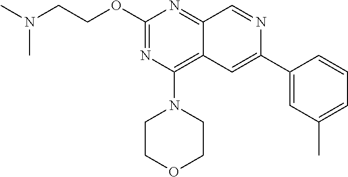 [SMILES: Cc1cccc(-c2cc3c(N4CCOCC4)nc(OCCN(C)C)nc3cn2)c1] | Dimethyl-[2-(4-morpholin-4- yl-6-m-tolyl-pyrido[3,4- d]pyrimidin-2-yloxy)-ethyl]- amine |
| 600 | 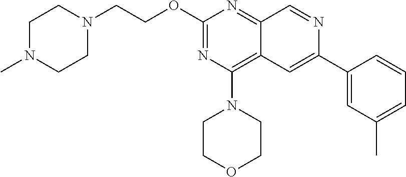 [SMILES: Cc1cccc(-c2cc3c(N4CCOCC4)nc(OCCN4CCN(C)CC4)nc3cn2)c1] | 2-[2-(4-Methyl-piperazin-1-yl)- ethoxy]-4-morpholin-4-yl-6-m- tolyl-pyrido[3,4-d]pyrimidine |
| 601 | 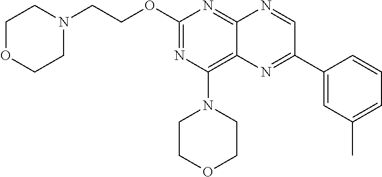 [SMILES: Cc1cccc(-c2cnc3nc(OCCN4CCOCC4)nc(N4CCOCC4)c3n2)c1] | 4-Morpholin-4-yl-2-(2- morpholin-4-yl-ethoxy)-6-m- tolyl-pteridine |
| 602 | 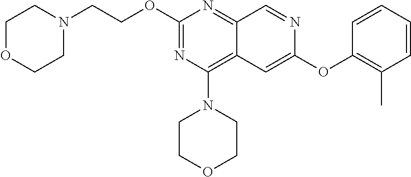 [SMILES: Cc1ccccc1Oc1cc2c(N3CCOCC3)nc(OCCN3CCOCC3)nc2cn1] | 4-Morpholin-4-yl-2-(2- morpholin-4-yl-ethoxy)-6-o- tolyloxy-pyrido[3,4- d]pyrimidine |
| 603 | 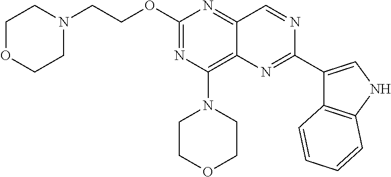 [SMILES: c1ccc2c(-c3ncc4nc(OCCN5CCOCC5)nc(N5CCOCC5)c4n3)c[nH]c2c1] | 6-(1H-Indol-3-yl)-4-morpholin- 4-yl-2-(2-morpholin-4-yl- ethoxy)-pyrimido[5,4- d]pyrimidine |
| 604 | 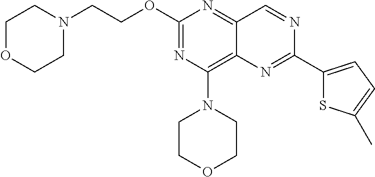 [SMILES: Cc1ccc(-c2ncc3nc(OCCN4CCOCC4)nc(N4CCOCC4)c3n2)s1] | 6-(5-Methyl-thiophen-2-yl)-4- morpholin-4-yl-2-(2- morpholin-4-yl-ethoxy)- pyrimido[5,4-d]pyrimidine |
| 605 | 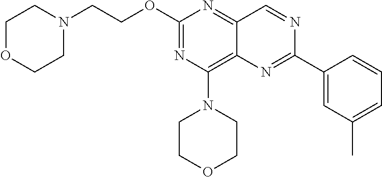 [SMILES: Cc1cccc(-c2ncc3nc(OCCN4CCOCC4)nc(N4CCOCC4)c3n2)c1] | 4-Morpholin-4-yl-2-(2- morpholin-4-yl-ethoxy)-6-m- tolyl-pyrimido[5,4- d]pyrimidine |
| 606 | 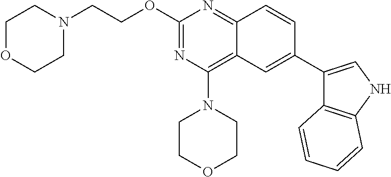 [SMILES: c1ccc2c(-c3ccc4nc(OCCN5CCOCC5)nc(N5CCOCC5)c4c3)c[nH]c2c1] | 6-(1H-Indol-3-yl)-4-morpholin- 4-yl-2-(2-morpholin-4-yl- ethoxy)-quinazoline |
| 607 | 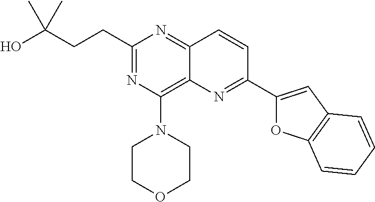 [SMILES: CC(C)(O)CCc1nc(N2CCOCC2)c2nc(-c3cc4ccccc4o3)ccc2n1] | 4-(6-Benzofuran-2-yl-4- morpholin-4-yl-pyrido[3,2- d]pyrimidin-2-yl)-2-methyl- butan-2-ol |
| 608 | 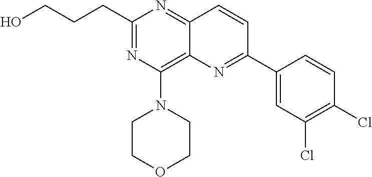 [SMILES: OCCCc1nc(N2CCOCC2)c2nc(-c3ccc(Cl)c(Cl)c3)ccc2n1] | 3-[6-(3,4-Dichloro-phenyl)-4- morpholin-4-yl-pyrido[3,2- d]pyrimidin-2-yl]-propan-1-ol |
| 609 | 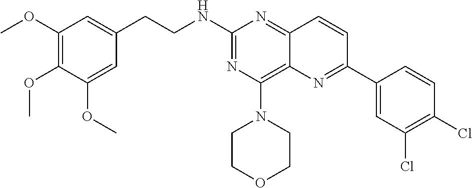 [SMILES: COc1cc(CCNc2nc(N3CCOCC3)c3nc(-c4ccc(Cl)c(Cl)c4)ccc3n2)cc(OC)c1OC] | [6-(3,4-Dichloro-phenyl)-4- morpholin-4-yl-pyrido[3,2- d]pyrimidin-2-yl]-[2-(3,4,5- trimethoxy-phenyl)-ethyl]- amine |
| 610 | 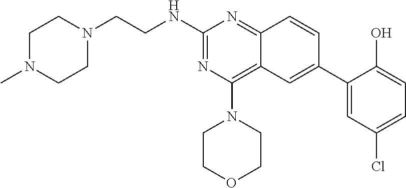 [SMILES: CN1CCN(CCNc2nc(N3CCOCC3)c3cc(-c4cc(Cl)ccc4O)ccc3n2)CC1] | 4-Chloro-2-{2-[2-(4-methyl- piperiazin-1-yl)-ethylamino]-4- morpholin-4-yl-quinazolin-6- yl}-phenol |
| 611 | 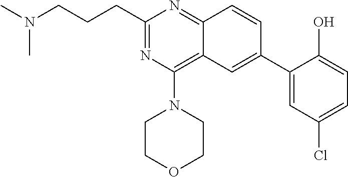 [SMILES: CN(C)CCCc1nc(N2CCOCC2)c2cc(-c3cc(Cl)ccc3O)ccc2n1] | 4-Chloro-2-{2-[2-(4-methyl- piperazin-1-yl)-ethylamino]-4- morpholin-4-yl-quinazolin-6- yl}-phenol |
| 612 | 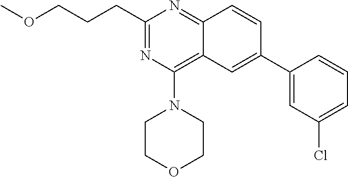 [SMILES: COCCCc1nc(N2CCOCC2)c2cc(-c3cccc(Cl)c3)ccc2n1] | 6-(3-Chloro-phenyl)-2-(3- methoxy-propyl)-4-morpholin- 4-yl-quinazoline |
| 613 | 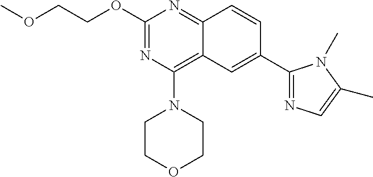 | 6-(1,5-Dimethyl-1H-imidazol- 2-yl)-2-(2-methoxy-ethoxy)-4- morpholin-4-yl-quinazoline |
| 614 | 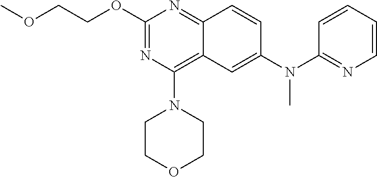 [SMILES: COCCOc1nc(N2CCOCC2)c2cc(N(C)c3ccccn3)ccc2n1] | [2-(2-Methoxy-ethoxy)-4- morpholin-4-yl-quinazolin-6- yl]-methyl-pyridin-2-yl-amine |
| 615 | 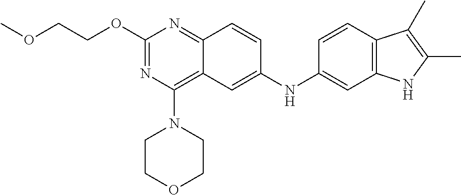 | (2,3-Dimethyl-1H-indol-6-yl)- [2-(2-methoxy-ethoxy)-4- morpholin-4-yl-quinazolin-6- yl]-amine |
| 616 | 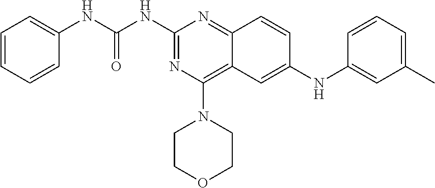 [SMILES: Cc1cccc(Nc2ccc3nc(NC(=O)Nc4ccccc4)nc(N4CCOCC4)c3c2)c1] | 1-(4-Morpholin-4-yl-6-m- tolylamino-quinazolin-2-yl)-3- phenyl-urea |
| 617 | 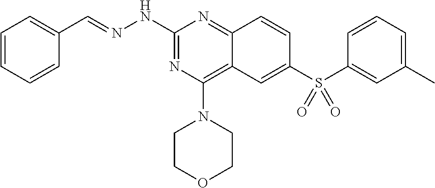 [SMILES: Cc1cccc(S(=O)(=O)c2ccc3nc(N/N=C/c4ccccc4)nc(N4CCOCC4)c3c2)c1] | N-Benzylidene-N′-[4- morpholin-4-yl-6-(toluene-3- sulfonyl)-quinazolin-2-yl]- hydrazine |
| 618 | 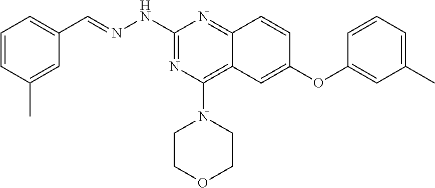 [SMILES: Cc1cccc(/C=N/Nc2nc(N3CCOCC3)c3cc(Oc4cccc(C)c4)ccc3n2)c1] | N-(3-Methyl-benzylidene)-N′- (4-morpholin-4-yl-6-m- tolyloxy-quinazolin-2-yl)- hydrazine |
| 619 | 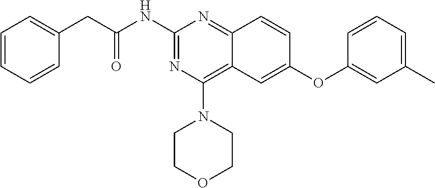 [SMILES: Cc1cccc(Oc2ccc3nc(NC(=O)Cc4ccccc4)nc(N4CCOCC4)c3c2)c1] | N-(4-Morpholin-4-yl-6-m- tolyloxy-quinazolin-2-yl)-2- phenyl-acetamide |
| 620 | 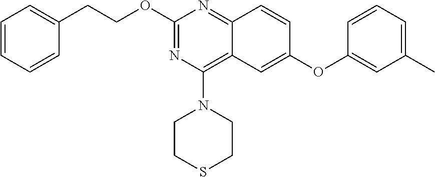 [SMILES: Cc1cccc(Oc2ccc3nc(OCCc4ccccc4)nc(N4CCSCC4)c3c2)c1] | 2-Phenethyloxy-4- thiomorpholin-4-yl-6-m- tolyloxy-quinazoline |
| 621 | 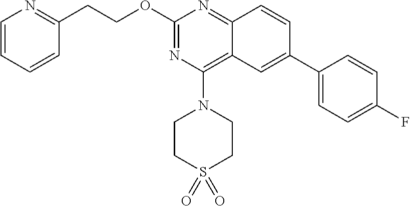 [SMILES: O=S1(=O)CCN(c2nc(OCCc3ccccn3)nc3ccc(-c4ccc(F)cc4)cc23)CC1] | 4-(1,1-Dioxo-116- thiomorpholin-4-yl)-6-(4- fluoro-phenyl)-2-(2-pyridin-2- yl-ethoxy)-quinazoline |
| 622 | 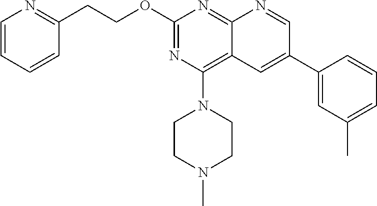 [SMILES: Cc1cccc(-c2cnc3nc(OCCc4ccccn4)nc(N4CCN(C)CC4)c3c2)c1] | 4-(4-Methyl-piperazin-1-yl)-2- (2-pyridin-2-yl-ethoxy)-6-m- tolyl-pyrido[2,3-d]pyrimidine |
| 623 | 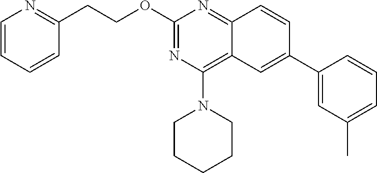 [SMILES: Cc1cccc(-c2ccc3nc(OCCc4ccccn4)nc(N4CCCCC4)c3c2)c1] | 4-Piperidin-1-yl-2-(2-pyridin- 2-yl-ethoxy)-6-m-tolyl- pyrido[3,2-d]pyrimidine |
| 624 | 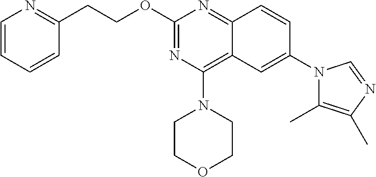 [SMILES: Cc1ncn(-c2ccc3nc(OCCc4ccccn4)nc(N4CCOCC4)c3c2)c1C] | 6-(4,5-Dimethyl-imidazol-1- yl)-4-morpholin-4-yl-2-(2- pyridin-2-yl-ethoxy)- quinazoline |
| 625 | 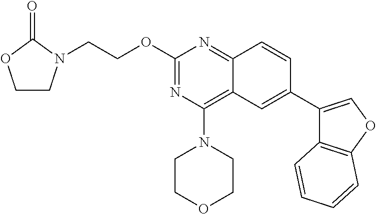 [SMILES: O=C1OCCN1CCOc1nc(N2CCOCC2)c2cc(-c3coc4ccccc34)ccc2n1] | 3-[2-(6-Benzofuran-3-yl-4- morpholin-4-yl-quinazolin-2- yloxy)-ethyl]-oxazolidin-2-one |
| 626 | 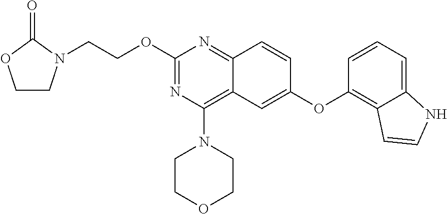 [SMILES: O=C1OCCN1CCOc1nc(N2CCOCC2)c2cc(Oc3cccc4[nH]ccc34)ccc2n1] | 3-{2-[6-(1H-Indol-4-yloxy)-4- morpholin-4-yl-quinazolin-2- yloxy]-ethyl}-oxazolidin-2-one |
| 627 | 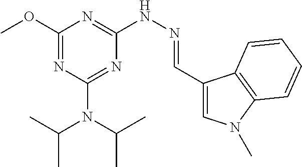 [SMILES: COc1nc(N/N=C/c2cn(C)c3ccccc23)nc(N(C(C)C)C(C)C)n1] | Diisopropyl-{4-methoxy-6-[N′- (1-methyl-1H-indol-3- ylmethylene)-hydrazino]- [1,3,5]triazin-2-yl}-amine |
| 628 | 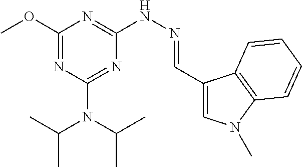 [SMILES: COc1nc(N/N=C/c2cn(C)c3ccccc23)nc(N(C(C)C)C(C)C)n1] | {4-[N′-(1H-Indol-3- ylmethylene)-hydrazino]-6- methoxy-[1,3,5]triazin-2-yl}- diisopropyl-amine |
| 629 | 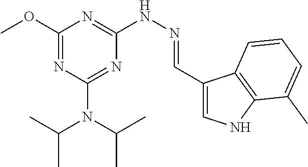 | Diisopropyl-{4-methoxy-6-[N′- (7-methyl-1H-indol-3- ylmethylene)-hydrazino]- [1,3,5]triazin-2-yl}-amine |
| 630 | 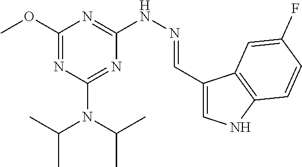 [SMILES: COc1nc(N/N=C/c2c[nH]c3ccc(F)cc23)nc(N(C(C)C)C(C)C)n1] | {4-[N′-(5-Fluoro-1H-indol-3- ylmethylene)-hydrazino]-6- methoxy-[1,3,5]triazin-2-yl}- diisopropyl-amine |
| 631 | 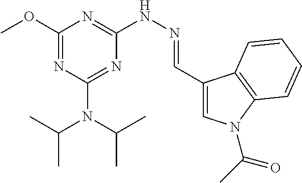 [SMILES: COc1nc(N/N=C/c2cn(C(C)=O)c3ccccc23)nc(N(C(C)C)C(C)C)n1] | 1-{3-[(4-Diisopropylamino-6- methoxy-[1,3,5]triazin-2-yl)- hydrazonomethyl]-indol-1-yl}- ethanone |
| 632 | 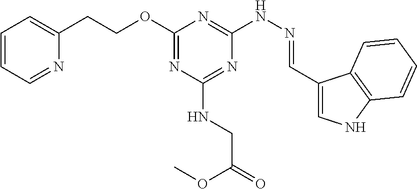 [SMILES: COC(=O)CNc1nc(N/N=C/c2c[nH]c3ccccc23)nc(OCCc2ccccn2)n1] | [4-[N′-(1H-Indol-3- ylmethylene)-hydrazino]-6-(2- pyridin-2-yl-ethoxy)- [1,3,5]triazin-2-ylamino]-acetic acid methyl ester |
| 633 | 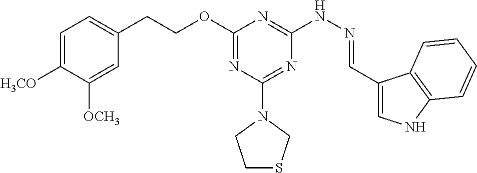 [SMILES: COc1ccc(CCOc2nc(N/N=C/c3c[nH]c4ccccc34)nc(N3CCSC3)n2)cc1OC] | N-{4-[2-(3,4-Dimethoxy- phenyl)-ethoxy]-6-thiazolidin- 3-yl-[1,3,5]triazin-2-yl}-N′- (1H-indol-3-ylmethylene)- hydrazine |
| 634 | 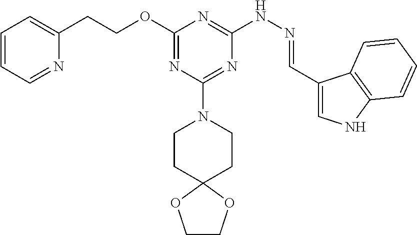 [SMILES: C(=N\Nc1nc(OCCc2ccccn2)nc(N2CCC3(CC2)OCCO3)n1)/c1c[nH]c2ccccc12] | N-[4-(1,4-Dioxa-8-aza- spiro[4.5]dec-8-yl)-6-(2- pyridin-2-yl-ethoxy)- [1,3,5]triazin-2-yl]-N′-(1H- indol-3-ylmethylene)-hydrazine |
| 635 | 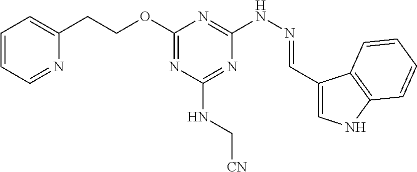 [SMILES: N#CCNc1nc(N/N=C/c2c[nH]c3ccccc23)nc(OCCc2ccccn2)n1] | [4-[N′-(1H-Indol-3- ylmethylene)-hydrazino]-6-(2- pyridin-2-yl-ethoxy)- [1,3,5]triazin-2-ylamino]- acetonitrile |
| 636 | 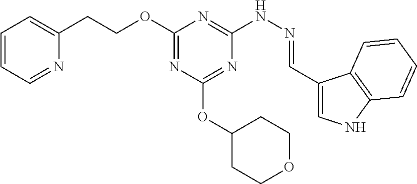 [SMILES: C(=N\Nc1nc(OCCc2ccccn2)nc(OC2CCOCC2)n1)/c1c[nH]c2ccccc12] | N-(1H-Indol-3-ylmethylene)- N′-[4-(2-pyridin-2-yl-ethoxy)- 6-(tetrahydro-pyran-4-yloxy)- [1,3,5]triazin-2-yl]-hydrazine |
| 637 | 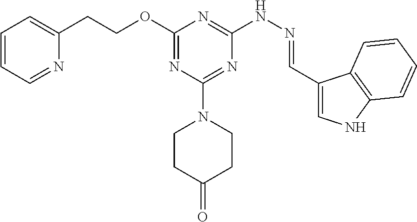 [SMILES: O=C1CCN(c2nc(N/N=C/c3c[nH]c4ccccc34)nc(OCCc3ccccn3)n2)CC1] | 1-[4-[N′-(1H-Indol-3- ylmethylene)-hydrazino]-6-(2- pyridin-2-yl-ethoxy)- [1,3,5]triazin-2-yl]-piperidin-4- one |
| 638 | 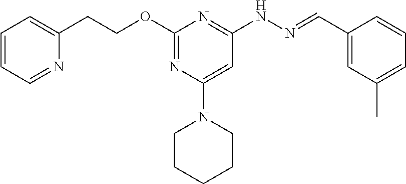 [SMILES: Cc1cccc(/C=N/Nc2cc(N3CCCCC3)nc(OCCc3ccccn3)n2)c1] | N-(3-Methyl-benzylidene)-N′- [6-piperidin-1-yl-2-(2-pyridin- 2-yl-ethoxy)-pyrimidin-4-yl]- hydrazine |
| 639 | 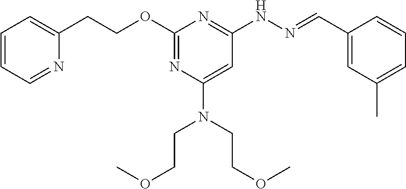 [SMILES: COCCN(CCOC)c1cc(N/N=C/c2cccc(C)c2)nc(OCCc2ccccn2)n1] | Bis-(2-methoxy-ethyl)-[6-[N′- (3-methyl-benzylidene)- hydrazino]-2-(2-pyridin-2-yl- ethoxy)-pyrimidin-4-yl]-amine |
| 640 | 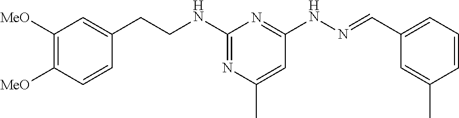 [SMILES: COc1ccc(CCNc2nc(C)cc(N/N=C/c3cccc(C)c3)n2)cc1OC] | [2-(3,4-Dimethoxy-phenyl)- ethyl]-{4-methyl-6-[N′-(3- methyl-benzylidene)- hydrazino]-pyrimidin-2-yl}- amine |
| 641 | 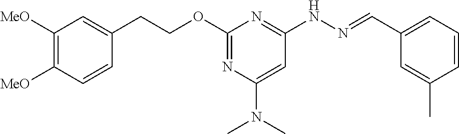 [SMILES: COc1ccc(CCOc2nc(N/N=C/c3cccc(C)c3)cc(N(C)C)n2)cc1OC] | {2-[2-(3,4-Dimethoxy-phenyl)- ethoxy]-6-[N′-(3-methyl- benzylidene)-hydrazino]- pyrimidin-4-yl}-dimethyl- amine |
| 642 | 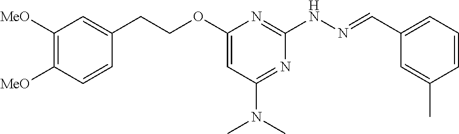 [SMILES: COc1ccc(CCOc2cc(N(C)C)nc(N/N=C/c3cccc(C)c3)n2)cc1OC] | {6-[2-(3,4-Dimethoxy-phenyl)- ethoxy]-2-[N′-(3-methyl- benzylidene)-hydrazino]- pyrimidin-4-yl}-dimethyl- amine |
| 643 | 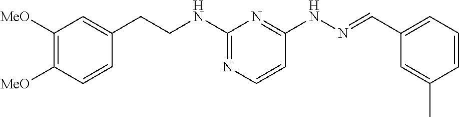 [SMILES: COc1ccc(CCNc2nccc(N/N=C/c3cccc(C)c3)n2)cc1OC] | [2-(3,4-Dimethoxy-phenyl)- ethyl]-{4-[N′-(3-methyl- benzylidene)-hydrazino]- pyrimidin-2-yl}-amine |
| 644 | 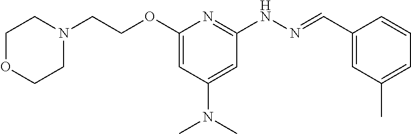 [SMILES: Cc1cccc(/C=N/Nc2cc(N(C)C)cc(OCCN3CCOCC3)n2)c1] | Dimethyl-[2-[N′-(3-methyl- benzylidene)-hydrazino]-6-(2- morpholin-4-yl-ethoxy)- pyridin-4-yl]-amine |
| 645 | 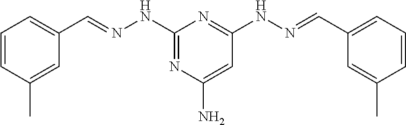 [SMILES: Cc1cccc(/C=N/Nc2cc(N)nc(N/N=C/c3cccc(C)c3)n2)c1] | 2,6-Bis-[N′-(3-methyl- benzylidene)-hydrazino]- pyrimidin-2-ylamine |
| 646 | 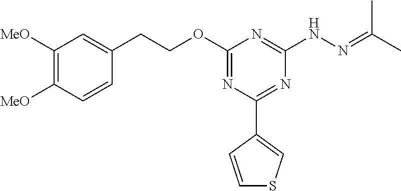 [SMILES: COc1ccc(CCOc2nc(NN=C(C)C)nc(-c3ccsc3)n2)cc1OC] | N-{4-[2-(3,4-Dimethoxy- phenyl)-ethoxy]-6-thiophen-3- yl-[1,3,5]triazin-2-yl}-N′- isopropylidene-hydrazine |
| 647 | 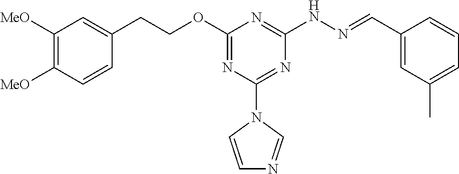 [SMILES: COc1ccc(CCOc2nc(N/N=C/c3cccc(C)c3)nc(-n3ccnc3)n2)cc1OC] | N-{4-[2-(3,4-Dimethoxy- phenyl)-ethoxy]-6-imidazol-1- yl-[1,3,5]triazin-2-yl}-N′-(3- methyl-benzylidene)- hydrazine |
| 648 | 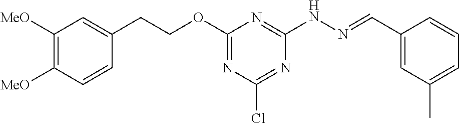 [SMILES: COc1ccc(CCOc2nc(Cl)nc(N/N=C/c3cccc(C)c3)n2)cc1OC] | N-{4-Chloro-6-[2-(3,4- dimethoxy-phenyl)-ethoxy]- [1,3,5]triazin-2-yl}-N′-(3- methyl-benzylidene)- hydrazine |
| 649 | 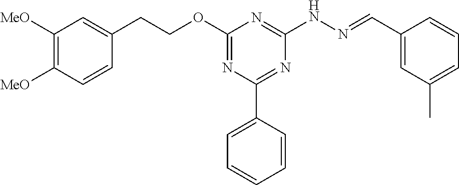 [SMILES: COc1ccc(CCOc2nc(N/N=C/c3cccc(C)c3)nc(-c3ccccc3)n2)cc1OC] | N-{4-[2-(3,4-Dimethoxy- phenyl)-ethoxy]-6-phenyl- [1,3,5]triazin-2-yl}-N′-(3- methyl-benzylidene)- hydrazine |
| 650 | 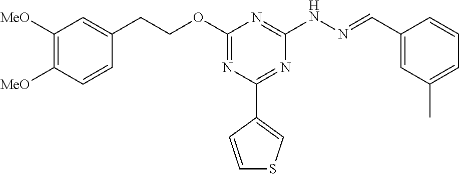 [SMILES: COc1ccc(CCOc2nc(N/N=C/c3cccc(C)c3)nc(-c3ccsc3)n2)cc1OC] | N-{4-[2-(3,4-Dimethoxy- phenyl)-ethoxy]-6-thiophen-3- yl-[1,3,5]triazin-2-yl}-N′-(3- methyl-benzylidene)- hydrazine |
| 651 | 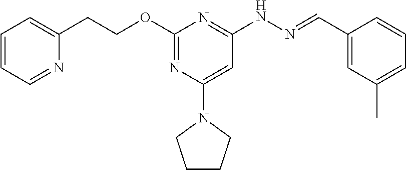 [SMILES: Cc1cccc(/C=N/Nc2cc(N3CCCC3)nc(OCCc3ccccn3)n2)c1] | N-(3-Methyl-benzylidene)-N′- [2-(2-pyridin-2-yl-ethoxy)-6- pyrrolidin-1-yl-pyrimidin-4- yl]-hydrazine |
| 652 | 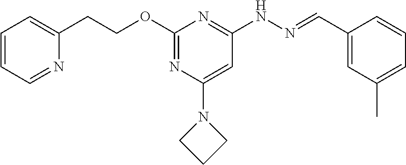 [SMILES: Cc1cccc(/C=N/Nc2cc(N3CCC3)nc(OCCc3ccccn3)n2)c1] | N-[6-Azetidin-1-yl-2-(2- pyridin-2-yl-ethoxy)- pyrimidin-4-yl]-N′-(3-methyl- benzylidene)-hydrazine |
| 653 | 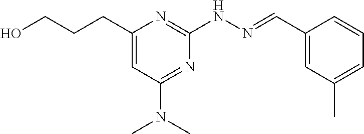 [SMILES: Cc1cccc(/C=N/Nc2nc(CCCO)cc(N(C)C)n2)c1] | 3-{6-Dimethylamino-2-[N′-(3- methyl-benzylidene)- hydrazino]-pyrimidin-4-yl}- propan-1-ol |
| 654 | 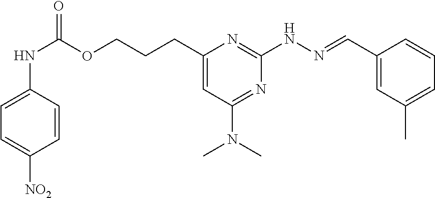 [SMILES: Cc1cccc(/C=N/Nc2nc(CCCOC(=O)Nc3ccc([N+](=O)[O-])cc3)cc(N(C)C)n2)c1] | (4-Nitro-phenyl)-carbamic acid 3-{6-dimethylamino-2-[N′-(3- methyl-benzylidene)- hydrazino]-pyrimidin-4-yl}- propyl ester |
| 655 | 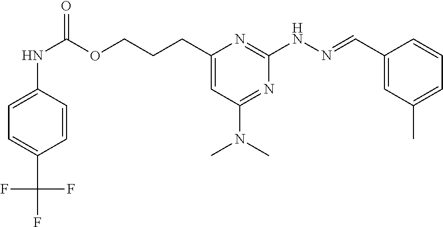 [SMILES: Cc1cccc(/C=N/Nc2nc(CCCOC(=O)Nc3ccc(C(F)(F)F)cc3)cc(N(C)C)n2)c1] | (4-Trifluoromethyl-phenyl)- carbamic acid 3-{6- dimethylamino-2-[N′-(3- methyl-benzylidene)- hydrazino]-pyrimidin-4-yl}- propyl ester |
| 656 | 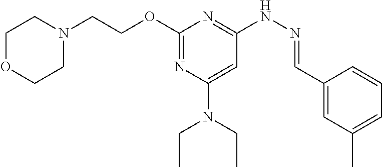 [SMILES: CCN(CC)c1cc(N/N=C/c2cccc(C)c2)nc(OCCN2CCOCC2)n1] | Diethyl-[6-[N′-(3-methyl- benzylidene)-hydrazino]-2-(2- morpholin-4-yl-ethoxy)- pyrimidin-4-yl]-amine |
| 657 | 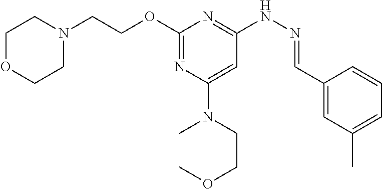 [SMILES: COCCN(C)c1cc(N/N=C/c2cccc(C)c2)nc(OCCN2CCOCC2)n1] | (2-Methoxy-ethyl)-methyl-[6- [N′-(3-methyl-benzylidene)- hydrazino]-2-(2-morpholin-4- yl-ethoxy)-pyrimidin-4-yl]- amine |
| 658 | 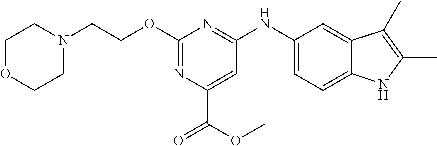 [SMILES: COC(=O)c1cc(Nc2ccc3[nH]c(C)c(C)c3c2)nc(OCCN2CCOCC2)n1] | 6-(2,3-Dimethyl-1H-indol-5- ylamino)-2-(2-morpholin-4-yl- ethoxy)-pyrimidine-4- carboxylic acid methyl ester |
| 659 | 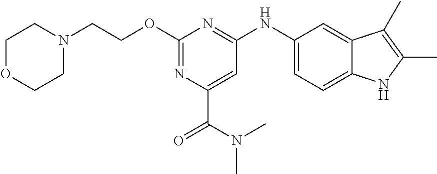 [SMILES: Cc1[nH]c2ccc(Nc3cc(C(=O)N(C)C)nc(OCCN4CCOCC4)n3)cc2c1C] | 6-(2,3-Dimethyl-1H-indol-5- ylamino)-2-(2-morpholin-4-yl- ethoxy)-pyrimidine-4- carboxylic acid dimethylamide |
| 660 | 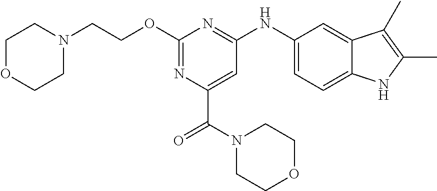 [SMILES: Cc1[nH]c2ccc(Nc3cc(C(=O)N4CCOCC4)nc(OCCN4CCOCC4)n3)cc2c1C] | [6-(2,3-Dimethyl-1H-indol-5- ylamino)-2-(2-morpholin-4-yl- ethoxy)-pyrimidin-4-yl]- morpholin-4-yl-methanone |
| 661 | 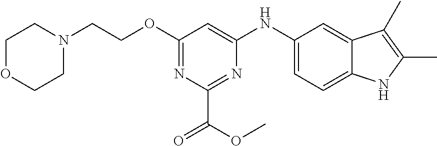 [SMILES: COC(=O)c1nc(Nc2ccc3[nH]c(C)c(C)c3c2)cc(OCCN2CCOCC2)n1] | 4-(2,3-Dimethyl-1H-indol-5- ylamino)-6-(2-morpholin-4-yl- ethoxy)-pyrimidine-2- carboxylic acid methyl ester |
| 662 | 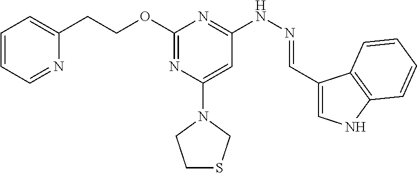 [SMILES: C(=N\Nc1cc(N2CCSC2)nc(OCCc2ccccn2)n1)/c1c[nH]c2ccccc12] | N-(1H-Indol-3-ylmethylene)- N′-[2-(2-pyridin-2-yl-ethoxy)- 6-thiazolidin-3-yl- pyrimidin-4-yl]-hydrazine |
| 663 | 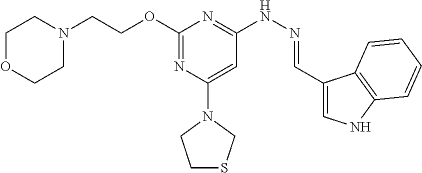 [SMILES: C(=N\Nc1cc(N2CCSC2)nc(OCCN2CCOCC2)n1)/c1c[nH]c2ccccc12] | N-(1H-Indol-3-ylmethylene)- N′-[2-(2-morpholin-4-yl- ethoxy)-6-thiazolidin-3- yl-pyrimidin-4-yl]-hydrazine |
| 664 | 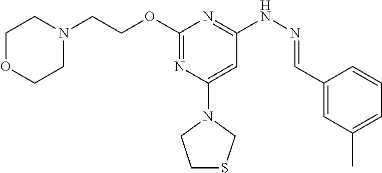 [SMILES: Cc1cccc(/C=N/Nc2cc(N3CCSC3)nc(OCCN3CCOCC3)n2)c1] | N-(3-Methyl-benzylidene)-N′- [2-(2-morpholin-4-yl-ethoxy)- 6-thiazolidin- 3-yl-pyrimidin-4-yl]-hydrazine |
| 665 | 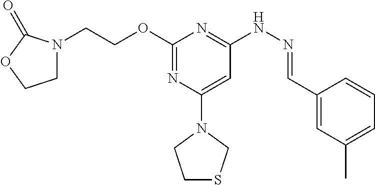 [SMILES: Cc1cccc(/C=N/Nc2cc(N3CCSC3)nc(OCCN3CCOC3=O)n2)c1] | 3-(2-{4-[N′-(3-Methyl- benzylidene)-hydrazino]-6- thiazolidin-3-yl-pyrimidin- 2-yloxy}-ethyl)-oxazolidin-2- one |
| 666 | 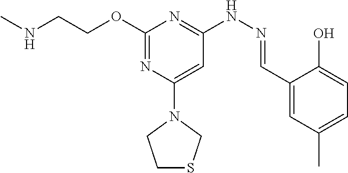 [SMILES: CNCCOc1nc(N/N=C/c2cc(C)ccc2O)cc(N2CCSC2)n1] | 4-Methyl-2-{[2-(2- methylamino-ethoxy)-6- thiazolidin-3-yl-pyrimidin-4- yl]-hydrazonomethyl}-phenol |
| 667 | 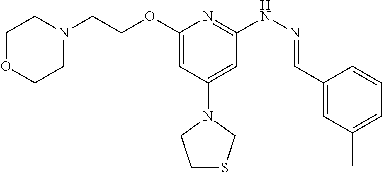 [SMILES: Cc1cccc(/C=N/Nc2cc(N3CCSC3)cc(OCCN3CCOCC3)n2)c1] | N-(3-Methyl-benzylidene)-N′- [6-(2-morpholin-4-yl-ethoxy)- 4-thiazolidin-3-yl-pyridin-2- yl]-hydrazine |
| 668 | 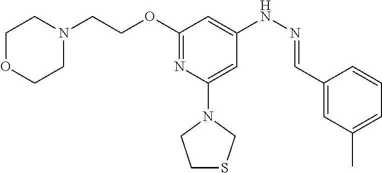 [SMILES: Cc1cccc(/C=N/Nc2cc(OCCN3CCOCC3)nc(N3CCSC3)c2)c1] | N-(3-Methyl-benzylidene)-N′- [2-(2-morpholin-4-yl-ethoxy)- 6-thiazolidin-3-yl-pyridin-4- yl]-hydrazine |
| 669 | 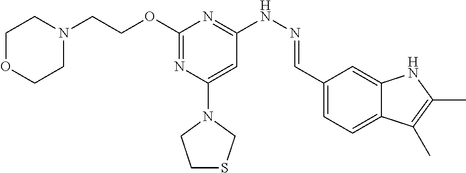 [SMILES: Cc1[nH]c2cc(/C=N/Nc3cc(N4CCSC4)nc(OCCN4CCOCC4)n3)ccc2c1C] | (2,3-Dimethyl-1H-indol-6-yl)- [2-(2-morpholin-4-yl-ethoxy)- 6-thiazolidin-3-yl-pyrimidin-4- yl]-amine |
| 670 | 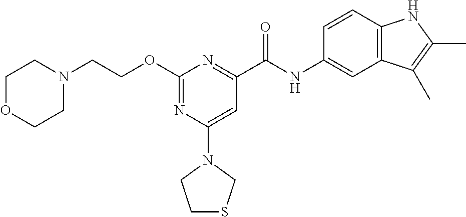 [SMILES: Cc1[nH]c2ccc(NC(=O)c3cc(N4CCSC4)nc(OCCN4CCOCC4)n3)cc2c1C] | 2-(2-Morpholin-4-yl-ethoxy)-6- thiazolidin-3-yl-pyrimidine-4- carboxylic acid (2,3-dimethyl- 1H-indol-5-yl)-amide |
| 671 | 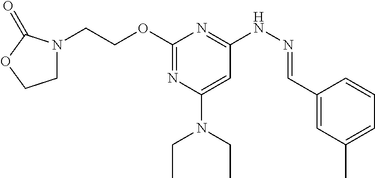 [SMILES: CCN(CC)c1cc(N/N=C/c2cccc(C)c2)nc(OCCN2CCOC2=O)n1] | 3-(2-{4-Diethylamino-6-[N′-(3- methyl-benzylidene)- hydrazino]-pyrimidin-2- yloxy}-ethyl)-oxazolidin-2-one |
| 672 | 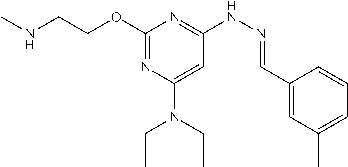 [SMILES: CCN(CC)c1cc(N/N=C/c2cccc(C)c2)nc(OCCNC)n1] | Diethyl-{2-(2-methylamino- ethoxy)-6-[N′-(3-methyl- benzylidene)-hydrazino]- pyrimidin-4-yl}-amine |
| 673 | 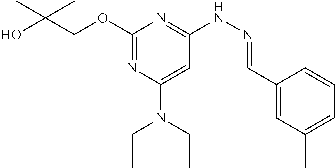 [SMILES: CCN(CC)c1cc(N/N=C/c2cccc(C)c2)nc(OCC(C)(C)O)n1] | 1-{4-Diethylamino-6-[N′-(3- methyl-benzylidene)- hydrazino]-pyrimidin-2- yloxy}-2-methyl-propan-2-ol |
| 674 | 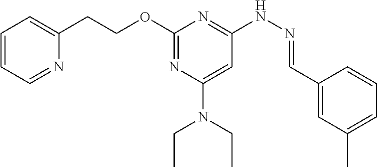 [SMILES: CCN(CC)c1cc(N/N=C/c2cccc(C)c2)nc(OCCc2ccccn2)n1] | Diethyl-[6-[N′-(3-methyl- benzylidene)-hydrazino]-2-(2- pyridin-2-yl-ethoxy)- pyrimidin-4-yl]-amine |
| 675 | 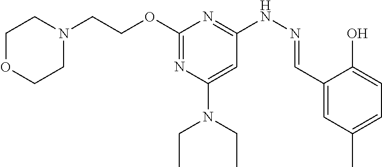 [SMILES: CCN(CC)c1cc(N/N=C/c2cc(C)ccc2O)nc(OCCN2CCOCC2)n1] | 2-{[6-Diethylamino-2-(2- morpholin-4-yl-ethoxy)- pyrimidin-4-yl]- hydrazonomethyl}-4-methyl- phenol |
| 676 | 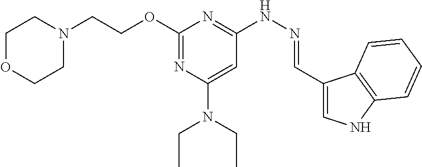 [SMILES: CCN(CC)c1cc(N/N=C/c2c[nH]c3ccccc23)nc(OCCN2CCOCC2)n1] | Diethyl-[6-[N′-(1H-indol-3- ylmethylene)-hydrazino]-2-(2- morpholin-4-yl-ethoxy)- pyrimidin-4-yl]-amine |
| 677 | 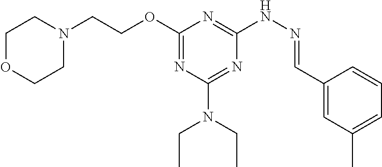 [SMILES: CCN(CC)c1nc(N/N=C/c2cccc(C)c2)nc(OCCN2CCOCC2)n1] | Diethyl-[4-[N′-(3-methyl- benzylidene)-hydrazino]-6-(2- morpholin-4-yl-ethoxy)- [1,3,5]triazin-2-yl]-amine |
| 678 | 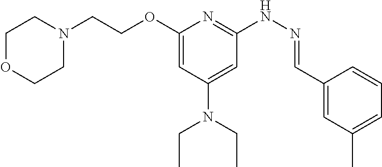 [SMILES: CCN(CC)c1cc(N/N=C/c2cccc(C)c2)nc(OCCN2CCOCC2)c1] | Diethyl-[2-[N′-(3-methyl- benzylidene)-hydrazino]-6-(2- morpholin-4-yl-ethoxy)- pyridin-4-yl]-amine |
| 679 | 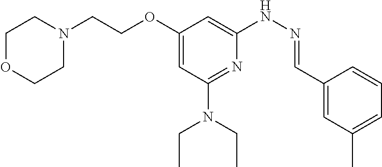 [SMILES: CCN(CC)c1cc(OCCN2CCOCC2)cc(N/N=C/c2cccc(C)c2)n1] | Diethyl-[6-[N′-(3-methyl- benzylidene)-hydrazino]-4-(2- morpholin-4-yl-ethoxy)- pyridin-2-yl]-amine |
| 680 | 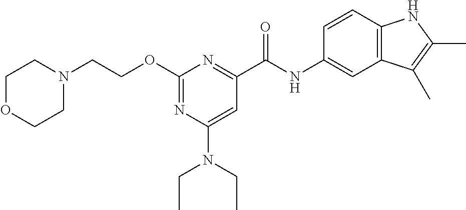 [SMILES: CCN(CC)c1cc(C(=O)Nc2ccc3[nH]c(C)c(C)c3c2)nc(OCCN2CCOCC2)n1] | 6-Diethylamino-2-(2- morpholin-4-yl-ethoxy)- pyrimidine-4-carboxylic acid (2,3-dimethyl-1H-indol-5-yl)- amide |
| 681 | 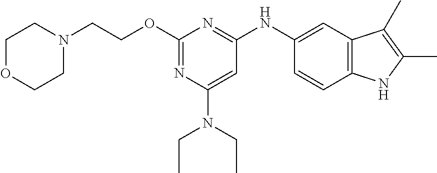 [SMILES: CCN(CC)c1cc(Nc2ccc3[nH]c(C)c(C)c3c2)nc(OCCN2CCOCC2)n1] | 6-Diethylamino-2-(2- morpholin-4-yl-ethoxy)-4- [(2,3-dimethyl-1H-indol-5-yl)- amino]-pyrimidine |
| 682 | 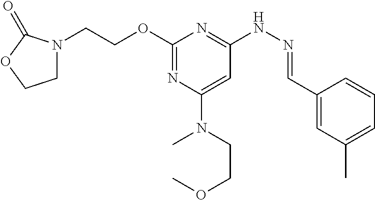 [SMILES: COCCN(C)c1cc(N/N=C/c2cccc(C)c2)nc(OCCN2CCOC2=O)n1] | 3-(2-{4-[(2-Methoxy-ethyl)- methyl-amino]-6-[N′-(3- methyl-benzylidene)- hydrazino]-pyrimidin-2- yloxy}-ethyl)-oxazolidin-2-one |
| 683 | 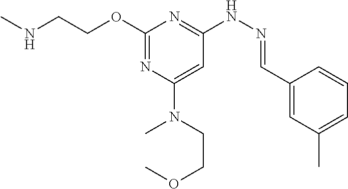 [SMILES: CNCCOc1nc(N/N=C/c2cccc(C)c2)cc(N(C)CCOC)n1] | (2-Methoxy-ethyl)-methyl-{2- (2-methylamino-ethoxy)-6-[N′- (3-methyl-benzylidene)- hydrazino]-pyrimidin-4-yl}- amine |
| 684 | 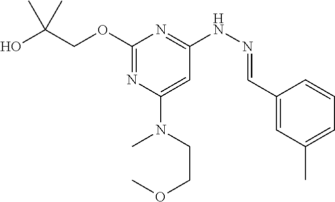 [SMILES: COCCN(C)c1cc(N/N=C/c2cccc(C)c2)nc(OCC(C)(C)O)n1] | 1-{4-[(2-Methoxy-ethyl)- methyl-amino]-6-[N′-(3- methyl-benzylidene)- hydrazino]-pyrimidin-2- yloxy}-2-methyl-propan-2-ol |
| 685 | 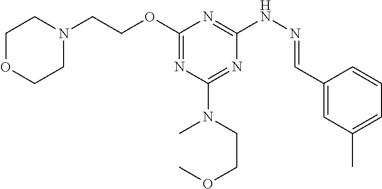 [SMILES: COCCN(C)c1nc(N/N=C/c2cccc(C)c2)nc(OCCN2CCOCC2)n1] | (2-Methoxy-ethyl)-methyl-[4- [N′-(3-methyl-benzylidene)- hydrazino]-6-(2-morpholin-4- yl-ethoxy)-[1,3,5]triazon-2-yl]- amine |
| 686 | 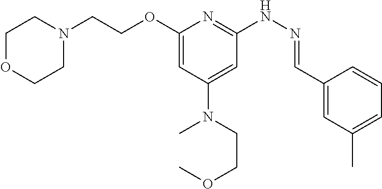 [SMILES: COCCN(C)c1cc(N/N=C/c2cccc(C)c2)nc(OCCN2CCOCC2)c1] | (2-Methoxy-ethyl)-methyl-[2- [N′-(3-methyl-benzylidene)- hydrazino]-6-(2-morpholin-4- yl-ethoxy)-pyridin-4-yl]-amine |
| 687 | 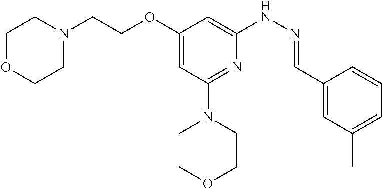 [SMILES: COCCN(C)c1cc(OCCN2CCOCC2)cc(N/N=C/c2cccc(C)c2)n1] | (2-Methoxy-ethyl)-methyl-[6- [N′-(3-methyl-benzylidene)- hydrazino]-4-(2-morpholin-4- yl-ethoxy)-pyridin-2-yl]-amine |
| 688 | 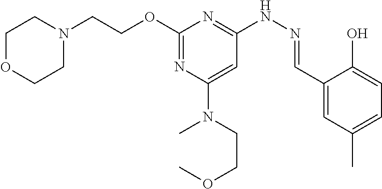 [SMILES: COCCN(C)c1cc(N/N=C/c2cc(C)ccc2O)nc(OCCN2CCOCC2)n1] | 2-{[6-[(2-Methoxy-ethyl)- methyl-amino]-2-(2-morpholin- 4-yl-ethoxy)-pyrimidin-4-yl]- hydrazonomethyl}-4-methyl- phenol |
| 689 | 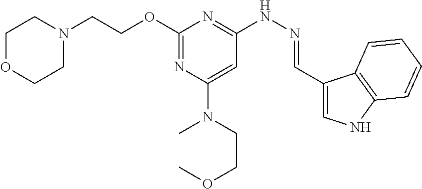 [SMILES: COCCN(C)c1cc(N/N=C/c2c[nH]c3ccccc23)nc(OCCN2CCOCC2)n1] | [6-[N′-(1H-Indol-3- ylmethylene)-hydrazino]-2-(2- morpholin-4-yl-ethoxy)- pyrimidin-4-yl]-(2-methoxy- ethyl)-methyl-amine |
| 690 | 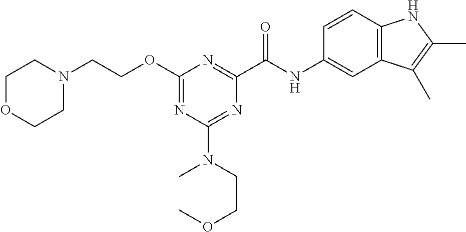 [SMILES: COCCN(C)c1nc(OCCN2CCOCC2)nc(C(=O)Nc2ccc3[nH]c(C)c(C)c3c2)n1] | 4-[(2-Methoxy-ethyl)-methyl- amino]-6-(2-morpholin-4-yl- ethoxy)-[1,3,5]triazine-2- carboxylic acid (2,3-dimethyl- 1H-indol-5-yl)-amide |
| 691 | 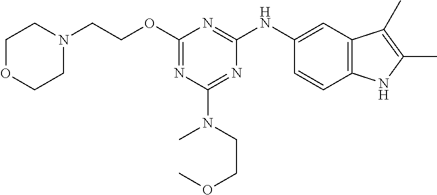 [SMILES: COCCN(C)c1nc(Nc2ccc3[nH]c(C)c(C)c3c2)nc(OCCN2CCOCC2)n1] | N-(2,3-Dimethyl-1H-indol-5- yl)-N′-(2-methoxy-ethyl)-N′- methyl-6-(2-morpholin-4-yl- ethoxy)-[1,3,5]triazine-2,4- diamine |
| 692 | 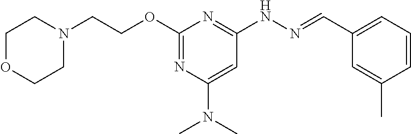 [SMILES: Cc1cccc(/C=N/Nc2cc(N(C)C)nc(OCCN3CCOCC3)n2)c1] | Dimethyl-[6-[N′-(3-methyl- benzylidene)-hydrazino]-2-(2- morpholin-4-yl-ethoxy)- pyrimidin-4-yl]-amine |
| 693 | 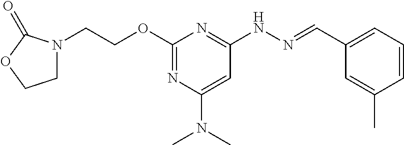 [SMILES: Cc1cccc(/C=N/Nc2cc(N(C)C)nc(OCCN3CCOC3=O)n2)c1] | 3-(2-{4-Dimethylamino-6-[N′- (3-methyl-benzylidene)- hydrazino]-pyrimidin-2- yloxy}-ethyl)-oxazolidin-2-one |
| 694 | 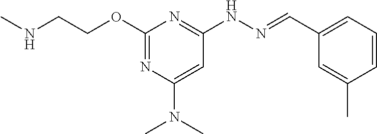 [SMILES: CNCCOc1nc(N/N=C/c2cccc(C)c2)cc(N(C)C)n1] | Dimethyl-{2-(2-methylamino- ethoxy)-6-[N′-(3-methyl- benzylidene)-hydrazino]- pyrimidin-4-yl}-amine |
| 695 | 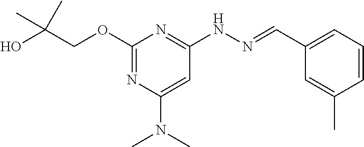 [SMILES: Cc1cccc(/C=N/Nc2cc(N(C)C)nc(OCC(C)(C)O)n2)c1] | 1-{4-Dimethylamino-6-[N′-(3- methyl-benzylidene)- hydrazino]-pyrimidin-2- yloxy}-2-methyl-propan-2-ol |
| 696 | 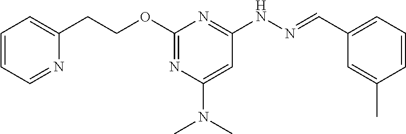 [SMILES: Cc1cccc(/C=N/Nc2cc(N(C)C)nc(OCCc3ccccn3)n2)c1] | Dimethyl-[6-[N′-(3-methyl- benzylidene)-hydrazino]-2-(2- pyridin-2-yl-ethoxy)- pyrimidin-4-yl]-amine |
| 697 | 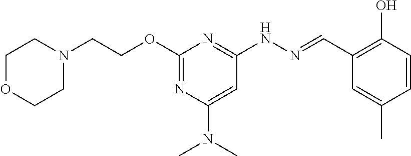 [SMILES: Cc1ccc(O)c(/C=N/Nc2cc(N(C)C)nc(OCCN3CCOCC3)n2)c1] | 2-{[6-Dimethylamino-2-(2- morpholin-4-yl-ethoxy)- pyrimidin-4-yl]- hydrazonomethyl}-4-methyl- phenol |
| 698 | 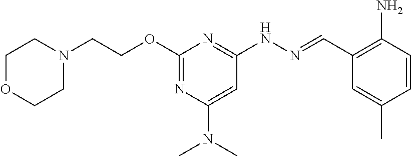 [SMILES: Cc1ccc(N)c(/C=N/Nc2cc(N(C)C)nc(OCCN3CCOCC3)n2)c1] | [6-[N′-(2-Amino-5-methyl- benzylidene)-hydrazino]-2-(2- morpholin-4-yl-ethoxy)- pyrimidin-4-yl]-dimethyl- amine |
| 699 | 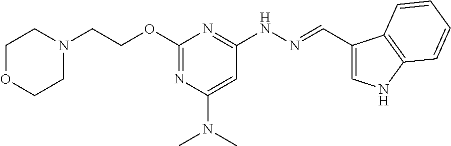 [SMILES: CN(C)c1cc(N/N=C/c2c[nH]c3ccccc23)nc(OCCN2CCOCC2)n1] | [6-[N′-(1H-Indol-3- ylmethylene)-hydrazino]-2-(2- morpholin-4-yl-ethoxy)- pyrimidin-4-yl]-dimethyl- amine |
| 700 | 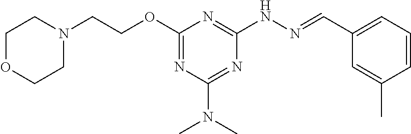 [SMILES: Cc1cccc(/C=N/Nc2nc(OCCN3CCOCC3)nc(N(C)C)n2)c1] | Dimethyl-[4-[N′-(3-methyl- benzylidene)-hydrazino]-6-(2- morpholin-4-yl-ethoxy)- [1,3,5]triazin-2-yl]-amine |
| 701 | 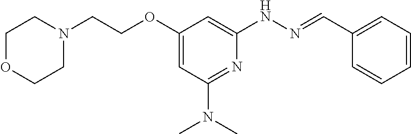 [SMILES: CN(C)c1cc(OCCN2CCOCC2)cc(N/N=C/c2ccccc2)n1] | Dimethyl-[6-[N′-(3-methyl- benzylidene)-hydrazino]-4-(2- morpholin-4-yl-ethoxy)- pyridin-2-yl]-amine |
| 702 | 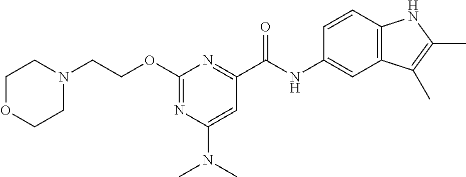 [SMILES: Cc1[nH]c2ccc(NC(=O)c3cc(N(C)C)nc(OCCN4CCOCC4)n3)cc2c1C] | 6-Dimethylamino-2-(2- morpholin-4-yl-ethoxy)- pyrimidine-4-carboxylic acid (2,3-dimethyl-1H-indol-5-yl)- amide |
| 703 | 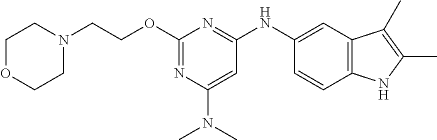 [SMILES: Cc1[nH]c2ccc(Nc3cc(N(C)C)nc(OCCN4CCOCC4)n3)cc2c1C] | 6-Dimethylamino-2-(2- morpholin-4-yl-ethoxy)-4- [(2,3-dimethyl-1H-indol-5-yl)- amino]pyrimidine |
| 704 | 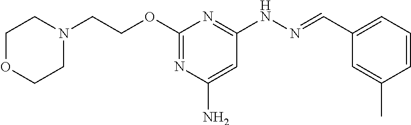 [SMILES: Cc1cccc(/C=N/Nc2cc(N)nc(OCCN3CCOCC3)n2)c1] | 6-[N′-(3-Methyl-benzylidene)- hydrazino]-2-(2-morpholin-4- yl-ethoxy)-pyrimidin-4- ylamine |
| 705 | 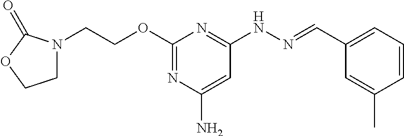 [SMILES: Cc1cccc(/C=N/Nc2cc(N)nc(OCCN3CCOC3=O)n2)c1] | 3-(2-{4-Amino-6-[N′-(3- methyl-benzylidene)- hydrazino]-pyrimidin-2- yloxy}-ethyl)-oxazolidin-2-one |
| 706 | 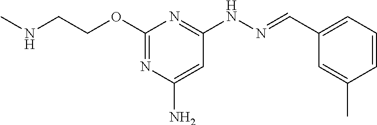 [SMILES: CNCCOc1nc(N)cc(N/N=C/c2cccc(C)c2)n1] | 2-(2-Methylamino-ethoxy)-6- [N′-(3-methyl-benzylidene)- hydrazino]-pyrimidin-4- ylamine |
| 707 | 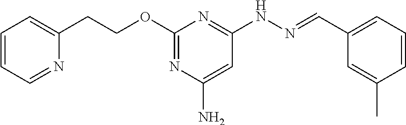 [SMILES: Cc1cccc(/C=N/Nc2cc(N)nc(OCCc3ccccn3)n2)c1] | 6-[N′-(3-Methyl-benzylidene)- hydrazino]-2-(2-pyridin-2-yl- ethoxy)-pyrimidin-4-ylamine |
| 708 | 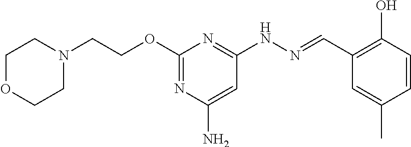 [SMILES: Cc1ccc(O)c(/C=N/Nc2cc(N)nc(OCCN3CCOCC3)n2)c1] | 2-{[6-Amino-2-(2-morpholin- 4-yl-ethoxy)-pyrimidin-4-yl]- hydrazonomethyl}-4-methyl- phenol |
| 709 | 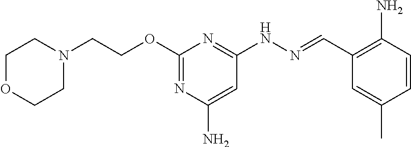 [SMILES: Cc1ccc(N)c(/C=N/Nc2cc(N)nc(OCCN3CCOCC3)n2)c1] | 6-[N′-(2-Amino-5-methyl- benzylidene)-hydrazino]-2-(2- morpholin-4-yl-ethoxy)- pyrimidin-4-ylamine |
| 710 | 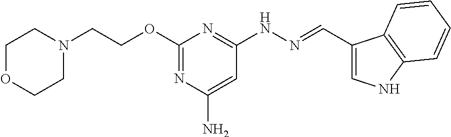 [SMILES: Nc1cc(N/N=C/c2c[nH]c3ccccc23)nc(OCCN2CCOCC2)n1] | 6-[N′-(1H-Indol-3- ylmethylene)-hydrazino]-2-(2- morpholin-4-yl-ethoxy)- pyrimidin-4-ylamine |
| 711 | 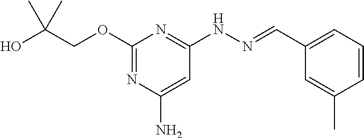 [SMILES: Cc1cccc(/C=N/Nc2cc(N)nc(OCC(C)(C)O)n2)c1] | 1-{4-Amino-6-[N′-(3-methyl- benzylidene)-hydrazino]- pyrimidin-2-yloxy}-2-methyl- propan-2- |
| 712 | 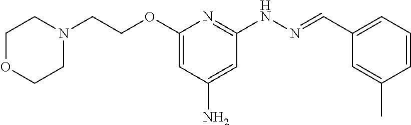 [SMILES: Cc1cccc(/C=N/Nc2cc(N)cc(OCCN3CCOCC3)n2)c1] | 2-[N′-(3-Methyl-benzylidene)- hydrazino]-6-(2-morpholin-4- yl-ethoxy)-pyridin-4-ylamine |
| 713 | 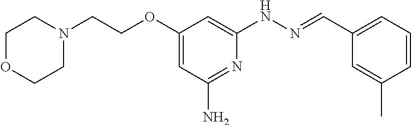 [SMILES: Cc1cccc(/C=N/Nc2cc(OCCN3CCOCC3)cc(N)n2)c1] | 6-[N′-(3-Methyl-benzylidene)- hydrazino]-4-(2-morpholin-4- yl-ethoxy)-pyridin-2-ylamine |
| 714 | 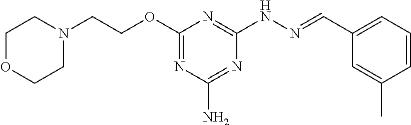 [SMILES: Cc1cccc(/C=N/Nc2nc(N)nc(OCCN3CCOCC3)n2)c1] | 4-[N′-(3-Methyl-benzylidene)- hydrazino]-6-(2-morpholin-4- yl-ethoxy)-[1,3,5]triazin-2- ylamine |
| 715 | 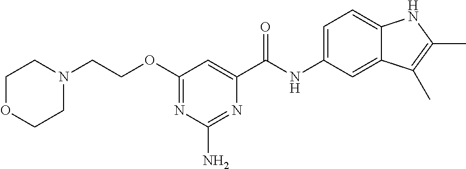 [SMILES: Cc1[nH]c2ccc(NC(=O)c3cc(OCCN4CCOCC4)nc(N)n3)cc2c1C] | 2-Amino-6-(2-morpholin-4-yl- ethoxy)-pyrimidine-4- carboxylic acid (2,3-dimethyl- 1H-indol-5-yl)-amide |
| 716 | 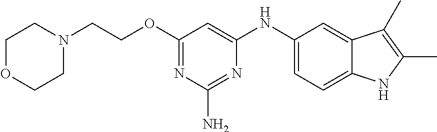 [SMILES: Cc1[nH]c2ccc(Nc3cc(OCCN4CCOCC4)nc(N)n3)cc2c1C] | N4-(2,3-Dimethyl-1H-indol-5- yl)-6-(2-morpholin-4-yl- ethoxy)-pyrimidine-2,4- diamine |
| 717 | 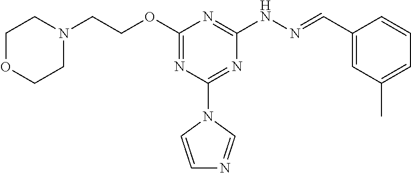 [SMILES: Cc1cccc(/C=N/Nc2nc(OCCN3CCOCC3)nc(-n3ccnc3)n2)c1] | N-[4-Imidazol-1-yl-6-(2- morpholin-4-yl-ethoxy)- [1,3,5]triazin-2-yl]-N′-(3- methyl-benzylidene)-hydrazine |
| 718 | 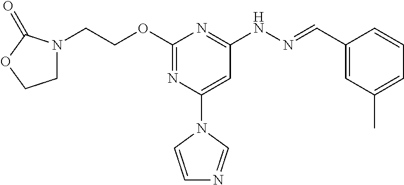 [SMILES: Cc1cccc(/C=N/Nc2cc(-n3ccnc3)nc(OCCN3CCOC3=O)n2)c1] | 3-(2-{4-Imidazol-1-yl-6-[N′-(3- methyl-benzylidene)- hydrazino]-pyrimidin-2- yloxy}-ethyl)-oxazolidin-2-one |
| 719 | 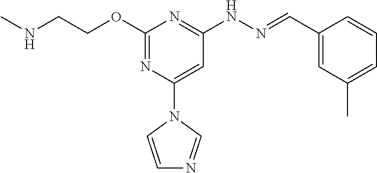 [SMILES: CNCCOc1nc(N/N=C/c2cccc(C)c2)cc(-n2ccnc2)n1] | (2-{4-Imidazol-1-yl-6-[N′-(3- methyl-benzylidene)- hydrazino]-pyrimidin-2- yloxy}-ethyl)-methyl-amine |
| 720 | 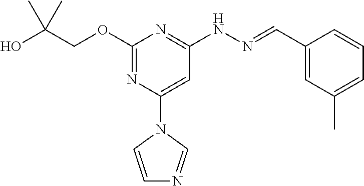 [SMILES: Cc1cccc(/C=N/Nc2cc(-n3ccnc3)nc(OCC(C)(C)O)n2)c1] | 1-{4-Imidazol-1-yl-6-[N′-(3- methyl-benzylidene)- hydrazino]-pyrimidin-2- yloxy}-2-methyl-propan-2-ol |
| 721 | 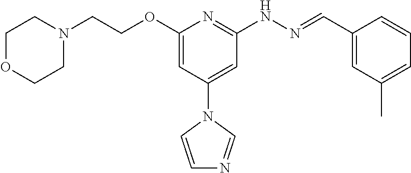 [SMILES: Cc1cccc(/C=N/Nc2cc(-n3ccnc3)cc(OCCN3CCOCC3)n2)c1] | N-[4-Imidazol-1-yl-6-(2- morpholin-4-yl-ethoxy)- pyridin-2-yl]-N′-(3-methyl- benzylidene)-hydrazine |
| 722 | 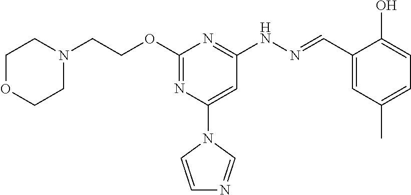 [SMILES: Cc1ccc(O)c(/C=N/Nc2cc(-n3ccnc3)nc(OCCN3CCOCC3)n2)c1] | 2-{[6-Imidazol-1-yl-2-(2- morpholin-4-yl-ethoxy)- pyrimidin-4-yl]- hydrazonomethyl}-4-methyl- phenol |
| 723 | 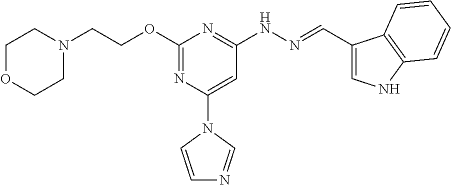 [SMILES: C(=N\Nc1cc(-n2ccnc2)nc(OCCN2CCOCC2)n1)/c1c[nH]c2ccccc12] | N-[6-Imidazol-1-yl-2-(2- morpholin-4-yl-ethoxy)- pyrimidin-4-yl]-N′-(1H-indol- 3-ylmethylene)-hydrazine |
| 724 | 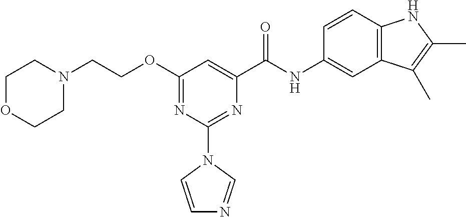 [SMILES: Cc1[nH]c2ccc(NC(=O)c3cc(OCCN4CCOCC4)nc(-n4ccnc4)n3)cc2c1C] | 2-Imidazol-1-yl-6-(2- morpholin-4-yl-ethoxy)- pyrimidine-4-carboxylic acid (2,3-dimethyl-1H-indol-5-yl)- amide |
| 725 | 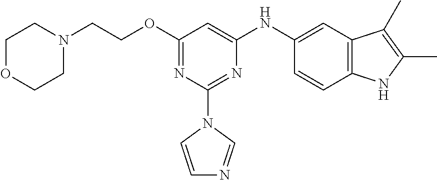 [SMILES: Cc1[nH]c2ccc(Nc3cc(OCCN4CCOCC4)nc(-n4ccnc4)n3)cc2c1C] | (2,3-Dimethyl-1H-indol-5-yl)- [2-imidazol-1-yl-6-(2- morpholin-4-yl-ethoxy)- pyrimidin-4-yl]-amine |
| 726 | 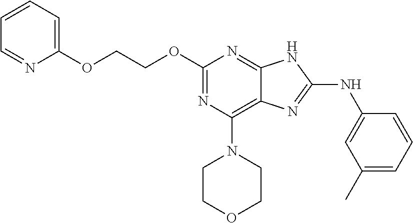 [SMILES: Cc1cccc(Nc2nc3c(N4CCOCC4)nc(OCCOc4ccccn4)nc3[nH]2)c1] | {6-morpholin-4-yl-2-[2- (pyridin-2-yloxy)-ethoxy]-9H- purin-8-yl}-m-tolyl-amine |
| 727 | 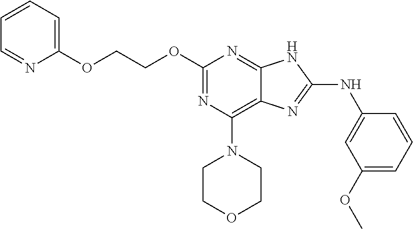 [SMILES: COc1cccc(Nc2nc3c(N4CCOCC4)nc(OCCOc4ccccn4)nc3[nH]2)c1] | (3-Methoxyphenyl)-{6- morpholin-4-yl-2-[2-(pyridin- 2-yloxy)-ethoxy]-9H-purin-8- yl}-amine |
| 728 | 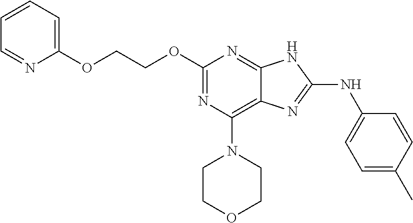 [SMILES: Cc1ccc(Nc2nc3c(N4CCOCC4)nc(OCCOc4ccccn4)nc3[nH]2)cc1] | {6-Morpholin-4-yl-2-[2- (pyridin-2-yloxy)-ethoxy]-9H- purin-8-yl}-p-tolyl-amine |
| 729 | 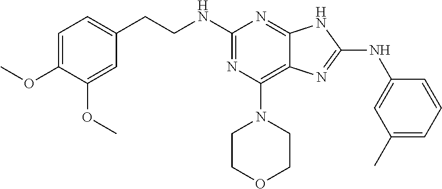 [SMILES: COc1ccc(CCNc2nc(N3CCOCC3)c3nc(Nc4cccc(C)c4)[nH]c3n2)cc1OC] | N²-{2-(3,4-Dimethoxy- phenyl)-ethyl]-6-morpholin-4- yl-N⁸-m-tolyl-9H-purine-2,8- diamine |
| 730 | 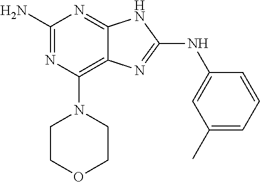 [SMILES: Cc1cccc(Nc2nc3c(N4CCOCC4)nc(N)nc3[nH]2)c1] | 6-morpholin-4-yl-N⁸-m-tolyl- 9H-purine-2,8-diamine |
| 731 | 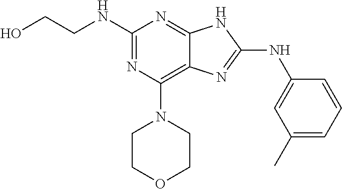 [SMILES: Cc1cccc(Nc2nc3c(N4CCOCC4)nc(NCCO)nc3[nH]2)c1] | 2-(6-morpholin-4-yl-8-m- tolylamino-9H-purin-2- ylamino)-ethanol |
| 732 | 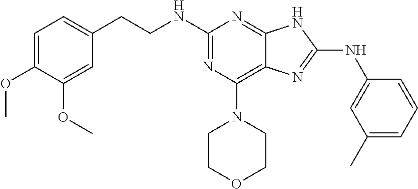 [SMILES: COc1ccc(CCNc2nc(N3CCOCC3)c3nc(Nc4cccc(C)c4)[nH]c3n2)cc1OC] | N²-[2-(3,4-Dimethoxy-phenyl)- ethyl]-6-morpholin-4-yl-N⁸-m- tolyl-9H-purine-2,8-diamine |
| 733 | 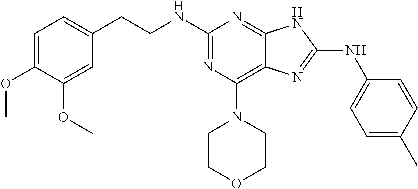 [SMILES: COc1ccc(CCNc2nc(N3CCOCC3)c3nc(Nc4ccc(C)cc4)[nH]c3n2)cc1OC] | N²-[2-(3,4-Dimethoxy-phenyl)- ethyl]-6-morpholin-4-yl-N⁸-m- tolyl-9H-purine-2,8-diamine |
| 734 | 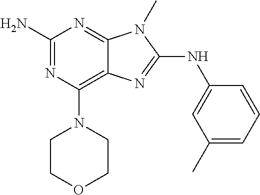 [SMILES: Cc1cccc(Nc2nc3c(N4CCOCC4)nc(N)nc3n2C)c1] | 9-Methyl-6-morpholin-4-yl-N⁸- m-tolyl-9H-purine-2,8-diamine |
| 735 | 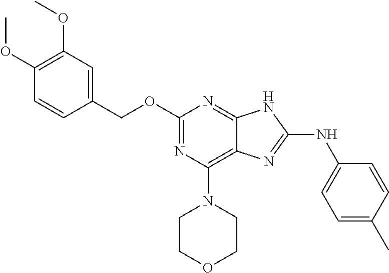 [SMILES: COc1ccc(COc2nc(N3CCOCC3)c3nc(Nc4ccc(C)cc4)[nH]c3n2)cc1OC] | [2-(3,4-dimethoxy-benzyloxy)- 6-morpholin-4-yl-9H-purine-8- yl-p-tolyl-amine |
| 736 | 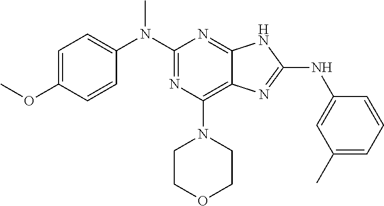 [SMILES: COc1ccc(N(C)c2nc(N3CCOCC3)c3nc(Nc4cccc(C)c4)[nH]c3n2)cc1] | N²-(4-methoxy-phenyl)-N²- methyl-6-morpholin-4-yl-N⁸- m-tolyl-9H-purine-2,8-diamine |
| 737 | 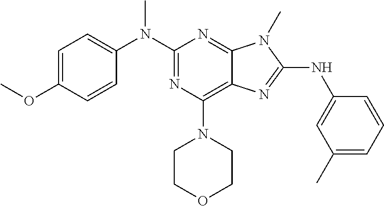 [SMILES: COc1ccc(N(C)c2nc(N3CCOCC3)c3nc(Nc4cccc(C)c4)n(C)c3n2)cc1] | N²-(4-methoxy-phenyl)-N²- methyl-9-methyl-6-morpholin- 4-yl-N⁸-m-tolyl-9H-purine-2,8- diamine |
| 738 | 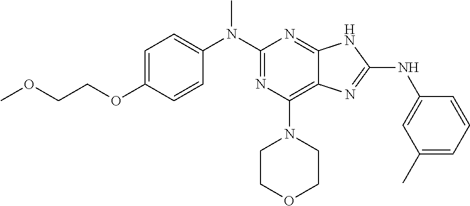 [SMILES: COCCOc1ccc(N(C)c2nc(N3CCOCC3)c3nc(Nc4cccc(C)c4)[nH]c3n2)cc1] | N²-[4-(2-Methoxy-ethoxy)- phenyl]-N²-methyl-6- morpholin-4-yl-N⁸-m-tolyl-9H- purine-2,8-diamine |
| 739 | 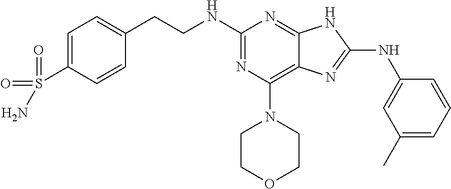 [SMILES: Cc1cccc(Nc2nc3c(N4CCOCC4)nc(NCCc4ccc(S(N)(=O)=O)cc4)nc3[nH]2)c1] | 4-[2-(6-Morpholin-4-yl-8-m- tolylamino-9H-purine-2- ylamino)-ethyl]- benzenesulfonamide |
| 740 | 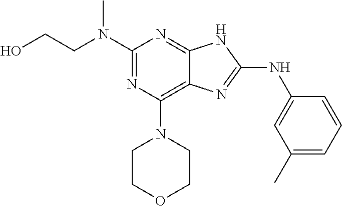 [SMILES: Cc1cccc(Nc2nc3c(N4CCOCC4)nc(N(C)CCO)nc3[nH]2)c1] | 2-[Methyl-(6-morpholin-4-yl- 8-m-tolylamino-9H-purin-2- ylamino)-amino]-ethanol |
| 741 | 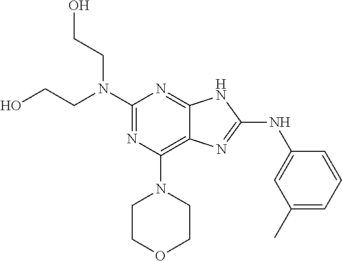 [SMILES: Cc1cccc(Nc2nc3c(N4CCOCC4)nc(N(CCO)CCO)nc3[nH]2)c1] | 2-[(2-Hydroxy-ethyl)-(6- morpholin-4-yl-8-m- tolylamino-9H-purin-2- ylamino)amino]-ethanol |
| 742 | 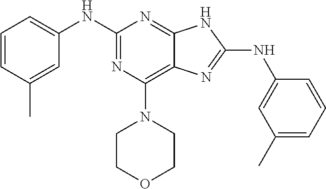 [SMILES: Cc1cccc(Nc2nc(N3CCOCC3)c3nc(Nc4cccc(C)c4)[nH]c3n2)c1] | 6-Morpholin-4-yl-N²,N⁸-di-m- tolyl-9H-purine-2,8-diamine |
| 743 | 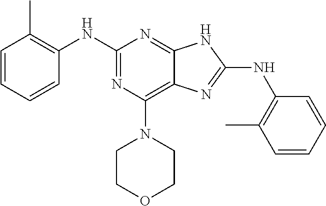 [SMILES: Cc1ccccc1Nc1nc(N2CCOCC2)c2nc(Nc3ccccc3C)[nH]c2n1] | 6-Morpholin-4-yl-N²,N⁸-di-o- tolyl-9H-purine-2,8-diamine |
| 744 | 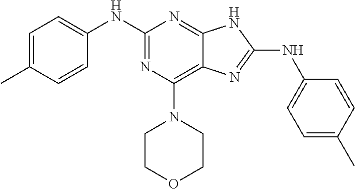 | 6-Morpholin-4-yl-N²,N⁸-di-p- tolyl-9H-purine-2,8-diamine |
| 745 | 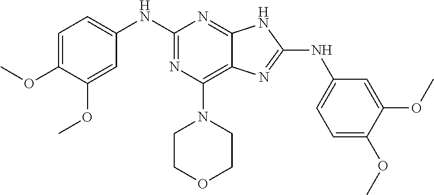 | N²,N⁸-bis-(3,4-dimethoxy- phenyl)-6-morpholin-4-yl-9H- purine-2,8-diamine |
| 746 | 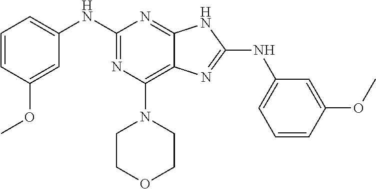 [SMILES: COc1cccc(Nc2nc(N3CCOCC3)c3nc(Nc4cccc(OC)c4)[nH]c3n2)c1] | N²,N⁸-bis-(3-methoxy-phenyl)- 6-morpholin-4-yl-9H-purine- 2,8-diamine |
| 747 | 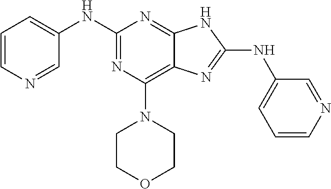 [SMILES: c1cncc(Nc2nc(N3CCOCC3)c3nc(Nc4cccnc4)[nH]c3n2)c1] | 6-morpholin-4-yl-N²,N⁸-di- pyridine-9H-purine-2,8- diamine |
| 748 | 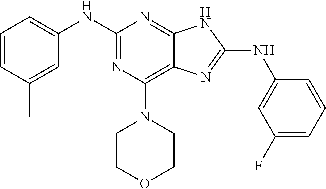 [SMILES: Cc1cccc(Nc2nc(N3CCOCC3)c3nc(Nc4cccc(F)c4)[nH]c3n2)c1] | N²,N⁸-bis-(3-fluoro-phenyl)-6- morpholin-4-yl-9H-purine-2,8- diamine |
| 749 | 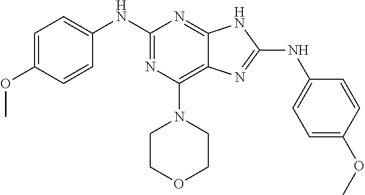 [SMILES: COc1ccc(Nc2nc(N3CCOCC3)c3nc(Nc4ccc(OC)cc4)[nH]c3n2)cc1] | N²,N⁸-bis-(4-methoxy-phenyl)- 6-morpholin-4-yl-9H-purine- 2,8-diamine |
| 750 | 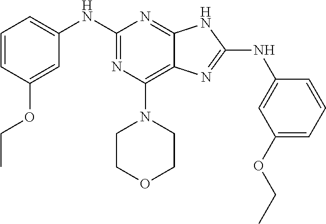 [SMILES: CCOc1cccc(Nc2nc(N3CCOCC3)c3nc(Nc4cccc(OCC)c4)[nH]c3n2)c1] | N²,N⁸-bis-(3-ethoxy-phenyl)- 6-morpholin-4-yl-9H-purine- 2,8-diamine |
| 751 | 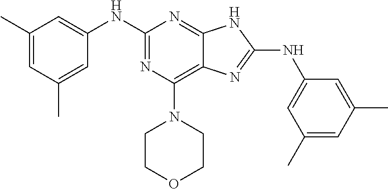 | N²,N⁸-bis-(3,5-dimethyl- phenyl)-6-morpholin-4-yl-9H- purine-2,8-diamine |
| 752 | 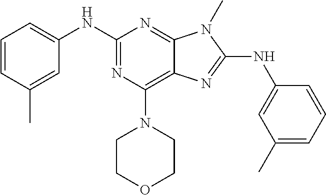 [SMILES: Cc1cccc(Nc2nc(N3CCOCC3)c3nc(Nc4cccc(C)c4)n(C)c3n2)c1] | 9-Methyl-6-morpholin-4-yl- N²,N⁸-di-m-tolyl-9H-purine- 2,8-diamine |
| 753 | 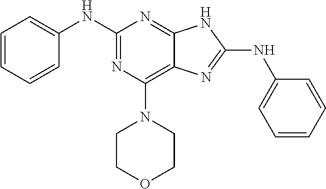 [SMILES: c1ccc(Nc2nc(N3CCOCC3)c3nc(Nc4ccccc4)[nH]c3n2)cc1] | 6-morpholin-4-yl-N²,N⁸- diphenyl-9H-purine-2,8- diamine |
| 754 | 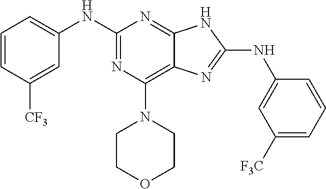 [SMILES: FC(F)(F)c1cccc(Nc2nc(N3CCOCC3)c3nc(Nc4cccc(C(F)(F)F)c4)[nH]c3n2)c1] | 6-morpholin-4-yl-N²,N⁸-bis-(3- trifluoromethyl-phenyl)-9H- purine-2,8-diamine |
| 755 | 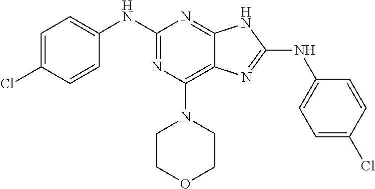 [SMILES: Clc1ccc(Nc2nc(N3CCOCC3)c3nc(Nc4ccc(Cl)cc4)[nH]c3n2)cc1] | 6-morpholin-4-yl-N²,N⁸-bis-(4- chloro-phenyl)-9H-purine-2,8- diamine |
| 756 | 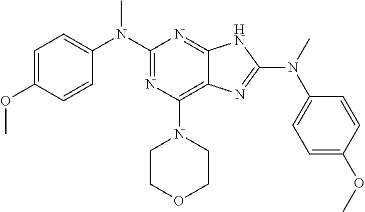 [SMILES: COc1ccc(N(C)c2nc(N3CCOCC3)c3nc(N(C)c4ccc(OC)cc4)[nH]c3n2)cc1] | N²,N⁸-bis-(4-methoxy-phenyl)- N²,N⁸-dimethyl-6- morpholin-4-yl-9H- purine-2,8-diamine |
| 757 | 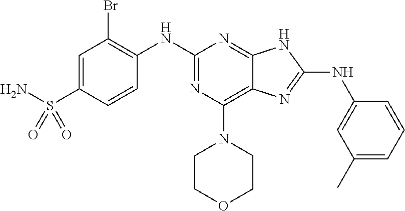 [SMILES: Cc1cccc(Nc2nc3c(N4CCOCC4)nc(Nc4ccc(S(N)(=O)=O)cc4Br)nc3[nH]2)c1] | 3-Bromo-4-(6-morpholin-4-yl- 8-m-tolylamino-9H-purin-2- ylamino)-benzenesulfonamide |
| 758 | 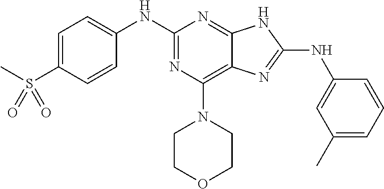 [SMILES: Cc1cccc(Nc2nc3c(N4CCOCC4)nc(Nc4ccc(S(C)(=O)=O)cc4)nc3[nH]2)c1] | N²-(4-Methanesulfonyl-phenyl)- 6-morpholin-4-yl-N⁸-m-tolyl- 9H-purine-2,8-diamine |
| 759 | 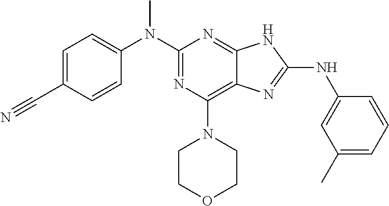 [SMILES: Cc1cccc(Nc2nc3c(N4CCOCC4)nc(N(C)c4ccc(C#N)cc4)nc3[nH]2)c1] | 4-[Methyl-(6-morpholin-4-yl- 8-m-tolylamino-9H-purin-2- yl)-amino]-benzonitrile |
| 760 | 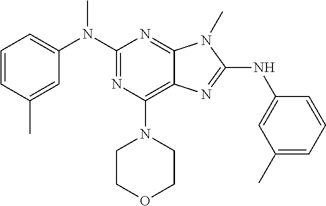 [SMILES: Cc1cccc(Nc2nc3c(N4CCOCC4)nc(N(C)c4cccc(C)c4)nc3n2C)c1] | 9,N²-Dimethyl-6-morpholin-4- yl-N²,N⁸-di-m-tolyl-9H-purine- 2,8-diamine |
| 761 | 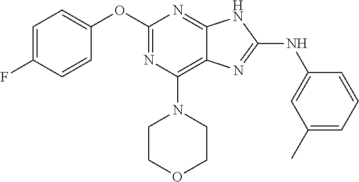 [SMILES: Cc1cccc(Nc2nc3c(N4CCOCC4)nc(Oc4ccc(F)cc4)nc3[nH]2)c1] | [2-(4-Fluoro-phenoxy)-6- morpholin-4-yl-9H-purin-8-yl]- m-tolyl-amine |
| 762 | 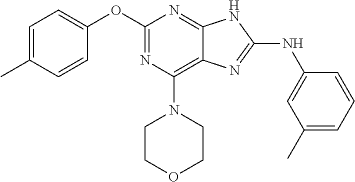 [SMILES: Cc1ccc(Oc2nc(N3CCOCC3)c3nc(Nc4cccc(C)c4)[nH]c3n2)cc1] | 6-morpholin-4-yl-2-p-tolyloxy- 9H-purin-8-yl)-m-tolyl-amine |
| 763 | 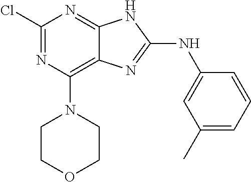 [SMILES: Cc1cccc(Nc2nc3c(N4CCOCC4)nc(Cl)nc3[nH]2)c1] | 2-Chloro-6-morpholin-4-yl-9H- purin-8-yl)-m-tolyl-amine |
| 764 | 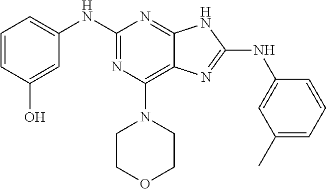 [SMILES: Cc1cccc(Nc2nc3c(N4CCOCC4)nc(Nc4cccc(O)c4)nc3[nH]2)c1] | 3-(6-morpholin-4-yl-8-m- tolylamino-9H-purin-2- ylamino)-phenol |
| 765 | 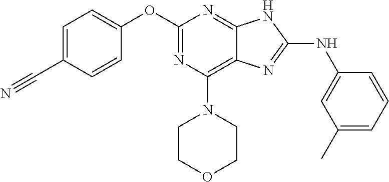 [SMILES: Cc1cccc(Nc2nc3c(N4CCOCC4)nc(Oc4ccc(C#N)cc4)nc3[nH]2)c1] | 4-(6-morpholin-4-yl-8-m- tolylamino-9H-purin-2-yloxy)- benzonitrile |
| 766 | 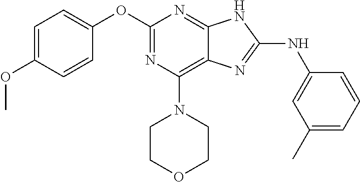 [SMILES: COc1ccc(Oc2nc(N3CCOCC3)c3nc(Nc4cccc(C)c4)[nH]c3n2)cc1] | [2-(4-Methoxy-phenoxy)-6- morpholin-4-yl-9H-purin-2-yl]- m-tolyl-amine |
| 767 | 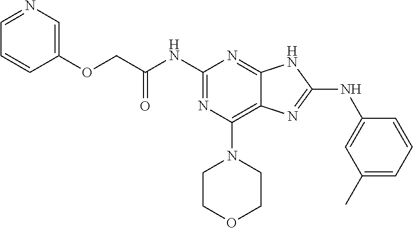 [SMILES: Cc1cccc(Nc2nc3c(N4CCOCC4)nc(NC(=O)COc4cccnc4)nc3[nH]2)c1] | N-(6-morpholin-4-yl-8-m- tolylamino-9H-purin-2-yl)-2- (pyridin-3-yloxy)-acetamide |
| 768 | 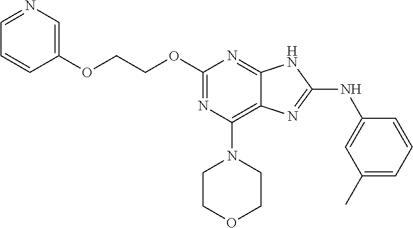 [SMILES: Cc1cccc(Nc2nc3c(N4CCOCC4)nc(OCCOc4cccnc4)nc3[nH]2)c1] | {6-morpholin-4-yl-2-[2- pyridin-3-yloxy)-ethoxy]-9H- purin-2-yl}-m-toly-amine |
| 769 | 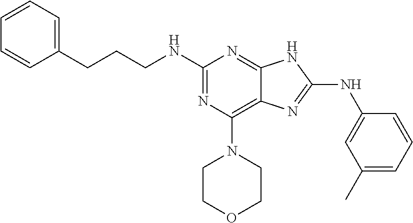 [SMILES: Cc1cccc(Nc2nc3c(N4CCOCC4)nc(NCCCc4ccccc4)nc3[nH]2)c1] | 6-morpholin-4-yl-N²-(3- phenyl-propyl)-N⁸-m-tolyl-9H- purine-2,8-diamine |
| 780 | 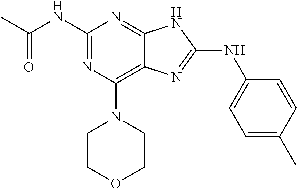 [SMILES: CC(=O)Nc1nc(N2CCOCC2)c2nc(Nc3ccc(C)cc3)[nH]c2n1] | N-(6-morpholin-4-yl-8-p- tolylamino-9H-purin-2-yl)- acetamide |
| 781 | 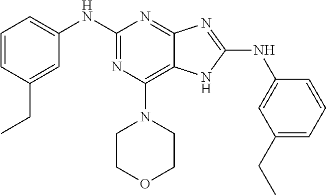 [SMILES: CCc1cccc(Nc2nc(N3CCOCC3)c3[nH]c(Nc4cccc(CC)c4)nc3n2)c1] | N-2′,N-8′-Bis(3-ethyl- phenyl)-6-morpholin-4-yl-7H- purine-2,8-diamine |
| 782 | 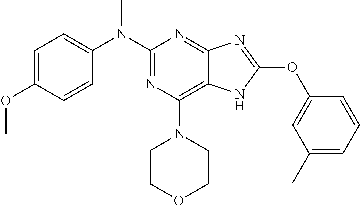 [SMILES: COc1ccc(N(C)c2nc(N3CCOCC3)c3[nH]c(Oc4cccc(C)c4)nc3n2)cc1] | (4-Methoxy-phenyl)-methyl-(6- morpholin-4-yl-8-m-tolyloxy- 7H-purin-2-yl)-amine |
| 783 | 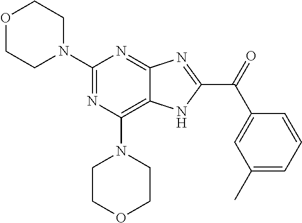 [SMILES: Cc1cccc(C(=O)c2nc3nc(N4CCOCC4)nc(N4CCOCC4)c3[nH]2)c1] | (2,6-di-morpholin-4-yl-7H- purin-8-yl)-m-tolyl-methanone |
| 784 | 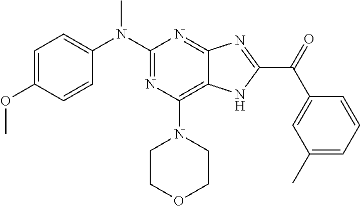 [SMILES: COc1ccc(N(C)c2nc(N3CCOCC3)c3[nH]c(C(=O)c4cccc(C)c4)nc3n2)cc1] | {2-[(4-Methoxy-phenyl)- methyl-amino]-6-morpholin-4- yl-7H-purin-8-yl)-m-tolyl- methanone |
| 785 | 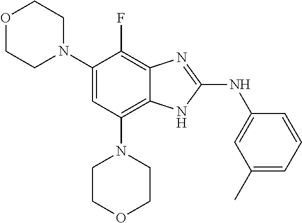 [SMILES: Cc1cccc(Nc2nc3c(F)c(N4CCOCC4)cc(N4CCOCC4)c3[nH]2)c1] | (4-Fluoro-5,7-di-morpholin-4- yl-1H-benzoimdazol-2-yl)-m- tolyl-amine |
| 786 | 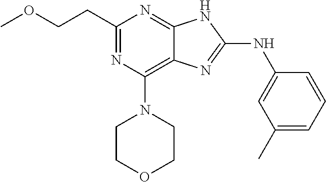 [SMILES: COCCc1nc(N2CCOCC2)c2nc(Nc3cccc(C)c3)[nH]c2n1] | [2-(2-methoxy-ethyl)-6- morpholin-4-yl-9H-purin-8- yl]-m-tolyl-amine |
| 787 | 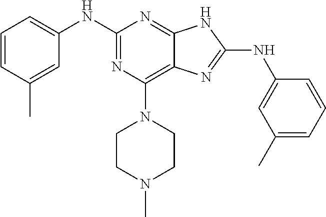 [SMILES: Cc1cccc(Nc2nc(N3CCN(C)CC3)c3nc(Nc4cccc(C)c4)[nH]c3n2)c1] | N²,N⁸-bis-(3-methylphenyl)-6- (4-methylpiperidinyl)-9H- purine-2,8-diamine |
| 788 | 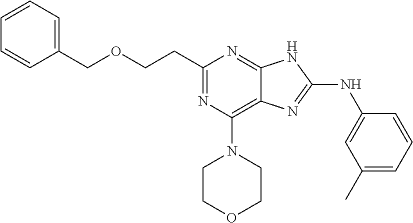 [SMILES: Cc1cccc(Nc2nc3c(N4CCOCC4)nc(CCOCc4ccccc4)nc3[nH]2)c1] | [2-(2-Benzyloxy-ethyl)-6- morpholin-4-yl-9H-purin-8-yl]- m-tolyl-amine |
| 789 | 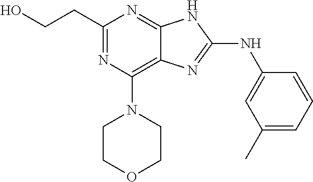 [SMILES: Cc1cccc(Nc2nc3c(N4CCOCC4)nc(CCO)nc3[nH]2)c1] | 2-(6-morpholin-4-yl-8-m- tolylamino-9H-purin-2- yl)-ethanol |
| 790 | 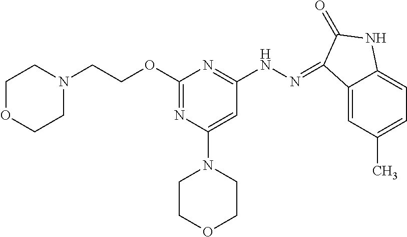 [SMILES: Cc1ccc2c(c1)/C(=N/Nc1cc(N3CCOCC3)nc(OCCN3CCOCC3)n1)C(=O)N2] | 5-Methyl-3-{[6-morpholin-4- yl-2-(2-morpholin-4-yl- ethoxy)-pyrimidin-4-yl]- hydrazono}-1,3-dihydro-indol- 2-one |
| 791 | 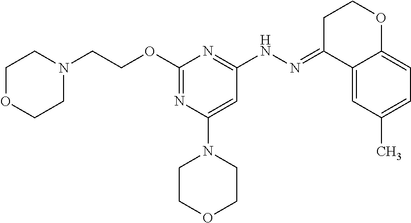 [SMILES: Cc1ccc2c(c1)/C(=N/Nc1cc(N3CCOCC3)nc(OCCN3CCOCC3)n1)CCO2] | N-(6-Methyl-chroman-4- ylidene)-N′-[6-morpholin-4-yl- 2-(2-morpholin-4-yl-ethoxy)- pyrimidin-4-yl]-hydrazine |
| 792 | 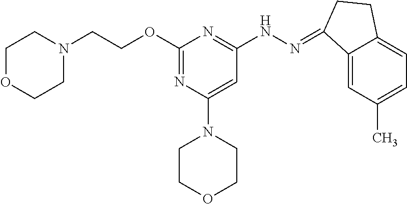 [SMILES: Cc1ccc2c(c1)/C(=N/Nc1cc(N3CCOCC3)nc(OCCN3CCOCC3)n1)CC2] | N-(6-Methyl-indan-1-ylidene)- N′-[6-morpholin-4-yl-2-(2- morpholin-4-yl-ethoxy)- pyrimidin-4-yl]-hydrazine |
| 793 | 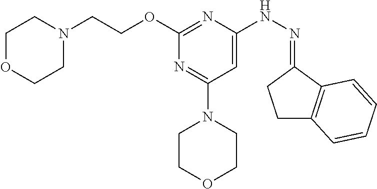 [SMILES: c1ccc2c(c1)CC/C2=N\Nc1cc(N2CCOCC2)nc(OCCN2CCOCC2)n1] | N-(Indan-1-ylidene)-N′-[6- morpholin-4-yl-2-(2- morpholin-4-yl-ethoxy)- pyrimidin-4-yl]-hydrazine |
| 794 | 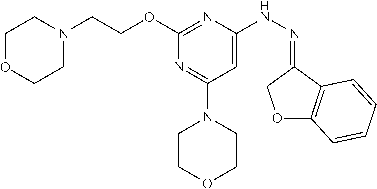 [SMILES: c1ccc2c(c1)OC/C2=N\Nc1cc(N2CCOCC2)nc(OCCN2CCOCC2)n1] | N-(Benzofuran-3-ylidene)-N′- [6-morpholin-4-yl-2-(2- morpholin-4-yl-ethoxy)- pyrimidin-4-yl]-hydrazine |
| 795 | 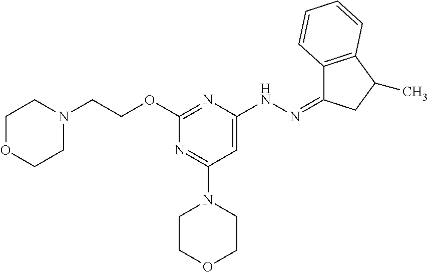 [SMILES: CC1C/C(=N/Nc2cc(N3CCOCC3)nc(OCCN3CCOCC3)n2)c2ccccc21] | N-(3-Methyl-indan-1-ylidene)- N′-[6-morpholin-4-yl-2-(2- morpholin-4-yl-ethoxy)- pyrimidin-4-yl]-hydrazine |
| 796 | 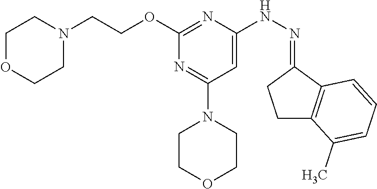 [SMILES: Cc1cccc2c1CC/C2=N\Nc1cc(N2CCOCC2)nc(OCCN2CCOCC2)n1] | N-(4-Methyl-indan-1-ylidene)- N′-[6-morpholin-4-yl-2-(2- morpholin-4-yl-ethoxy)- pyrimidin-4-yl]-hydrazine |
| 797 | 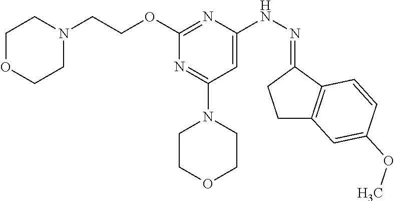 [SMILES: COc1ccc2c(c1)CC/C2=N\Nc1cc(N2CCOCC2)nc(OCCN2CCOCC2)n1] | N-(5-Methoxy-indan-1- ylidene)-N′-[6-morpholin-4-yl- 2-(2-morpholin-4-yl-ethoxy)- pyrimidin-4-yl]-hydrazine |
| 798 | 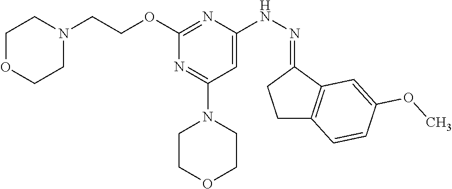 [SMILES: COc1ccc2c(c1)/C(=N/Nc1cc(N3CCOCC3)nc(OCCN3CCOCC3)n1)CC2] | N-(6-Methoxy-indan-1- ylidene)-N′-[6-morpholin-4-yl- 2-(2-morpholin-4-yl-ethoxy)- pyrimidin-4-yl]-hydrazine |
| 799 | 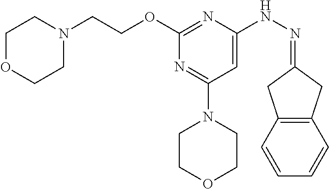 [SMILES: c1ccc2c(c1)CC(=NNc1cc(N3CCOCC3)nc(OCCN3CCOCC3)n1)C2] | N-(Indan-2-ylidene)-N′-[6- morpholin-4-yl-2-(2- morpholin-4-yl-ethoxy)- pyrimidin-4-yl]-hydrazine |
| 800 | 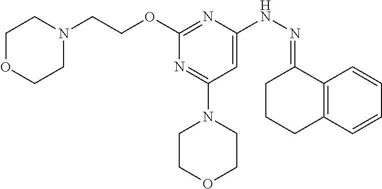 [SMILES: c1ccc2c(c1)CCC/C2=N\Nc1cc(N2CCOCC2)nc(OCCN2CCOCC2)n1] | N-(3,4-Dihydro-2H- naphthalen-1-ylidene)-N′-[6- morpholin-4-yl-2-(2- morpholin-4-yl-ethoxy)- pyrimidin-4-yl]-hydrazine |
| 801 | 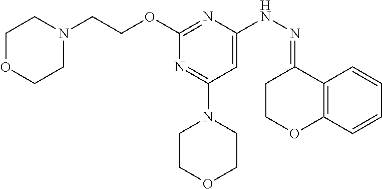 [SMILES: c1ccc2c(c1)OCC/C2=N\Nc1cc(N2CCOCC2)nc(OCCN2CCOCC2)n1] | N-(Chroman-4-ylidene)-N′-[6- morpholin-4-yl-2-(2- morpholin-4-yl-ethoxy)- pyrimidin-4-yl]-hydrazine |
| 802 | 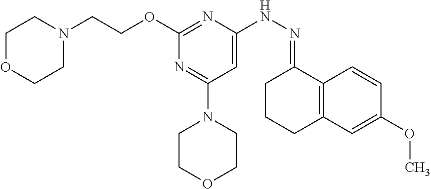 [SMILES: COc1ccc2c(c1)CCC/C2=N\Nc1cc(N2CCOCC2)nc(OCCN2CCOCC2)n1] | N-(6-Methoxy-3,4-dihydro-2H- naphthalen-1-ylidene)-N′-[6- morpholin-4-yl-2-(2- morpholin-4-yl-ethoxy)- pyrimidin-4-yl]-hydrazine |
| 803 | 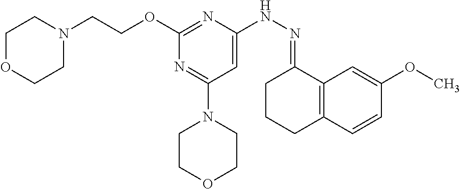 [SMILES: COc1ccc2c(c1)/C(=N/Nc1cc(N3CCOCC3)nc(OCCN3CCOCC3)n1)CCC2] | N-(7-Methoxy-3,4-dihydro-2H- naphthalen-1-ylidene)-N′-[6- morpholin-4-yl-2-(2- morpholin-4-yl-ethoxy)- pyrimidin-4-yl]-hydrazine |
| 804 | 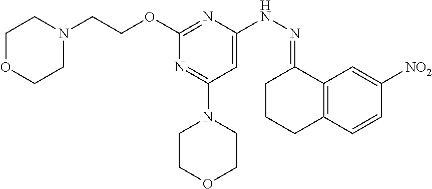 [SMILES: O=[N+]([O-])c1ccc2c(c1)/C(=N/Nc1cc(N3CCOCC3)nc(OCCN3CCOCC3)n1)CCC2] | N-(7-Nitro-3,4-dihydro-2H- naphthalen-1-ylidene)-N′-[6- morpholin-4-yl-2-(2- morpholin-4-yl-ethoxy)- pyrimidin-4-yl]-hydrazine |
| 805 | 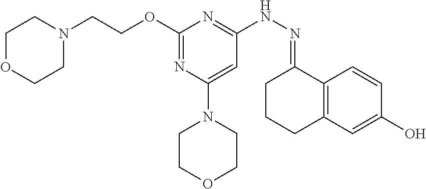 [SMILES: Oc1ccc2c(c1)CCC/C2=N\Nc1cc(N2CCOCC2)nc(OCCN2CCOCC2)n1] | N-(6-Hydroxy-3,4-dihydro-2H- naphthalen-1-ylidene)-N′-[6- morpholin-4-yl-2-(2- morpholin-4-yl-ethoxy)- pyrimidin-4-yl]-hydrazine |
| 806 | 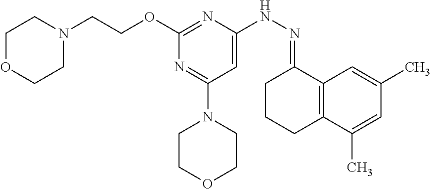 [SMILES: Cc1cc(C)c2c(c1)/C(=N/Nc1cc(N3CCOCC3)nc(OCCN3CCOCC3)n1)CCC2] | N-(5,7-Dimethyl-3,4-dihydro- 2H-naphthalen-1-ylidene)-N′- [6-morpholin-4-yl-2-(2- morpholin-4-yl-ethoxy)- pyrimidin-4-yl]-hydrazine |
| 807 | 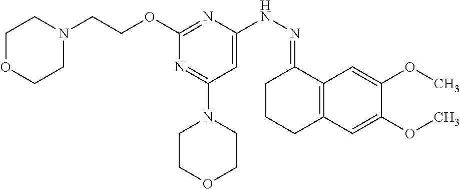 [SMILES: COc1cc2c(cc1OC)/C(=N/Nc1cc(N3CCOCC3)nc(OCCN3CCOCC3)n1)CCC2] | N-(6,7-Dimethoxy-3,4- dihydro-2H-naphthalen-1- ylidene)-N′-[6-morpholin-4-yl- 2-(2-morpholin-4-yl-ethoxy)- pyrimidin-4-yl]-hydrazine |
| 808 | 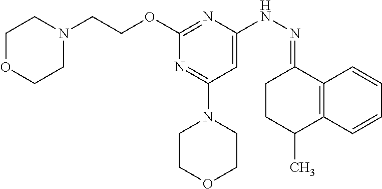 [SMILES: CC1CC/C(=N\Nc2cc(N3CCOCC3)nc(OCCN3CCOCC3)n2)c2ccccc21] | N-(4-Methyl-3,4-dihydro-2H- naphthalen-1-ylidene)-N′-[6- morpholin-4-yl-2-(2- morpholin-4-yl-ethoxy)- pyrimidin-4-yl]-hydrazine |
| 809 | 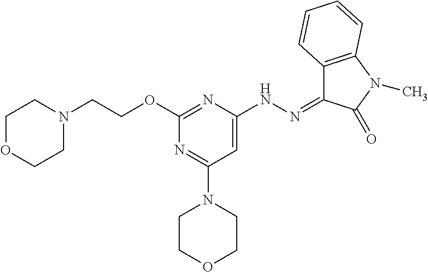 [SMILES: CN1C(=O)/C(=N/Nc2cc(N3CCOCC3)nc(OCCN3CCOCC3)n2)c2ccccc21] | 1-Methyl-3-{[6-morpholin-4- yl-2-(2-morpholin-4-yl- ethoxy)-pyrimidin-4-yl]- hydrazono}-1,3-dihydro-indol- 2-one |
| 810 | 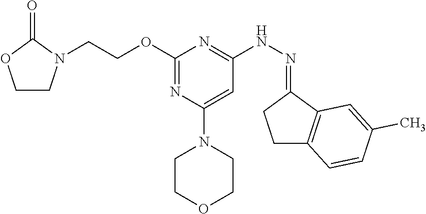 [SMILES: Cc1ccc2c(c1)/C(=N/Nc1cc(N3CCOCC3)nc(OCCN3CCOC3=O)n1)CC2] | 3-(2-{4-[N′-(6-Methyl-indan- 1-ylidene)-hydrazino]-6- morpholin-4-yl-pyrimidin-2- yloxy}-ethyl)-oxazolidin-2-one |
| 811 | 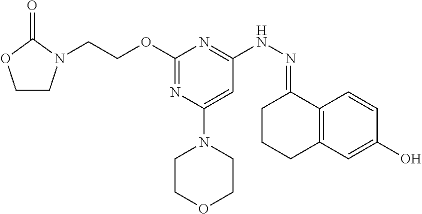 [SMILES: O=C1OCCN1CCOc1nc(N/N=C2\CCCc3cc(O)ccc32)cc(N2CCOCC2)n1] | 3-(2-{4-[N′-(6-Hydroxy-3,4- dihydro-2H-naphthalen-1- ylidene)-hydrazino]-6- morpholin-4-yl-pyrimidin-2- yloxy}-ethyl)-oxazolidin-2-one |
| 812 | 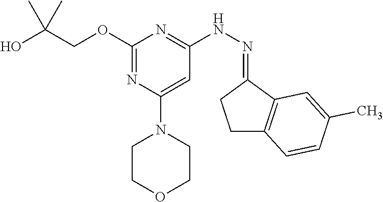 [SMILES: Cc1ccc2c(c1)/C(=N/Nc1cc(N3CCOCC3)nc(OCC(C)(C)O)n1)CC2] | 2-Methyl-1-{4-[N′-(6-methyl- indan-1-ylidene)-hydrazino]-6- morpholin-4-yl-pyrimidin-2- yloxy}-propan-2-ol |
| 813 | 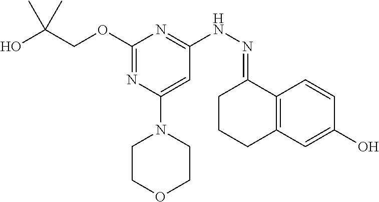 [SMILES: CC(C)(O)COc1nc(N/N=C2\CCCc3cc(O)ccc32)cc(N2CCOCC2)n1] | 5-{[2-(2-Hydroxy-2-methyl- propoxy)-6-morpholin-4-yl- pyrimidin-4-yl]-hydrazono}- 5,6,7,8-tetrahydro-naphthalen- 2-ol |
| 814 | 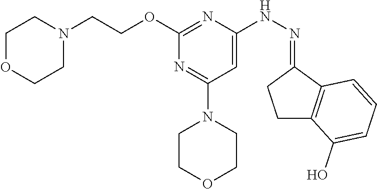 [SMILES: Oc1cccc2c1CC/C2=N\Nc1cc(N2CCOCC2)nc(OCCN2CCOCC2)n1] | N-(4-Hydroxy-indan-1- ylidene)-N′-[6-morpholin-4-yl- 2-(2-morpholin-4-yl-ethoxy)- pyrimidin-4-yl]-hydrazine |
| 815 | 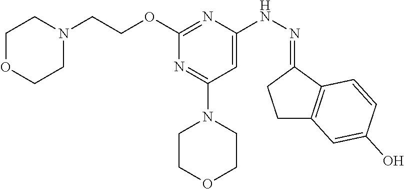 [SMILES: Oc1ccc2c(c1)CC/C2=N\Nc1cc(N2CCOCC2)nc(OCCN2CCOCC2)n1] | N-(5-Hydroxy-indan-1- ylidene)-N′-[6-morpholin-4-yl- 2-(2-morpholin-4-yl-ethoxy)- pyrimidin-4-yl]-hydrazine |
| 816 | 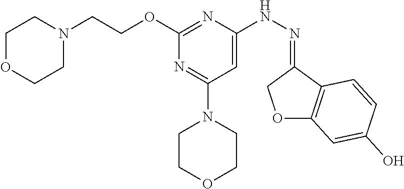 [SMILES: Oc1ccc2c(c1)OC/C2=N\Nc1cc(N2CCOCC2)nc(OCCN2CCOCC2)n1] | 3-{[6-Morpholin-4-yl-2-(2- morpholin-4-yl-ethoxy)- pyrimidin-4-yl]-hydrazono}- 2,3-dihydro-benzofuran-6-ol |
| 817 | 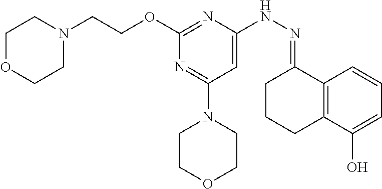 [SMILES: Oc1cccc2c1CCC/C2=N\Nc1cc(N2CCOCC2)nc(OCCN2CCOCC2)n1] | N-(5-Hydroxy-3,4-dihydro- naphthalen-1-ylidene)-N′-[6- morpholin-4-yl-2-(2- morpholin-4-yl-ethoxy)- pyrimidin-4-yl]-hydrazine |
| 818 | 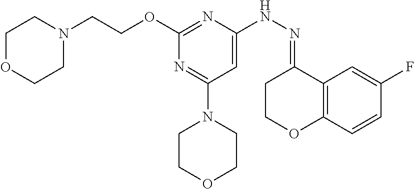 [SMILES: Fc1ccc2c(c1)/C(=N/Nc1cc(N3CCOCC3)nc(OCCN3CCOCC3)n1)CCO2] | N-(6-Fluoro-chroman-4- ylidene)-N′-[6-morpholin-4-yl- 2-(2-morpholin-4-yl-ethoxy)- pyrimidin-4-yl]-hydrazine |
| 819 | 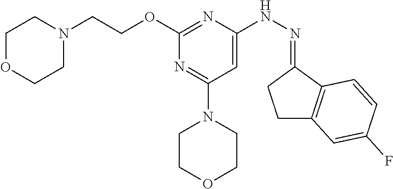 [SMILES: Fc1ccc2c(c1)CC/C2=N\Nc1cc(N2CCOCC2)nc(OCCN2CCOCC2)n1] | N-(5-Fluoro-indan-1-ylidene)- N′-[6-morpholin-4-yl-2-(2- morpholin-4-yl-ethoxy)- pyrimidin-4-yl]-hydrazine |
| 820 | 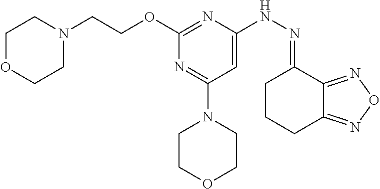 [SMILES: c1c(N/N=C2\CCCc3nonc32)nc(OCCN2CCOCC2)nc1N1CCOCC1] | N-(6,7-Dihydro-5H- benzo[1,2,5]oxadiazol-4- ylidene)-N′-[6-morpholin-4-yl- 2-(2-morpholin-4-yl-ethoxy)- pyrimidin-4-yl]-hydrazine |
| 821 | 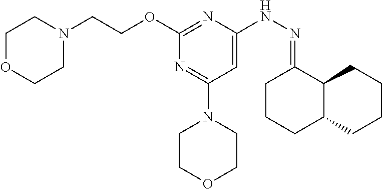 [SMILES: c1c(N/N=C2\CCC[C@H]3CCCC[C@H]23)nc(OCCN2CCOCC2)nc1N1CCOCC1] | N-[6-Morpholin-4-yl-2-(2- morpholin-4-yl-ethoxy)- pyrimidin-4-yl]-N′-(octahydro- naphhalen-1-ylidene)- hydrazine |
| 822 | 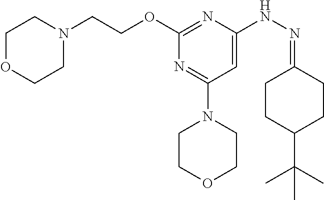 [SMILES: CC(C)(C)C1CCC(=NNc2cc(N3CCOCC3)nc(OCCN3CCOCC3)n2)CC1] | N-(4-tert-Butyl- cyclohexylidene)-N′-[6- morpholin-4-yl-2-(2- morpholin-4-yl-ethoxy)- pyrimidin-4-yl]-hydrazine |
| 823 | 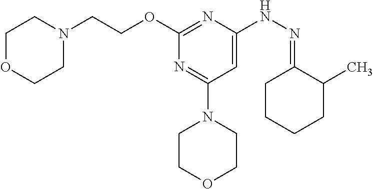 [SMILES: CC1CCCC/C1=N\Nc1cc(N2CCOCC2)nc(OCCN2CCOCC2)n1] | N-(2-Methyl-cyclohexylidene)- N′-[6-morpholin-4-yl-2-(2- morpholin-4-yl-ethoxy)- pyrimidin-4-yl]-hydrazine |
| 824 | 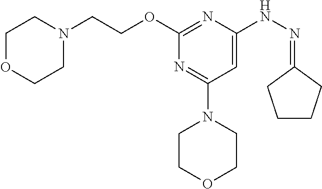 [SMILES: c1c(NN=C2CCCC2)nc(OCCN2CCOCC2)nc1N1CCOCC1] | N-Cyclopentylidene-N′-[6- morpholin-4-yl-2-(2- morpholin-4-yl-ethoxy)- pyrimidin-4-yl]-hydrazine |
| 825 | 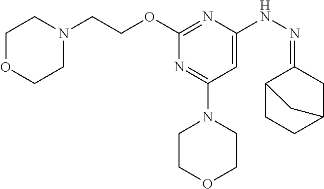 [SMILES: c1c(N/N=C2/CC3CCC2C3)nc(OCCN2CCOCC2)nc1N1CCOCC1] | N-Bicyclo[2.2.1]hept-2- ylidene-N′-[6-morpholin-4-yl- 2-(2-morpholin-4-yl-ethoxy)- pyrimidin-4-yl]-hydrazine |
| 826 | 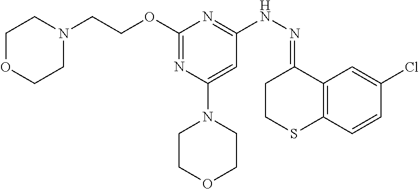 [SMILES: Clc1ccc2c(c1)/C(=N/Nc1cc(N3CCOCC3)nc(OCCN3CCOCC3)n1)CCS2] | N-(6-Chloro-thiochroman-4- ylidene)-N′-[6-morpholin-4-yl- 2-(2-morpholin-4-yl-ethoxy)- pyrimidin-4-yl]-hydrazine |
| 827 | 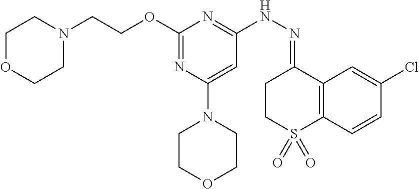 [SMILES: O=S1(=O)CC/C(=N\Nc2cc(N3CCOCC3)nc(OCCN3CCOCC3)n2)c2cc(Cl)ccc21] | N-(6-Chloro-1,1-dioxo-1λ⁶- thiochroman-4-ylidene)-N′-[6- morpholin-4-yl-2-(2- morpholin-4-yl-ethoxy)- pyrimidin-4-yl]-hydrazine |
| 828 | 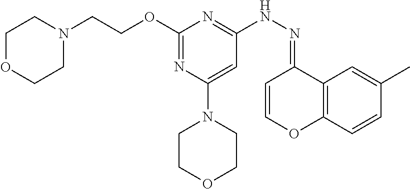 [SMILES: Cc1ccc2occ/c(=N\Nc3cc(N4CCOCC4)nc(OCCN4CCOCC4)n3)c2c1] | N-(6-Methyl-chromen-4- ylidene)-N′-[6-morpholin-4-yl- 2-(2-morpholin-4-yl-ethoxy)- pyrimidin-4-yl]-hydrazine |
| 829 | 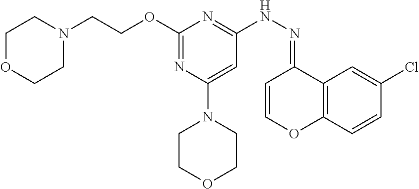 [SMILES: Clc1ccc2occ/c(=N\Nc3cc(N4CCOCC4)nc(OCCN4CCOCC4)n3)c2c1] | N-(6-Chloro-chromen-4- ylidene)-N′-[6-morpholin-4-yl- 2-(2-morpholin-4-yl-ethoxy)- pyrimidin-4-yl]-hydrazine |

All of the features, specific embodiments and particular substituents disclosed herein may be combined in any combination. Each feature, embodiment or substituent disclosed in this specification may be replaced by an alternative feature, embodiment or substituent serving the same, equivalent, or similar purpose. In the case of chemical compounds, specific values can be combined in any combination resulting in a stable structure. Furthermore, specific values (whether preferred or not) for substituents in one type of chemical structure may be combined with values for other substituents (whether preferred or not) in the same or different type of chemical structure. Thus, unless expressly stated otherwise, each feature, embodiment or substituent disclosed is only an example of a generic series of equivalent or similar features feature, embodiments or substituents.

c-Rel has been shown to play a role in the proliferation and survival of B-cells. The c-Rel protein is expressed at all stages of B-cell development, but is expressed at the highest levels in mature B-cells. c-Rel knockout mice develop normally and have no gross defects in hemopoiesis. However, they show immuno-deficiencies which primarily stem from defects in B-cells proliferation and survival in response to mitogenic activation, such as LPS, anti-IgM, antigens, and CD40. In addition, they show reduced antibody production in response to an antigen. Without wishing to be bound by any theory, because the experimental evidence appears to indicate that the compounds of the invention inhibit the activity of c-Rel which has been shown to be necessary for B-cell proliferation and survival, it is believed that compounds of the invention are useful in treating B-cell regulated autoimmune disorders.

An “autoimmune disease” herein is a non-malignant disease or disorder arising from and directed against an individual's own (self) antigens and/or tissues.

Lymphocytes are one of several populations of white blood cells; they specifically recognize and respond to foreign antigen. The three major classes of lymphocytes are B lymphocytes (B cells), T lymphocytes (T cells) and natural killer (NK) cells. B lymphocytes are the cells responsible for antibody production and provide humoral immunity. B cells mature within the bone marrow and leave the marrow expressing an antigen-binding antibody on their cell surface. When a naive B cell first encounters the antigen for which its membrane-bound antibody is specific, the cell begins to divide rapidly and its progeny differentiate into memory B cells and effector cells called “plasma cells”. Memory B cells have a longer life span and continue to express membrane-bound antibody with the same specificity as the original parent cell. Plasma cells do not produce membrane-bound antibody but instead produce secreted form of the antibody. Secreted antibodies are the major effector molecules of humoral immunity. As used herein, “a B-cell regulated autoimmune disorder” is an autoimmune disorder that involves misregulation of B-cells.

Examples of a B-cell regulated autoimmune disorder that can be treated by administering one or more compound of the invention, or a pharmaceutically acceptable salt, solvate, clathrate, hydrate, polymorph, or prodrug, include lystemic lupus erythematosis (SLE), Sjogren's syndrome, graft-versus-host disease, systemic sclerosis, myasthenia gravis, juvenile rheumatoid arthritis, osteoarthritis, psoriatic arthritis, dermatitis, atopic dermatitis, chronic autoimmune urticaria, polymyositis/dermatomyositis, toxic epidermal necrolysis, systemic scleroderma and sclerosis, respiratory distress syndrome, adult respiratory distress syndrome (ARDS), meningitis, allergic rhinitis, encephalitis, uveitis, colitis, glomerulonephritis, allergic conditions, eczema, asthma, atherosclerosis, autoimmune myocarditis, leukocyte adhesion deficiency, lupus (nephritis, non-renal, discoid, alopecia), allergic encephalomyelitis, tuberculosis, sarcoidosis, granulomatosis, Wegener's granulomatosis, agranulocytosis, vasculitis, aplastic anemia, Coombs positive anemia, Diamond Blackfan anemia, immune hemolytic anemia, hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, multiple organ injury syndrome, myasthenia gravis, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection, graft versus host disease (GVHD), pemphigoid bullous, pemphigus, vulgaris, foliaceus, autoimmune polyendocrinopathies, Reiter's disease, stiff-man syndrome, giant cell arteritis, immune complex nephritis, IgA nephropathy, IgM polyneuropathies, IgM mediated neuropathy, idiopathic thrombocytopenic purpura (ITP), thrombotic thrombocytopenic purpura (TTP), autoimmune thrombocytopenia, autoimmune orchitis, autoimmune oophoritis, primary hypothyroidism; autoimmune endocrine diseases, autoimmune thyroiditis, chronic thyroiditis (Hashimoto's Thyroiditis), subacute thyroiditis, idiopathic hypothyroidism, Addison's disease, Grave's disease, polyglandular endocrinopathy syndromes, Sheehan's syndrome, autoimmune hepatitis, Lymphoid interstitial pneumonitis (HIV), non-transplant bronchiolitis obliterans, Guillain-Barre' Syndrome, Large Vessel Vasculitis, Polymyalgia Rheumatica, Giant Cell (Takayasu's) Arteritis, Medium Vessel Vasculitis, Kawasaki's Disease, Polyarteritis Nodosa, ankylosing spondylitis, Berger's Disease, Rapidly Progressive Glomerulonephritis, Primary biliary cirrhosis, Celiac sprue, Cryoglobulinemia, ALS, and coronary artery disease.

In one embodiment, B-cell regulated autoimmune disorder are selected from the group consisting of systemic sclerosis, toxic epidermal necrolysis, encephalitis, glomerulonephritis, leukocyte adhesion deficiency, tuberculosis, agranulocytosis, Factor VII deficiency, hemophilia A, pancytopenia, leukopenia, diseases involving leukocyte diapedesis, multiple organ injury syndrome, anti-glomerular basement membrane disease, allergic neuritis, Castleman's syndrome, Goodpasture's Syndrome, Lambert-Eaton Myasthenic Syndrome, Reynaud's syndrome, pemphigoid bullous, foliaceus, Reiter's disease, stiff-man syndrome, primary hypothyroidism, Sheehan's syndrome, non-transplant bronchiolitis obliterans, Polymyalgia Rheumatica, Kawasaki's Disease, Polyarteritis Nodosa, Berger's Disease, Rapidly Progressive Glomerulonephritis, Celiac sprue, Cryoglobulinemia, ALS, and coronary artery disease.

Without wishing to be bound by any theory, it is believed that the compounds of the invention can be used to inhibit proliferation and/or induce apoptosis in B-cells since it has been shown that c-Rel is necessary for proliferation and survival of B-cells. Therefore, in one aspect, the invention provides a method of inhibiting the proliferation of and/or inducing apoptosis in B-cells, comprising contacting the B-cells with a compound that inhibits the accumulation of c-Rel in the nucleus of the cells and does not materially inhibit the accumulation of other NF-kB family members in the nucleus of the cells. In one embodiment, the compound is any compound disclosed herein. In another embodiment, the compound inhibits post-translational phosphorylation of c-Rel. In another embodiment, the compound inhibits the post-translational acetylation of c-Rel.

In another aspect, the invention provides a method of inhibiting the proliferation of and/or inducing apoptosis in B-cells, comprising contacting the B-cells with a compound that alters the post-translational modification of c-Rel and does not materially inhibit the post-translational phosphorylation of other NF-κB family members. In one embodiment, the compound inhibits the post-translational phosphorylation of c-Rel. In another embodiment, the compound inhibits the post-translational acetylation of c-Rel. In another embodiment, the compound is any compound disclosed herein.

In another aspect, the invention provides a method of inhibiting the proliferation of and/or inducing apoptosis in B-cells, comprising contacting B-cells with a compound that inhibits DNA binding of c-Rel to a KB site and does not inhibit the DNA binding of other NF-κB family members. In one embodiment, the compound is any compound disclosed herein.

As used interchangeably herein, “c-rel activity,” “biological activity of c-rel,” or “activity of c-rel,” include an activity exerted by c-rel protein on a c-rel responsive cell or tissue, e.g., a T cell, dendritic cells, NK cells, or on a c-rel target molecule, e.g., a nucleic acid molecule or protein target molecule, as determined in vivo, or in vitro, according to standard techniques. In one embodiment, c-rel activity is a direct activity, such as an association with a c-rel-target molecule. Alternatively, a c-rel activity is an indirect activity, such as a downstream biological event mediated by interaction of the c-rel protein with a c-rel target molecule.

As used herein, the term “contacting” (i.e., contacting a cell e.g. a cell, with a compound) includes incubating the compound and the cell together in vitro (e.g., adding the compound to cells in culture) as well as administering the compound to a subject such that the compound and cells of the subject are contacted in vivo. The term “contacting” does not include exposure of cells to a c-rel modulator that may occur naturally in a subject (i.e., exposure that may occur as a result of a natural physiological process).

As used herein, the term “modulate” with respect to c-rel includes changing the expression, activity or function of c-rel in such a manner that it differs from the naturally-occurring expression, function or activity of c-rel under the same conditions. For example, the expression, function or activity can be greater or less than that of naturally occurring c-rel, e.g., owing to a change in binding specificity, etc. As used herein, the various forms of the term “modulate” include stimulation (e.g., increasing or upregulating a particular response or activity) and inhibition (e.g., decreasing or downregulating a particular response or activity).

In the context of NF-κB and/or IκB (including IκBα and IκBβ) expression and/or amount, the term “without materially inhibiting” as used herein means a smaller than 40%, preferably smaller than 30%, 20%, 10%, 5%, 4%, 3%, 2%, 1%, or 0% change in the level of expression of NFκB and/or amount of IκB.

In the context of alterations in the post-translational modification state or phosphorylation state of Nf-κB family members, the term “without materially altering” as used herein means that there is a quantitative difference of no more than 30-fold, preferably 15-fold, more preferably 10-fold, more preferably two-fold, and most preferably not detectably altered. The alteration can either be increased or decreased as compared to wild-type (e.g. state in an unstimulated cell) or control/reference samples.

As used herein, the term “post-translational modification” means any type of protein or polypeptide modifications that can be made to the native polypeptide sequence after its initial translation, enzyme-catalyzed or not, such as e.g. a acylation, phosphorylation, dephosphorylation, SUMOylation, ubiquitinylation, carboxymethylation, formylation, acetylation, deacetylation, gamma carboxyglutamic acid, norleucine, amidation, deamidation, carboxylation, carboxyamylation, sulfation, methylation, demethylation, hydroxylation, ADP-ribosylation, maturation, adenylation, O-linked glycosylation, N-linked glycosylation, methionine oxidation, myristoylation, formation of disulphide bonds, changes in oxidation/reduction, and addition of lipid (prenylation).

As used herein, the term “stimulus” means a growth factor, a cytokine, a hormone, a steroid, a lipid, an antigen, a small molecule (e.g., Ca²⁺, cAMP, cGMP), an osmotic shock, a heat or cold shock, a pH change, a change in ionic strength, a mechanical force, a viral or bacterial infection, or an attachment or detachment from a neighboring cell or a surface with or without a coated protein.

As used herein, the term “Nf-κB family members” refers to RelA (or p65), RelB, NF-κB1 (or p105/p50), NF-κB2 (or p100/p52), and cRel.

In another aspect, this invention features a pharmaceutical composition that includes a pharmaceutically acceptable carrier and at least one compound that inhibits the accumulation of c-Rel in the nucleus of the B-cells and does not inhibit the accumulation of other NF-κB family members in the nucleus of the B-cells. In one embodiment, the compound in the pharmaceutical composition that inhibits the accumulation of c-Rel is not a compound disclosed in the patents or patent applications listed in Table 2. In one embodiment, the compound in the pharmaceutical composition that inhibits the accumulation of c-Rel is not a compound disclosed in the patents or patent applications listed in Table 3.

In another aspect, this invention features a pharmaceutical composition that includes a pharmaceutically acceptable carrier and at least one compound that alters the post-translational modification of c-Rel and does not materially inhibit the post-translational phosphorylation of other NF-κB family members. In one embodiment, the compound in the pharmaceutical composition that alters the post-translational modification of c-Rel is not a compound disclosed in the patents and patent applications listed in Table 2. In one embodiment, the compound in the pharmaceutical composition that alters the post-translational modification of c-Rel is not a compound disclosed in the patents and patent applications listed in Table 3.

In another aspect, this invention features a pharmaceutical composition that includes a pharmaceutically acceptable carrier and at least one compound that inhibits DNA binding of c-Rel to a κB site and does not inhibit the DNA binding of other NF-κB family members. In one embodiment, the compound in the pharmaceutical composition that inhibits DNA binding of c-Rel to a κB site is not a compound disclosed in the patents and patent applications listed in Table 2. In one embodiment, the compound in the pharmaceutical composition that inhibits DNA binding of c-Rel to a κB site is not a compound disclosed in the patents and patent applications listed in Table 3.

TABLE 2

| | | | Publication |
|---|---|---|---|
| Ser. No. | Filing Date | Publication No. | Date |
| U.S. Pat. No. | Jun. 15, 2000 | | |
| 6,384,032 | | | |
| U.S. Pat. No. | Nov. 30, 2001 | | |
| 6,680,315 | | | |
| U.S. Pat. No. | Nov. 30, 2001 | | |
| 6,693,097 | | | |
| U.S. Pat. No. | Jul. 10, 2002 | | |
| 6,660,733 | | | |
| U.S. Pat. No. | Nov. 26, 2002 | | |
| 6,858,606 | | | |
| U.S. Application No. | Sep. 5, 2003 | 2004-0053926 | Mar. 18, 2004 |
| 10/656,360 | | | |
| U.S. Application No. | Sep. 5, 2003 | 2004-0048873 | Mar. 11, 2004 |
| 10/656,671 | | | |
| U.S. Application No. | Sep. 5, 2003 | 2004-0053937 | Mar. 18, 2004 |
| 10/655,672 | | | |
| U.S. Application No. | Oct. 14, 2003 | 2004-0198725 | Oct. 7, 2004 |
| 10/686,505 | | | |
| PCT Application No. | May 28, 2004 | WO 2005/ | Jan. 6, 2005 |
| PCT/US2004/017064 | | 000404 | |

TABLE 3

| | | | Publication |
|---|---|---|---|
| Ser. No. | Filing Date | Publication No. | Date |
| U.S. Pat. No. | Jun. 15, 2000 | | |
| 6,384,032 | | | |
| U.S. Pat. No. | Nov. 30, 2001 | | |
| 6,680,315 | | | |
| U.S. Pat. No. | Nov. 30, 2001 | | |
| 6,693,097 | | | |
| U.S. Pat. No. | Jul. 10, 2002 | | |
| 6,660,733 | | | |
| U.S. Pat. No. | Nov. 26, 2002 | | |
| 6,858,606 | | | |
| U.S. Application No. | Sep. 5, 2003 | 2004-0053926 | Mar. 18, 2004 |
| 10/656,360 | | | |
| U.S. Application No. | Sep. 5, 2003 | 2004-0048873 | Mar. 11, 2004 |
| 10/656,671 | | | |
| U.S. Application No. | Sep. 5, 2003 | 2004-0053937 | Mar. 18, 2004 |
| 10/655,672 | | | |
| U.S. Application No. | Oct. 14, 2003 | 2004-0198725 | Oct. 7, 2004 |
| 10/686,505 | | | |
| PCT Application No. | May 28, 2004 | WO 2005/ | Jan. 6, 2005 |
| PCT/US2004/017064 | | 000404 | |
| U.S. Provisional | Jul. 1, 2004 | | |
| Application No. | | | |
| 60/585,124 | | | |
| U.S. Application No. | Nov. 10, 2004 | | |
| 10/985,696 | | | |
| U.S. Application No. | Nov. 10, 2004 | | |
| 10/985,716 | | | |
| U.S. Application No. | Nov. 10, 2004 | | |
| 10/985,627 | | | |
| U.S. Provisional | Nov. 19, 2004 | | |
| Application No. | | | |
| 60/629,505 | | | |
| U.S. Provisional | Nov. 10, 2004 | | |
| Application No. | | | |
| 60/626,609 | | | |
| U.S. Provisional | Nov. 10, 2004 | | |
| Application No. | | | |
| 60/627,001 | | | |
| U.S. Provisional | Nov. 10, 2004 | | |
| Application No. | | | |
| 60/626,761 | | | |
| U.S. Application No. | Nov. 10, 2004 | | |
| 10/986,553 | | | |
| U.S. Application No. | Jan. 21, 2005 | | |
| 11/041,537 | | | |
| U.S. Provisional | Jan. 28, 2005 | | |
| Application No. | | | |
| 60/648,645 | | | |
| PCT Application No. | Apr. 13, 2005 | | |
| PCT/US05/12578 | | | |
| U.S. Provisional | May 13, 2005 | | |
| Application No. <not | | | |
| yet assigned> | | | |
| Title: “IL-12 | | | |
| Modulatory | | | |
| Compounds” | | | |
| Attorney Docket No. | | | |
| ILI-015-01PR-00 | | | |

Methods for making the compounds of the invention have been disclosed in the U.S. patents and patent applications listed in Table 3. The entire teachings of these patents and patent applications are incorporated herein by reference.

As used herein, the term “alkyl” refers to a straight-chained or branched hydrocarbon group containing 1 to 12 carbon atoms. The term “lower alkyl” refers to a C1-C6 alkyl chain. Examples of alkyl groups include methyl, ethyl, n-propyl, isopropyl, tert-butyl, and n-pentyl. Alkyl groups may be optionally substituted with one or more substituents.

The term “alkenyl” refers to an unsaturated hydrocarbon chain that may be a straight chain or branched chain, containing 2 to 12 carbon atoms and at least one carbon-carbon double bond. Alkenyl groups may be optionally substituted with one or more substituents.

The term “alkynyl” refers to an unsaturated hydrocarbon chain that may be a straight chain or branched chain, containing the 2 to 12 carbon atoms and at least one carbon-carbon triple bond. Alkynyl groups may be optionally substituted with one or more substituents.

The sp² or sp carbons of an alkenyl group and an alkynyl group, respectively, may optionally be the point of attachment of the alkenyl or alkynyl groups.

The term “alkoxy,” as used herein, refers to an alkyl or a cycloalkyl group which is linked to another moiety though an oxygen atom. Alkoxy groups can be optionally substituted with one or more substituents.

The term “mercapto” refers to a —SH group.

The term “alkyl sulfanyl,” as used herein, refers to an alkyl or a cycloalkyl group which is linked to another moiety though a divalent sulfer atom. Alkyl sulfanyl groups can be optionally substituted with one or more substituents.

As used herein, the term “halogen” or “halo” means —F, —Cl, —Br or —I.

As used herein, the term “haloalkyl” means and alkyl group in which one or more (including all) the hydrogen radicals are replaced by a halo group, wherein each halo group is independently selected from —F, —Cl, —Br, and —I. The term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

The term “cycloalkyl” refers to a hydrocarbon 3-8 membered monocyclic or 7-14 membered bicyclic ring system which is completely saturated ring. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclooctyl, cyclononyl, cyclodecyl, and bicyclo[2.1.1]hexyl.

The term “cyclyl” refers to a hydrocarbon 3-8 membered monocyclic or 7-14 membered bicyclic ring system having at least one non-aromatic ring, wherein the non-aromatic ring has some degree of unsaturation. Cyclyl groups may be optionally substituted with one or more substituents. In one embodiment, 0, 1, 2, 3, or 4 atoms of each ring of a cyclyl group may be substituted by a substituent. Examples of cyclyl groups include cyclohexenyl, bicyclo[2.2.1]hept-2-enyl, dihydronaphthalenyl, benzocyclopentyl, cyclopentenyl, cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl and the like.

The term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

As used herein, the term “aralkyl” means an aryl group that is attached to another group by a (C₁-C₆)alkylene group. Aralkyl groups may be optionally substituted, either on the aryl portion of the aralkyl group or on the alkylene portion of the aralkyl group, with one or more substituent. Representative aralkyl groups include benzyl, 2-phenyl-ethyl, naphth-3-yl-methyl and the like.

As used herein, the term “alkylene” refers to an alkyl group that has two points of attachment. The term “(C₁-C₆)alkylene” refers to an alkylene group that has from one to six carbon atoms. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups may be optionally substituted.

As used herein, the term “cycloalkylene” refers to a cycloalkyl group that has two points of attachment. Cycloalkylene groups may be optionally substituted.

As used herein, the term “cyclylene” refers to a cyclyl group that has two points of attachment. Cyclylene groups may be optionally substituted.

As used herein, the term “arylene” refers to an aryl group that has two points of attachment. Arylene groups may be optionally substituted.

As used herein, the term “aralkylene” refers to an aralkyl group that has two points of attachment. Aralkylene groups may be optionally substituted.

The term “arylalkoxy” refers to an alkoxy substituted with an aryl.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon. Heteroaryl groups may be optionally substituted with one or more substituents. In one embodiment, 0, 1, 2, 3, or 4 atoms of each ring of a heteroaryl group may be substituted by a substituent. Examples of heteroaryl groups include pyridyl, 1-oxo-pyridyl, furanyl, benzo[1,3]dioxolyl, benzo[1,4]dioxinyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, and benzo(b)thienyl, 3H-thiazolo[2,3-c][1,2,4]thiadiazolyl, imidazo[1,2-d]-1,2,4-thiadiazolyl, imidazo[2,1-b]-1,3,4-thiadiazolyl, 1H,2H-furo[3,4-d]-1,2,3-thiadiadiazolyl, 1H-pyrazolo[5,1-c]-1,2,4-triazolyl, pyrrolo[3,4-d]-1,2,3-triazolyl, cyclopentatriazolyl, 3H-pyrrolo[3,4-c]isoxazolyl, 1H,3H-pyrrolo[1,2-c]oxazolyl, pyrrolo[2,1b]oxazolyl, and the like.

As used herein, the term “heteroaralkyl” or “heteroarylalkyl” means a heteroaryl group that is attached to another group by a (C₁-C₆)alkylene. Heteroaralkyl groups may be optionally substituted, either on the heteroaryl portion of the heteroaralkyl group or on the alkylene portion of the heteroaralkyl group, with one or more substituent. Representative heteroaralkyl groups include 2-(pyridin-4-yl)-propyl, 2-(thien-3-yl)-ethyl, imidazol-4-yl-methyl and the like.

As used herein, the term “heteroarylene” refers to a heteroaryl group that has two points of attachment. Heteroarylene groups may be optionally substituted.

As used herein, the term “heteroaralkylene” refers to a heteroaralkyl group that has two points of attachment. Heteroaralkylene groups may be optionally substituted.

The term “heterocycloalkyl” refers to a nonaromatic, completely saturated 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, 2-oxopiperazinyl, 2-oxopiperidinyl, 2-oxopyrrolidinyl, 4-piperidonyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolane, tetrahydrofuranyl, tetrahydrothienyl, an thiirene.

The term “heterocyclyl” refers to a nonaromatic 5-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system has some degree of unsaturation. Heterocyclyl groups may be optionally substituted with one or more substituents. In one embodiment, 0, 1, 2, 3, or 4 atoms of each ring of a heterocyclyl group may be substituted by a substituent. Examples of these groups include thiirenyl, thiadiazirinyl, dioxazolyl, 1,3-oxathiolyl, 1,3-dioxolyl, 1,3-dithiolyl, oxathiazinyl, dioxazinyl, dithiazinyl, oxadiazinyl, thiadiazinyl, oxazinyl, thiazinyl, 1,4-oxathiin, 1,4-dioxin, 1,4-dithiin, 1H-pyranyl, oxathiepinyl, 5H-1,4-dioxepinyl, 5H-1,4-dithiepinyl, 6H-isoxazolo[2,3-d]1,2,4-oxadiazolyl, 7H-oxazolo[3,2-d]1,2,4-oxadiazolyl, and the like.

As used herein, the term “heterocycloalkylene” refers to a heterocycloalkyl group that has two points of attachment. Heterocycloalkylene groups may be optionally substituted.

As used herein, the term “heterocyclylene” refers to a heterocyclyl group that has two points of attachment. Heterocyclylene groups may be optionally substituted.

When a cycloalkyl, cyclyl, heterocycloalkyl, or heterocyclyl is fused to another ring (e.g., a cycloalkyl, cyclyl, heterocycloalkyl, heterocyclyl, aryl, heteroaryl), it shares two or more ring atoms, preferably two to four ring atoms, with the other ring.

The term “amino” refers to —NH₂. The term “alkylamino” refers to an amino in which one hydrogen is replaced by an alkyl group. The term “dialkylamino” refers to an amino in which each of the hydrogens is replaced by an independently selected alkyl group. The term “aminoalkyl” refers to an alkyl substituent which is further substituted with one or more amino groups.

The term “mercaptoalkyl” refers to an alkyl substituent which is further substituted with one or more mercapto groups.

The term “hydroxyalkyl” or “hydroxylalkyl” refers to an alkyl substituent which is further substituted with one or more hydroxy groups.

The term “sulfonylalkyl” refers to an alkyl substituent which is further substituted with one or more sulfonyl groups.

The term “sulfonylaryl” refers to an aryl substituent which is further substituted with one or more sulfonyl groups.

The term alkylcarbonyl refers to an —C(O)-alkyl.

The term “mercaptoalkoxy” refers to an alkoxy substituent which is further substituted with one or more mercapto groups.

The term “alkylcarbonylalkyl” refers to an alkyl substituent which is further substituted with —C(O)-alkyl. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

Suitable substituents for an alkyl, alkoxy, alkyl sulfanyl, alkylamino, dialkylamino, alkylene, alkenyl, alkynyl, cycloalkyl, cyclyl, heterocycloalkyl, heterocyclyl, aryl, aralkyl, heteroaryl, heteroaralkyl, cycloalkylene, cyclylene, heterocycloalkylene, heterocyclylene, arylene, aralkylene, heteroalkylene and heteroaryalkylene groups include any substituent which will form a stable compound of the invention. Examples of substituents for an alkyl, alkoxy, alkylsulfanyl, alkylamino, dialkylamino, alkylene, alkenyl, alkynyl, cycloalkyl, cyclyl, heterocycloalkyl, heterocyclyl, aryl, aralkyl, heteroaryl, heteroaralkyl, cycloalkylene, cyclylene, heterocycloalkylene, heterocyclylene, arylene, aralkylene, heteroalkylene and heteroaryalkylene include an optionally substituted alkyl, an optionally substituted alkoxy, an optionally substituted alkyl sulfanyl, an optionally substituted alkenyl, an optionally substituted alkynyl, an optionally substituted cycloalkyl, an optionally substituted cyclyl, an optionally substituted heterocyclyl, an optionally substituted heterocycloalkyl, an optionally substituted aryl, an optionally substituted heteroaryl, an optionally substituted aralkyl, an optionally substituted heteraralkyl, a haloalkyl, halo, cyano, nitro, haloalkoxy, ═O, ═S, ═NR, —OR^{k}, —NR^{h}R^{j}, —SR^{k}, —C(O)R^{k}, —C(O)NR^{h}R^{j}, —NR^{k}C(O)R^{k}, —C(O)OR^{k}, —OC(O)R^{k}, —NR^{k}C(O)NR^{h}R^{j}, —OC(O)NR^{h}R^{j}, —NR^{k}C(O)OR^{k}, —C(NR)R^{k}, —C(NR)NR^{h}R^{j}, —NR^{k}C(NR)R^{k}, —C(NR)OR^{k}, —OC(NR)R^{k}, —NR^{k}C(NR)NR^{h}R^{j}, —OC(NR)NR^{h}R^{j}, —NR^{k}C(NR)OR^{k}, —C(S)R^{k}, —C(S)NR^{h}R^{j}, —NR^{k}C(S)R^{k}, —C(S)OR^{k}, —OC(S)R^{k}, —NR^{k}C(S)NR^{h}R^{j}, —OC(S)NR^{h}R^{j}, —NR^{k}C(S)OR^{k}, —C(O)SR^{k}, —SC(O)R^{k}, —S(O)ₚR^{k}, —S(O)ₚNR^{h}R^{j}, —OS(O)ₚR^{k}, —S(O)ₚOR^{k}, —OS(O)OR^{k}, —P(O)(OR^{k})₂, —OP(O)(OR^{k})₂, —P(S)(OR^{k})₂, —SP(O)(OR^{k})₂, —P(O)(SR^{k})(OR^{k}), —OP(O)(SR^{k})(OR^{k}), —P(O)(SR^{k})₂, or —OP(O)(SR^{k})₂, wherein p is 1 or 2.

In addition, alkyl, cycloalkyl, alkylene, a heterocycloalkyl, a and any saturated portion of a alkenyl, a cyclyl, alkynyl, heterocyclyl, aralkyl, and heteroaralkyl groups, may also be substituted with ═O, ═S, or ═NR.

When a heterocyclyl, heteroaryl, or heteroaralkyl group contains a nitrogen atom, it may be substituted or unsubstituted. When a nitrogen atom in the aromatic ring of a heteroaryl group has a substituent the nitrogen may be a quaternary nitrogen.

Choices and combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject). Typically, such compounds are stable at a temperature of 40° C. or less, in the absence of excessive moisture, for at least one week. Such choices and combinations will be apparent to those of ordinary skill in the art and may be determined without undue experimentation.

As used herein, the term “lower” refers to a group having up to six atoms. For example, a “lower alkyl” refers to an alkyl radical having from 1 to 6 carbon atoms, and a “lower alkenyl” or “lower alkynyl” refers to an alkenyl or alkynyl radical having from 2 to 6 carbon atoms, respectively. A “lower alkoxy” or “lower alkyl sulfanyl” group refers to an alkoxy or alkyl sulfanyl group that has from 1 to 6 carbon atoms.

The compounds of the invention are defined herein by their chemical structures and/or chemical names. Where a compound is referred to by both a chemical structure and a chemical name, and the chemical structure and chemical name conflict, the chemical structure is determinative of the compound's identity.

The compounds of this invention include the compounds themselves, as well as their salts, solvate, clathrate, hydrate, polymorph, or prodrugs, if applicable. As used herein, the term “pharmaceutically acceptable salt,” is a salt formed from, for example, an acid and a basic group of a compound of any one of the formulae disclosed herein. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The term “pharmaceutically acceptable salt” also refers to a salt prepared from a compound of any one of the formulae disclosed herein having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of any one of the formulae disclosed herein having a basic functional group, such as an amino functional group, and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, and p-toluenesulfonic acid.

As used herein, the term “polymorph” means solid crystalline forms of a compound of the present invention or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a compound of the present invention or a salt thereof, which further includes a stoichiometric or non-stoichiometric amount of water bound by non-covalent intermolecular forces.

As used herein, the term “clathrate” means a compound of the present invention or a salt thereof in the form of a crystal lattice that contains spaces (e.g., channels) that have a guest molecule (e.g., a solvent or water) trapped within.

As used herein and unless otherwise indicated, the term “prodrug” means a derivative of a compound that can hydrolyze, oxidize, or otherwise react under biological conditions (in vitro or in vivo) to provide a compound of this invention. Prodrugs may only become active upon such reaction under biological conditions, or they may have activity in their unreacted forms. Examples of prodrugs contemplated in this invention include, but are not limited to, analogs or derivatives of compounds of any one of the formulae disclosed herein that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of any one of the formulae disclosed herein that comprise —NO, —NO₂, —ONO, or —ONO₂ moieties. Prodrugs can typically be prepared using well-known methods, such as those described by 1 BURGER'S MEDICINAL CHEMISTRY AND DRUG DISCOVERY (1995) 172-178, 949-982 (Manfred E. Wolff ed., 5^{th} ed).

As used herein and unless otherwise indicated, the terms “biohydrolyzable amide”, “biohydrolyzable ester”, “biohydrolyzable ca”, “biohydrolyzable carbamate”, “biohydrolyzable carbonate”, “biohydrolyzable ureide” and “biohydrolyzable phosphate analogue” mean an amide, ester, carbamate, carbonate, ureide, or phosphate analogue, respectively, that either: 1) does not destroy the biological activity of the compound and confers upon that compound advantageous properties in vivo, such as uptake, duration of action, or onset of action; or 2) is itself biologically inactive but is converted in vivo to a biologically active compound. Examples of biohydrolyzable amides include, but are not limited to, lower alkyl amides, α-amino acid amides, alkoxyacyl amides, and alkylaminoalkylcarbonyl amides. Examples of biohydrolyzable esters include, but are not limited to, lower alkyl esters, alkoxyacyloxy esters, alkyl acylamino alkyl esters, and choline esters. Examples of biohydrolyzable carbamates include, but are not limited to, lower alkylamines, substituted ethylenediamines, aminoacids, hydroxyalkylamines, heterocyclic and heteroaromatic amines, and polyether amines.

In addition, some of the compounds of this invention have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

Further, the aforementioned compounds also include their N-oxides. The term “N-oxides” refers to one or more nitrogen atoms, when present in a heterocyclic or heteroaryl compound, are in N-oxide form, i.e., N→O. For example, in compounds of any one of the formula d or Table 1 when one of Q, U, or V is N, also included are compounds in which Q, U, or V, respectively, is N→O.

As used herein, the term “pharmaceutically acceptable solvate,” is a solvate formed from the association of one or more solvent molecules to one of the compounds of any of the formulae disclosed herein. The term solvate includes hydrates (e.g., hemi-hydrate, mono-hydrate, dihydrate, trihydrate, tetrahydrate, and the like).

The method can also include the step of identifying that the subject is in need of treatment for a B-cell regulated autoimmune disorder. The identification can be in the judgment of a subject or a health professional and can be subjective (e.g., opinion) or objective (e.g., measurable by a test or a diagnostic method).

As used herein, the terms “treat”, “treatment” and “treating” refer to the reduction or amelioration of the progression, severity and/or duration of a B-cell regulated autoimmune disorder or the amelioration of one or more symptoms (preferably, one or more discernible symptoms) of a B-cell regulated autoimmune disorder resulting from the administration of one or more therapies (e.g., one or more therapeutic agents such as a compound of the invention).

As used herein, the terms “prevent”, “prevention” and “prevention” and “preventing” refer to the reduction in the risk of acquiring or developing a given a B-cell regulated autoimmune disorder, or the reduction or inhibition of the recurrence, onset or development of one or more symptoms of a given a B-cell regulated autoimmune disorder. In a preferred embodiment, a compound of the invention is administered as a preventative measure to a patient, preferably a human, having a genetic predisposition to any of the disorders described herein.

As used herein, the term “effective amount” refers to an amount of a compound of this invention which is sufficient to reduce or ameliorate the severity, duration, progression, or onset of a B-cell regulated autoimmune disorder, prevent the advancement of an a B-cell regulated autoimmune disorder, cause the regression of a B-cell regulated autoimmune disorder, prevent the recurrence, development, onset or progression of a symptom associated with a B-cell regulated autoimmune disorder, or enhance or improve the prophylactic or therapeutic effect(s) of another therapy. In certain preferred embodiments, treatment according to the invention provides a reduction in, or prevention of, at least one symptom or manifestation of a B-cell regulated autoimmune disorder, as determined in vivo or in vitro of at least about 10%, more preferably 20%, 30%, 40%, 50%, 60%, 70%, 80%, 90%, 95%, 98% or 99%.

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardley, N.Y., 1970, 537. An effective amount of a compound of this invention can range from about 0.001 mg/kg to about 1000 mg/kg, more preferably 0.01 mg/kg to about 100 mg/kg, more preferably 0.1 mg/kg to about 10 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents.

To practice a method of the present invention, a compound of the invention, alone, or as a component of a pharmaceutical composition, can be administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A sterile injectable composition, for example, a sterile injectable aqueous or oleaginous suspension, can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A composition for oral administration can be any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well-known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art. A compound of this invention can also be administered in the form of suppositories for rectal administration.

The carrier in the pharmaceutical composition must be “acceptable” in the sense of being compatible with the active ingredient of the formulation (and preferably, capable of stabilizing it) and not deleterious to the subject to be treated. For example, solubilizing agents such as cyclodextrins, which form specific, more soluble complexes with the compounds of this invention, or one or more solubilizing agents, can be utilized as pharmaceutical excipients for delivery of the compounds of the invention. Examples of other carriers include colloidal silicon dioxide, magnesium stearate, cellulose, sodium lauryl sulfate, and D&C Yellow #10.

As used herein, the terms “animal”, “subject,” “mammal” and “patient”, include, but are not limited to, a cow, monkey, horse, sheep, pig, chicken, turkey, quail, cat, dog, mouse, rat, rabbit, guinea pig and human (preferably, a human).

The methods for treating or preventing a B-cell regulated autoimmune disorder in a patient in need thereof can further comprise administering to the patient being administered a compound of this invention, an effective amount of one or more other therapeutic agents. Such therapeutic agents may include other therapeutic agents such as those conventionally used to prevent or treat a B-cell regulated autoimmune disorder or symptoms thereof.

In such combination therapy treatment, both the compounds of this invention and the other drug agent(s) are administered to mammals (e.g., humans, male or female) by conventional methods. The agents may be administered in a single dosage form or in separate dosage forms. Effective amounts of the other therapeutic agents are well known to those skilled in the art. However, it is well within the skilled artisan's purview to determine the other therapeutic agent's optimal effective-amount range. In one embodiment of the invention where another therapeutic agent is administered to an animal, the effective amount of the compound of this invention is less than its effective amount would be where the other therapeutic agent is not administered. In another embodiment, the effective amount of the conventional agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In such combination therapy treatment, at least one additional active agent can be administer with a compound of the invention. Additional active agents can be selected from a TNF antagonist (e.g., but not limited to a TNF antibody or fragment, a soluble TNF receptor or fragment, fusion proteins thereof, or a small molecule TNF antagonist), an antirheumatic (e.g., methotrexate, auranofin, aurothioglucose, azathioprine, etanercept, gold sodium thiomalate, hydroxychloroquine sulfate, leflunomide, sulfasalazine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, domase alpha (Pulmozyme), a cytokine or a cytokine antagonism. Suitable dosages are well known in the art. See, e.g., Wells et al., eds., Pharmacotherapy Handbook, 2.sup.nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

TNF antagonists suitable for compositions, combination therapy, co-administration, devices and/or methods of the present invention include, but are not limited to, anti-TNF antibodies (such as, Remicade (Infliximab) or Humira (adalimumab)) for example, or, antigen-binding fragments thereof, and receptor molecules which bind specifically to TNF (such as, for example, Enbrel (Etanercept)); compounds which prevent and/or inhibit TNF synthesis, TNF release or its action on target cells, such as thalidomide, tenidap, phosphodiesterase inhibitors (e.g., pentoxifylline and rolipram), A2b adenosine receptor agonists and A2b adenosine receptor enhancers; compounds which prevent and/or inhibit TNF receptor signalling, such as mitogen activated protein (MAP) kinase inhibitors; compounds which block and/or inhibit membrane TNF cleavage, such as metalloproteinase inhibitors; compounds which block and/or inhibit TNF activity, such as angiotensin converting enzyme (ACE) inhibitors (e.g., captopril); and compounds which block and/or inhibit TNF production and/or synthesis, such as MAP kinase inhibitors.

For clarification, a “tumor necrosis factor antibody,” “TNF antibody,” or fragment and the like decreases, blocks, inhibits, abrogates or interferes with TNF activity in vitro, in situ and/or preferably in vivo. For example, a suitable TNF human antibody of the present invention can bind TNF-α and includes anti-TNF antibodies, antigen-binding fragments thereof, and specified mutants or domains thereof that bind specifically to TNF-α. A suitable TNF antibody or fragment can also decrease block, abrogate, interfere, prevent and/or inhibit TNF DNA transcription, or prevent and/or inhibit TNF RNA or protein synthesis, TNF release, TNF receptor signaling, membrane TNF cleavage, TNF activity, TNF production and/or synthesis.

The foregoing and other useful combination therapies will be understood and appreciated by those of skill in the art. Potential advantages of such combination therapies include the ability to use less of each of the individual active ingredients to minimize toxic side effects, synergistic improvements in efficacy, improved ease of administration or use and/or reduced overall expense of compound preparation or formulation.

Without further elaboration, it is believed that the above description has adequately enabled the present invention. The following specific embodiments are, therefore, to be construed as merely illustrative, and not limitative of the remainder of the disclosure in any way whatsoever. All of the references and publications cited herein are hereby incorporated by reference in their entirety.

### EXAMPLES

### General Experimental Procedures

Cell Lines and Culture Conditions:

The THP-1 cell, Jurkat cell and RAW264.7 cell line were obtained from American Type Culture Collection (Manassas, Va.). The TH-IP-1 Jurkat cells were cultured in RPMI 1640 (ATCC, Manassas, Va.), supplemented with 10% FCS (ATCC, Manassas, Va.), and 1% penicillin/Streptomycin (Gibco-BRL, New York, N.Y.). The RAW264.7 cells were cultured in DMEM (ATCC, Manassas, Va.) supplemented with 1 (% FCS (ATCC, Manassas, Va.), and 1% penicillin/Streptomycin (Gibco-BRL, New York, N.Y.). The cells were stimulated with human or murine recombinant IFNγ (100 ng/ml) for 10 h followed by LPS (1 μg/ml) or SAC (0.025%) CpG (1 mM) in the presence or absence of Compound 50 at different concentrations for an additional incubation.

Isolation of Nuclear Extracts:

THP-1 cells were suspended in 20 volumes of buffer A containing 10 mM KCl, 10 mM HEPES (pH 7.9), 1 mM MgCl₂, 1 mM dithiothreitol (DTT), 0.1% Nonidet p40 (NP-40), and 0.5 mM phenylmethylsulfonyl fluoride (PMSF) and homogenized and centrifuged at 10,000 rpm at 4 C a for 5 min. Nuclear pellets were then suspended in buffer C containing 400 mM NaCl, 20 mM HEPES 9, pH 7.9), 15 mM MgCl₂, 0.2 mM EDTA, 1 mM DTT, 25% glycerol, 1 mM PMSF, and 10 ug of leupeptin, 20 ug of pepstatin, and 10 ug/ml antipain, incubated for 30 min at 4 C, and centrifuged at 14,000 rpm for 20 min. The supernatants were dialyzed against buffer D containing 100 mM NaCl, 20 mM HEPES (pH 7.9), 20% glycerol, 1 mM PMSF, and 1 mM DTT.

Isolation of Whole Cell Extracts:

The whole cell extracts were prepared using Cell Lysis Buffer (Cell Signaling, Beverly, Mass., USA) according to the manufacture's instruction.

Western Blot:

The 10% SDS Polyacrylamide gels (Inveitrogen) were transferred to Pure Nitrocellulose membrane (BioRed, Hercules, Calif.). The membranes were blocked with 5% milk in TBST buffer and incubated with anti-c-Rel, anti-p65, anti-p50, anti-ICSBP or anti-PU-1 antibody (all the antibodies were purchased from Santa Cruz) at a dilution of 1:500 for 1 h at room temperature or overnight at 4 C. The membranes were washed and incubated with Horseradish Peroxidase-conjugated anti-rabbit IgG or anti-mouse IgG (Amersham, England) at a dilution of 1:2000 at room temperature for 1 h.

Immunoprecipitation:

Five hundred mg of the precleared whole cell protein was incubated with 20 ul of the agarose conjugated anti-c-Rel antibody (sc-6955) for overnight at 4 C. Immunoprecipitated proteins were washed 3 times with PBS, and eluted with electrophoreses sample buffer. Western blotting of immunoprecipitated protein was performed as described above.

### Example 1

### Effect of Compound 50 on c-Rel and ICSBP (Measuring the Level of Both in the Nucleus)

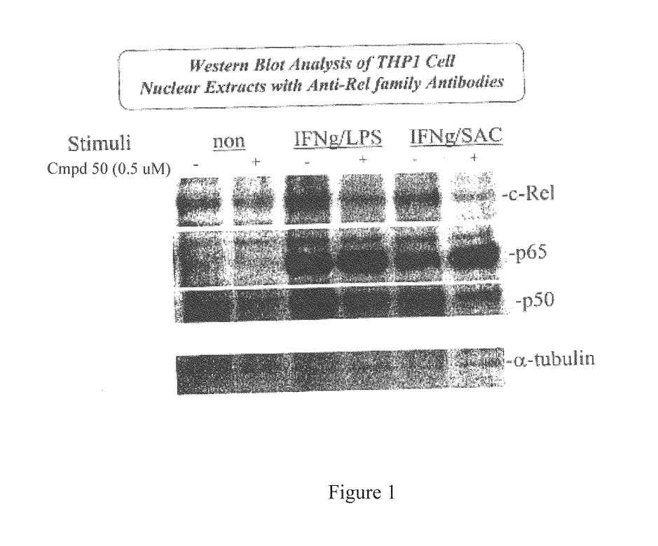

Of the transcription factors that have been analyzed, two factors, ICSBP and c-Rel, seem to be affected by Compound 4/Compound 50 treatment. ICSBP binds indirectly to the Ets-2 site. The primary NF-κB trans-activator for IL-12 is the c-Rel/p50 heterodimer. Other dimers (p65/p50 and p50/p50) either lack activity or have inhibitory functions. Thus, c-Rel plays a role in IL-12 transcription as a result of both activation through NF-κB and its interaction with ICSBP. Both Western blot analysis and DNA binding studies showed a decrease in nuclear c-Rel levels following Compound 50 treatment. As seen in FIG. 1, a western blot assay of THP1 nuclear c-Rel, p50 and p65 proteins was carried out by the following method: 10% SDS polyacrylamide gels (Invitrogen) were transferred to a Pure nitrocellulose membrane (BioRed, Hercules, Calif.). The membranes were blocked with 5% milk in TBST buffer and then incubated with anti-c-Rel, anti-p65, anti-p50, anti-ICSBP or anti-PU-1 antibody (all the antibodies were purchased from Santa Cruz) at a dilution of 1:500 for 1 h at room temperature or overnight at 4° C. The membranes were washed and incubated with Horseradish Peroxidase-conjugated anti-rabbit IgG or anti-mouse IgG (Amersham, England.) at a dilution of 1:2000 at room temperature for 1 h.

Both IFN-γ plus LPS and IFN-γ plus SAC treatment strongly increased the amount of nuclear c-Rel, p65 and p50. Compound 50 treatment significantly reduced the levels of c-Rel, with the post-treatment nuclear c-Rel level being equal to or below the non-stimulated level. In contrast, nuclear p65 protein increased following Compound 50 treatment. p50 levels decreased slightly following Compound 50 treatment, but remained above the non-stimulated levels. Thus, it is shown that Compound 50 treatment causes a reduction in the amount of nuclear c-Rel/p50, the primary IL-12 activating NFκB dimer.

ICSBP, whose expression was reduced by Compound 50, was over-expressed using co-transfection with the IL-12 promoter-Luc report system. The over-expression construct of ICSBP was generated by PCR from cDNA of human PBMC using primers as follow: ICSBP-exp-F: 5′-CCGGAATTCAGGATGTGTGACCGGAATGG-3′ (SEQ ID NO:1) and ICSBP-exp-R: 5′-ATATCTAGAATGGAGGATGCAGGACGCAGAC-3′ (SEQ ID NO:2), the resulting PCR products was ligated to pCI vector (Promega). ICSBP over-expression increased the level of p40 expression and decreased the inhibition by Compound 50.

### Example 2

### Compound 50 Blocks Accumulation of c-Rel, but not p5, in the Nucleus of LPS Stimulated Cells

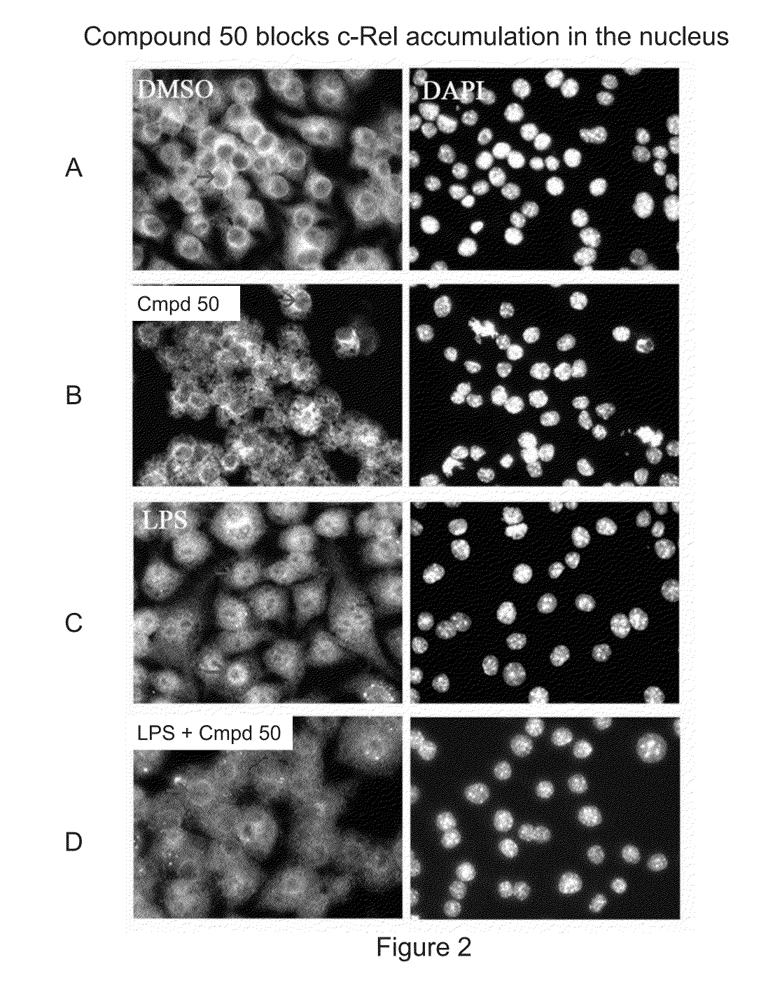

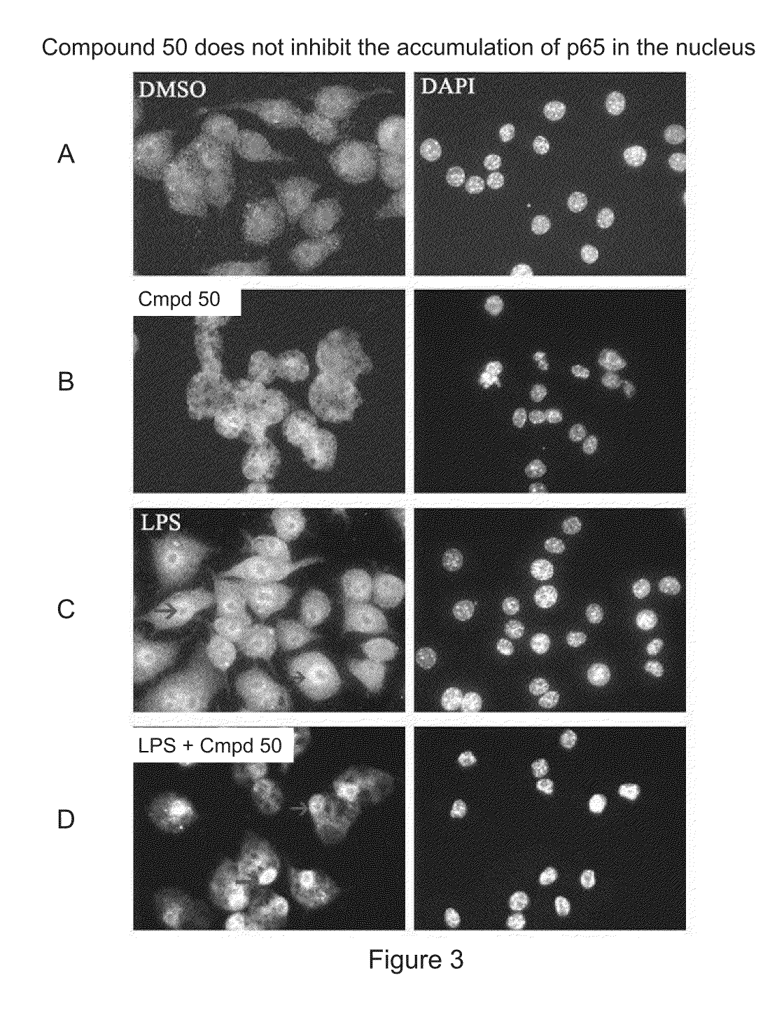

We next examined whether compound 50 can block the accumulation of c-Rel in the nucleus of cells induced by LPS (FIG. 2). RAW264.7 cells cultured in DMEM with 10% BCS were split and seeded into 4-well chambered slides at 80,000 cells/well density. The cells were then treated with DMSO, Compound 50 (100 nM), LPS (Sigma, 5 μg/mL), LPS (5 μg/mL)+Compound 50 (100 nM) for 4 hours and fixed with 3% paraformaldehyde solution (1×PBS) after 1× quick rinse with 1×PBS. Fixed cells were permeabalized with 0.2% TX100 and immunostained with anti-cRel antibody (SC70, Santa Cruz, 1:200 dilution) or anti-NF-κB p65 antibody (SC109, Santa Cruz, 1:100 dilution), and subsequently stained with Alexa Fluor 488 Goat-anti-Rabbit secondary antibody and DAPI (Molecular Probes, 1.1 μM). Images were obtained with CoolSNAP monochrome CCD camera on a Nikon inverted microscope TE300 using identical imaging parameters and were processed identically with Photoshop CS software. As observed previously, c-Rel localized to the cytoplasm in DMSO-treated cells and to the nucleus in LPS-treated cells. In the absence of LPS, Compound 50 treatment (4 h) did not alter the nuclear/cytoplasmic distribution of c-Rel; Treatment of LPS-stimulated cells with Compound 50 inhibited the accumulation of c-Rel in the nucleus resulting in a striking reduction of nuclear c-Rel staining. These data demonstrate that Compound 50 blocks LPS-induced nuclear accumulation of c-Rel. We also examined whether Compound 50 blocks the nuclear accumulation of another NF-κB/Rel family member, p65, in LPS-stimulated RAW cells (FIG. 3). As observed for c-Rel, p65 was localized to the cytoplasm in DMSO and Compound 50-treated cells and to the nucleus in LPS-stimulated cells. However, in contrast to c-Rel, p65 nuclear accumulation induced by LPS was not blocked by Compound 50. These data demonstrate the Compound 50 blocks c-Rel but not p65 nuclear translocation in LPS-stimulated cells.

### Example 3

### Effect of Compound 50 on IκB

IκB degradation is one of the steps in the signaling pathway of NF-κB dependent genes. The activity of Compound 50 in inducible degradation of IκBα □and IκBβ was investigated in THP-1 cells using Western blot and FACS analysis. The amount of IκBα and IκBβ in the cytoplasm of THP-1 and RAW267.4 cells was significantly reduced at 30 min in response to induction by IFN-γ/LPS or IFN-γ/SAC. However, there was no significant difference observed between the samples which were treated with or without Compound 50 (500 nM) at 30 min and 2 hrs. Similar results were observed from the Compound 50 pre-treatment samples in which Compound 50 was added 30 min before stimulation. These results show that Compound 50 does not induce the degradation of IκBα and IκBβ to allow free NF-κB to translocate into the nucleus where it can act as a transcription factor.

### Example 4

### Kinetics of the Members of NF-kB Nuclear Translocation in Compound 50-Treated Cells

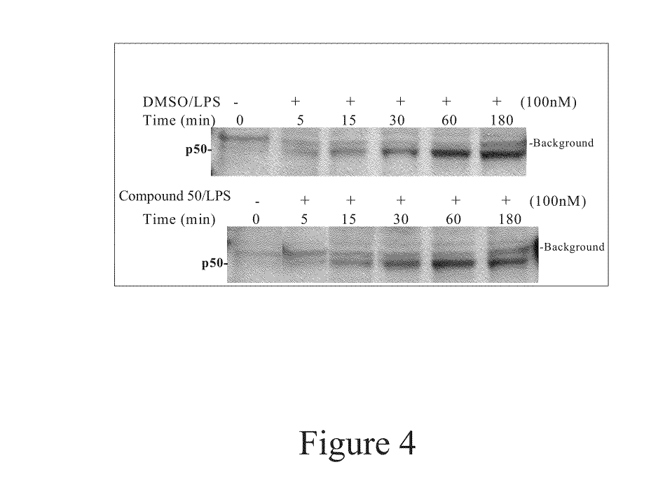

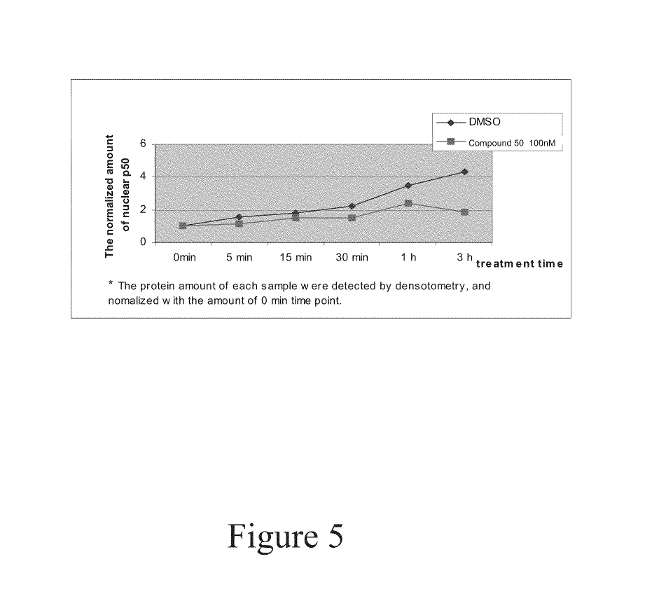

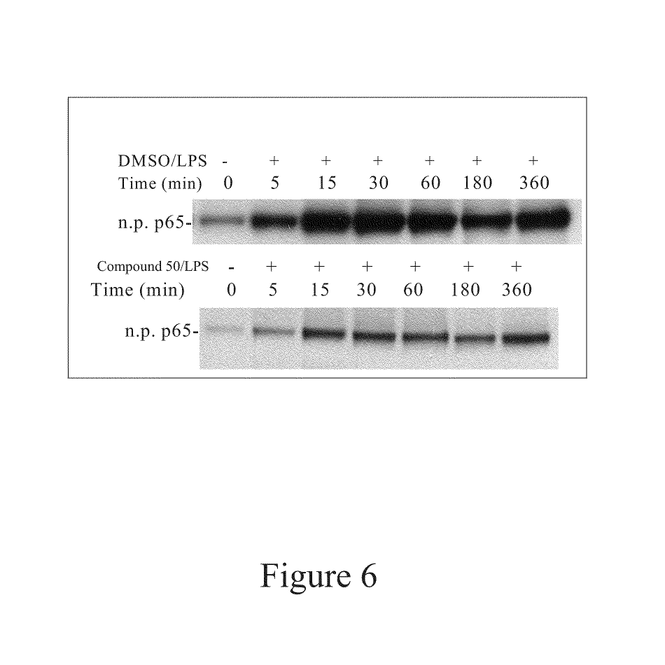

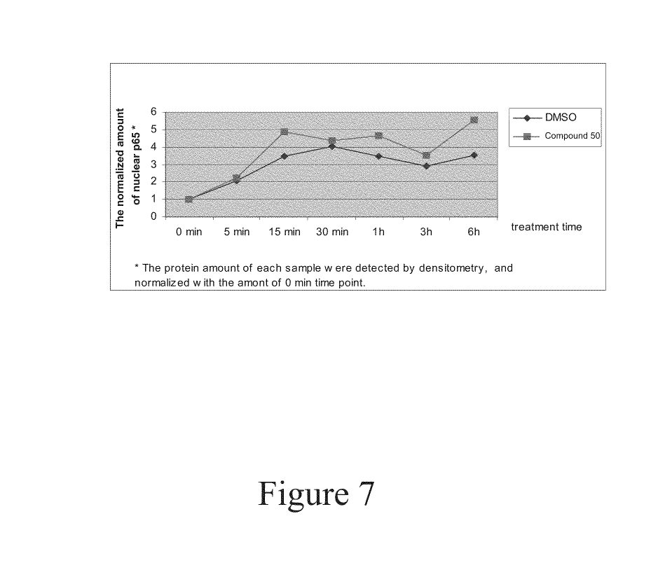

Compound 50 impairs nuclear accumulation of c-Rel and slightly reduces nuclear accumulation of p50. We examined the nuclear translocation kinetics of NF-kB family members in LPS stimulated cells treated with Compound 50. THP1 cells were stimulated with LPS in either the presence or absence of 100 nM Compound 50, and the distribution of the NF-κB Rel family members was determined by immunoblotting nuclear (n.p.) extracts collected at 5 min, 15 min, 30 min, 1 h, 3 h and 6 h post-treatment. In response to LPS stimulation, p50 translocated into the nucleus as early as 5 minutes post-stimulation and accumulates as time goes on (FIG. 4, immunoblots and FIG. 5 densitometry). Treatment of LPS-stimulated cells with Compound 50 had no effect on the kinetics of p50 nuclear entry at 5 minutes to 1 hr post-stimulation, and showed a small decrease in nuclear protein levels at 3 hours. The experiment examining p65 nuclear translocation is shown in FIG. 6 (immunoblots) and FIG. 7 (densitometry). In LPS stimulated cells, p65 translocated into the nucleus as early as 5 minutes post-stimulation and accumulated to maximum levels at 15-30 minutes post-stimulation. Treatment of LPS-stimulated cells with Compound 50 had no effect on the kinetics of p65 nuclear entry. The level of nuclear p65 at later times (6 hours) showed a small increase in Compound 50 treated cells relative to untreated cells.

Without wishing to be bound by theory, Compound 50 does not affect the kinetics of p50 and p65 nuclear accumulation in response to LPS stimulation. At later times, Compound 50 impairs nuclear translocation of p50 (at 3 h time point), and enhances nuclear translocation of p65 (at 6 h time point), indicating a selective effect on the NF-κB family.

### Example 5

### The Effects of Compound 50 on Nuclear Translocation of p52 and Rel-B

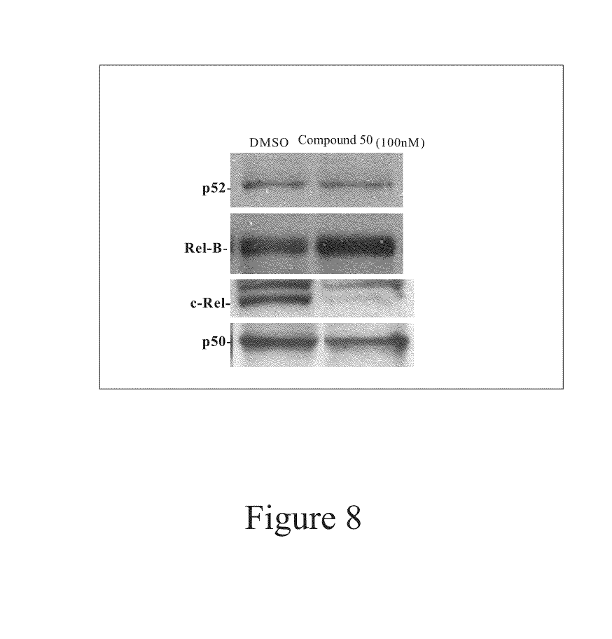

Rel B and p52 are two members of Rel family, which are preferentially complexed with each other. To determine the effect of Compound 50 on p52 and Rel-B nuclear translocation, THP1 cells were stimulated with IFNγ+LPS in either the presence or absence of 100 nM Compound 50 and the distribution of p52 and Rel-B was determined by immunoblotting of nuclear at 6 h post-treatment. As shown in FIG. 8, the nuclear Rel-B was slightly increased in the presence of Compound 50. No significant difference was found in p52. This result indicates that Compound 50 specifically inhibits c-Rel and p50 nuclear translocation, but not other NF-kB p52 and Rel-B nuclear translocation.

### Example 6

### Compound 50 does not Block Phosphorylation of IKKβ

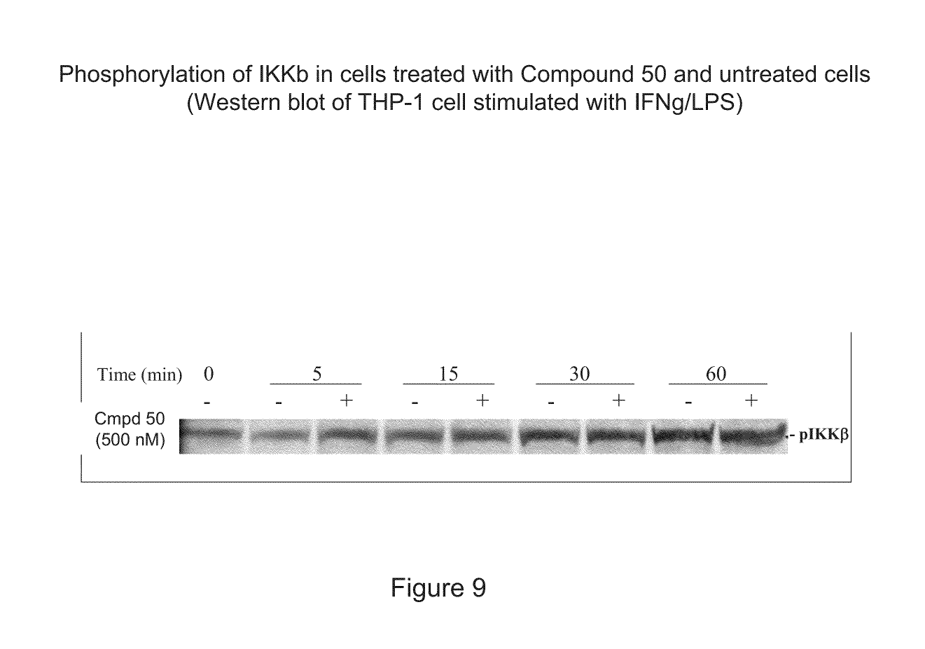

The phosphorylation of IKK is an early step in NF-κB activation. To determine whether Compound 50 inhibits the activation of the IKK complex, the level of phosphorylated IKKβ was investigated in drug-treated, LPS-stimulated cells. Whole cell extracts were prepared from THP-1 cells that had been stimulated with IFNγ/LPS for 5 min, 15 min 30 min and 1 hr in the either the absence or presence of 500 nM Compound 50. Phosphorylated IKKβ was determined by immunoblot analysis using an anti-phospho IKKβ antibody. As shown in FIG. 9, the amount of phosphorylated IKKβ accumulated with time in response to IFNγ/LPS stimulation. Compound 50 treatment had no effect on the induction of phosphorylated IKKβ. These data demonstrate the Compound 50 does not block activation of the IKK complex.

### Example 7

### Compound 50 does not Block LPS-Induced Phosphorylation of p65 or p105/p50 NF-κB Family Members

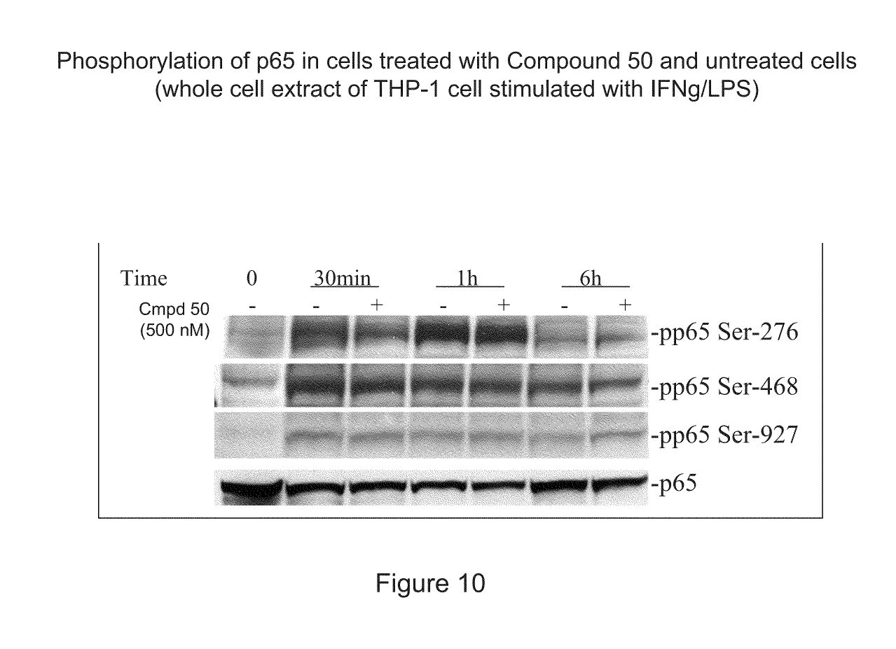

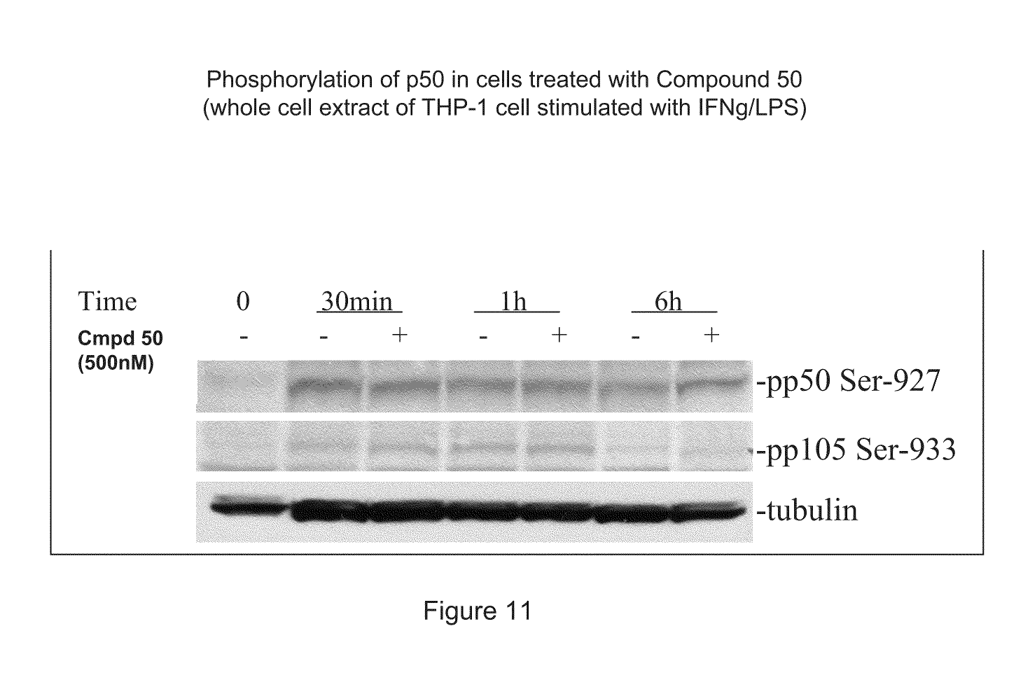

In this study, we examined the effect of Compound 50 on LPS-induced phosphorylation of the NF-κB members p65 and p105/p50. THP1 monocytic cells were stimulated with IFNγ plus LPS in the presence or absence of 100 nM Compound 50 (30 min, 1 h and 3 h) and whole-cell extracts were immunoblotted using anti-phospho p65 and p105/p50 antibodies to detect the phosphorylated forms of these proteins. FIG. 10 shows the effect of Compound 50 on p65 phosphorylation. LPS/IFNγ induced phosphorylation of p65 as early as 30 minutes on residues Ser-276, Ser-468 and Ser-927. Compound 50 had no effect on LPS/IFNγ induced phosphorylation at these sites. FIG. 11 shows the effect of Compound 50 on p105 (the precursor of p50) phosphorylation. LPS/IFNγ induced phosphorylation of p105 as early as 30 minutes on residues Ser-927 and Ser-933. Compound 50 had no effect on LPS/IFNγ induced phosphorylation at these sites. We conclude that Compound 50 does not interfere with signaling pathways that phosphorylate p65 and p105/p50 in response to LPS/IFNγ stimulation.

### Example 8

### Compound 50 Inhibits the Accumulation of Nuclear c-Rel in PMA Plus Ionomycin Stimulated Jurkat T Cells

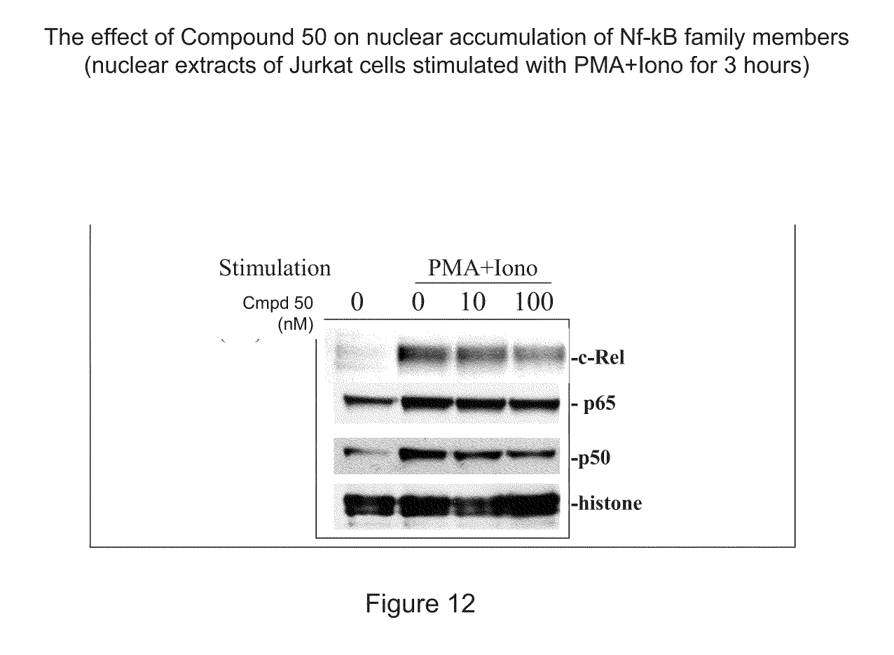

We previously showed that Compound 50 impairs the induction of the c-Rel dependent cytokine IL-2 in PMA+ionomycin stimulated Jurkat T cells. We therefore examined the accumulation of nuclear c-Rel in these cells by immunoblot analysis. As shown in FIG. 12, the levels of nuclear c-Rel were reduced at a concentration of 100 nM Compound 50. As observed previously with other cell types, the nuclear levels of p50 were slightly reduced whereas nuclear p65 levels remained unchanged. These data demonstrate that Compound 50 is able to reduce nuclear c-Rel accumulation in T cells stimulated with PMA+ionomycin.

### Example 9

### Compound 50 Reduces the DNA Binding Activity of Nuclear c-Rel

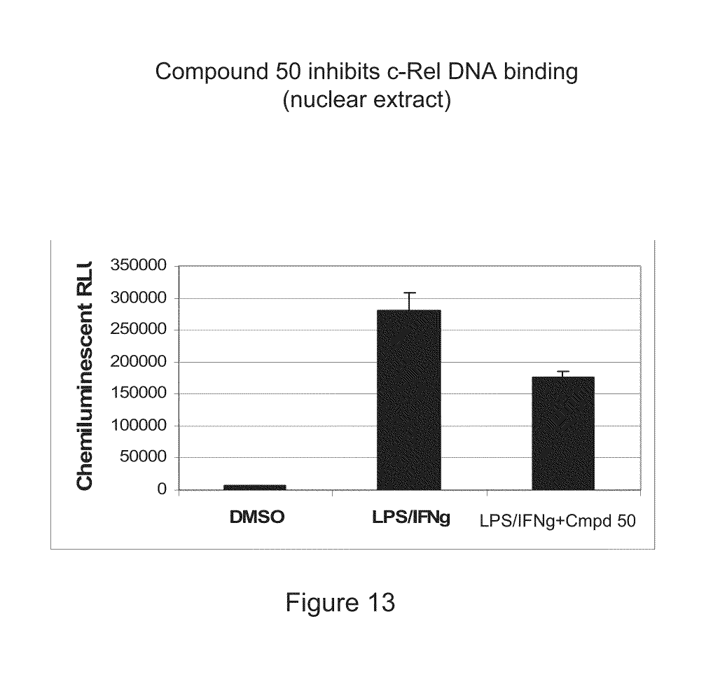

Previous work has demonstrated that Compound 50 blocks c-Rel translocation into the nucleus. In this study, we examined the effect of Compound 50 on the DNA-binding activity of nuclear c-Rel. The BD transfactor assay (a non-radioactive version of a super-shift assay) was used to measure the DNA-binding activity of c-Rel. In this assay, nuclear extracts are added to biotinylated double-stranded oligonucleotides containing the NF-κB binding site bound to a streptavidin 96-well plate. Detection of the transcription factor-DNA complex is performed with a specific primary antibody for c-Rel. The 96-well format allows for simultaneous measurement of multiple conditions and proteins using HRP-conjugated secondary antibodies whose enzymatic product can be measured using a luminometer. The level of c-Rel DNA-binding activity increased 40-fold (relative to DMSO control) in nuclear extracts from RAW cells stimulated with LPS/IFNγ. Compound 50 (1000 nM) treatment resulted in a 40% reduction in the level of c-Rel DNA-binding activity induced by stimulation with LPS/IFNγ (FIG. 13).

Methods: 20×10⁶ Raw 264.7 cells were treated with either DMSO, LPS/IFNγ, or LPS/IFNγ/Compound 50. (LPS conc. 1 ug/ml f.c.; mouse IFNγ mouse 100 U/ml f.c, Compound 50 1 uM f.c). (LPS: Sigma Cat #L2654. Mouse IFNγ; Cat #R+D 485-MICF). Cells were pre-treated with Compound 50 for 30 min, then LPS/IFNγ was added. After 3 hrs, nuclear and cytoplasmic extracts were prepared according to the BD™ TransFactor Extraction Kit and user manual. Briefly, cells were washed in PBS, harvested and lysed in hypotonic lysis buffer on ice. Cells were then disrupted by drawing the cell suspension through a No. 27 gauge needle 10 times. Next, the cell suspension was centrifuged, and the cytoplasmic extract (supernatant) was collected. The nuclear pellet was then disrupted by resuspension in high salt extraction buffer and was drawn through the needle 10 times. The suspension was centrifuged at high speed, and the nuclear extract was collected.

After measurement of protein concentration using BioRad assay, the nuclear extract was used in a Chemiluminescent NF-κB TransFactor Kit (BD) according to the user manual. Briefly, 2 ug of nuclear extract from either DMSO, LPS/IFNγ or LPS/IFNγ/Compound 50 treated cells was incubated in the wells of a 96 well plate that was coated with biotin labeled NF-κB consensus ds oligos. After washing, kit provided c-Rel specific primary antibody at a 1:500 dilution was incubated in each well. After further washing, kit provided rabbit polyclonal secondary antibody was incubated in each well at a 1:10,000 dilution. Finally, amount of bound antibody to the plate was detected by incubation with chemiluminescent substrate and subsequent detection with a luminometer. The experiment was performed in duplicate.

Western Blot Method:

After treatment with Compound 50, nuclear extract and cytoplasmic extract were prepared from 20×10⁶ Raw264.7 cells by using Extraction kits from BD Biosciences (Cat.631921), and above for experimental details. 20 ug of each extract was dissolved with 4× sample buffer and run on a 4-12% gradient SDS-PAGE gel, and blotted onto a nitrocellulose membrane by using semi-dry transfer. Non-specific binding to nitrocellulose was blocked with 5% skim milk in TBS with 0.5% Tween at room temperature for 1 hour, then probed with anti-c-Rel(C) mAb (rabbit IgG, SC-71) and anti-beta Actin(I-19) (goat IgG, sc-1616) as a control. HRP-conjugated goat anti-rabbit IgG (H+L) (#7074, Cell Signaling) and HRP-conjugated bovine anti-goat IgG (H+L) (sc-2350) were used as secondary Abs. LumiGLO reagent, 20× Peroxide (#7003, Cell Signaling) was used for visualization. Densitometry analysis was performed using Quantity One software from BioRad.

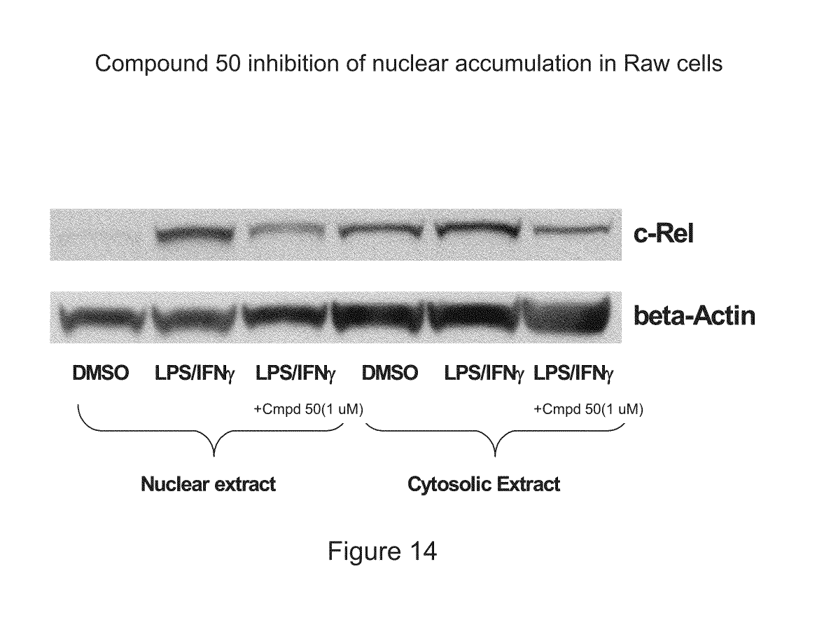

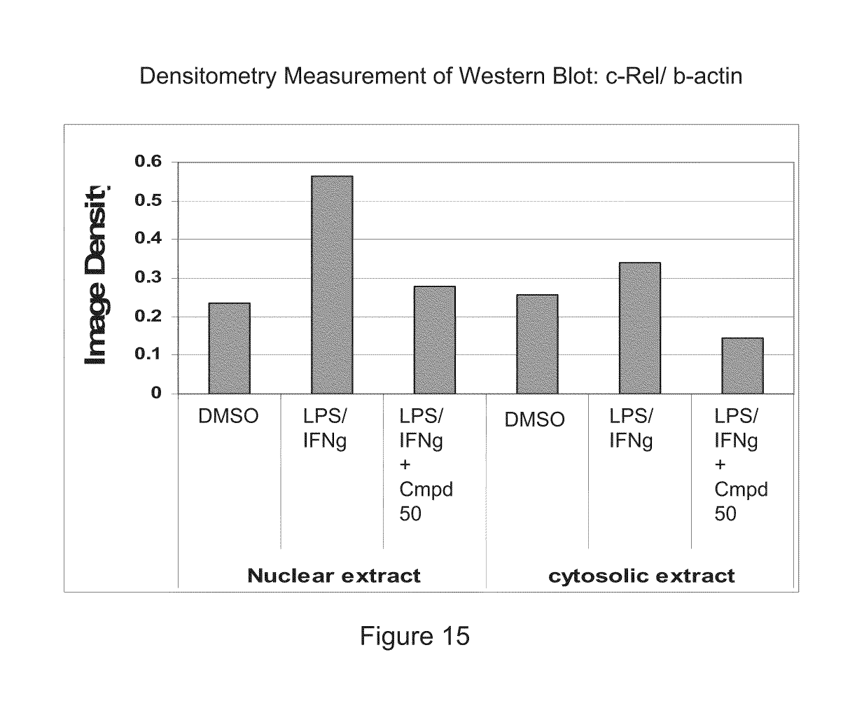

The reduction in c-Rel DNA-binding activity correlated with a 40-50% reduction in the levels of nuclear c-Rel as detected by immunoblot analysis (see immunoblot FIG. 14 and densitometry FIG. 15). We therefore conclude that Compound 50 reduces the accumulation of c-Rel in the nucleus resulting in a concomitant decrease in c-Rel DNA-binding activity.

### Example 10

### Compound 50 Interferes with Primary Mouse B Cell Survival

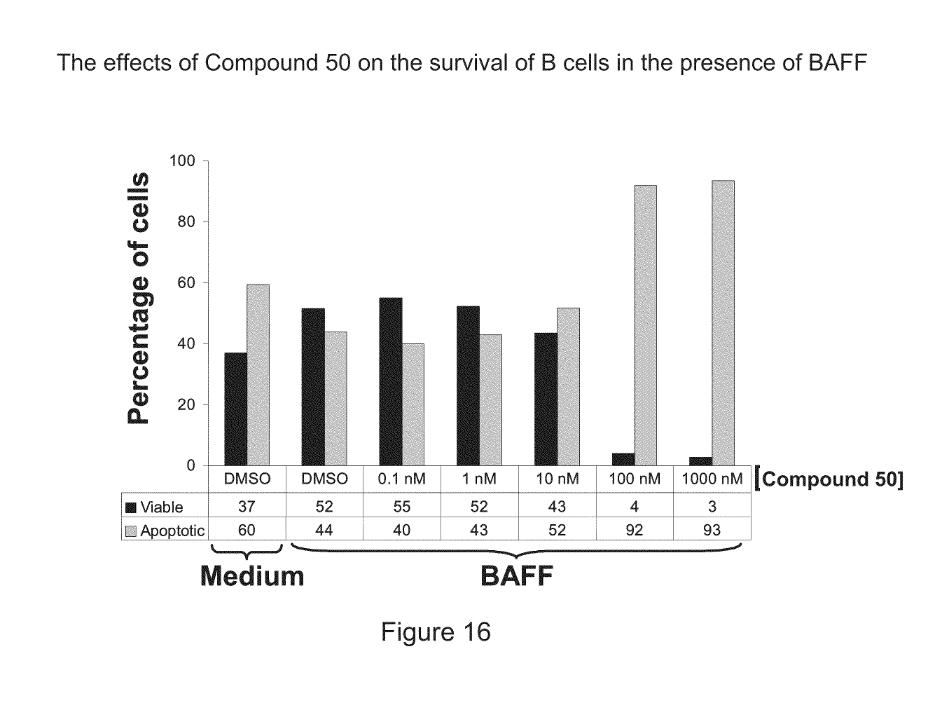

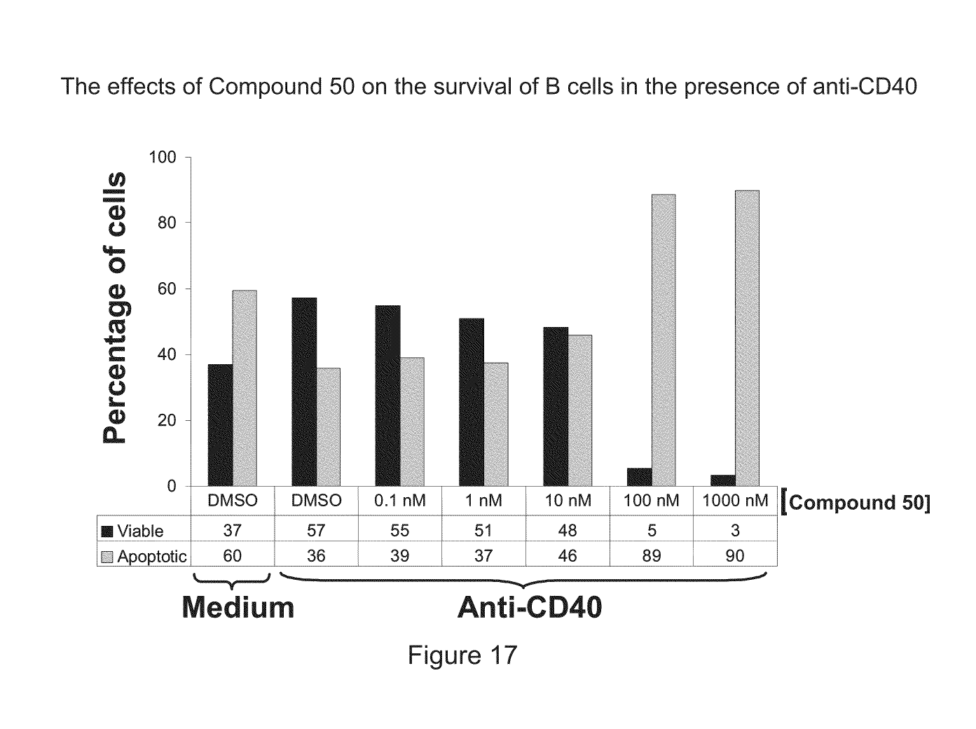

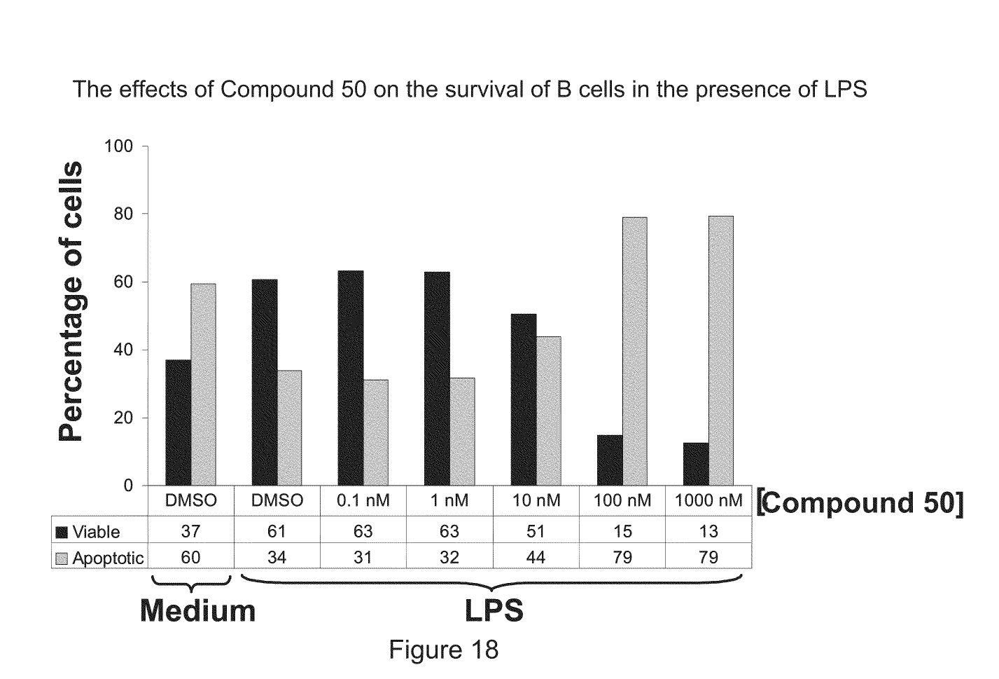

The analysis of c-Rel knockout mice has revealed a defect in B cell proliferation. Therefore, the effect of Compound 50 on the activation and survival of homogeneous populations of primary B and T lymphocytes has been explored. As a first step in this process, we evaluated the effect of Compound 50 on the survival of purified mouse splenic (CD19⁺) B cells. When cultured ex vivo, primary B cells undergo spontaneous apoptosis within 24-48 h in the absence of any survival signals. Examples of such stimuli include those mediated by anti-CD40 (or CD40L), BAFF, or B cell receptor signals provided by either anti-IgM or LPS. Murine primary B cells were isolated from mouse spleen using anti-CD19 coated magnetic beads (Miltenyi Biotec) according to the manufacturer's recommendations. Purified CD19⁺ B cells were cultured at 100,000 cells/well in 96-well microtiter plates in culture medium (RPMI 1640 supplemented with 10% fetal bovine serum, 2 mM L-glutamine, 1 mM sodium pyruvate, 20 mM HEPES, and 55 μM β-mercaptoethanol) in the presence of 10 μg/ml anti-CD40, 5 μg/ml LPS or 100 ng/ml BAFF in the presence or absence of Compound 50 for 24 h and measured the frequency of viable and apoptotic cells using Annexin V/propidium iodide flow cytometric analysis. To perform this analysis, cells were washed with PBS and labeled with Annexin V-FITC and propidium iodide (BioVision) according to the manufacturer's instructions. Apoptotic cell (Annexin V-positive) and live cell (Annexin V-negative/propidium iodide-negative) percentages were determined using a flow cytometer. The data are shown in FIGS. 16, 17, and 18. These results are summarized in Table 4.

| Stimulus | Compound 50 | % Viable | % Apoptotic |
|---|---|---|---|
| — | — | 37 | 60 |
| Anti-CD40 (10 μg/ml) | — | 57 | 36 |
| Anti-CD40 (10 μg/ml) | 0.1 nM | 55 | 39 |
| Anti-CD40 (10 μg/ml) | 1 nM | 51 | 37 |
| Anti-CD40 (10 μg/ml) | 10 nM | 48 | 46 |
| Anti-CD40 (10 μg/ml) | 100 nM | 5 | 89 |
| Anti-CD40 (10 μg/ml) | 1000 nM | 3 | 90 |
| LPS (5 μg/ml) | — | 61 | 34 |
| LPS (5 μg/ml) | 0.1 nM | 63 | 31 |
| LPS (5 μg/ml) | 1 nM | 63 | 32 |
| LPS (5 μg/ml) | 10 nM | 51 | 44 |
| LPS (5 μg/ml) | 100 nM | 15 | 79 |
| LPS (5 μg/ml) | 1000 nM | 13 | 79 |
| BAFF (100 ng/ml) | — | 52 | 44 |
| BAFF (100 ng/ml) | 0.1 nM | 55 | 40 |
| BAFF (100 ng/ml) | 1 nM | 52 | 43 |
| BAFF (100 ng/ml) | 10 nM | 43 | 52 |
| BAFF (100 ng/ml) | 100 nM | 4 | 92 |
| BAFF (100 ng/ml) | 1000 nM | 3 | 93 |

Each of the survival factors tested increased the proportion of viable cells recovered after the 24 hour culture period. Whereas 60% of B cells cultured in medium alone were apoptotic at this time point, the proportion of apoptotic cells was reduced to 34-44% when cells were cultured with either anti-CD40, LPS or BAFF. Dramatically, 80-90% of B cells cultured in the presence of Compound 50 at concentrations 100 nM were apoptotic, indicating that at these concentrations of Compound 50 apoptosis is enhanced beyond what occurs spontaneously. Moreover, this induction of cell death overcame any cell survival signals induced by anti-CD40, LPS, or BAFF. This result indicates that Compound 50 may interferes directly with the anti-apoptotic signals induced by these survival factors or it may induces apoptosis via an independent mechanism.

### OTHER EMBODIMENTS

From the above description, one skilled in the art can easily ascertain the essential characteristics of the present invention, and without departing from the spirit and scope thereof, can make various changes and modifications of the invention to adapt it to various usages and conditions. For example, compounds structurally analogous to a heterocyclic compound described in the specification also can be made, screened for their inhibiting c-Rel activities, and used to practice this invention. Thus, other embodiments are also within the claims.

1-274. (canceled)

275. A method of inhibiting the accumulation of c-Rel in the nucleus of a cell, comprising contacting the cell with an effective amount of a compound of formula (I): 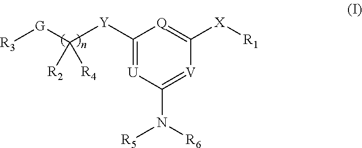
or a pharmaceutically acceptable salt thereof, wherein:
R₁ is optionally substituted aryl, optionally substituted heteroaryl, or a group represented by the following formula: 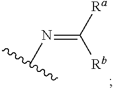
R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;
R₃ is R^{g};
R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;
X is O, S, S(O), S(O)₂, or NR^{k};
Y is (CH(R^{g}))ₘ, C(O), C(NR), O, S, S(O), S(O)₂, N(R^{k}), or absent;
G is a bond, —C(O)NR^{k}NR^{k}—, —NR^{k}NR^{k}C(O)—, —NR^{k}N═CR^{k}, —CR^{k}═NNR^{k}—, —NR^{k}NR^{k}—, —N(OH)—, —NR^{k}O—, —ONR^{k}—, —C(O)—, —C(NR)—, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}—C(NR)—NR^{k}—, —NR^{k}—C(O)—NR^{k}—, —NR^{k}—C(S)—NR^{k}—, —NR^{k}—S(O)₂—NR^{k}, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k})—, —C(NR)—NR^{k}—, —NR^{k}—CR^{g}R^{g}—C(O)—, —C(O)—ONR^{k}—, —C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}—, —OC(NR)—NR^{k}NR^{k}—, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c})NR^{k}—, —NR^{k}P(O)(R^{c})NR^{k}—, —P(O)(R^{c})NR^{k}—, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}—CHR^{g}—C(O)—, —NR^{k}—C(O)—CHR^{g}—, or —C(O)—NR^{k}—CHR^{g}—C(O)—; and
each of Q, U, and V are independently N or CR^{g}, wherein at least one of Q, U, or V is N; and each CR^{g} may be the same or different;
R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, —NR^{h}R^{j}, hydroxylalkyl, nitro, cyano, haloalkyl, aminoalkyl, or —S(O)₂R^{c};
each of R^{a} and R^{b}, independently, is H, optionally substituted alkyl, an optionally substituted cycloalkyl, an optionally substituted cyclyl, an optionally substituted heterocycloalkyl, an optionally substituted heterocyclyl, optionally substituted aryl, or optionally substituted heteroaryl;
R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;
R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;
R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;
R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;
n is 0, 1, 2, 3, 4, 5, 6 or 7; and
m is 0, 1, 2, 3, or 4;

276. The method of claim 275, wherein one of Q, U, or V is CR^{g}, and the other two are N.

277. The method of claim 276, wherein V is CR^{g}, Q and U are N.

278. The method of claim 276, wherein U is CR^{g}, V and Q are N.

279. The method of claim 275 wherein —NR₅R₆ is an optionally substituted morpholino, an optionally substituted thiomorpholino, an optionally substituted 1-oxo-thiomorpholino, an optionally substituted 1,1-dioxo-thiomorpholino, an optionally substituted piperidinyl, or an optionally substituted piperazinyl.

280. The method of claim 279, wherein X is —NR^{k}—.

281. The method of claim 280, wherein the R^{k} of group X is —H or a lower alkyl.

282. The method of claim 281, wherein R₁ is an optionally substituted aryl or an optionally substituted heteroaryl.

283. The method of claim 282, wherein R₁ is an optionally substituted phenyl, an optionally substituted indolyl, an optionally substituted indanyl, an optionally substituted carbazolyl, or an optionally substituted 1,2,3,4-tetrahydro-carbazolyl.

284. The method of claim 279, wherein Y is O.

285. The method of claim 279, wherein R₃ is an optionally substituted aryl or an optionally substituted heteroaryl.

286. The method of claim 279, wherein R₃ is a hydroxy, an optionally substituted heterocycloalkyl, an optionally substituted heterocyclyl, or an optionally substituted heteroaryl.

287. The method of claim 279, wherein each of R₂ and R₄ is, independently, H, an optionally substituted alkyl, an optionally substituted alkylcarbonyl, an optionally substituted aryl, an optionally substituted heteroaryl, an optionally substituted cycloalkyl, an optionally substituted cyclyl, an optionally substituted heterocycloalkyl, or an optionally substituted heterocyclyl.

288. The method of claim 279, wherein G is an optionally substituted heteroaryl or an optionally substituted heterocyclyl.

289. The method of claim 279, wherein G is —C(O)NHNH—, —NHNHC(O)—, —CH═N—NH—, —NH—N═CH—, —NHNH—, —NHO—, —O—NH—, —NR^{k}—O—, —CH═N—O—, —O—N═CH—, —O—C(S)—NH—, or —NH—C(S)—O—.

290. The method of claim 279, wherein G is —O—C(O)—NH—, —NH—C(NH)—NH—, —NR^{k}—C(NH)—NH—, —NR^{k}—C(NR^{k})—NH—, —NH—C(N(CN))—NH—, —NH—C(NSO₂R^{c})—NH—, —NR^{k}—C(NSO₂R^{c})—NH—, —NH—C(NNO₂)—NH—, NH—C(NC(O)R^{c})—NH—, —NH—C(O)—NH—, or —NH—C(S)—NH—.

291. The method of claim 279, wherein G is an optionally substituted aryl, an optionally substituted heteroaryl, an optionally substituted heteroaralkyl, —C(N—CN)—NH—, —Si(OH)₂—, —C(NH)—NR^{k}—, or —NR^{k}—CH₂—C(O)—.

292. A method of identifying a molecule capable of selectively inhibiting the accumulation of c-Rel in the nucleus of a cell, said method comprising:
(a) contacting the cell with the molecule;
(b) determining the level of c-Rel in the nucleus of the cell; and
(c) measuring the level of expression of NFκB in the nucleus of the cell, wherein an increase or decrease in the amount of c-Rel localized to the nucleus of the cell without a material alteration in the level of expression of NFκB and/or amount of IκB, relative to the amount of c-Rel localized to the nucleus in a cell that has not so contacted with the molecule, indicates that the molecule selectively inhibits the accumulation of c-Rel in the nucleus of the cell.

293. The method according to claim 292 wherein the cell is a cultured cell.

294. The method according to claim 292, wherein the molecule inhibits the accumulation of c-Rel more than the accumulation of any other member of NFkB family in the nucleus of the cell.


SEQUENCE LISTING (2 sequences)

SEQ ID NO: 1 - DNA, 29 bp, Artificial Sequence
  Description of Artificial Sequence Synthetic primer
ccggaattcaggatgtgtgaccggaatgg

SEQ ID NO: 2 - DNA, 28 bp, Artificial Sequence
  Description of Artificial Sequence Synthetic primer
atatctagaatggatgcaggacgcagac